(19) United States
(12) Patent Application Publication
(10) Pub. No.: US 2023/0183177 A1
(43) Pub. Date: Jun. 15, 2023

(54) ENANTIOSELECTIVE CHEMO-ENZYMATIC SYNTHESIS OF OPTICALLY ACTIVE AMINO AMIDE COMPOUNDS

(71) Applicant: Pharmazell GmbH, Raubling (DE)

(72) Inventors: Birgit GRILL, Goessendorf (AT); Margit WINKLER, Graz (AT); Helmut SCHWAB, Graz (AT); Gernot STROHMEIER, Hengsberg (AT); Kai DONSBACH, Traunstein (DE); Siegfried R. WALDVOGEL, Gau-Algesheim (DE); Sebastian ARNDT, Mainz (DE); Dominik WEIS, Mainz (DE)

(21) Appl. No.: 17/996,863

(22) PCT Filed: Apr. 23, 2021
(86) PCT No.: PCT/EP2021/060648

§ 371 Date: Oct. 21, 2022

(30) Foreign Application Priority Data
Apr. 24, 2020 (EP) 20171351.8
May 5, 2020 (EP) 20172908.4
Oct. 2, 2020 (EP) 20199842.4
Dec. 11, 2020 (EP) 20213425.0

(51) Int. Cl. C07D 207/27; C07D 207/12; B01J 23/46

(52) U.S. Cl. CPC C07D 207/27; C07D 207/12; B01J 23/462

(57) ABSTRACT

The present invention relates to a novel biocatalytic process for the stereoselective preparation of alpha amino amide compounds catalyzed by NHase enzymes. A further aspect of the invention relates to novel NHase enzymes as well as further improved NHase enzyme mutants, nucleic acid molecules encoding these enzymes, recombinant microorganisms suitable for preparing such enzymes and mutants. Another aspect of the invention relates to a chemo-biocatalytic process for the preparation of lactam compounds comprising the new catalytic process for the preparation of alpha amino amide compounds catalyzed by NHase enzymes, as well as the chemical oxidation of the alpha amino amide by applying certain chemical oxidation catalysts suitable for converting the alpha amino amide under retention of its stereochemical configuration to the respective lactam. The novel chemo-biocatalytic process is particularly suited for the synthesis of valuable pharmaceutical compounds, like in particular (S)-Levetiracetam.

The present invention relates to a novel biocatalytic process for the stereoselective preparation of alpha amino amide compounds catalyzed by NHase enzymes. A further aspect of the invention relates to novel NHase enzymes as well as further improved NHase enzyme mutants, nucleic acid molecules encoding these enzymes, recombinant microorganisms suitable for preparing such enzymes and mutants. Another aspect of the invention relates to a chemo-biocatalytic process for the preparation of lactam compounds comprising the new catalytic process for the preparation of alpha amino amide compounds catalyzed by NHase enzymes, as well as the chemical oxidation of the alpha amino amide by applying certain chemical oxidation catalysts suitable for converting the alpha amino amide under retention of its stereochemical configuration to the respective lactam. The novel chemo-biocatalytic process is particularly suited for the synthesis of valuable pharmaceutical compounds, like in particular (S)-Levetiracetam.

### BACKGROUND OF THE INVENTION

Nitrile hydratases (NHase; EC 4.2.1.84) catalyze the hydration of nitriles to the corresponding amides [S. Prasad, T. C. Bhalla, Nitrile hydratases (NHases): At the interface of academia and industry, Biotechnol. Adv. 28 (2010) 725-741]. Two types of NHases can be distinguished, the non-corrin cobalt-containing and non-heme iron-containing NHases. Both types are composed of an α- and a β-subunit and functional heterologous expression depends on the action of an accessory protein [E. T. Yukl, C. M. Wilmot, Cofactor biosynthesis through protein post-translational modification, Curr. Opin. Chem. Biol. 16 (2012) 54-59.].

Successful heterologous expression has been described for the NHases from Klebsiella oxytoca [F.-M. Guo, J.-P. Wu, L.-R. Yang, G. Xu, Overexpression of a nitrile hydratase from Klebsiella oxytoca KCTC 1686 in Escherichia coli and its biochemical characterization, Biotechnol. Bioprocess Eng. 20 (2015) 995-1004] and Bacillus sp. RAPc [R. A. Cameron, M. Sayed, D. A. Cowan, Molecular analysis of the nitrile catabolism operon of the thermophile Bacillus pallidus RAPc8, Biochim. Biophys. Acta. 1725 (2005) 35-46], the thermostable NHases from Pseudomonas thermophile [A. Miyanaga, S. Fushinobu, K. Ito, T. Wakagi, Crystal Structure of Cobalt-Containing Nitrile Hydratase, Biochem. Biophys. Res. Commun. 288 (2001) 1169-1174] and Aurantimonas manganoxydans [X. Pei, H. Zhang, L. Meng, G. Xu, L. Yang, J. Wu, Efficient cloning and expression of a thermostable nitrile hydratase in Escherichia coli using an auto-induction fed-batch strategy, Process Biochem. 48 (2013) 1921-1927]. Pei et al, also describe therein that NHase from M. manganoxydans, if co-expressed in E. coli with Chaperones GroEL/ES or DnaK/J-GrpE, the expressed protein is obtained in significantly higher yield.

In literature, enantioselective NHases have already been described from different organisms as Nitriliruptor alkaliphilus, Bradyrhizobium japonicum and Pseudomonas putida [S. Wu, R. D. Fallon, M. S. Payne, Over-production of stereoselective nitrile hydratase from Pseudomonas putida 5B in Escherichia coli: activity requires a novel downstream protein, Appl. Microbiol. Biotechnol. 48 (1997) 704-708; J. L. Tucker, L. Xu, W. Yu, R. W. Scott, L. Zao, N. Ran, Chemoenzymatic processes for preparation of Levetiracetam, WO2009009117, 2009; S. van Pelt, M. Zhang, L. G. Otten, J. Holt, D. Y. Sorokin, F. van Rantwijk, G. W. Black, J. J. Perry, R. A. Sheldon, Probing the enantioselectivity of a diverse group of purified cobalt-centred nitrile hydratases, Org. Biomol. Chem. 9 (2011) 3011].

(S)-Selective nitrile hydratases have been described in WO2009009117 in the context of Levetiracetam (3) production, however, in a classical enzymatic kinetic resolution (EKR) of the racemic 2-(2-oxopyrrolidin-1-yl)-butanenitrile (7),

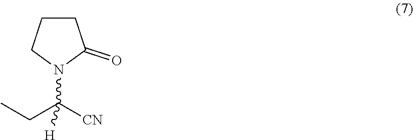

leading to a theoretical yield of (4) of maximum 50% only.

A first problem to be solved by the invention is the provision of a novel synthetic approach allowing the production of Levetiracetam (4) and related lactams, in particular with improved yield.

Another problem to be solved by the present invention was to find (S)-selective NHases capable of utilizing 2-(2-pyrrolidin-1-yl)-butanenitrile (1), in particular (S)-1 as substrate.

Another problem to be solved by the present invention was to find robust, (S)-selective NHases for use in such a novel process.

Still another problem to be solved by the invention was to provide enzyme mutants of such NHase enzymes showing improved properties. More particularly such improved mutants should show improvement, such as improved enantioselectivity and/or improved conversion of a substrate.

### SUMMARY OF THE INVENTION

The biocatalytic synthesis of heterocyclic alpha amino amides of the type (S)-2-(pyrrolidine-1-yl)butaneamide ((S)-2) and structurally related heterocyclic compounds with a cyclic tertiary amino group has not yet been described.

The present invention is based on the surprising finding, that (S)-2-(pyrrolidine-1-yl)butaneamide ((S)-2) (or related amide compounds) may be obtained from the respective racemic nitrile (rac-1) (or from related nitriles) by an enantioselective (S)-nitrile hydratase in superior yields (Scheme 1). As opposed to the prior art approach as described in WO2009009117 the present inventors surprisingly observed a dynamic kinetic resolution (DKR) of the starting material (rac-1). Racemization of (rac-1) is observed under appropriate conditions forming equilibrium between the racemic nitrile and its chemical constituents pyrrolidine, propanal and HCN. As a result of this (S)-1 which is removed from the racemic mixture through the action of said (S)—NHase is continuously supplemented by said racemization reaction which ultimately results in product yields above 50%.

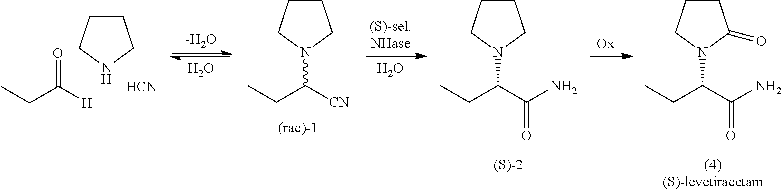

### Scheme 1. Chemoenzymatic Reaction Route to (S)-Levetiracetam (4).

The reaction conditions allowing racemization of the substrate (rac)-1 were prior to the present invention undetermined.

As opposed to the NHase substrate of the present invention the racemic oxo-substrate 2-(2-oxopyrrolidin-1-yl)-butanenitrile (7) of prior art does not allow racemization as it does not decompose and does not form an equilibrium with its respective constituents and consequently does not allow DKR of the racemic substrate, but only kinetic resolution.

First of all, a NHase panel was established comprising of 17 Co-type and 4 Fe-type NHases. Nineteen nitrile hydratase were expressed in soluble form under the applied conditions and fourteen showed activity for methacrylonitrile hydration. Seven candidates were capable of conversion of rac-1; the Co-type CtNHase, KoNHase and NaNHase as well as the Fe-type GhNHase, PkNHase, PmNHase and PkNHase. These seven NHases were characterized in more detail.

Temperature and pH studies revealed that Co-type NHases are more stable than Fe-type NHases. Especially CtNHase was quite resistant to elevated temperature and pH values. CtNHase was the most promising enzyme among the Co-type NHases not only because of its high stability. The wild type showed already ee values of 84% for (S)-2 formation.

The best Fe-type NHase was GhNHase with its outstanding conversion levels of rac-1, however, stability and enantioselectivity were inferior compared to CtNHase. Eventually, CtNHase was chosen for mutation experiments for following reasons: Its enantioselectivity towards (S)-2 was already high, it handled high substrate concentrations better than GhNHase, it was not inhibited by propanal and its high starting stability is an advantage for mutational studies.

Rational engineering was applied to specifically alter the substrate binding pocket of CtNHase. Using structural biology methods, amino acid residues within 4 Å of the docked product were identified. All positions not involved in metal binding or the reaction mechanism were targeted by site-saturation libraries: αQ93, αW120, αP126, αK131, αR169, βM34, βF37, βL48, βF51 and βY68. Approximately 200 clones of each library were screened for enhanced (S)-2 production in a liquid assay. Substitutions in βM34 led to clones with enhanced enanto-selectivity and βF51 mutants showed both increased conversion and ee values. Combination mutants of these two positions were constructed but no further improvement could be gained. The most suitable rationally designed mutant was CtNHase-βF51L with a production of 34.2% of 2 and an enantiomeric excess of 91.5% towards (S)-2 for the hydration of 150 mM of rac-1.

Four sequence stretches, lining the active site of the enzyme, were targeted by random engineering. The residues with most beneficiary impact on the hydration of rac-1 were discovered in region β1: βL48, βF51 and βG54. Only one beneficial amino acid exchange was found per stretch for α1 and α2, αV110I and αP121, respectively. An advantageous combination mutant was revealed in region β2: βH146L/βF167Y. Whereas alterations in the α subunit predominantly affected the enzymatic activity, amino acid exchanges in the β subunit had a strong impact on the enantioselectivity. However, mutants with extremely high enantioselectivities showed mostly low conversion levels.

Positions αP121, βL48 and βF51 were investigated in more detail and with all our knowledge of the key residues, 28 CtNHase combination mutants were constructed. Screening for rac-1 conversion found CtNHase mutants with extremely high enantioselectivities when amino acid exchanges in βL48 were combined with others. Up to 67% conversion of rac-1 were reached by CtNHase-αP121T/βL48R at a high enantiomeric excess of 97.6%. The mutants CtNHase-βL48P combined with βG54C, βG54R or βG54V achieved exceptionally high ee values 99.8% at conversion rate of more than 35.7% in reactions with additional propanal. This success can predominantly be attributed to the use of (S)-selective amidase in the screening.

### DESCRIPTION OF THE FIGURES

FIG. 1: Vector map of pMS470d8.

FIG. 2: Activity of methacrylonitrile hydration. Enzymatic activity of NHases of different origin was calculated per mg cell-free extract (black bars) and per mg NHase (hatched bars) (amount of NHase in CFE estimated via SDS-PAGE). In the assay, 114 mM substrate are converted at pH 7.2 and 25° C.

FIG. 3: Substrate decomposition experiment. When α-ethyl-1-pyrrolidineacetonitrile, an α-aminonitrile, dissociates, the released cyanide is detected on the sensitive Feigl-Anger filter paper. The substrate was dissolved in ethanol and six different buffers, ranging from pH 5 to 10.

FIG. 4: Activity of GhNHase (upper diagram) and CtNHase (lower diagram) in presence of potassium cyanide. The conversion of 114 mM methacrylonitrile at 25° C. and pH 7.2 was followed in the presence of up to 50 mM KCN spectrophotometrically at 224 nm.

FIG. 5: Activity of GhNHase (upper diagram) and CtNHase (lower diagram) in the presence of propanal. The conversion of 114 mM methacrylonitrile at 25° C. and pH 7.2 in the presence of up to 50 mM propanal was followed spectrophotometrically at 224 nm.

FIG. 6: Conversion of rac-1 at a lower reaction temperature. 50 mM substrate were converted by 20% (v/v) NHase-CFE in 50/40 mM sodium phosphate buffer, pH 7.2, at 5 (white bar) or 25° C. (black bar) and 300 rpm overnight for GhNHase (right pair of bars) and CtNHase (left pair of bars).

FIG. 7: Conversion rates by four NHases in enzyme feeding reactions at different reaction conditions. Two buffers were tested and enzyme feeding reactions were performed. 50 mM rac-1 was applied in overnight at 5° C. Reactions were started with 10% (v/v) of CFE and feeding reactions were supplemented with additional 10% after 1 h.

FIG. 8: Target reaction by CtNHase-CFE at different pH and temperatures. The bars represent the conversion at 25° C. (white bar, left) and 5° C. (black bar, right). The diamonds indicate the enantiomeric excess towards the (S)-enantiomer at 25° C. (white) and 5° C. (black). 50 mM of rac-1 were converted by 10% (v/v) CFE at 25° C. or 5° C. and 500 rpm in 2 h.

FIG. 9: Target reaction by GhNHase-CFE at different pH. The bars represent the conversion at 25° C. The diamonds indicate the enantiomeric excess towards the (S)-enantiomer. 50 mM of rac-1 were converted by 20% (v/v) CFE at 25° C. or 5° C. and 500 rpm in 2 h. 10% CFE were equal to 324 μL/mL GhNHase.

FIG. 10: Effect of catalyst amount for the conversion of rac-1 by GhNHase-CFE. The reactions were performed at 25° C. and 500 rpm for 2 h. 10% CFE were equal to 324 μL/mL GhNHase. Amount of amide 2 produced (in % of substrate) is illustrated by circles; enantiomeric excess of (S)-enantiomer is shown by diamonds.

FIG. 11: Effect of catalyst amount for the conversion of rac-1 by CtNHase-CFE. The reactions were performed at 5° C. and 500 rpm for 2 h. 10% CFE were equal to 520 μL/mL CtNHase. Amount of amide 2 produced (in % of substrate) is illustrated by circles; enantiomeric excess of (S)-enantiomer is shown by diamonds.

FIG. 12: Time course for production of 2 by 2% GhNHase. 50 mM rac-1 were converted in 200 mM Tris-HCl buffer, pH 7, at 25° C. and 500 rpm for 2 h. 2% CFE were equal to 64.8 μL/mL GhNHase.

FIG. 13: Conversions by GhNHase cells for different rac-1 concentrations and pH values. The bars represent the conversion and the diamonds the ee values.

FIG. 14: Conversions by CtNHase cells for different rac-1 concentrations and pH values. The bars represent the conversion and the diamonds the ee values.

FIG. 15: Conversion levels of 150 mM α-ethyl-1-pyrrolidineacetonitrile to the respective amide by CtNHase in form of resting cells with additional pyrrolidine and propanal. 1: only nitrile, 2: with 150 mM pyrrolidine, 3: with 150 mM propanal, 4: with 75 mM pyrrolidine, 5: with 75 mM propanal, 6: with 75 mM pyrrolidine and 75 mM propanal.

FIG. 16: Conversion of rac-1 by single CtNHase variants in target reaction. Conversion of 150 mM rac-1 with (white bar of each pair of bars) and without (black bar of each pair of bars) 150 mM propanal by 8.5 mg/mL CtNHase cells, for 2 h at 25° C. and 700 rpm in 500 mM Tris-HCl buffer, pH 7. Reactions were performed in triplicates and analyzed by HPLC-UV.

FIG. 17: GC Calibration lines for the precursor rac-1 and for Levetiracetam 3 using caffeine as internal standard.

FIG. 18: LC-PDA calibration lines for NaIO₄ and NaIO₃.

FIG. 19: Preparative scale fed batch hydration of rac-1 by CtNHase double mutant αP121/βL48R. Black arrows indicate points of substrate and propanal addition.

### ABBREVIATIONS

bp base pair
CFE Cell free extract
CWW Cell wet weight
DNA deoxyribonucleic acid
cDNA complementary DNA
ee Enantiomeric excess
HPLC High performance liquid chromatograph
kb kilo base

### MAN Methacrylonitrile

NHase Nitrile hydratase
OD Optical density
ON Over night
PCR Polymerase chain reaction
rpm rotations per minute
RNA ribonucleic acid

### Definitions

### a) General Terms

For the descriptions herein and the appended claims, the use of “or” means “and/or” unless stated otherwise. Similarly, “comprise”, “comprises”, “comprising”, “include”, “includes”, and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”.

The terms “purified”, “substantially purified”, and “isolated” as used herein refer to the state of being free of other, dissimilar compounds with which a compound of the invention is normally associated in its natural state, so that the “purified”, “substantially purified”, and “isolated” subject comprises at least 0.1%, 0.5%, 1%, 5%, 10%, or 20%, or at least 50% or 75% of the mass, by weight, of a given sample. In one embodiment, these terms refer to the compound of the invention comprising at least 95, 96, 97, 98, 99 or 100%, of the mass, by weight, of a given sample. As used herein, the terms “purified,” “substantially purified,” and “isolated” when referring to a nucleic acid or protein, or nucleic acids or proteins, also refers to a state of purification or concentration different than that which occurs naturally, for example in an prokaryotic or eukaryotic environment, like, for example in a bacterial or fungal cell, or in the mammalian organism, especially human body. Any degree of purification or concentration greater than that which occurs naturally, including (1) the purification from other associated structures or compounds or (2) the association with structures or compounds to which it is not normally associated in said prokaryotic or eukaryotic environment, are within the meaning of “isolated”. The nucleic acid or protein or classes of nucleic acids or proteins, described herein, may be isolated, or otherwise associated with structures or compounds to which they are not normally associated in nature, according to a variety of methods and processes known to those of skill in the art.

The term “about” indicates a potential variation of ±25% of the stated value, in particular ±15%, ±10%, more particularly ±5%, ±2% or ±1%.

The term “substantially” describes a range of values of from about 80 to 100%, such as, for example, 85-99.9%, in particular 90 to 99.9%, more particularly 95 to 99.9%, or 98 to 99.9% and especially 99 to 99.9%.

“Predominantly” refers to a proportion in the range of above 50%, as for example in the range of 51 to 100%, particularly in the range of 75 to 99.9%, more particularly 85 to 98.5%, like 95 to 99%.

A “main product” in the context of the present invention designates a single compound or a group of at least 2 compounds, like 2, 3, 4, 5 or more, particularly 2 or 3 compounds, which single compound or group of compounds is “predominantly” prepared by a reaction as described herein, and is contained in said reaction in a predominant proportion based on the total amount of the constituents of the product formed by said reaction. Said proportion may be a molar proportion, a weight proportion or, particularly based on chromatographic analytics, an area proportion calculated from the corresponding chromatogram of the reaction products.

A “side product” in the context of the present invention designates a single compound or a group of at least 2 compounds, like 2, 3, 4, 5 or more, particularly 2 or 3 compounds, which single compound or group of compounds is not “predominantly” prepared by a reaction as described herein.

Because of the reversibility of enzymatic reactions, the present invention relates, unless otherwise stated, to the enzymatic or biocatalytic reactions described herein in both directions of reaction.

“Functional mutants” of herein described polypeptides include the “functional equivalents” of such polypeptides as defined below.

The term “stereoisomers” includes conformational isomers and in particular configuration isomers.

Included in general are, according to the invention, all “stereoisomeric forms” of the compounds described herein, such as “constitutional isomers” and “stereoisomers”.

“Stereoisomeric forms” encompass in particular, “stereoisomers” and mixtures thereof, e.g. configuration isomers (optical isomers), such as enantiomers, like (R)- and (S) enantiomer, or geometric isomers (diastereomers), such as E- and Z-isomers, and combinations thereof. If one or more asymmetric centers are present in one molecule, the invention encompasses all combinations of different conformations of these asymmetry centers, e.g. enantiomeric pairs.

The term “regiospecificity or “regiospecific” describes the orientation of a reaction that involves a reactant containing at least two possible reaction sites. If such reaction takes place and produces two or more products and one of the products “predominates”, the reaction is said to be “regioselective”. If merely one of the products is produced or “essentially” produced then the reaction is said to be “regiospecific” (i.e. proceed under retention of configuration).

The term “stereo-conserving” reaction describes the influence of a chemical, electrochemical or biochemical reaction on an asymmetrical reactant containing at least one asymmetrical carbon atom. If such reaction takes place and produces a product wherein the stereochemical configuration is not changed at the asymmetrical carbon atom, or is “essentially” not changed at the asymmetrical carbon atom, then the reaction may be classified as “stereo-conserving” or, synonymously, as reaction performed under “stereo retention”.

“Stereoselectivity” describes the ability to produce a particular stereoisomer of a compound in a stereoisomerically pure or enriched form or to specifically or predominantly convert a particular stereoisomer in an enzyme catalyzed method as described herein out of a plurality of stereoisomers. More specifically, this means that a product of the invention is enriched with respect to a specific stereoisomer, or an educt may be depleted with respect to a particular stereoisomer. This may be quantified via the purity % ee-parameter calculated according to the formula:

% ee=[X_{A}−X_{V}]/[X_{A}+X_{B}]*100,

wherein X_{A} and X_{B} represent the molar ratio of the stereoisomers A and B.

The % ee-parameter may also be applied to quantify the so-called “enantiomeric excess” or “stereoisomeric excess” of a particular enantiomer formed or converted or non-converted by a particular enzyme. Particular ee-% values are in the range of 50 to 100%, like more particularly 60 to 99.9% even more particularly 70 to 99%, 80 to 98% or 85 to 97%.

The term “essentially stereoisomerically pure” refers to a relative proportion of a particular stereoisomer at least 90%, 91%, 92%, 93% 94%, 95%, 96%, 97%, 98%, 99%, 99.5%, 99.9% or 100% relative to the total amount of stereoisomers of a particular compound.

The terms “selectively converting” or “increasing the selectivity” in general means that a particular stereoisomeric form, as for example the (S)-form, of an asymmetric chemical compound, is converted in a higher proportion or amount (compared on a molar basis) than the corresponding other stereoisomeric form, as for example (R)-form. This is observed either during the entire course of said reaction (i.e. between initiation and termination of the reaction), at a certain point of time of said reaction, or during an “interval” of said reaction. In particular, said selectivity may be observed during an “interval” corresponding 1 to 99%, 2 to 95%, 3 to 90%, 5 to 85%, 10 to 80%, 15 to 75%, 20 to 70%, 25 to 65%, 30 to 60%, or 40 to 50% conversion of the initial amount of the substrate. Said higher proportion or amount may, for example, be expressed in terms of:
a higher maximum yield of an isomer observed during the entire course of the reaction or said interval thereof;
a higher relative amount of an isomer at a defined % degree of conversion value of the substrate; and/or
an identical relative amount of an isomer at a higher % degree of conversion value;

each of which particularly being observed relative to a reference method, said reference method being performed under otherwise identical conditions with known chemical or biochemical means.

Generally also comprised in accordance with the invention are all “isomeric forms” of the compounds described herein, such as constitutional isomers and in particular stereoisomers and mixtures of these, such as, for example, optical isomers, such as (R) and (S)-form, or geometric isomers, such as E- and Z-isomers, and combinations of these. If several centers of asymmetry are present in a molecule, then the invention comprises all combinations of different conformations of these centers of asymmetry, such as, for example, pairs of enantiomers, or any mixtures of stereoisomeric forms.

“Yield” and/or the “conversion rate” of a reaction according to the invention is determined over a defined period of, for example, 4, 6, 8, 10, 12, 16, 20, 24, 36 or 48 hours, in which the reaction takes place. In particular, the reaction is carried out under precisely defined conditions, for example at “standard conditions” as herein defined.

The different yield parameters (“Yield” or Y_{P/S}; “Specific Productivity Yield”; or Space-Time-Yield (STY)) are well known in the art and are determined as described in the literature.

“Yield” and “Y_{P/S}” (each expressed in mass of product produced/mass of material consumed) are herein used as synonyms.

The specific productivity-yield describes the amount of a product that is produced per h and L fermentation broth per g of biomass. The amount of wet cell weight stated as WCW describes the quantity of biologically active microorganism in a biochemical reaction. The value is given as g product per g WCW per h (i.e. g/gWCW⁻¹ h⁻¹). Alternatively, the quantity of biomass can also be expressed as the amount of dry cell weight stated as DCW. Furthermore, the biomass concentration can be more easily determined by measuring the optical density at 600 nm (OD₆₀₀) and by using an experimentally determined correlation factor for estimating the corresponding wet cell or dry cell weight, respectively.

If the present disclosure refers to features, parameters and ranges thereof of different degree of preference (including general, not explicitly preferred features, parameters and ranges thereof) then, unless otherwise stated, any combination of two or more of such features, parameters and ranges thereof, irrespective of their respective degree of preference, is encompassed by the disclosure of the present description.

### b) Biochemical Terms

The term “biocatalytic process” refers to any process carried out in the presence of catalytic activity of at least one enzyme according to the invention, i.e. processes in the presence of raw, or purified, dissolved, dispersed or immobilized enzyme, or in the presence of whole microbial living, or resting or inactivated, disrupted cells, which have or express such enzyme activity. Biocatalytic processes therefore include both enzymatic and microbial processes.

“Kinetic resolution” is a means of differentiating two enantiomers in a mixture of enantiomers, such as a racemic mixture. In kinetic resolution, two enantiomers react with different reaction rates in a chemical reaction with a chiral catalyst or reagent, resulting in an enantiomer-enriched sample of the less reactive (or non-reactive) enantiomer. Kinetic resolution relies upon differences in reactivity between enantiomers. The enantiomeric excess (ee) of the unreacted starting material continually rises as more product is formed.

The term “enzymatic kinetic resolution” (EKR) as used herein refers to a kinetic resolution of a mixture of enantiomers based on an enzyme catalyzed reaction, wherein the enzyme preferentially or exclusively converts a single enantiomer of a mixture of enantiomers to the respective (enantiomeric) product.

The term “chemo-enzymatic dynamic kinetic resolution” or “dynamic kinetic resolution” or “dynamic resolution” (DKR) as used herein refers to an “enzymatic kinetic resolution” as defined above, coupled to a chemical racemization process of the less reactive or non-reactive enantiomer, thus re-supplementing the enantiomeric form of the enzymatic substrate which is preferentially or exclusively converted by the enzyme as applied. Suitable reaction conditions for performing a DKR of the present invention are further specified below in the subsequent description. More particularly, such conditions shall favor the formation of an equilibrium between a racemic nitrile starting material and its chemical constituents (i.e. heterocyclic amine, aldehyde and HCN, such as for example pyrrolidine, propanal and HCN). In particular, suitable reaction conditions comprise an optionally buffered aqueous or aqueous/organic reaction medium at a pH in the range of 6 to 10, more particularly 6.5 and 8.5 and a reaction temperature in the range of 0 to 70° C., in particular 5 to 35° C. Different methods for further shifting the equilibrium of a reversible chemical or biochemical reaction in a particular direction, in particular to the product side, are well known. More particularly, such suitable DKR reaction conditions may also encompass an excess or added amount of aldehyde, as for example propanal (cf. also description in sections below). Said excess or added amount of aldehyde may be present for example during the entire course or at least during a certain phase or phases of the reaction, like the start of the reaction. Said excess may in particular be in the range of more than 1 equivalent, like 1.1 equivalent to 10 equivalents, in particular 1.5 to 5 equivalents, especially 2 to 3 equivalents relative to the cyclic amine, as for example pyrrolidine.

The term “domain” refers to a set of amino acids or a partial sequence of amino acids residues conserved at specific positions along an alignment of sequences of evolutionarily related proteins. While amino acids at other positions can vary between protein homologues, amino acids that are highly conserved at specific positions of such domain indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” or “signature” refers to a short conserved region in the sequence of evolutionarily related proteins. Motifs are frequently highly conserved parts of domains, but may also include only part of the domain,

A “protein family” is defined as a group of proteins that share a common evolutionary origin reflected by their related functions, similarities in sequence, or similar primary, secondary or tertiary structure. Proteins within protein families are usually homologous and have similar structure of conserved functional domains and motifs.

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). Domains or motifs may also be identified using routine techniques, such as by sequence alignment.

A “precursor” molecule of a target compound as described herein is converted to said target compound, particularly through the enzymatic action of a suitable polypeptide performing at least one structural change on said precursor molecule.

A “nitrile hydratase” or “NHase” refers to a polypeptide having hydratase activity that converts a cyano group of a substrate molecule via addition of molecular water into an amide group. NHases in the context of the present invention belong to the enzyme class (EC 4.2.1.84). A NHase of the invention comprises one or more, in particular two identical or different, in particular different, polypeptide chains forming an enzymatically active quaternary structure. Said two different polypeptide chains are herein also referred to as alpha (α) and beta (β) subunits. More particularly, a NHase of the invention has the ability to convert alpha amino nitrile substrates to the corresponding alpha amino amide products.

A “(S)-nitrile hydratase” or “(S)—NHase” refers to a polypeptide having hydratase activity that predominantly, substantially or exclusively converts a cyano group of a particular stereoisomer of a substrate molecule containing at least one asymmetric carbon atom via addition of molecular water into an amide group under retention of the stereochemical configuration. (S)—NHases in the context of the present invention belong to the enzyme class (EC 4.2.1.84). More particularly, a NHase of the invention is categorized as (S)—NHase of the invention if it has the ability to convert a particular reference (S)-substrate molecule to the corresponding reference (S)-product molecule. A particular reference substrate in the context of the invention is (S)-pyrrolidine butanenitrile ((S)-1), and a particular reference product is (S)pyrrolidine butaneamide ((S)-2).

“NHase activity” or “(S)—NHase activity” is determined under “standard conditions” as described herein below. It can be determined using recombinant NHase expressing host cells, disrupted NHase expressing cells, fractions of these or enriched or purified NHase. It can be determined in a culture medium or reaction medium, particularly buffered, having a pH in the range of 6 to 10, particularly 6 to 8, at a temperature in the range of about 0 to 50° C., like about 5 to 35° C., particularly 5 to 25° C. and in the presence of a reference substrate added at an initial concentration in the range of 1 to 200 mM, particularly 5 to 150 mM, in particular 25 to 100 mM. The conversion reaction to form the respective product, is conducted from 1 min to 24 h, particularly 5 min to 5 h, more particularly about 10 min to 2 h. The reaction product may then be determined in conventional matter, for example via HPLC of the reaction medium, optionally after removal of non-dissolved or solid constituents. A particular reference substrate is pyrrolidine butanenitrile. For determining “(S)—NHase activity” the reference substrate as used herein is in particular (S)-1 and the reference product is in particular (S)-2.

An “Accessory Protein” in the context of the present invention encompasses any type of protein, which improves the recombinant expression of a NHase as described herein. The correct folding of an enzyme and the correct integration of the active site metal in the host organism is critical for catalytic activity. Non-correctly folded enzymes or enzymes lacking the essential metal ion have reduced or no catalytic activity as they are prone to aggregation (e.g. inclusion bodies) or degradation in the host. Different strategies can be applied to ensure correctly folded enzyme in the host organism. For example, a non-correctly folded enzyme can sometimes get unfolded by strong denaturing chemicals, followed by refolding under physiological conditions. However, such procedure is time consuming and expensive. Co-expression of a so-called “accessory protein” or also designated in literature as “activator proteins” represents another, more efficient approach.

“Co-expression” or “co-expressing” should be understood broadly as long as it is performed in a manner, which results in an appropriate expression of the alpha and the beta polypeptide subunits of a functional NHase. In the case of the presence of an accessory protein, “co-expression” or “co-expressing” should also be understood broadly as long as it is performed in a manner which results in a cooperative action of such helper polypeptide assisting the functional expression of the polypeptide having NHAse activity, in particular the supporting action for introduction of the essential metal ion into the active site and the correct folding of said expressed NHase polypeptide. A simultaneous or substantially simultaneous co-expression of both polypeptides represents one non-limiting alternative among others. Another non-limiting alternative might be seen in the timely sequential expression of both polypeptides starting with expression of the helper polypeptide followed by the NHase polypeptide expression. Another non-limiting alternative might be seen a timely overlapping co-expression of both polypeptides, wherein in the initial phase merely the helper polypeptide is expressed and in the overlapping phase both polypeptides are expressed. Other alternatives may be developed by a skilled reader without inventive effort.

In molecular biology, the large class of accessory proteins, also designated as molecular “chaperones” represents proteins that assist the covalent folding or unfolding and the assembly or disassembly of other macromolecular structures.

The group of “chaperonin proteins” belongs to said large class of chaperon molecules. The structure of these chaperonins resembles two donut-like structures stacked on top of one another to create a barrel. Each ring is composed of either 7, 8 or 9 subunits depending on the organism in which the chaperonin is found.

Group I chaperonins are found in bacteria as well as organelles of endosymbiotic origin: chloroplasts and mitochondria. Group II chaperonins, as found in the eukaryotic cytosol and in archaea, are more poorly characterized. The GroEL/GroES complex is a Group I chaperonin. Group II chaperonins are not thought to utilize a GroES-type cofactor to fold their substrates.

The chaperonin system GroES/GroEL forms a barrel like structure with a cavity that allows the up-take of misfolded proteins for refolding at the expense of ATP [Gragerov A, E Nudler, N Komissarova, G A Gaitanaris, M E Gottesman, V Nikiforov. 1992. Proc Nat Acad Sci 89, 10341-10344; Keskin O, Bahar I, Flatow D, Covell D G, Jernigan R L. 2002. Biochem 41, 491-501].

Further “accessory proteins” different from such chaperonins are described in literature which upon co-expression with, for example, NHase structural genes significantly enhance specific activity of the recombinantly expressed NHases. Particular examples of “accessory proteins” for use in the context of the present invention are those, which are selected from the heterogeneous group of proteins, which are found in operons also comprising the alpha and beta subunits of nitrile hydratases (E.C. 4.2.1.84) as described above. Examples thereof are also described herein below.

The terms “biological function,” “function”, “biological activity” or “activity” of a NHase refer to the ability of a NHase as described herein to catalyze the formation of at least one amide from the corresponding precursor nitrile.

As used herein, the term “host cell” or “transformed cell” refers to a cell (or organism) altered to harbor at least one nucleic acid molecule, for instance, one or more recombinant genes encoding one or more desired proteins or one or more nucleic acid sequences which upon transcription yield at least one functional polypeptide of the present invention, in particular a NHase as defined herein above. The host cell is particularly a bacterial cell, like for example cyanobacterial cell, a fungal cell or a plant cell or plants. The host cell may contain a recombinant gene or several genes, as for example organized as an operon, which has been integrated into the nuclear or organelle genomes of the host cell. Alternatively, the host may contain the recombinant gene setup extra-chromosomally.

The term “organism” refers to any non-human multicellular or unicellular organism such as a plant, or a microorganism. Particularly, a microorganism is a bacterium, a yeast, an algae or a fungus.

A particular organism or cell is meant to be “capable of producing an alpha amino amide” when it produces an alpha amino amide as defined herein naturally or when it does not produce said ester naturally but is transformed to produce said alpha amino amide with a nucleic acid as described herein. Organisms or cells transformed to produce a higher amount of alpha amino amide than the naturally occurring organism or cell are also encompassed by the “organisms or cells capable of producing an alpha amino amide”.

A particular organism or cell is meant to be “capable of producing a target product” when it produces a target product as defined herein (for example the alpha amino amide type compounds) naturally or when it does not produce said target product naturally but is transformed to produce said target product with a nucleic acid as described herein. Organisms or cells transformed to produce a higher amount of target product than the naturally occurring organism or cell are also encompassed by the “organisms or cells capable of producing a target product”.

The term “fermentative production” or “fermentation” refers to the ability of a microorganism (assisted by enzyme activity contained in or generated by said microorganism) to produce a chemical compound in cell culture utilizing at least one carbon source added to the incubation.

The term “fermentation broth” is understood to mean a liquid, particularly aqueous or aqueous/organic solution, which is based on a fermentative process and has not been worked up or has been worked up, for example, as described herein.

An “enzymatically catalyzed” or “biocatalytic” method means that said method is performed under the catalytic action of an enzyme, including enzyme mutants, as herein defined. Thus the method can either be performed in the presence of said enzyme in isolated (purified, enriched) or crude form or in the presence of a cellular system, in particular, natural or recombinant microbial cells containing said enzyme in active form, and having the ability to catalyze the conversion reaction as disclosed herein.

An “enzyme” as described herein can be a native or recombinantly produced enzyme, it may be the wild type enzyme or genetically modified by suitable mutations or by C- and/or N-terminal amino acid sequence extensions, like His-tag containing sequences. The enzyme can basically be mixed with cellular, for example protein impurities, but particularly is in pure form. Suitable methods of detection are described for example in the experimental section given below or are known from the literature.

A “pure form” or a “pure” or “substantially pure” enzyme is to be understood according to the invention as an enzyme with a degree of purity above 80, particularly above 90, especially above 95, and quite particularly above 99 wt %, relative to the total protein content, determined by means of usual methods of detecting proteins, for example the biuret method or protein detection according to Lowry et al. (cf. description in R. K. Scopes, Protein Purification, Springer Verlag, New York, Heidelberg, Berlin (1982)).

“Proteinogenic” amino acids comprise in particular (single-letter code): G, A, V, L, I, F, P, M, W, S, T, C, Y, N, Q, D, E, K, R and H.

“Immobilization” means, according to the invention, the covalent or noncovalent binding of a biocatalyst used according to the invention, for example a NHase on a solid, i.e. essentially insoluble in the surrounding liquid medium, carrier material. According to the invention, whole cells, such as the recombinant microorganisms used according to the invention, can correspondingly also be immobilized by means of such carriers.

The term “improved enantioselectivity” as observed for a particular enzyme or enzyme mutant for the production of a particular stereoisomer and/or for the conversion of a stereoisomer refers to an improvement of enantioselectivity observed relative to a reference enzyme, in particular the non-mutated parent enzyme or an enzyme mutant differing with respect to number and/or a type of mutations as contained in the mutant showing said improved enantioselectivity. A suitable parameter for expressing enantioselectivity is the herein defined ee % value.

The term “improved activity for conversion of a substrate” as observed for a particular enzyme or enzyme mutant for the conversion of a particular substrate refers to an improvement of conversion observed relative to a reference enzyme, in particular the non-mutated parent enzyme or an enzyme mutant differing with respect to number and/or a type of mutations as contained in the mutant showing said improved conversion. A suitable parameter for expressing conversion is the decrease of substrate concentration expressed in %, as for example mole %, or increase of product concentration expressed in %, as for example mole %. In the case of a substrate comprising a mixture of stereoisomers the substrate concentration refers to the overall concentration of all stereoisomers.

The term “improved cyanide tolerance” as observed for a particular enzyme or enzyme mutant for the cyanide tolerance of its enzyme activity refers to an improvement of said tolerance observed relative to a reference enzyme, in particular the non-mutated parent enzyme or an enzyme mutant differing with respect to number and/or a type of mutations as contained in the mutant showing said improved tolerance. A suitable parameter for expressing cyanide tolerance is the residual specific enzyme activity (U/mg) observed at a particular cyanide concentration during a conversion reaction of said enzyme or enzyme mutant, expressed in % of the initial specific activity in the absence of cyanide.

The term “reduced substrate inhibition” as observed for a particular enzyme or enzyme mutant for the substrate tolerance of its enzyme activity refers to an improvement of its resistance to substrate inhibition observed relative to a reference enzyme, in particular the non-mutated parent enzyme or an enzyme mutant differing with respect to number and/or a type of mutations as contained in the mutant showing said improved tolerance. A suitable parameter for expressing substrate inhibition is the respective Kᵢ value for a particular substrate, and a reduced substrate inhibition is represented by an increase of the respective Kᵢ value.

The term “reduced product inhibition” as observed for a particular enzyme or enzyme mutant for the product tolerance of its enzyme activity refers to an improvement of its resistance to product inhibition observed relative to a reference enzyme, in particular the non-mutated parent enzyme or an enzyme mutant differing with respect to number and/or a type of mutations as contained in the mutant showing said improved tolerance. A suitable parameter for expressing product inhibition is the respective K, value for a particular product, and a reduced product inhibition is represented by an increase of the respective K, value.

The term “improved operational stability” as observed for a particular enzyme or enzyme mutant for the operational stability of its enzyme activity refers to an improvement of the total amount of product formed per molecule of enzyme observed relative to a reference enzyme, in particular the non-mutated parent enzyme or an enzyme mutant differing with respect to number and/or a type of mutations as contained in the mutant showing said improved tolerance. A suitable parameter for expressing operational stability is the total turnover number.

### c) Chemical Terms

The term “lactam derivative” in the context of the present invention in particular refers to chemical compounds which are obtained from a chemical precursor compound comprising a cyclic amino group by an enzymatic or, in particular, chemical oxidation reaction converting said cyclic amino group to a lactam (or intramolecular amide) group.

A “hydrocarbon” group is a chemical group, which essentially is composed of carbon and hydrogen atoms and may be a non-cyclic, linear or branched, saturated or unsaturated moiety, or a cyclic saturated or unsaturated moiety, aromatic or non-aromatic moiety. A hydrocarbon group comprises 1 to 30, 1 to 25, 1 to 20, 1 to 15 or 1 to 10 or 1 to 6 or 1 to 3 carbon atoms in the case of a non-cyclic structure. It comprises 3 to 30, 3 to 25, 3 to 20, 3 to 15, 3 to 10 or in particular 3, 4, 5, 6 or 7 carbon atoms in the case of a cyclic structure. Particularly, it is a non-cyclic, linear or branched, saturated or unsaturated, particularly saturated moiety, comprises 1 to 10 or particularly 1 to 6 or more particularly 1 to 3 carbon atoms

Said hydrocarbon groups may be non-substituted or may carry at least one, like 1, 2, 3, 4 or 5, 2 substituents; particularly it is non-substituted.

Particular examples of such hydrocarbon groups are noncyclic linear or branched alkyl or alkenyl residues as defined below;

An “alkyl” residue represents linear or branched, saturated hydrocarbon residues. The term comprises long chain and short chain alkyl groups. It comprises 1 to 30, 1 to 25, 1 to 20, 1 to 15 or 1 to 10 or 1 to 7, particularly 1 to 6, 1 to 5, or 1 to 4 or more particularly 1 to 3 carbon atoms.

An “alkenyl” residue represents linear or branched, mono- or polyunsaturated hydrocarbon residues. The term comprises long chain and short chain alkenyl groups. It comprises 2 to 30, 2 to 25, 2 to 20, 2 to 15 or 2 to 10 or 2 to 7, particularly 2 to 6, 2 to 5, or more particularly 2 to 4 carbon atoms. I may have up to 10, like 1, 2, 3, 4 or 5, particularly 1 or 2, more particularly 1 C═C double bonds.

The term “lower alkyl” or “short chain alkyl” represents saturated, straight-chain or branched hydrocarbon radicals having 1 to 3, 1 to 4, 1 to 5, 1 to 6, or 1 to 7, in particular 1 to 3 carbon atoms. As examples there may be mentioned: methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; and also n-heptyl, and the singly or multiply branched analogs thereof.

“Long-chain alkyl” represents, for example, saturated straight-chain or branched hydrocarbyl radicals having 8 to 30, for example 8 to 20 or 8 to 15, carbon atoms, such as octyl, nonyl, decyl, undecyl, dodecyl, tridecyl, tetradecyl, pentadecyl, hexadecyl, heptadecyl, octadecyl, nonadecyl, eicosyl, hencosyl, docosyl, tricosyl, tetracosyl, pentacosyl, hexacosyl, heptacosyl, octacosyl, nonacosyl, squalyl, constitutional isomers, especially singly or multiply branched isomers thereof.

“Long-chain alkenyl” represents the mono- or polyunsaturated analogues of the above mentioned “long-chain alkyl” groups,

“Short chain alkenyl” (or “lower alkenyl”) represents mono- or polyunsaturated, especially monounsaturated, straight-chain or branched hydrocarbon radicals having 2 to 4, 2 to 6, or 2 to 7 carbon atoms and one double bond in any position, e.g. C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The “substituent” of the above mentioned residues contains one hetero atom, like O or N. Particularly the substituents are independently selected from —OH, C═O, or —COOH.

A cyclic saturated or unsaturated moiety as referred to above particularly refers to monocyclic hydrocarbon groups comprising one optionally substituted, saturated or unsaturated hydrocarbon ring groups (or “carbocyclic” groups). The cycle may comprise 3 to 8, in particular 5 to 7, more particularly 6 ring carbon atoms. As examples of monocyclic residues there may be mentioned “cycloalkyl” groups which are carbocyclic radicals having 3 to 7 ring carbon atoms, such as cyclopropyl, cyclobutyl, cyclopentyl, cyclohexyl, cycloheptyl, and cyclooctyl; and the corresponding “cycloalkenyl” groups. Cycloalkenyl” (or “mono- or polyunsaturated cycloalkyl”) represents, in particular, monocyclic, mono- or polyunsaturated carbocyclic groups having 5 to 8, particularly up to 6, carbon ring members, for example monounsaturated cyclopentenyl, cyclohexenyl, cycloheptenyl and cyclooctenyl radicals.

The number of substituents in such monocyclic hydrocarbon residues may vary from 1 to 5, in particular 1 or 2 substituents. Suitable substituents of such cyclic residues are selected from lower alkyl, lower alkenyl, or residues containing one hetero atom, like O or N as for example —OH or —COOH. In particular the substituents are independently selected from —OH, —COOH or methyl.

Unsaturated cyclic groups may contain 1 or more, as for example 1, 2 or 3 C═C bonds and are aromatic, or in particular nonaromatic.

The above-mentioned cyclic groups may also contain at least one, like 1, 2, 3 or 4, preferably 1 or 2 ring heteroatoms, such as O, N or S, particularly N or O.

The term “salt” as used herein, refers in particular to alkali metal salts such as Li, Na and K salts of a compound, alkaline earth metal salts, such as Be, Mg, Ca, Sr and Ba salts of a compound; and ammonium salts, wherein an ammonium salt comprises the NH₄⁺ salt or those ammonium salts in which at least one hydrogen atom can be replaced with a C₁-C₆-alkyl residue. Typical alkyl residues are, in particular, C₁-C₄-alkyl residues, such as methyl, ethyl, nor i-propyl-, n-, sec- or tert-butyl, and n-pentyl and n-hexyl and the singly or multiply branched analogs thereof.

The term “alkyl esters” of compounds according to the invention are, in particular, lower alkyl esters, for example C₁-C₆-alkyl esters. As nonlimiting examples, we may mention methyl, ethyl, n- or i-propyl, n-, sec- or tert-butyl esters, or longer-chain esters, for example n-pentyl and n-hexyl esters and the singly or multiply branched analogs thereof.

### Special Embodiments of the Invention

The present invention refers to the following particular embodiments:
1. A biocatalytic process for preparing an alpha-amino amide of the general formula I

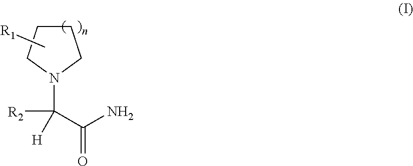

wherein
n is 0 or an integer of 1 to 4; in particular 1 or 2, more particularly 1, and
R₁ and R₂ independently of each other represent H or a hydrocarbon group, in particular a straight-chain or branched, saturated or non-saturated hydrocarbon group, having 1 to 6 carbon atoms; in particular H or C₁-C₆ alkyl or C₁-C₃ alkyl, more particularly H or C₁-C₃ alkyl, like in particular methyl;
optionally in essentially stereoisomerically pure form or as a mixture of stereoisomers; like in particular in a proportion of at least 90%, 91%, 92%, 93% 94%, more particularly 95%, 96%, 97%, 98%, 99%, 99.5%, 99.9% or 100% relative to the total amount of stereoisomers of a compound of formula I, in particular in case R2 is different from H,

which method comprises

1) contacting an alpha-amino nitrile of the general formula II

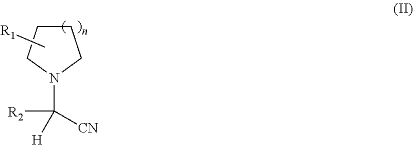

wherein n, R₁ and R₂ are as defined above,
with a polypeptide having nitrile hydratase (NHase) (E.C. 4.2.1.84) activity, whereby in particular said nitrile compound of the general formula II is converted, in particular hydrated, to said compound of general formula I, optionally in essentially stereoisomerically pure form or as a mixture of stereoisomers; in particular in essentially stereoisomerically pure form, like in particular in a proportion of at least 90%, 91%, 92%, 93% 94%, more particularly 95%, 96%, 97%, 98%, 99%, 99.5%, 99.9% or 100% relative to the total amount of stereoisomers of a compound of formula I
and

2) optionally isolating a compound of formula I.

Such process may be performed batchwise, semi-batchwise or continuously.
2. The process of embodiment 1, wherein a nitrile of formula II is applied, wherein n and R₁ are as defined above and R₂ represents a straight-chain or branched, saturated or non-saturated hydrocarbon group having 1 to 6 carbon atoms, in particular C₁-C₆ or C₁-C₃ alkyl.
3. The process of embodiment 2, wherein a nitrile of the general formula IIa

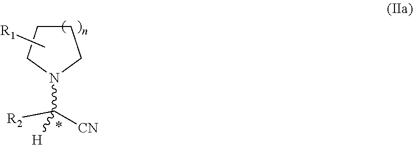

is applied, which comprises an asymmetric carbon atom in alpha-position to the cyano group
and
wherein
n and R₁ are as defined above and
R₂ represents a straight-chain or branched, saturated or non-saturated hydrocarbon group, in particular having 1 to 6 carbon atoms, in particular C₁-C₆ or C₁-C₃ alkyl,
wherein said nitrile is applied in the form of a mixture of stereoisomers, in particular as mixture of isomers comprising an (S)- or (R)-configuration at the carbon atom in alpha-position to the cyano group, and wherein said stereoisomeric mixture is converted via chemo-enzymatic dynamic kinetic resolution (DKR) to a reaction product containing a stereoisomeric excess either of a compound of formula Ia or of a compound of formula Ib; in particular of a compound of formula Ia, and in particular in essentially stereoisomerically pure form, like in a proportion of at least 90%, 91%, 92%, 93% 94%, more particularly 95%, 96%, 97%, 98%, 99%, 99.5%, 99.9% or 100% relative to the total amount of stereoisomers of a compound of formula I

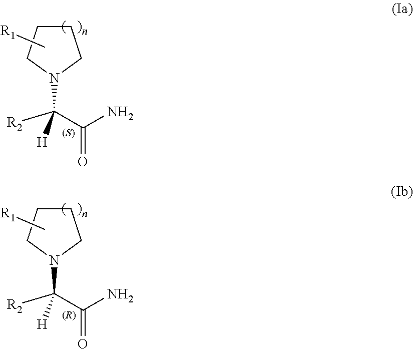

wherein
n, R₁ and R₂ are as defined above.
4. The process of embodiment 3, wherein a reaction product is obtained containing a stereoisomeric excess either of a compound of formula I-1a or of a compound of formula I-1b

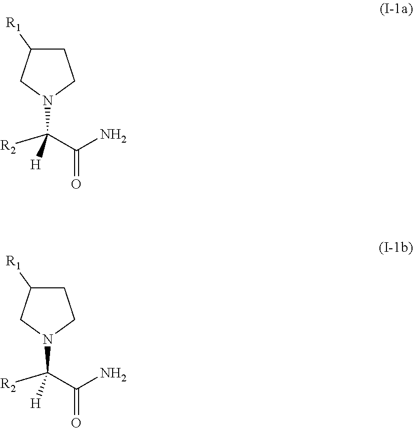

wherein
R₁ and R₂ are as defined above,
and in particular in a stereoisomeric excess corresponding to a proportion of at least 90%, 91%, 92%, 93% 94%, more particularly 95%, 96%, 97%, 98%, 99%, 99.5%, 99.9% or 100% relative to the total amount of stereoisomers I-1a and I-1b.
5. The process of embodiment 4, wherein a reaction product is obtained containing a stereoisomeric excess either of a compound of formula XIa or of a compound of formula XIb

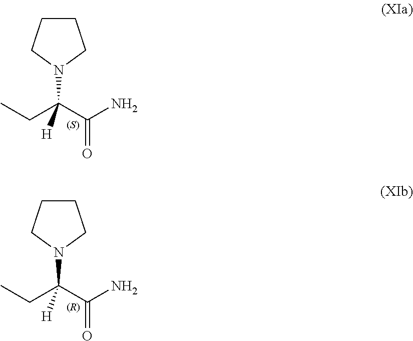

and in particular in a stereoisomeric excess corresponding to a proportion of at least 90%, 91%, 92%, 93% 94%, more particularly 95%, 96%, 97%, 98%, 99%, 99.5%, 99.9% or 100% relative to the total amount of stereoisomers XIa and XIb.
6. The process of embodiment 4, wherein a reaction product containing a stereoisomeric excess either of a compound of formula XXIa or of a compound of formula XXIb is obtained

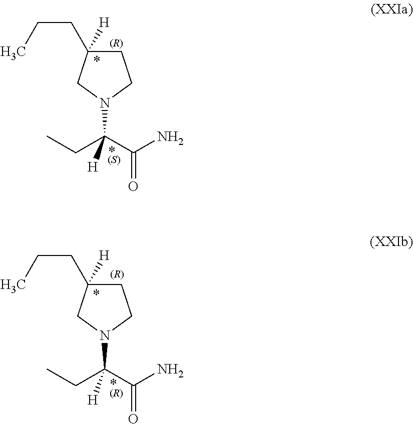

and in particular in a stereoisomeric excess corresponding to a proportion of at least 90%, 91%, 92%, 93% 94%, more particularly 95%, 96%, 97%, 98%, 99%, 99.5%, 99.9% or 100% relative to the total amount of stereoisomers XXIa and XXIb.
7. The process of embodiment 1, wherein a reaction product containing a compound of formula XX is obtained

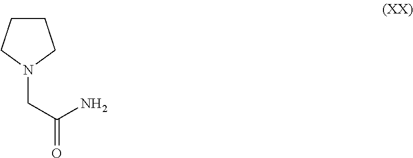

8. The process of any one of the preceding embodiments, wherein step 1) is performed in the presence of an isolated, enriched or crude NHase enzyme, or in the presence of a recombinant organism, in particular microorganism, functionally expressing said enzymes, in particular resting cells of such recombinant microorganism, or non-viable cells, disrupted cells or a cell homogenate obtained therefrom, or a cell-free in vitro expression system.
9. The process of one of the preceding embodiments, wherein the NHase is an (S)—NHase as defined above and the obtained product is a compound of formula Ia.
10. The process of one of the preceding embodiments, wherein the (S)—NHase is selected from polypeptides which are members of the family of non-corrin cobalt-containing NHases or non-heme iron-containing NHases.
11. The process of embodiment 10, wherein the (S)—NHase is a hetero-dimer composed of one α-polypeptide subunit and one β-polypeptide subunit.
12. The process of embodiment 11, wherein the (S)—NHase is selected from the enzymes:
   a) CtNHase, comprising an α-polypeptide subunit according to SEQ ID NO: 15 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 15 and a β-polypeptide subunit according to SEQ ID NO: 2 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 2, while retaining (S)—NHase activity;
   b) KoNHase, comprising an α-polypeptide subunit according to SEQ ID NO: 17 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 17 and a β-polypeptide subunit according to SEQ ID NO: 4 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 4, while retaining (S)—NHase activity;
   c) NaNHase, comprising an α-polypeptide subunit according to SEQ ID NO: 19 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 19 and a β-polypeptide subunit according to SEQ ID NO: 6 or a sequence having at least 50%, 55%, 60%, 65%, 70%, 75%, 80%, 85%, 90%, 95%, 96%, 97%, 98%, 99% 99.5% or 99.9% sequence identity to SEQ ID NO: 6, while retaining (S)—NHase activity;
   d) GhNHase, comprising an α-polypeptide subunit according to SEQ ID NO: 21 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 21 and a β-polypeptide subunit according to SEQ ID NO: 8 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 8, while retaining (S)—NHase activity;
   e) PkNHase, comprising an α-polypeptide subunit according to SEQ ID NO: 27 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 27 and a β-polypeptide subunit comprising a partial polypeptide sequence according to SEQ ID NO: 13 or a sequence having at least 50%, 55%, 60%, 65%, 70%, 75%, 80%, 85%, 90%, 95%, 96%, 97%, 98%, 99%, 99.5% or 99.9% sequence identity to said partial sequence of SEQ ID NO: 13, while retaining (S)—NHase activity;
   f PmNHase comprising an α-polypeptide subunit according to SEQ ID NO: 23 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 23 and a β-polypeptide subunit according to SEQ ID NO: 10 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 10, while retaining (S)—NHase activity; and
   g) ReNHase. comprising an α-polypeptide subunit according to SEQ ID NO: 25 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 25 and a β-polypeptide subunit according to SEQ ID NO: 12 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 12, while retaining (S)—NHase activity.
13. The process of embodiment 12, wherein the (S)—NHase is selected from CtNHase mutants, containing at least one, as for example 1 to 10, in particular 1, 2, 3, 4 or 5 amino acid mutations, in particular substitutions, in its α-polypeptide subunit according to SEQ ID NO: 15, and/or at least one, as for example 1 to 10, in particular 1, 2, 3, 4 or 5 amino acid mutations, in particular substitutions, in its β-polypeptide subunit according to SEQ ID NO: 2, while retaining (S)—NHase activity.
   Optionally said mutation shows at least one of the following improvements in comparison to the non-mutated parent enzyme, comprising an α-polypeptide subunit according to SEQ ID NO: 15 and a β-polypeptide subunit according to SEQ ID NO: 2:
   a) improved enantioselectivity for the production of a compound of formula (I-1a), in particular of formula XIa, in particular showing ee % values for the production of the enantiomer (S)-2 corresponding to formula XIa of >84%, like 85 to about 100% or particularly, 90-99.9% or even more particularly 95 to 99.9%;
   b) improved activity for conversion of a substrate of formula II, in particular of a racemic substrate rac-1 of at least 1 to 1000%, in particular 1 to 500%, as for example 10%, 20%, 50%, 100%, 150%, 200%, 250%, or 300%;
   c) improved cyanide tolerance, so that for example enzyme activity is retained to 100% at a cyanide concentration in the range of 1 to 100 mM, in particular 5 to 75 mM, as for example 5 mM, 10 mM, 20 mM, 30 mM, 40 mM, 50 mM or 60 mM;
   d) reduced substrate inhibition by at least one substrate of formula II, so that for example the initial activity rate is retained in the presence of 10 to 750 mM, in particular 50 to 500 mM, as for example 50 mM, 75 mM, 100 mM, 125 mM, 150 mM, 200 mM, 300 mM, 400 mM, 500 mM substrate;
   e) reduced product inhibition by at least one compound of formula (I-1a), in particular a compound of formula (XIa), so that for example the initial activity rate is retained in the presence of 10 to 750 mM, in particular 50 to 500 mM, as for example 50 mM, 75 mM, 100 mM, 125 mM, 150 mM, 200 mM, 300 mM, 400 mM, 500 mM product;
   f) improved thermal stability and/or stability against sheer forces; and
   g) reduced side activity for hydrolysis of 2-OH-butanenitrile,
   wherein these properties a) to g) may be present individually or in any combination.
   A first particular group of mutants shows an improved enantioselectivity as defined above under a).
   A second particular group of mutants shows an improved conversion of substrate as defined above under b).
   A third particular group of mutants shows an improved enantioselectivity as defined above under a) and an improved conversion of substrate as defined above under b).
   A fourth particular group of mutants shows an improved enantioselectivity as defined above under a), an improved conversion of substrate as defined above under b) and an improved cyanide tolerance as defined above under c).
   A fifth particular group of mutants shows an improved enantioselectivity as defined above under a), an improved conversion of substrate as defined above under b) and a reduced substrate inhibition as defined above under d).
   A sixth particular group of mutants shows an improved enantioselectivity as defined above under a), an improved conversion of substrate as defined above under b) and a reduced product inhibition as defined above under e).
   A seventh particular group of mutants shows an improved enantioselectivity as defined above under a), an improved conversion of substrate as defined above under b) and an improved operational stability and/or stability against shear forces as defined above under f).
   An eighth particular group of mutants shows an improved enantioselectivity as defined above under a), an improved conversion of substrate as defined above under b) and a reduced side activity for hydrolysis of 2-OH-butanenitrile as defined above under g).
   A ninth particular group of mutants shows improvement in all points a) to g).
14. The process of embodiment 13, wherein the CtNHase mutant is selected from mutants having at least one mutation, in particular amino acid substitution, in its α-polypeptide subunit according to SEQ ID NO: 15 in a sequence position selected from
   the α sequence positions
      αA71X, αK73X, αD79X, αT81X, αL87X, αG94X, αV98X,
      αE101X, αN102X, αT103X, αA105X, αV106X, αV110X;
      αP121X, αG124X, αY135X, αV140X, αL147X, αV153X, αA156X, αL173X,
      αP174X;
   in particular αV110X, and αP121X.
   wherein each X is independently selected from natural amino acids; and in particular mutants of said α-polypeptide subunit which at least improve substrate conversion of a substrate of formula II, in particular of a racemic substrate rac-1
   and/or
   at least one mutation, in particular amino acid substitution, in its α-polypeptide subunit according to SEQ ID NO: 2 in a sequence position selected from
   the β sequence positions
      βT32X, βV33X, βM34X, βS35X, βL36X, βL40X, βA42X, βN43X, βN45X, βF46X,
      βN47X, βL48X, βE50X, βF51X, βR52X, βH53X, βG54X, βE56X, βR57X, βN59X,
      β161X, βD62X, βL64X, βK65X, βG66X, βT67X, βE70X;
      βG125X, βA126X, βR127X, βA128X, βR129X, βA131X, βV132X, βG133X,
      βV136X, βR137X, βK141X, βP143X, βV144X, βG145X, βH146X, βP150X,
      βY152X, βT153X, βG155X, βK156X, βV157X, βT159X, βI162X, βH164X,
      βG165X, βV166X, βF167X, βV168X, βT169X, βP170X;
   in particular βL48X, βF51X, βG54X, βH146X, and βF167X,
   wherein each X is independently selected from natural amino acids; and in particular mutants of said ß-polypeptide subunit which at least improve enantioselectivity for the production of a compound of formula (I-1a), in particular of formula XIa, in particular showing ee % values for the production of the enantiomer (S)-2 corresponding to formula XIa of >84%, like 85 to about 100% or particularly, 90-99.9% or more particularly 95 to 99.9%, or even more particularly 99 to 99.9%.
15. The process of embodiment 14, wherein the CtNHase mutant is selected from:

a) the single mutants: βF51L, βF51I, βF51V, βL48R and βL48P

b) the double mutants:
αV110I/βF51L,
αP121T/βF51L,
βF51V/βG54V,
βF51V/βG54I,
βF51V/βG54R,
βF51I/βG54R,
βN43I/βG54C
βF51I/βE70L,
βH53L/βG54V,
αV110I/βL48R,
αV110I/βL48P,
αV110I/βL48F,
αP121T/βL48R,
αP121T/βL48P,
αP121T/βL48F,
βH146L/βF167Y,
βL48R/βG54C,
βL48R/βG54R,
βL48R/βG54V,
βL48P/βG54C,
βL48P/βG54R,
βL48P/βG54V,
βL48F/βG54C,
βL48F/βG54R, and
βL48F/βG54V;
in particular
αV110I/βF51L,
αP121T/βF51L,
βF51V/βG54V,
βN43I/βG54C
βF51I/βE70L,
βH53L/βG54V,
αP121T/βL48R,
βH146L/βF167Y, and
βL48R/βG54V.

c) the triple mutants:
βF51L/βH146L/βF167Y,
βL48R/βH146L/βF167Y,
βL48P/βH146L/βF167Y,
βL48F/βH146L/βF167Y,
αV110I/βF51V/βG54I,
αP121T/βF51V/βG54I,
βL48P/βF51V/βG54V and
βL48R/βF51I/βG54I;
in particular βL48R/βF51I/βG54I

d) the multiple mutants:
βF51I/βG54R/βH146L/βF167Y,
βF51V/βG54I/βH146L/βF167Y,
βF51V/βG54R/βH146L/βF167Y,
βF51V/βG54V/pH146L/βF167Y,
αV110I/αP121T/βF51I/βH146L/βF167Y,
αV110I/αP121T/βF51L/pH146L/βF167Y
16. The process of one of the preceding embodiments, wherein the enantioselective conversion of the racemic compound of formula II is carried out continuously or discontinuously, under at least one of the following conditions:
   a) aqueous or aqueous-organic reaction medium;
   b) presence of an added amount of aldehyde, in particular an aldehyde as formed during the spontaneous decomposition of a nitrile of formula II in aqueous medium, as in particular propanal as formed from a compound of formula rac-1, in a concentration range of 1-200 mM, more particularly 5-150 mM, in particular 25-100 mM;
   c) pH control, in particular by applying a pH in the range of 6-10, more particularly 6-8, in particular 6.5-7.5;
   d) temperature control, in particular by applying a temperature in the range of 0-50° C., more particularly 5-35° C., in particular 5-25° C.;
   e) control of substrate concentration, in particular by applying a concentration in the range of 1-200 mM, more particularly 5-150 mM, in particular 25-100 mM; especially continuous or stepwise control of substrate concentration, in particular by applying a concentration in the range of 1-200 mM, more particularly 5-150 mM, in particular 25-100 mM;
   f) control of aldehyde concentration, as in particular of propanal as formed from a compound of formula rac-1, in particular by applying an aldehyde concentration in the range of 1-200 mM, more particularly 5-150 mM, in particular 25-100 mM; especially the continuous or stepwise control of aldehyde concentration (as in particular of propanal as formed from a compound of formula rac-1), in particular by applying an aldehyde concentration in the range of 1-200 mM, more particularly 5-150 mM, in particular 25-100 mM;
   g) catalyst concentration in a range of 0.01-100 mg/mi CWW, particularly 0.1-20 mg/ml, more particularly 2-10 mg/ml CWW;
   h) presence of an added amount of cyclic amine, in particular a cyclic amine as formed during the spontaneous decomposition of a nitrile of formula II in aqueous medium, as in particular of pyrrolidine as formed from a compound of formula rac-1, in a concentration range of 1-200 mM, more particularly 5-150 mM, in particular 25-100 mM; or
   i) control of cyclic amine concentration in particular a cyclic amine as formed during the spontaneous decomposition of a nitrile of formula II in aqueous medium, as in particular of pyrrolidine as formed from a compound of formula rac-1, in particular by applying a cyclic amine concentration in a concentration range of 1-200 mM, more particularly 5-150 mM, in particular 25-100 mM; especially the continuous or stepwise control of the cyclic amine concentration, as in particular of pyrrolidine as formed from a compound of formula rac-1, in particular by applying an aldehyde concentration in the range of 1-200 mM, more particularly 5-150 mM, in particular 25-100 mM.
   Particular processes are carried out under anyone of the following combinations of the above conditions:
      a)+b)
      a)+b)+c)
      a)+b)+c)+d)
      a)+b)+c)+d)+e) or
      a)+b)+c)+d)+f)
      each optionally in combination with g);
      or
      a)+h)
      a)+c)+h)
      a)+c)+d)+h)
      a)+c)+d)+e)+h) or
      a)+c)+d)+f)+h)
      each optionally in combination with g);
      or
      a)+h)+i)
      a)+c)+h)+i)
      a)+c)+d)+h)+i)
      a)+c)+d)+e)+h)+i) or
      a)+c)+d)+f)+h)+i)
      each optionally in combination with g);
      or
      a)+b)+h)
      a)+b)+c)+h)
      a)+b)+c)+d)+h)
      a)+b)+c)+d)+e)+h) or
      a)+b)+c)+d)+f)+h)
      each optionally in combination with g);
      or
      a)+b)+h)
      a)+b)+c)+h)+f)+i)
      a)+b)+c)+d)+h)+f)+i) or
      a)+b)+c)+d)+e)+h)+f)+i)
      each optionally in combination with g);
      or
      in particular
      a)+b)+c)+d)+e)+f)+g); or
      a)+b)+c)+d)+e)+f)+g)+h)+i).
   As used herein the term “control” encompasses the continuous, discontinuous or stepwise measurement of and optionally the supplementation of the respective compound in order to maintain the initial concentration of said compound or to maintain its concentration in the intended concentration range.
   Aldehyde, as in particular propanal, or cyclic amine, as in particular pyrrolidine, or both of them may be added to the reaction mixture, and their concentration may be controlled in order to shift the equilibrium of decomposition and new formation of nitrile (II) or IIa, in particular of the R-1 decomposition, and new formation, in particular of rac-1, so that free cyanide is bound and thus minimize the concentration of free cyanide anions, so that NHase inhibition by cyanide is minimized or even avoided.
17. The process of one of the preceding embodiments, wherein the NHase enzyme is recombinantly expressed under co-expression of at least one NHase α-subunit and at least one NHase β-subunit with at least one accessory protein, in particular with E. coli as expression host, more particularly E. coli BL21.
18. The process of embodiment 17, wherein the accessory protein is selected from
   a) accessory proteins of the same organism of origin as the α-subunit and the β-subunit of the NHase, in particular an accessory protein comprising an amino acid sequence selected from SEQ ID NO: 137, 139, 141, 143 and 145, or a sequence having at least 50%, 55%, 60%, 65%, 70%, 75%, 80%, 85%, 90%, 95%, 96%, 97%, 98%, 99%, 99.5% or 99.9% sequence identity to anyone of SEQ ID NO: 137, 139, 141, 143 and 145, while retaining its activity as accessory protein; and
   b) chaperones, in particular GroES/EL or DnaK/j-GrpE.
19. The process of embodiment 18, wherein
   a) CtNHase or a mutant thereof as defined in anyone of the embodiments 12 to 15 is co-expressed with an accessory protein comprising a polypeptide sequence according to SEQ ID NO: 137 or a sequence having at least 50%, 55%, 60%, 65%, 70%, 75%, 80%, 85%, 90%, 95%, 96%, 97%, 98%, 99%, 99.5% or 99.9% sequence identity to SEQ ID NO: 137, while retaining its activity as accessory protein;
   b) KoNHase as defined in embodiment 12 is co-expressed with an accessory protein comprising a polypeptide sequence according to SEQ ID NO: 139 or a sequence having at least 50%, 55%, 60%, 65%, 70%, 75%, 80%, 85%, 90%, 95%, 96%, 97%, 98%, 99%; 99.5% or 99.9% sequence identity to SEQ ID NO: 139, while retaining its activity as accessory protein;
   c) NaNHase as defined in embodiment 12 is co-expressed with an accessory protein comprising a polypeptide sequence according to SEQ ID NO: 141 or a sequence having at least 50%, 55%, 60%, 65%, 70%, 75%, 80%, 85%, 90%, 95%, 96%, 97%, 98%, 99%; 99.5% or 99.9% sequence identity to SEQ ID NO: 141, while retaining its activity as accessory protein;
   d) GhNHase as defined in embodiment 12 is co-expressed with an accessory protein comprising a polypeptide sequence according to SEQ ID NO: 143 or a sequence having at least 50%, 55%, 60%, 65%, 70%, 75%, 80%, 85%, 90%, 95%, 96%, 97%, 98%, 99%; 99.5% or 99.9% sequence identity to SEQ ID NO: 143, while retaining its activity as accessory protein;
   e) PmNHase as defined in embodiment 12 is co-expressed with an accessory protein comprising a polypeptide sequence according to SEQ ID NO: 144 or a sequence having at least 50%, 55%, 60%, 65%, 70%, 75%, 80%, 85%, 90%, 95%, 96%, 97%, 98%, 99%; 99.5% or 99.9% sequence identity to SEQ ID NO: 144, while retaining its activity as accessory protein; and
   f) ReNHase as defined in embodiment 12 is co-expressed with an accessory protein comprising a polypeptide sequence according to SEQ ID NO: 145 or a sequence having at least 50%, 55%, 60%, 65%, 70%, 75%, 80%, 85%, 90%, 95%, 96%, 97%, 98%, 99%; 99.5% or 99.9% sequence identity to SEQ ID NO: 145 while retaining its activity as accessory protein.
20. An isolated (S)—NHase enzyme is selected from
   a) KoNHase, comprising an α-polypeptide subunit according to SEQ ID NO: 17 or a sequence having at least 50%, 55%, 60%, 65%, 70%, 75%, 80%, 85%, 90%, 95%, 96%, 97%, 98%, 99%; 99.5% or 99.9% sequence identity to SEQ ID NO: 17 and/or a β-polypeptide subunit according to SEQ ID NO: 4 or a sequence having at least 50%, 55%, 60%, 65%, 70%, 75%, 80%, 85%, 90%, 95%, 96%, 97%, 98%, 99%; 99.5% or 99.9% sequence identity to SEQ ID NO: 4, while retaining (S)—NHase activity; and
   b) a mutant of CtNHase retaining (S)—NHase activity, and comprising a mutated α-polypeptide subunit, differing from SEQ ID NO: 15 in at least one amino acid residue and having at least 50%, 55%, 60%, 65%, 70%, 75%, 80%, 85%, 90%, 95%, 96%, 97%, 98%, 99%; 99.5% or 99.9% sequence identity to SEQ ID NO: 15 and/or a mutated β-polypeptide subunit, differing from SEQ ID NO: 2 in at least one amino acid residue and having at least 50%, 55%, 60%, 65%, 70%, 75%, 80%, 85%, 90%, 95%, 96%, 97%, 98%, 99%; 99.5% or 99.9% sequence identity to SEQ ID NO: 2.
21. The isolated (S)—NHase mutant of embodiment 20 b), which is selected from mutants of CtNHase as defined in one of the embodiments 13 to 15.
22. A nucleic acid molecule comprising a nucleotide sequence encoding the polypeptide-subunits of a functional (S)—NHase enzyme as defined in one of the embodiments 20 and 21.
23. The nucleic acid molecule of embodiment 22, further comprising a nucleotide sequence encoding at least one accessory polypeptide assisting the assembly of the polypeptide subunits of the (S)—NHase including its non-corrin cobalt or non-heme iron center, in particular selected from nucleic acid molecules encoding an accessory protein comprising an comprising a nucleic acid sequence selected from SEQ ID NO: 136, 138, 140, 142 and 144, or a nucleic acid sequence having at least 50%, 55%, 60%, 65%, 70%, 75%, 80%, 85%, 90%, 95%, 96%, 97%, 98%, 99%; 99.5% or 99.9% sequence identity to anyone of from SEQ ID NO: 136, 138, 140, 142 and 144, while retaining its ability to encode a polypeptide having activity as accessory protein.
24. An expression cassette, comprising at least one nucleotide sequence as defined in embodiments of 22 or 23 under the control of at least one regulative nucleotide sequence.
25. An expression vector, comprising at least one expression cassette as defined in embodiment 24.
26. A recombinant microorganism, which carries at least one nucleic acid as defined in the embodiments 22 or 23 or at least one expression cassette according to embodiment 24 or at least one expression vector according to embodiment 25.
   Particular embodiments of such recombinant microorganisms encompass intact (living), inactivated, non-viable or resting cell microorganisms.
27. A chemo-biocatalytic process for the preparation of a lactam compound of the formula IIIa or IIIb

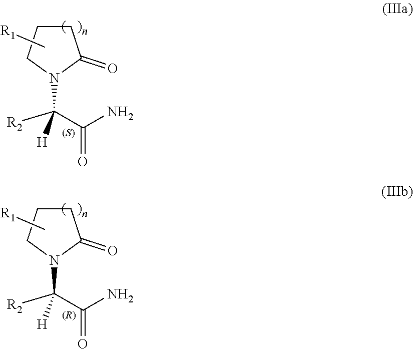

wherein
n is 0 or an integer of 1 to 4; in particular 1 or 2, more particularly 1, and
R₁ and R₂ independently of each other represent H or a hydrocarbon group, in particular a straight-chain or branched, saturated or non-saturated hydrocarbon group, having 1 to 6 carbon atoms; in particular H or C₁-C₆ alkyl or C₁-C₃ alkyl, more particularly H or C₁-C₃ alkyl, like in particular methyl;
optionally an essentially stereoisomerically pure form or as a mixture of stereoisomers; like in particular in a proportion of at least 90%, 91%, 92%, 93%, 94%, more particularly 95%, 96%, 97%, 98%, 99%, 99.5%, 99.9% or 100% relative to the total amount of stereoisomers of the formula IIIa and IIIb,

which process comprises the following steps:
1) optionally the chemical synthesis of a stereoisomeric mixture of an alpha amino nitrile of the formula IIc

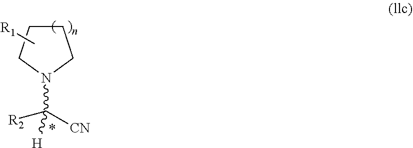

wherein n, R₁ and R₂ are as defined above,
by a Strecker synthesis, in particular by reacting a cyanide compound, in particular HCN or an alkali or alkaline earth metal cyanide, like more particularly NaCN or KCN, an aldehyde of the formula R₂—CHO, wherein R₂ is as defined above, and a cyclic amine of the formula (IV)

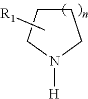

wherein n and R₁ are as defined above;
2) the enantioselective biocatalytic conversion of the compound of formula IIc, optionally as obtained according to step 1), by a process as defined in any one of the embodiments 1 to 19 via chemo-enzymatic dynamic kinetic resolution in order to obtain a reaction product containing a stereoisomeric excess either of a compound of formula Ia or of a compound of formula Ib.:

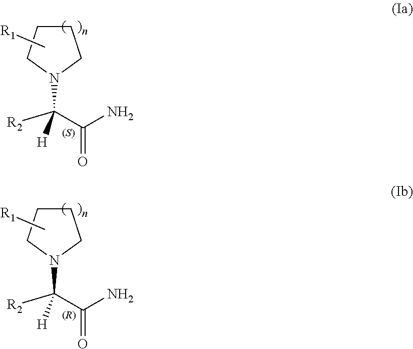

wherein n, R₁ and R₂ are as defined above;
and
3) the chemical oxidation of said alpha-amino amide of the formula Ia or Ib to the corresponding lactam derivative of the general formula IIIa or IIIb.

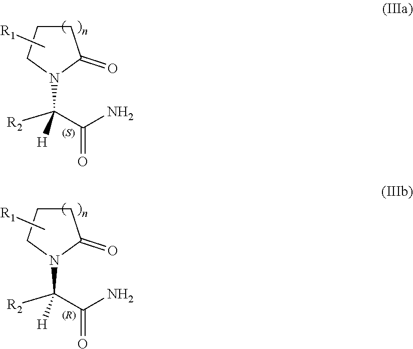

wherein n, R₁ and R₂ are as defined above.
28. The process of embodiment 27, wherein the chemical oxidation of step 3) is performed with a heterogeneous or homogenous oxidation catalyst, in particular a homogeneous catalyst, capable of oxidizing the heterocyclic alpha-amino group in a compound of formula (la) or (Ib) under substantial retention of the stereochemistry at the asymmetric carbon atom in α-position to the amide group.
29. The process of embodiment 28, wherein the oxidation catalyst is selected from combinations of an inorganic ruthenium salt, in particular (+III), (+IV), (+V) or (+VI) salt, more particularly (+III) or (+IV) salt, and at least one oxidant capable of in situ oxidizing a ruthenium salt, in particular (+III), (+IV), (+V) or (+VI) salt, more particularly (+III) or (+IV), in particular to a ruthenium (+VIII) salt, and optionally in the presence of a mono- or polyvalent metal ligand, as for example sodium oxalate.
30. The process of embodiment 29, wherein the inorganic ruthenium (+III) or (+IV) salt is selected from RuCl₃, RuO₂ and the respective hydrates, in particular monohydrates, thereof; and wherein the oxidant is selected from alkali perhalogenates, alkali hypochlorites, and their hydrates; or combinations thereof.
31. The process of embodiment 30, wherein the oxidant is selected from
   a) alkali periodates, particularly alkali meta-periodates, in particular NaIO₄
   b) alkali chlorates, in particular NaOCl, the hydrates thereof, in particular NaOCl*5 H₂O; and
   c) mixtures of a) and b).
32. The process of anyone of the embodiments 29 to 31, wherein the oxidation catalyst is selected from
   a) RuO₂/NaIO₄
   b) RuO₂*H₂O/NaIO₄
   c) RuCl₃*H₂O/NaIO₄
   d) RuCl₃*H₂O/NaOCl*5 H₂O
   e) RuCl₃*H₂O/NaIO₄/NaOCl*5 H₂O and
   f) each of a) to d) in combination with a mono- or polyvalent metal ligand, as for example sodium oxalate.
33. The process of anyone of the embodiments 29 to 32, wherein the chemical oxidation is performed by reacting an aqueous or aqueous-organic solution of said compound of formula Ia or said compound of formula Ib at a temperature in the range of 0 to 30° C. with the oxidation catalyst.
34. The process of anyone of the embodiments 29 to 33, wherein the chemical oxidation is performed by reacting said compound of formula Ia or said compound of formula Ib with a catalytic amount of said inorganic ruthenium salt, in particular said (+III) or (+IV) salt and said oxidant, wherein the initial molar ratio of compound of formula Ia or Ib and oxidant is in the range of 1:1 to 1:5, in particular 1:1.5 to 1:3.
35. The process of anyone of the embodiments 29 to 34, wherein the mono- or polyvalent metal ligand is added to the reaction mixture, so that the molar ratio of ruthenium (+III) or (+IV) salt to ligand is in the range of 1:1 to 1:5, in particular 1:1.5 to 1:2.5.
36. The process of anyone of the embodiments 27 to 35, wherein the obtained lactam derivative is selected from Levetiracetam of the formula XIIIa and Brivaracetam of the formula XXIa and Piracetam of the formula XX.

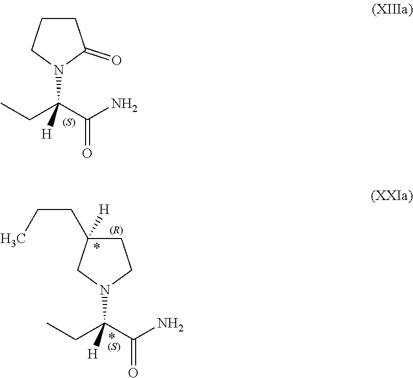

37. The process of anyone of the embodiments 27 to 36, further comprising the recovering, for example by precipitation from the reaction medium, and electrochemical recycling of the spent oxidant, in particular of the electrochemical oxidation of an alkali halogenate back to an alkali perhalogenate oxidant, more particularly the electrochemical oxidation of an alkali iodate, especially sodium or potassium iodate, back to an alkali periodate oxidant, especially sodium or potassium periodate oxidant.
   The electrochemical recycling of sodium iodate to sodium periodate is preferred.
   In particular, the alkali halogenate, more particularly alkali iodate, especially sodium or potassium iodate, even more particularly sodium iodate, is isolated from the reaction mixture as described in more detail below. For example isolation by precipitation, in particular by applying a water soluble organic solvent, as for example alcohol precipitation is performed. More particularly, methanol or iso-propanol is added to form a precipitate. This precipitate may then be isolated, for example by filtration, optionally by decantation. The thus obtained halogenate, in particular iodate, even more particularly sodium iodate, is then subjected to the electrochemical recycling process.
   In analogy, the present invention allows the recycling of any alkali perhalogenate oxidant spent in any other chemical and/or biochemical oxidation reaction, and in particular the electrochemical oxidation of an alkali halogenate back to an alkali perhalogenate oxidant, and more particularly the electrochemical oxidation of an alkali iodate, especially sodium or potassium iodate, even more particularly sodium iodate, back to an alkali periodate oxidant, especially sodium or potassium periodate oxidant, even more particularly back to sodium periodate, which may then be utilized again in said chemical or biochemical oxidation process.
38. The process of embodiment 37, wherein the electrochemical recycling comprises an anodic oxidation of said alkali halogenate more particularly alkali iodate, especially sodium or potassium iodate, even more particularly sodium iodate, back to said alkali perhalogenate oxidant especially back to an alkali periodate oxidant, like sodium or potassium periodate oxidant, even more particularly back to sodium periodate oxidant.
39. The process of embodiment 37 or 38, wherein a boron-doped diamond anode is applied.
40. The method of anyone of the embodiments 27 to 39, wherein the anodic oxidation is performed under at least one of the following conditions:
   a) (1) aqueous solution of at least one alkali halogenate, more particularly alkali iodate, especially sodium or potassium iodate, at an initial concentration co of 0.001 to 5 M, more preferably from 0.001 to 2.5 M or 0.001 to 2 M or 0.001 to 1 M, in particular from 0.01 to 1 M or 0.01 to 0.5 M, and specifically from 0.1 to 0.3 M; (2) initial molarity of the base in the alkaline solution in the range of 0.3 to 5 M or 0.5 to 5 M, preferably 0.6 to 4 M, 0.8 to 4 M or 0.6 to 3 M, in particular 0.9 to 2 M and specifically 1 M, (3) optionally ratio of base to halogenate being in the range of 10:1 to 1:1, more preferably from 8:1 to 2:1, in particular 6:1 to 3:1, more particularly the ratio of base to halogenate being at least 2.5:1 or higher, specifically 5:1 to 4:1, wherein the base is selected from alkali metal and alkaline earth metal hydroxides or carbonates, in particular K₂CO₃, LiOH, NaOH, KOH, CsOH and Ba(OH)₂, more preferably NaOH, KOH, and most preferred NaOH,
   b) pH of the aqueous solution of 7 or more, like at a pH of at least 8, preferably of at least 10, in particular of at least 12, more particular at least 13, and specifically of at least 14,
   c) temperature in the range of 0 to 80° C., more preferably from 10 to 60° C., in particular from 20 to 30° C. and specifically from 20 to 25° C.,
   d) voltage in the range of 1 to 30V particularly from 1 to 20 V and more particular from 1 to 10 V.,
   e) current density in the range of 10 to 500 mA/cm², like from 50 to 150 mA/cm², in particular from 80 to 120 mA/cm² and specifically of ca. 100 mA/cm²; and
   f) applied charge in the range of 1 to 10 Farad, more preferably from 2 to 6 F, in particular from 2.5 to 4 F, and specifically 2.75 to 3.5 Farad,
   in particular a combination comprising at least features a), b), e) and f).
   In particular, the optimum current density j can be determined with respect to the type of electrolysis applied by a skilled person in the art. Batch or divided batch electrolyses, may use current densities in the range of 10 to 500 mA/cm². If the oxidation is to be performed in an electrolytic flow cell, the flow rate determines the maximum current density to be applicable. For example, in a flow cell with 48 cm² anode surface area, an anode-membrane gap of 1 mm, and a flow rate of 7.5 L/h, the optimal current density j may be determined to be in a range of about 400-500 mA/cm², and specifically about 416 mA/cm².
   In general, at higher flow rates or higher halogenate (like iodate) concentrations, the current density j may be higher, while at lower flow rates or lower halogenate (like iodate) concentrations, the current density must be lower to maintain current efficiency (CE).
   In a particular embodiment, the initial molarity co of the base in the aqueous alkaline solution of the alkali halogenate is in the range of 0.3 to 5 M or 0.5 to 5 M, preferably 0.6 to 4 M, 0.8 to 4 M or 0.6 to 3 M, in particular 0.9 to 2 M and specifically 1 M. In particular the base is NaOH or KOH and the alkali halogenate is sodium or potassium iodate. More particularly the base is NaOH and the alkali halogenate is sodium iodate.
   In another particular embodiment, the pH of the aqueous solution is at least 12, at least 13, and specifically at least 14.
   In another particular embodiment, the initial concentration co of the at least one alkali halogenate, more particularly alkali iodate, especially sodium or potassium iodate, in said aqueous solution is low and is in the range of 0.001 to 1 M, in particular from 0.01 to 0.5 M or 0.01 to 0.4 M, and specifically from 0.05 to 0.25 M.
   In another particular embodiment, the ratio of co (NaOH):c₀ (NaIO₃) is set in the range of 10:1 to 1:1, preferably 8:1 to 2:1, in particular 6:1 to 3:1, specifically 5:1 to 4:1.
   In another particular embodiment, a feature combination comprising at least the above features a), b), e) and f) is applied. Here feature a) may comprise either features a(1) and a(2), or features a(2) and a(3), or more preferably features a(1), a(2) and a(3).
   In another particular embodiment, a feature combination comprising at least the above features a) and b) is applied. Here feature a) may comprise either features a(1) and a(2), or features a(2) and a(3), or more preferably features a(1), a(2) and a(3).
   According to a very particular embodiment thereof the alkali metal is sodium, and the recycled product is sodium periodate, obtained as sodium para-periodate.
   According to said very particular embodiment, the following particular parameters are applied alone or in combination:
      current density j in the range of 50 to 100 mA/cm² in batch electrolysis; or current density j in the range of 400 to 500 mA/cm² in flow electrolysis (as for example observed at a flow rate of 7.5 L/h and 48 cm² anode surface area)
      applied charge Q in the range of 3 to 4 F
      initial concentration cₒ (NaIO₃) of about 0.21 M
      initial concentration cₒ (NaOH) of about 1.0 M
      ratio of cₒ (NaIO₃):cₒ (NaOH) of about 1:5
   In a further particular embodiment of the iodate recycling process of the invention, the para-periodate as preferentially obtained by electrolysis is converted to meta-periodate.
      For this purpose, after electrolysis, para-periodate is isolated from the anolyte as described in more detail below. The precipitate is obtained from the liquid phase in the anode chamber by filtration or decantation. The precipitation may be completed by usual means, for example by the addition of sodium hydroxide or by concentration of the solvent. In order to obtain meta-periodate said para-periodate is neutralized by addition of acid, in particular sulfuric or nitric acid and is then recrystallized in a manner known per se.
41. A process for the preparation of an alkali perhalogenate, in particular periodate, which process comprises the electrochemical anodic oxidation of an alkali halogenate, in particular iodate, to an alkali perhalogenate, in particular periodate, wherein in particular a boron-doped diamond anode is applied. The alkali cation is, in particular, selected from sodium or potassium, especially sodium.
42. The process of embodiment 41, wherein a boron-doped diamond anode is applied.
43. The process of embodiment 41 or 42, wherein the anodic oxidation is performed under at least one of the following conditions:
   a) (1) aqueous solution of at least one alkali halogenate, in particular iodate, at an initial concentration of 0.001 to 5 M or 0.001 to 1 M, more preferably from 0.001 to 2 M, in particular from 0.01 to 1 M or 0.01 to 0.5 M or 0.01 to 0.4 M or 0.05 to 0.25 M, and specifically from 0.1 to 0.3 M or 0.1 to 0.25 M. (2) initial molarity of the base in the alkaline solution in the range of 0.3 to 5 M, preferably 0.6 to 3 M, in particular 0.9 to 2 M and specifically 1 M; (3) optionally the ratio of base to halogenate being 10:1 or higher, or particularly in the range of 10:1 to 1:1, more particularly from 8:1 to 2:1, even more particular 6:1 to 3:1, and specifically 5:1 to 4:1, wherein the base is selected from alkali metal and alkaline earth metal hydroxides or carbonates, in particular K₂CO₃, LiOH, NaOH, KOH, CsOH and Ba(OH)₂, more preferably NaOH, KOH, and most preferred NaOH,
   b) pH of the aqueous solution of 7 or more, like at a pH of at least 8, preferably of at least 10, in particular of at least 12, at least 13 and specifically of at least 14,
   c) temperature in the range of 0 to 80° C., more preferably from 10 to 60° C., in particular from 20 to 30° C. and specifically from 20 to 25° C.,
   d) voltage in the range of 1 to 30V, particularly from 1 to 20 V and more particular from 1 to 10 V.,
   e) current density in the range of 10 to 500 mA/cm², more preferably from 50 to 150 mA/cm², in particular from 80 to 120 mA/cm² and specifically of ca. 100 mA/cm²; or in the range of 10 to 1000 mA/cm², more preferably from 50 to 750 mA/cm², in particular from 100 to 500 mA/cm² and specifically of about 400 mA/cm; and
   f) applied charge in the range of 1 to 10 F, more preferably from 2 to 6 F, in particular from 2.5 to 4 F, and specifically 2.75 to 3.5 F,
   In particular, the optimum current density j can be determined with respect to the type of electrolysis applied by a skilled person in the art. Batch or divided batch electrolyses, use current densities in the range of 10 to 1000 mA/cm². If the oxidation is to be performed in an electrolytic flow cell, the flow rate determines the maximum current density to be applicable. For example, in a flow cell with 48 cm² anode surface area, an anode-membrane gap of 1 mm, and a flow rate of 7.5 L/h, the optimal current density may be determined to be in a range of about 400-500 mA/cm², and specifically about 416 mA/cm².
   In general, at higher flow rates or higher halogenate (like iodate) concentrations, the current density j may be higher, while at lower flow rates or lower halogenate (like iodate) concentrations, the current density must be lower to maintain current efficiency (CE).
   In a particular embodiment, the initial molarity cₒ of the base in the aqueous alkaline solution of the alkali halogenate is in the range of 0.3 to 5 M or 0.5 to 5 M, preferably 0.6 to 4 M, 0.8 to 4 M or 0.6 to 3 M, in particular 0.9 to 2 M and specifically 1 M. In particular the base is NaOH or KOH and the alkali halogenate is sodium or potassium iodate. More particularly the base is NaOH and the alkali halogenate is sodium iodate.
   In another particular embodiment, the pH of the aqueous solution is at least 12, at least 13 and specifically at least 14.
   In another particular embodiment, the initial concentration co of the at least one alkali halogenate, more particularly alkali iodate, especially sodium or potassium iodate, in said aqueous solution is low and is in the range of 0.001 to 1 M, in particular from 0.01 to 0.5 M or 0.01 to 0.4 M, and specifically from 0.05 to 0.25 M.
   In another particular embodiment, the ratio of cₒ (NaOH):cₒ (NaIO₃) is set in the range of 10:1 to 1:1, preferably 8:1 to 2:1, in particular 6:1 to 3:1, specifically 5:1 to 4:1.
   In another particular embodiment, a feature combination comprising at least features a), b), e) and f) is applied. Here feature a) may comprise either features a(1) and a(2), or features a(2) and a(3), or more preferably features a(1), a(2) and a(3).
   In another particular embodiment, a feature combination comprising at least the above features a) and b) is applied. Here feature a) may comprise either features a(1) and a(2), or features a(2) and a(3), or more preferably features a(1), a(2) and a(3).
   According to a particular embodiment thereof the alkali metal is sodium, and the obtained product is sodium periodate, obtained as sodium para-periodate.
   According to said particular embodiment the following particular parameters are applied alone or in combination:
      current density j in the range of 50 to 100 mA/cm² in batch electrolysis; or a current density j in the range of 400 to 500 mA/cm² in flow electrolysis (as for example observed at a flow rate of 7.5 L/h, an anode-membrane gap of 1 mm, and 48 cm² anode surface area);
      applied charge Q in the range of 3 to 4 F
      initial concentration cₒ (NaIO₃) of about 0.21 M
      initial concentration cₒ (NaOH) of about 1.0 M
      ratio of cₒ (NaIO₃):cₒ (NaOH) of about 1:5
   In a further particular embodiment of the iodate preparation process of the invention, the para-periodate as preferentially obtained by electrolysis is converted to meta-periodate.
   For this purpose, after electrolysis, para-periodate is isolated from the anolyte as described in more detail below. The precipitate is obtained from the liquid phase in the anode chamber by filtration or decantation. The precipitation may be completed by usual means, for example by the addition of sodium hydroxide or by concentration of the solvent. In order to obtain meta-periodate said para-periodate is neutralized by the addition of acid, in particular sulfuric or nitric acid and then recrystallized in a manner known per se.
44. A process for the preparation of a lactam compound of the formula IIIa or IIIb

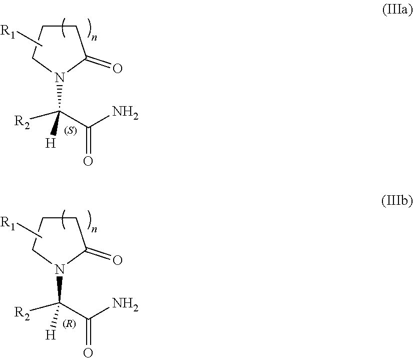

wherein
n is 0 or an integer of 1 to 4; and
R₁ and R₂ independently of each other represent H or a straight-chain or branched, saturated or non-saturated hydrocarbon group having 1 to 6 carbon atoms, in particular C₁-C₆ or C₁-C₃ alkyl;
which process comprises
the regioselective chemical oxidation of an alpha-amino amide of the formula Ia or Ib

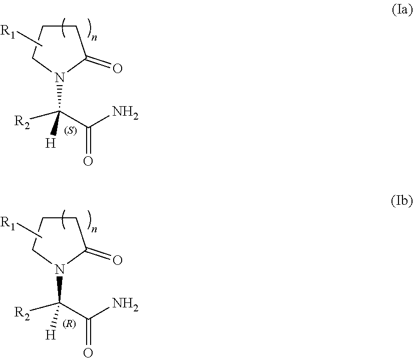

wherein n, R₁ and R₂ are as defined above;
to the corresponding lactam derivative of the general formula IIIa or IIIb.
45. The process of embodiment 44, wherein the chemical oxidation of step is performed with a heterogeneous or homogenous oxidation catalyst, in particular a homogeneous catalyst, capable of oxidizing the heterocyclic alpha-amino group in a compound of formula (Ia) or (Ib) under substantial retention of the stereochemistry at the asymmetric carbon atom in α-position to the amide group.
46. The process of embodiment 45, wherein the oxidation catalyst is selected from combinations of an inorganic ruthenium salt, in particular (+III), (+IV), (+V) or (+VI) salt, more particularly (+III) or (+IV) salt, and at least one oxidant capable of in situ oxidizing a ruthenium salt, in particular (+III), (+IV), (+V) or (+VI) salt, more particularly (+III) or (+IV), in particular to a ruthenium (+VIII) salt, and optionally in the presence of a mono- or polyvalent metal ligand, as for example sodium oxalate.
47. The process of embodiment 46, wherein the inorganic ruthenium (+III) or (+IV) salt is selected from RuCl₃, RuO₂ and the respective hydrates, in particular monohydrates, thereof; and wherein the oxidant is selected from alkali perhalogenates, alkali hypochlorites, and their hydrates; or combinations thereof.
48. The process of embodiment 47, wherein the oxidant is selected from
   a) alkali periodates, particularly alkali meta-periodates, in particular NaIO₄
   b) alkali chlorates, in particular NaOCl, the hydrates thereof, in particular NaOCl*5 H₂O; and
   c) mixtures of a) and b).
49. The process of anyone of the embodiments 46 to 48, wherein the oxidation catalyst is selected from
   a) RuO₂/NaIO₄
   b) RuO₂*H₂O/NaIO₄
   c) RuCl₃*H₂O/NaIO₄
   d) RuCl₃*H₂O/NaOCl*5 H₂O
   e) RuCl₃*H₂O/NaIO₄/NaOCl*5 H₂O and
   f) each of a) to d) in combination with a mono- or polyvalent metal ligand, as for example sodium oxalate.
50. The process of anyone of the embodiments 46 to 49, wherein the chemical oxidation is performed by reacting an aqueous or aqueous-organic solution of said compound of formula Ia or said compound of formula Ib at a temperature in the range of 0 to 30° C. with the oxidation catalyst.
51. The process of anyone of the embodiments 46 to 50, wherein the chemical oxidation is performed by reacting said compound of formula Ia or said compound of formula Ib with a catalytic amount of said inorganic ruthenium salt, in particular said (+III) or (+IV) salt and said oxidant, wherein the initial molar ratio of compound of formula Ia or Ib and oxidant is in the range of 1:1 to 1:5, in particular 1:1.5 to 1:3.
52. The process of anyone of the embodiments 46 to 51, wherein the mono- or polyvalent metal ligand is added to the reaction mixture, so that the molar ratio of ruthenium (+III) or (+IV) salt to ligand is in the range of 1:1 to 1:5, in particular 1:1.5 to 1:2.5.
53. The process of anyone of the embodiments 44 to 52, wherein the obtained lactam derivative is selected from Levetiracetam of the formula XIIIa and Brivaracetam of the formula XXIa and Piracetam of the formula XX.

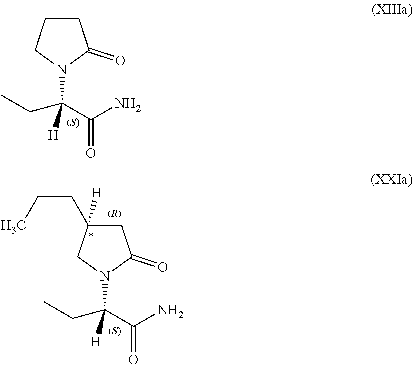

54. The process of anyone of the embodiments 44 to 53, further comprising the recovering and electrochemical recycling of the spent oxidant, in particular of the electrochemical oxidation of an alkali halogenate back to an alkali perhalogenate oxidant, more particularly the electrochemical oxidation of an alkali iodate, especially sodium or potassium iodate, back to an alkali periodate oxidant, especially sodium or potassium periodate oxidant.
55. The process of embodiment 54, wherein the electrochemical recycling comprises an anodic oxidation of said alkali halogenate back to said alkali perhalogenate oxidant.
56. The process of embodiment 54 or 55, wherein a boron-doped diamond anode is applied.
57. The method of anyone of the embodiments 54 to 56, wherein the anodic oxidation is performed under at least one of the following conditions:
   a) (1) aqueous solution of at least one alkali halogenate, in particular iodate, at an initial concentration of 0.001 to 5 M or 0.001 to 1 M, more preferably from 0.001 to 2 M, in particular from 0.01 to 1 M or 0.01 to 0.5 M or 0.01 to 0.4 M or 0.05 to 0.25 M, and specifically from 0.1 to 0.3 M or 0.1 to 0.25 M. (2) initial molarity of the base in the alkaline solution in the range of 0.3 to 5 M, preferably 0.6 to 3 M, in particular 0.9 to 2 M and specifically 1 M; (3) optionally the ratio of base to halogenate being 10:1 or higher, or particularly in the range of 10:1 to 1:1, more particularly from 8:1 to 2:1, even more particular 6:1 to 3:1, and specifically 5:1 to 4:1, wherein the base is selected from alkali metal and alkaline earth metal hydroxides or carbonates, in particular K₂CO₃, LiOH, NaOH, KOH, CsOH and Ba(OH)₂, more preferably NaOH, KOH, and most preferred NaOH,
   b) pH of the aqueous solution of 7 or more, like at a pH of at least 8, preferably of at least 10, in particular of at least 12,more particular at least 13, and specifically of at least 14,
   c) temperature in the range of 0 to 80° C., more preferably from 10 to 60° C., in particular from 20 to 30° C. and specifically from 20 to 25° C.,
   d) voltage in the range of 1 to 30V particularly from 1 to 20 V and more particular from 1 to 10 V,
   e) current density in the range of 10 to 500 mA/cm², like from 50 to 150 mA/cm², in particular from 80 to 120 mA/cm² and specifically of ca. 100 mA/cm²; and
   f) applied charge in the range of 1 to 10 Farad, more preferably from 2 to 6 F, in particular from 2.5 to 4 F, and specifically 2.75 to 3.5 Farad.
      In particular, the optimum current density j can be determined with respect to the type of electrolysis applied by a skilled person in the art. Batch or divided batch electrolyses, use current densities in the range of 10 to 1000 mA/cm². If the oxidation is to be performed in an electrolytic flow cell, the flow rate determines the maximum current density to be applicable. For example, in a flow cell with 48 cm² anode surface area, an anode-membrane gap of 1 mm, and a flow rate of 7.5 L/h, the optimal current density may be determined to be in a range of about 400-500 mA/cm², and specifically about 416 mA/cm².
      In general, at higher flow rates or higher halogenate (like iodate) concentrations, the current density j may be higher, while at lower flow rates or lower halogenate (like iodate) concentrations, the current density must be lower to maintain current efficiency (CE).
      In a particular embodiment, the initial molarity co of the base in the aqueous alkaline solution of the alkali halogenate is in the range of 0.3 to 5 M or 0.5 to 5 M, preferably 0.6 to 4 M, 0.8 to 4 M or 0.6 to 3 M, in particular 0.9 to 2 M and specifically 1 M. In particular the base is NaOH or KOH and the alkali halogenate is sodium or potassium iodate. More particularly the base is NaOH and the alkali halogenate is sodium iodate.
      In another particular embodiment, the pH of the aqueous solution is at least 12, at least 13 and specifically at least 14.
      In another particular embodiment, the initial concentration co of the at least one alkali halogenate, more particularly alkali iodate, especially sodium or potassium iodate, in said aqueous solution is low and is in the range of 0.001 to 1 M, in particular from 0.01 to 0.5 M or 0.01 to 0.4 M, and specifically from 0.05 to 0.25 M.
      In another particular embodiment, the ratio of c₀ (NaOH):c₀ (NaIO₃) is set in the range of 10:1 to 1:1, preferably 8:1 to 2:1, in particular 6:1 to 3:1, specifically 5:1 to 4:1.
      In another particular embodiment, a feature combination comprising at least features a), b), e) and f) is applied. Here feature a) may comprise either features a(1) and a(2), or features a(2) and a(3), or more preferably features a(1), a(2) and a(3).
      In another particular embodiment, a feature combination comprising at least the above features a) and b) is applied. Here feature a) may comprise either features a(1) and a(2), or features a(2) and a(3), or more preferably features a(1), a(2) and a(3).
      According to a particular embodiment thereof the alkali metal is sodium, and the obtained product is sodium periodate, obtained as sodium para-periodate.
      According to said particular embodiment the following particular parameters are applied alone or in combination:
         current density j in the range of 50 to 100 mA/cm² in batch electrolysis; or a current density j in the range of 400 to 500 mA/cm² in flow electrolysis (as for example observed at a flow rate of 7.5 L/h, an anode-membrane gap of 1 mm, and 48 cm² anode surface area);
         applied charge Q in the range of 3 to 4 F
         initial concentration cₒ (NaIO₃) of about 0.21 M
         initial concentration cₒ (NaOH) of about 1.0 M
         ratio of cₒ (NaIO₃):cₒ (NaOH) of about 1:5
      In a further particular embodiment of the iodate preparation process of the invention, the para-periodate as preferentially obtained by electrolysis is converted to meta-periodate.
      For this purpose, after electrolysis, para-periodate is isolated from the anolyte as described in more detail below. The precipitate is obtained from the liquid phase in the anode chamber by filtration or decantation. The precipitation may be completed by usual means, for example by the addition of sodium hydroxide or by concentration of the solvent. In order to obtain meta-periodate said para-periodate is neutralized by the addition of acid, in particular sulfuric or nitric acid and then recrystallized in a manner known per se.

### Further Aspects and Embodiments of the Invention

### 1. Polypeptides of the Invention

In this context the following definitions apply:

The generic terms “polypeptide” or “peptide”, which may be used interchangeably, refer to a natural or synthetic linear chain or sequence of consecutive, peptidically linked amino acid residues, comprising about 10 to up to more than 1.000 residues. Short chain polypeptides with up to 30 residues are also designated as “oligopeptides”.

The term “protein” refers to a macromolecular structure consisting of one or more polypeptides. The amino acid sequence of its polypeptide(s) represents the “primary structure” of the protein. The amino acid sequence also predetermines the “secondary structure” of the protein by the formation of special structural elements, such as alpha-helical and beta-sheet structures formed within a polypeptide chain. The arrangement of a plurality of such secondary structural elements defines the “tertiary structure” or spatial arrangement of the protein. If a protein comprises more than one polypeptide chains said chains are spatially arranged forming the “quaternary structure” of the protein. A correct spacial arrangement or “folding” of the protein is prerequisite of protein function. Denaturation or unfolding destroys protein function. If such destruction is reversible, protein function may be restored by refolding.

A typical protein function referred to herein is an “enzyme function”, i.e. the protein acts as biocatalyst on a substrate, for example a chemical compound, and catalyzes the conversion of said substrate to a product. An enzyme may show a high or low degree of substrate and/or product specificity.

A “polypeptide” referred to herein as having a particular “activity” thus implicitly refers to a correctly folded protein showing the indicated activity, as for example a specific enzyme activity.

Thus, unless otherwise indicated the term “polypeptide” also encompasses the terms “protein” and “enzyme”.

Similarly, the term “polypeptide fragment” encompasses the terms “protein fragment” and “enzyme fragment”.

The term “isolated polypeptide” refers to an amino acid sequence that is removed from its natural environment by any method or combination of methods known in the art and includes recombinant, biochemical and synthetic methods.

“Target peptide” refers to an amino acid sequence which targets a protein, or polypeptide to intracellular organelles, i.e., mitochondria, or plastids, or to the extracellular space (secretion signal peptide). A nucleic acid sequence encoding a target peptide may be fused to the nucleic acid sequence encoding the amino terminal end, e.g., N-terminal end, of the protein or polypeptide, or may be used to replace a native targeting polypeptide.

The present invention also relates to “functional equivalents” (also designated as “analogs” or “functional mutations”) of the polypeptides specifically described herein.

For example, “functional equivalents” refer to polypeptides which, in a test used for determining enzymatic NHase activity display at least a 1 to 10%, or at least 20%, or at least 50%, or at least 75%, or at least 90% higher or lower activity, as that of the polypeptides specifically described herein.

“Functional equivalents”, according to the invention, also cover particular mutants, which, in at least one sequence position of an amino acid sequences stated herein, have an amino acid that is different from that concretely stated one, but nevertheless possess one of the aforementioned biological activities, as for example enzyme activity. “Functional equivalents” thus comprise mutants obtainable by one or more, like 1 to 20, in particular 1 to 15 or 5 to 10 amino acid additions, substitutions, in particular conservative substitutions, deletions and/or inversions, where the stated changes can occur in any sequence position, provided they lead to a mutant with the profile of properties according to the invention. Functional equivalence is in particular also provided if the activity patterns coincide qualitatively between the mutant and the unchanged polypeptide, i.e. if, for example, interaction with the same agonist or antagonist or substrate, however at a different rate, (i.e. expressed by a EC₅₀ or IC₅₀ value or any other parameter suitable in the present technical field) is observed. Examples of suitable (conservative) amino acid substitutions are shown in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn ; Gln |
| Ile | Leu; Val |
| Leu | Ile; Val |
| Lys | Arg ; Gln ; Glu |
| Met | Leu ; Ile |
| Phe | Met; Leu ; Tyr |
| Ser | Thr |
| Thr | Ser |
| Trp | Tyr |
| Tyr | Trp ; Phe |
| Val | Ile; Leu |

“Functional equivalents” in the above sense are also “precursors” of the polypeptides described herein, as well as “functional derivatives” and “salts” of the polypeptides.

“Precursors” are in that case natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. Salts of acid addition, for example salts with inorganic acids, such as hydrochloric acid or sulfuric acid and salts with organic acids, such as acetic acid and oxalic acid, are also covered by the invention.

“Functional derivatives” of polypeptides according to the invention can also be produced on functional amino acid side groups or at their N-terminal or C-terminal end using known techniques. Such derivatives comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxyl groups, produced by reaction with acyl groups.

“Functional equivalents” naturally also comprise polypeptides that can be obtained from other organisms, as well as naturally occurring variants. For example, areas of homologous sequence regions can be established by sequence comparison, and equivalent polypeptides can be determined on the basis of the concrete parameters of the invention.

“Functional equivalents” also comprise “fragments”, like individual domains or sequence motifs, of the polypeptides according to the invention, or N- and or C-terminally truncated forms, which may or may not display the desired biological function. Particularly such “fragments” retain the desired biological function at least qualitatively.

“Functional equivalents” are, moreover, fusion proteins, which have one of the polypeptide sequences stated herein or functional equivalents derived there from and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

“Functional equivalents” which are also comprised in accordance with the invention are homologs to the specifically disclosed polypeptides. These have at least 60%, particularly at least 75%, in particular at least 80 or 85%, such as, for example, 90, 91, 92, 93, 94, 95, 96, 97, 98 or 99%, homology (or identity) to one of the specifically disclosed amino acid sequences, calculated by the algorithm of Pearson and Lipman, Proc. Natl. Acad, Sci. (USA) 85(8), 1988, 2444-2448. A homology or identity, expressed as a percentage, of a homologous polypeptide according to the invention means in particular an identity, expressed as a percentage, of the amino acid residues based on the total length of one of the amino acid sequences described specifically herein.

The identity data, expressed as a percentage, may also be determined with the aid of BLAST alignments, algorithm blastp (protein-protein BLAST), or by applying the Clustal settings specified herein below.

In the case of a possible protein glycosylation, “functional equivalents” according to the invention comprise polypeptides as described herein in deglycosylated or glycosylated form as well as modified forms that can be obtained by altering the glycosylation pattern.

Functional equivalents or homologues of the polypeptides according to the invention can be produced by mutagenesis, e.g. by point mutation, lengthening or shortening of the protein or as described in more detail below.

Functional equivalents or homologs of the polypeptides according to the invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are a great many methods that can be used for the production of databases of potential homologues from a degenerated oligonucleotide sequence. Chemical synthesis of a degenerated gene sequence can be carried out in an automatic DNA synthesizer, and the synthetic gene can then be ligated in a suitable expression vector. The use of a degenerated genome makes it possible to supply all sequences in a mixture, which code for the desired set of potential protein sequences. Methods of synthesis of degenerated oligonucleotides are known to a person skilled in the art.

In the prior art, several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologues according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologues.

An embodiment provided herein provides orthologs and paralogs of polypeptides disclosed herein as well as methods for identifying and isolating such orthologs and paralogs. A definition of the terms “ortholog” and “paralog” is given below and applies to amino acid and nucleic acid sequences.

The polypeptides of the invention include all active forms, including active subsequences, e.g., catalytic domains or active sites, of an enzyme of the invention. In one aspect, the invention provides catalytic domains or active sites as set forth below. In one aspect, the invention provides a peptide or polypeptide comprising or consisting of an active site domain as predicted through use of a database such as Pfam (http://pfam.wustl.edu/hmm-search.shtml) (which is a large collection of multiple sequence alignments and hidden Markov models covering many common protein families, The Pfam protein families database, A. Bateman, E. Birney, L. Cerruti, R. Durbin, L. Etwiller, S. R. Eddy, S. Griffiths-Jones, K. L. Howe, M. Marshall, and E. L. L. Sonnhammer, Nucleic Acids Research, 30(1):276-280, 2002) or equivalent, as for example InterPro and SMART databases (http://www.ebi.uc.uk/in-terpro/scan.html, http://smart.embl-heidelberg.de/).

The invention also encompasses “polypeptide variant” having the desired activity, wherein the variant polypeptide is selected from an amino acid sequence having at least 50%. 55%, 60%, 65%, 70%, 75%, 80%, 85%, 90%, 91%, 92%, 93%, 94%, 95%, 96%, 97%, 98% or 99%, sequence identity to a specific, in particular natural, amino acid sequence as referred to by a specific SEQ ID NO and contains at least one substitution modification relative said SEQ ID NO.

### 2. Nucleic Acids and Constructs

### 2.1 Nucleic Acids

In this context the following definitions apply:

The terms “nucleic acid sequence,” “nucleic acid,” “nucleic acid molecule” and “polynucleotide” are used interchangeably meaning a sequence of nucleotides. A nucleic acid sequence may be a single-stranded or double-stranded deoxyribonucleotide, or ribonucleotide of any length, and include coding and non-coding sequences of a gene, exons, introns, sense and anti-sense complimentary sequences, genomic DNA, cDNA, miRNA, siRNA, mRNA, rRNA, tRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference of thymine (T) being replaced by uracil (U). The term “nucleotide sequence” should also be understood as comprising a polynucleotide molecule or an oligonucleotide molecule in the form of a separate fragment or as a component of a larger nucleic acid.

An “isolated nucleic acid” or “isolated nucleic acid sequence” relates to a nucleic acid or nucleic acid sequence that is in an environment different from that in which the nucleic acid or nucleic acid sequence naturally occurs and can include those that are substantially free from contaminating endogenous material.

The term “naturally-occurring” as used herein as applied to a nucleic acid refers to a nucleic acid that is found in a cell of an organism in nature and which has not been intentionally modified by a human in the laboratory.

A “fragment” of a polynucleotide or nucleic acid sequence refers to contiguous nucleotides that is particularly at least 15 bp, at least 30 bp, at least 40 bp, at least 50 bp and/or at least 60 bp in length of the polynucleotide of an embodiment herein. Particularly the fragment of a polynucleotide comprises at least 25, more particularly at least 50, more particularly at least 75, more particularly at least 100, more particularly at least 150, more particularly at least 200, more particularly at least 300, more particularly at least 400, more particularly at least 500, more particularly at least 600, more particularly at least 700, more particularly at least 800, more particularly at least 900, more particularly at least 1000 contiguous nucleotides of the polynucleotide of an embodiment herein. Without being limited, the fragment of the polynucleotides herein may be used as a PCR primer, and/or as a probe, or for anti-sense gene silencing or RNAi.

As used herein, the term “hybridization” or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein below. Appropriate hybridization conditions can also be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

“Recombinant nucleic acid sequences” are nucleic acid sequences that result from the use of laboratory methods (for example, molecular cloning) to bring together genetic material from more than on source, creating or modifying a nucleic acid sequence that does not occur naturally and would not be otherwise found in biological organisms.

“Recombinant DNA technology” refers to molecular biology procedures to prepare a recombinant nucleic acid sequence as described, for instance, in Laboratory Manuals edited by Weigel and Glazebrook, 2002, Cold Spring Harbor Lab Press; and Sambrook et al., 1989, Cold Spring Harbor, N.Y., Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising, e.g., sequences involved in translation initiation, a coding region of cDNA or genomic DNA, introns, exons, and/or a 3′non-translated sequence comprising, e.g., transcription termination sites.

“Polycistronic” refers to nucleic acid molecules, in particular mRNAs, that can encode more than one polypeptide separately within the same nucleic acid molecule

A “chimeric gene” refers to any gene which is not normally found in nature in a species, in particular, a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription). The term “chimeric gene” also includes genes obtained through the combination of portions of one or more coding sequences to produce a new gene.

A “3′ UTR” or “3′ non-translated sequence” (also referred to as “3′ untranslated region,” or “3′end”) refers to the nucleic acid sequence found downstream of the coding sequence of a gene, which comprises, for example, a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly(A) tail may be added, which is involved in the transport of the mRNA to the site of translation, e.g., cytoplasm.

The term “primer” refers to a short nucleic acid sequence that is hybridized to a template nucleic acid sequence and is used for polymerization of a nucleic acid sequence complementary to the template.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

The invention also relates to nucleic acid sequences that code for polypeptides as defined herein.

In particular, the invention also relates to nucleic acid sequences (single-stranded and double-stranded DNA and RNA sequences, e.g. cDNA, genomic DNA and mRNA), coding for one of the above polypeptides and their functional equivalents, which can be obtained for example using artificial nucleotide analogs.

The invention relates both to isolated nucleic acid molecules, which code for polypeptides according to the invention or biologically active segments thereof, and to nucleic acid fragments, which can be used for example as hybridization probes or primers for identifying or amplifying coding nucleic acids according to the invention.

The present invention also relates to nucleic acids with a certain degree of “identity” to the sequences specifically disclosed herein. “Identity” between two nucleic acids means identity of the nucleotides, in each case over the entire length of the nucleic acid.

The “identity” between two nucleotide sequences (the same applies to peptide or amino acid sequences) is a function of the number of nucleotide residues (or amino acid residues) or that are identical in the two sequences when an alignment of these two sequences has been generated. Identical residues are defined as residues that are the same in the two sequences in a given position of the alignment. The percentage of sequence identity, as used herein, is calculated from the optimal alignment by taking the number of residues identical between two sequences dividing it by the total number of residues in the shortest sequence and multiplying by 100. The optimal alignment is the alignment in which the percentage of identity is the highest possible. Gaps may be introduced into one or both sequences in one or more positions of the alignment to obtain the optimal alignment. These gaps are then taken into account as nonidentical residues for the calculation of the percentage of sequence identity. Alignment for the purpose of determining the percentage of amino acid or nucleic acid sequence identity can be achieved in various ways using computer programs and for instance publicly available computer programs available on the world wide web.

Particularly, the BLAST program (Tatiana et al, FEMS Microbiol Lett., 1999, 174:247-250, 1999) set to the default parameters, available from the National Center for Biotechnology Information (NCBI) website at ncbi.nlm.nih.gov/BLAST/b12seq/wblast2.cgi, can be used to obtain an optimal alignment of protein or nucleic acid sequences and to calculate the percentage of sequence identity.

In another example the identity may be calculated by means of the Vector NTI Suite 7.1 program of the company Informax (USA) employing the Clustal Method (Higgins D G, Sharp P M. ((1989))) with the following settings:

| Multiple alignment parameters: | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |
| Pairwise alignment parameter: | |
| FAST algorithm | on |
| K-tuple size | 1 |
| Gap penalty | 3 |
| Window size | 5 |
| Number of best diagonals | 5 |

Alternatively the identity may be determined according to Chenna, et al. (2003), the web page: http://www.ebi.ac.uk/Tools/clustalw/index.html# and the following settings

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

All the nucleic acid sequences mentioned herein (single-stranded and double-stranded DNA and RNA sequences, for example cDNA and mRNA) can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al. (1989), see below.

The nucleic acid molecules according to the invention can in addition contain non-translated sequences from the 3′ and/or 5′ end of the coding genetic region.

The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Such probes or primers generally comprise a nucleotide sequence region which hybridizes under “stringent” conditions (as defined herein elsewhere) on at least about 12, particularly at least about 25, for example about 40, 50 or 75 successive nucleotides of a sense strand of a nucleic acid sequence according to the invention or of a corresponding antisense strand.

“Homologous” sequences include orthologous or paralogous sequences. Methods of identifying orthologs or paralogs including phylogenetic methods, sequence similarity and hybridization methods are known in the art and are described herein.

“Paralogs” result from gene duplication that gives rise to two or more genes with similar sequences and similar functions. Paralogs typically cluster together and are formed by duplications of genes within related plant species. Paralogs are found in groups of similar genes using pair-wise Blast analysis or during phylogenetic analysis of gene families using programs such as CLUSTAL. In paralogs, consensus sequences can be identified characteristic to sequences within related genes and having similar functions of the genes.

“Orthologs”, or orthologous sequences, are sequences similar to each other because they are found in species that descended from a common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in host cells or organisms, such as plants or microorganisms, overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making the host cells, organism such as plants or microorganisms producing enzymes of the invention.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

A nucleic acid molecule according to the invention can be recovered by means of standard techniques of molecular biology and the sequence information supplied according to the invention. For example, cDNA can be isolated from a suitable cDNA library, using one of the concretely disclosed complete sequences or a segment thereof as hybridization probe and standard hybridization techniques (as described for example in Sambrook, (1989)).

In addition, a nucleic acid molecule comprising one of the disclosed sequences or a segment thereof, can be isolated by the polymerase chain reaction, using the oligonucleotide primers that were constructed on the basis of this sequence. The nucleic acid amplified in this way can be cloned in a suitable vector and can be characterized by DNA sequencing. The oligonucleotides according to the invention can also be produced by standard methods of synthesis, e.g. using an automatic DNA synthesizer.

Nucleic acid sequences according to the invention or derivatives thereof, homologues or parts of these sequences, can for example be isolated by usual hybridization techniques or the PCR technique from other bacteria, e.g. via genomic or cDNA libraries. These DNA sequences hybridize in standard conditions with the sequences ac-cording to the invention.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These “standard conditions” vary depending on the nucleic acid used (oligonucleotide, longer fragment or complete sequence) or depending on which type of nucleic acid—DNA or RNA—is used for hybridization. For example, the melting temperatures for DNA:DNA hybrids are approx. 10° C. lower than those of DNA:RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA:DNA hybrids are 0.1×SSC and temperatures between about 20° C. to 45° C., particularly between about 30° C. to 45° C. For DNA:RNA hybrids the hybridization conditions are advantageously 0.1×SSC and temperatures between about 30° C. to 55° C., particularly between about 45° C. to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., 1989, and can be calculated using formulae that are known by a person skilled in the art, for example depending on the length of the nucleic acids, the type of hybrids or the G+C content. A person skilled in the art can obtain further information on hybridization from the following textbooks: Ausubel et al. (eds), (1985), Brown (ed) (1991).

“Hybridization” can in particular be carried out under stringent conditions. Such hybridization conditions are for example described in Sambrook (1989), or in Current Protocols in Molecular Biology, John Wiley & Sons, N.Y. (1989), 6.3.1-6.3.6.

As used herein, the term hybridization or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein.

Appropriate hybridization conditions can be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

As used herein, defined conditions of low stringency are as follows. Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 18-20 h at 40° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of moderate stringency are as follows. Filters containing DNA are pretreated for 7 h at 50° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 30 h at 50° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 μg/ml denatured salmon sperm DNA and 5-20×106 cpm of 32Plabeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes.

Other conditions of low, moderate, and high stringency well known in the art (e.g., as employed for cross-species hybridizations) may be used if the above conditions are inappropriate (e.g., as employed for cross-species hybridizations).

A detection kit for nucleic acid sequences encoding a polypeptide of the invention may include primers and/or probes specific for nucleic acid sequences encoding the polypeptide, and an associated protocol to use the primers and/or probes to detect nucleic acid sequences encoding the polypeptide in a sample. Such detection kits may be used to determine whether a plant, organism, microorganism or cell has been modified, i.e., transformed with a sequence encoding the polypeptide.

To test a function of variant DNA sequences according to an embodiment herein, the sequence of interest is operably linked to a selectable or screenable marker gene and expression of said reporter gene is tested in transient expression assays, for example, with microorganisms or with protoplasts or in stably transformed plants.

The invention also relates to derivatives of the concretely disclosed or derivable nucleic acid sequences.

Thus, further nucleic acid sequences according to the invention can be derived from the sequences specifically disclosed herein and can differ from it by one or more, like 1 to 20, in particular 1 to 15 or 5 to 10 additions, substitutions, insertions or deletions of one or several (like for example 1 to 10) nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism.

According to a particular embodiment of the invention variant nucleic acids may be prepared in order to adapt its nucleotide sequence to a specific expression system. For example, bacterial expression systems are known to more efficiently express polypeptides if amino acids are encoded by particular codons. Due to the degeneracy of the genetic code, more than one codon may encode the same amino acid sequence, multiple nucleic acid sequences can code for the same protein or polypeptide, all these DNA sequences being encompassed by an embodiment herein. Where appropriate, the nucleic acid sequences encoding the polypeptides described herein may be optimized for increased expression in the host cell. For example, nucleic acids of an embodiment herein may be synthesized using codons particular to a host for improved expression.

The invention also encompasses naturally occurring variants, e.g. splicing variants or allelic variants, of the sequences described therein.

Allelic variants may have at least 60% homology at the level of the derived amino acid, particularly at least 80% homology, quite especially particularly at least 90% homology over the entire sequence range (regarding homology at the amino acid level, reference should be made to the details given above for the polypeptides). Advantageously, the homologies can be higher over partial regions of the sequences.

The invention also relates to sequences that can be obtained by conservative nucleotide substitutions (i.e. as a result thereof the amino acid in question is replaced by an amino acid of the same charge, size, polarity and/or solubility).

The invention also relates to the molecules derived from the concretely disclosed nucleic acids by sequence polymorphisms. Such genetic polymorphisms may exist in cells from different populations or within a population due to natural allelic variation. Allelic variants may also include functional equivalents. These natural variations usually produce a variance of 1 to 5% in the nucleotide sequence of a gene. Said polymorphisms may lead to changes in the amino acid sequence of the polypeptides disclosed herein. Allelic variants may also include functional equivalents.

Furthermore, derivatives are also to be understood to be homologs of the nucleic acid sequences according to the invention, for example animal, plant, fungal or bacterial homologs, shortened sequences, single-stranded DNA or RNA of the coding and noncoding DNA sequence. For example, homologs have, at the DNA level, a homology of at least 40%, particularly of at least 60%, especially particularly of at least 70%, quite especially particularly of at least 80% over the entire DNA region given in a sequence specifically disclosed herein.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

### 2.2 Constructs for Expressing Polypeptides of the Invention

In this context the following definitions apply:

“Expression of a gene” encompasses “heterologous expression” and “over-expression” and involves transcription of the gene and translation of the mRNA into a protein. Overexpression refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

An “expression vector” as used herein includes any linear or circular recombinant vector including but not limited to viral vectors, bacteriophages and plasmids. The skilled person is capable of selecting a suitable vector according to the expression system. In one embodiment, the expression vector includes the nucleic acid of an embodiment herein operably linked to at least one “regulatory sequence”, which controls transcription, translation, initiation and termination, such as a transcriptional promoter, operator or enhancer, or an mRNA ribosomal binding site and, optionally, including at least one selection marker. Nucleotide sequences are “operably linked” when the regulatory sequence functionally relates to the nucleic acid of an embodiment herein.

An “expression system” as used herein encompasses any combination of nucleic acid molecules required for the expression of one, or the co-expression of two or more polypeptides either in vivo of a given expression host, or in vitro. The respective coding sequences may either be located on a single nucleic acid molecule or vector, as for example a vector containing multiple cloning sites, or on a polycistronic nucleic acid, or may be distributed over two or more physically distinct vectors. As a particular example there may be mentioned an operon comprising a promotor sequence, one or more operator sequences and one or more structural genes each encoding an enzyme as described herein

As used herein, the terms “amplifying” and “amplification” refer to the use of any suitable amplification methodology for generating or detecting recombinant of naturally expressed nucleic acid, as described in detail, below. For example, the invention provides methods and reagents (e.g., specific degenerate oligonucleotide primer pairs, oligo dT primer) for amplifying (e.g., by polymerase chain reaction, PCR) naturally expressed (e.g., genomic DNA or mRNA) or recombinant (e.g., cDNA) nucleic acids of the invention in vivo, ex vivo or in vitro.

“Regulatory sequence” refers to a nucleic acid sequence that determines expression level of the nucleic acid sequences of an embodiment herein and is capable of regulating the rate of transcription of the nucleic acid sequence operably linked to the regulatory sequence. Regulatory sequences comprise promoters, enhancers, transcription factors, promoter elements and the like.

A “promoter”, a “nucleic acid with promoter activity” or a “promoter sequence” is understood as meaning, in accordance with the invention, a nucleic acid which, when functionally linked to a nucleic acid to be transcribed, regulates the transcription of said nucleic acid. “Promoter” in particular refers to a nucleic acid sequence that controls the expression of a coding sequence by providing a binding site for RNA polymerase and other factors required for proper transcription including without limitation transcription factor binding sites, repressor and activator protein binding sites. The meaning of the term promoter also includes the term “promoter regulatory sequence”. Promoter regulatory sequences may include upstream and downstream elements that may influences transcription, RNA processing or stability of the associated coding nucleic acid sequence. Promoters include naturally-derived and synthetic sequences. The coding nucleic acid sequences is usually located downstream of the promoter with respect to the direction of the transcription starting at the transcription initiation site.

In this context, a “functional” or “operative” linkage is understood as meaning for example the sequential arrangement of one of the nucleic acids with a regulatory sequence. For example the sequence with promoter activity and of a nucleic acid sequence to be transcribed and optionally further regulatory elements, for example nucleic acid sequences which ensure the transcription of nucleic acids, and for example a terminator, are linked in such a way that each of the regulatory elements can perform its function upon transcription of the nucleic acid sequence. This does not necessarily require a direct linkage in the chemical sense. Genetic control sequences, for example enhancer sequences, can even exert their function on the target sequence from more remote positions or even from other DNA molecules. Preferred arrangements are those in which the nucleic acid sequence to be transcribed is positioned behind (i.e. at the 3′-end of) the promoter sequence so that the two sequences are joined together covalently. The distance between the promoter sequence and the nucleic acid sequence to be expressed recombinantly can be smaller than 200 base pairs, or smaller than 100 base pairs or smaller than 50 base pairs.

In addition to promoters and terminator, the following may be mentioned as examples of other regulatory elements: targeting sequences, enhancers, polyadenylation signals, selectable markers, amplification signals, replication origins and the like. Suitable regulatory sequences are described, for example, in Goeddel, Gene Expression Technology: Methods in Enzymology 185, Academic Press, San Diego, Calif. (1990).

The term “constitutive promoter” refers to an unregulated promoter that allows for continual transcription of the nucleic acid sequence it is operably linked to.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous. The nucleotide sequence associated with the promoter sequence may be of homologous or heterologous origin with respect to the plant to be transformed. The sequence also may be entirely or partially synthetic. Regardless of the origin, the nucleic acid sequence associated with the promoter sequence will be expressed or silenced in accordance with promoter properties to which it is linked after binding to the polypeptide of an embodiment herein. The associated nucleic acid may code for a protein that is desired to be expressed or suppressed throughout the organism at all times or, alternatively, at a specific time or in specific tissues, cells, or cell compartment. Such nucleotide sequences particularly encode proteins conferring desirable phenotypic traits to the host cells or organism altered or transformed therewith. More particularly, the associated nucleotide sequence leads to the production of the product or products of interest as herein defined in the cell or organism. Particularly, the nucleotide sequence encodes a polypeptide having an enzyme activity as herein defined.

The nucleotide sequence as described herein above may be part of an “expression cassette”. The terms “expression cassette” and “expression construct” are used synonymously. The (particularly recombinant) expression construct contains a nucleotide sequence which encodes a polypeptide according to the invention and which is under genetic control of regulatory nucleic acid sequences.

In a process applied according to the invention, the expression cassette may be part of an “expression vector”, in particular of a recombinant expression vector.

An “expression unit” is understood as meaning, in accordance with the invention, a nucleic acid with expression activity which comprises a promoter as defined herein and, after functional linkage with a nucleic acid to be expressed or a gene, regulates the expression, i.e. the transcription and the translation of said nucleic acid or said gene. It is therefore in this connection also referred to as a “regulatory nucleic acid sequence”. In addition to the promoter, other regulatory elements, for example enhancers, can also be present.

An “expression cassette” or “expression construct” is understood as meaning, in accordance with the invention, an expression unit which is functionally linked to the nucleic acid to be expressed or the gene to be expressed. In contrast to an expression unit, an expression cassette therefore comprises not only nucleic acid sequences which regulate transcription and translation, but also the nucleic acid sequences that are to be expressed as protein as a result of transcription and translation.

The terms “expression” or “overexpression” describe, in the context of the invention, the production or increase in intracellular activity of one or more polypeptides in a microorganism, which are encoded by the corresponding DNA. To this end, it is possible for example to introduce a gene into an organism, replace an existing gene with another gene, increase the copy number of the gene(s), use a strong promoter or use a gene which encodes for a corresponding polypeptide with a high activity; optionally, these measures can be combined.

Particularly such constructs according to the invention comprise a promoter 5′-upstream of the respective coding sequence and a terminator sequence 3′-downstream and optionally other usual regulatory elements, in each case in operative linkage with the coding sequence.

Nucleic acid constructs according to the invention comprise in particular a sequence coding for a polypeptide for example derived from the amino acid related SEQ ID NOs as described therein or the reverse complement thereof, or derivatives and homologs thereof and which have been linked operatively or functionally with one or more regulatory signals, advantageously for controlling, for example increasing, gene expression.

In addition to these regulatory sequences, the natural regulation of these sequences may still be present before the actual structural genes and optionally may have been genetically modified so that the natural regulation has been switched off and expression of the genes has been enhanced. The nucleic acid construct may, however, also be of simpler construction, i.e. no additional regulatory signals have been inserted before the coding sequence and the natural promoter, with its regulation, has not been removed. Instead, the natural regulatory sequence is mutated such that regulation no longer takes place and the gene expression is increased.

A preferred nucleic acid construct advantageously also comprises one or more of the already mentioned “enhancer” sequences in functional linkage with the promoter, which sequences make possible an enhanced expression of the nucleic acid sequence. Additional advantageous sequences may also be inserted at the 3′-end of the DNA sequences, such as further regulatory elements or terminators. One or more copies of the nucleic acids according to the invention may be present in a construct. In the construct, other markers, such as genes which complement auxotrophisms or antibiotic resistances, may also optionally be present so as to select for the construct.

Examples of suitable regulatory sequences are present in promoters such as cos, tac, trp, tet, trp-tet, lpp, lac, lpp-lac, lacI^{q}, T7, T5, T3, gal, trc, ara, rhaP (rhaP_{BAD})SP6, lambda-P_{R} or in the lambda-P_{L} promoter, and these are advantageously employed in Gram-negative bacteria. Further advantageous regulatory sequences are present for example in the Gram-positive promoters amy and SPO2, in the yeast or fungal promoters ADC1, MFalpha, AC, P-60, CYC1, GAPDH, TEF, rp28, ADH. Artificial promoters may also be used for regulation.

For expression in a host organism, the nucleic acid construct is inserted advantageously into a vector such as, for example, a plasmid or a phage, which makes possible optimal expression of the genes in the host. Vectors are also understood as meaning, in addition to plasmids and phages, all the other vectors which are known to the skilled worker, that is to say for example viruses such as SV40, CMV, baculovirus and adenovirus, transposons, IS elements, phasmids, cosmids and linear or circular DNA or artificial chromosomes. These vectors are capable of replicating autonomously in the host organism or else chromosomally. These vectors are a further development of the invention. Binary or cpo-integration vectors are also applicable.

Suitable plasmids are, for example, in E. coli μLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCl, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The abovementioned plasmids are a small selection of the plasmids which are possible. Further plasmids are well known to the skilled worker and can be found for example in the book Cloning Vectors (Eds. Pouwels P. H. et al. Elsevier, Amsterdam-New York-Oxford, 1985, ISBN 0 444 904018).

In a further development of the vector, the vector which comprises the nucleic acid construct according to the invention or the nucleic acid according to the invention can advantageously also be introduced into the microorganisms in the form of a linear DNA and integrated into the host organism's genome via heterologous or homologous recombination. This linear DNA can consist of a linearized vector such as a plasmid or only of the nucleic acid construct or the nucleic acid according to the invention.

For optimal expression of heterologous genes in organisms, it is advantageous to modify the nucleic acid sequences to match the specific “codon usage” used in the organism. The “codon usage” can be determined readily by computer evaluations of other, known genes of the organism in question.

An expression cassette according to the invention is generated by fusing a suitable promoter to a suitable coding nucleotide sequence and a terminator or polyadenylation signal. Customary recombination and cloning techniques are used for this purpose, as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which makes possible optimal expression of the genes in the host. Vectors are well known to the skilled worker and can be found for example in “cloning vectors” (Pouwels P. H. et al., Ed., Elsevier, Amsterdam-New York-Oxford, 1985).

An alternative embodiment of an embodiment herein provides a method to “alter gene expression” in a host cell. For instance, the polynucleotide of an embodiment herein may be enhanced or overexpressed or induced in certain contexts (e.g. upon exposure to certain temperatures or culture conditions) in a host cell or host organism.

Alteration of expression of a polynucleotide provided herein may also result in ectopic expression which is a different expression pattern in an altered and in a control or wild-type organism. Alteration of expression occurs from interactions of polypeptide of an embodiment herein with exogenous or endogenous modulators, or as a result of chemical modification of the polypeptide. The term also refers to an altered expression pattern of the polynucleotide of an embodiment herein which is altered below the detection level or completely suppressed activity.

In one embodiment, provided herein is also an isolated, recombinant or synthetic polynucleotide encoding a polypeptide or variant polypeptide provided herein.

In one embodiment, several polypeptide encoding nucleic acid sequences are co-expressed in a single host, particularly under control of different promoters. In another embodiment, several polypeptide encoding nucleic acid sequences can be present on a single transformation vector or be co-transformed at the same time using separate vectors and selecting transformants comprising both chimeric genes. Similarly, one or polypeptide encoding genes may be expressed in a single plant, cell, microorganism or organism together with other chimeric genes.

### 3. Hosts to be Applied for the Present Invention

Depending on the context, the term “host” can mean the wild-type host or a genetically altered, recombinant host or both.

In principle, all prokaryotic or eukaryotic organisms may be considered as host or recombinant host organisms for the nucleic acids or the nucleic acid constructs according to the invention.

Using the vectors according to the invention, recombinant hosts can be produced, which are for example transformed with at least one vector according to the invention and can be used for producing the polypeptides according to the invention. Advantageously, the recombinant constructs according to the invention, described above, are introduced into a suitable host system and expressed. Particularly common cloning and transfection methods, known by a person skilled in the art, are used, for example co-precipitation, protoplast fusion, electroporation, retroviral transfection and the like, for expressing the stated nucleic acids in the respective expression system. Suitable systems are described for example in Current Protocols in Molecular Biology, F. Ausubel et al., Ed., Wiley Interscience, New York 1997, or Sambrook et al. Molecular Cloning: A Laboratory Manual. 2nd edition, Cold Spring Harbor Laboratory, Cold Spring Harbor Laboratory Press, Cold Spring Harbor, N.Y., 1989.

Advantageously, microorganisms such as bacteria, fungi or yeasts are used as host organisms. Advantageously, gram-positive or gram-negative bacteria are used, particularly bacteria of the families Enterobacteriaceae, Pseudomonadaceae, Rhizobiaceae, Streptomycetaceae, Streptococcaceae or Nocardiaceae, especially particularly bacteria of the genera Escherichia, Pseudomonas, Streptomyces, Lactococcus, Nocardia, Burkholderia, Salmonella, Agrobacterium, Clostridium or Rhodococcus. The genus and species Escherichia coli is quite especially preferred. Furthermore, other advantageous bacteria are to be found in the group of alpha-Proteobacteria, beta-Proteobacteria or gamma-Proteobacteria. Advantageously also yeasts of families like Saccharomyces or Pichia are suitable hosts.

Alternatively, entire plants or plant cells may serve as natural or recombinant host. As non-limiting examples the following plants or cells derived therefrom may be mentioned the genera Nicotiana, in particular Nicotiana benthamiana and Nicotiana tabacum (tobacco); as well as Arabidopsis, in particular Arabidopsis thaliana.

Depending on the host organism, the organisms used in the method according to the invention are grown or cultured in a manner known by a person skilled in the art. Culture can be batchwise, semi-batchwise or continuously. Nutrients can be present at the beginning of fermentation or can be supplied later, semicontinuously or continuously. This is also described in more detail below.

### 4. Recombinant Production of the Enzymes and Mutants

The invention further relates to methods for recombinant production of polypeptides according to the invention or functional, biologically active fragments thereof, wherein a polypeptide-producing microorganism is cultured, optionally the expression of the polypeptides is induced by applying at least one inducer inducing gene expression and the expressed polypeptides are isolated from the culture. The polypeptides can also be produced in this way on an industrial scale, if desired.

The microorganisms produced according to the invention can be cultured continuously or discontinuously in the batch method or in the fed-batch method or repeated fed-batch method. A summary of known cultivation methods can be found in the textbook by Chmiel (Bioprozesstechnik 1. Einführung in die Bioverfahrenstechnik [Bioprocess technology 1. Introduction to bioprocess technology] (Gustav Fischer Verlag, Stuttgart, 1991)) or in the textbook by Storhas (Bioreaktoren and periphere Einrichtungen [Bioreactors and peripheral equipment] (Vieweg Verlag, Braunschweig/Wiesbaden, 1994)).

The culture medium to be used must suitably meet the requirements of the respective strains. Descriptions of culture media for various microorganisms are given in the manual “Manual of Methods for General Bacteriology” of the American Society for Bacteriology (Washington D. C., USA, 1981).

These media usable according to the invention usually comprise one or more carbon sources, nitrogen sources, inorganic salts, vitamins and/or trace elements.

Preferred carbon sources are sugars, such as mono-, di- or polysaccharides. Very good carbon sources are for example glucose, fructose, mannose, galactose, ribose, sorbose, ribulose, lactose, maltose, sucrose, raffinose, starch or cellulose. Sugars can also be added to the media via complex compounds, such as molasses, or other by-products of sugar refining. It can also be advantageous to add mixtures of different carbon sources. Other possible carbon sources are oils and fats, for example soybean oil, sunflower oil, peanut oil and coconut oil, fatty acids, for example palmitic acid, stearic acid or linoleic acid, alcohols, for example glycerol, methanol or ethanol and organic acids, for example acetic acid or lactic acid.

Nitrogen sources are usually organic or inorganic nitrogen compounds or materials that contain these compounds. Examples of nitrogen sources comprise ammonia gas or ammonium salts, such as ammonium sulfate, ammonium chloride, ammonium phosphate, ammonium carbonate or ammonium nitrate, nitrates, urea, amino acids or complex nitrogen sources, such as corn-steep liquor, soya flour, soya protein, yeast extract, meat extract and others. The nitrogen sources can be used alone or as a mixture.

Inorganic salt compounds that can be present in the media comprise the chloride, phosphorus or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, as well as organic sulfur compounds, such as mercaptans and thiols, can be used as the sulfur source.

Phosphoric acid, potassium dihydrogen phosphate or dipotassium hydrogen phosphate or the corresponding sodium-containing salts can be used as the phosphorus source.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention usually also contain other growth factors, such as vitamins or growth promoters, which include for example biotin, riboflavin, thiamine, folic acid, nicotinic acid, pantothenate and pyridoxine. Growth factors and salts often originate from the components of complex media, such as yeast extract, molasses, corn-steep liquor and the like. Moreover, suitable precursors can be added to the culture medium. The exact composition of the compounds in the medium is strongly dependent on the respective experiment and is decided for each specific case individually. Information on media optimization can be found in the textbook “Applied Microbiol. Physiology, A Practical Approach” (Ed. P.M. Rhodes, P. F. Stanbury, IRL Press (1997) p. 53-73, ISBN 0 19 963577 3). Growth media can also be obtained from commercial suppliers, such as Standard 1 (Merck) or BHI (brain heart infusion, DIFCO) and the like.

All components of the medium are sterilized, either by heat (20 min at 1.5 bar and 121° C.) or by sterile filtration. The components can either be sterilized together, or separately if necessary. All components of the medium can be present at the start of culture or can be added either continuously or batchwise.

The culture temperature is normally between 15° C. and 45° C., particularly 25° C. to 40° C. and can be varied or kept constant during the experiment. The pH of the medium should be in the range from 5 to 8.5, particularly around 7.0. The pH for growing can be controlled during growing by adding basic compounds such as sodium hydroxide, potassium hydroxide, ammonia or ammonia water or acid compounds such as phosphoric acid or sulfuric acid. Antifoaming agents, for example fatty acid polyglycol esters, can be used for controlling foaming. To maintain the stability of plasmids, suitable selective substances, for example antibiotics, can be added to the medium. To maintain aerobic conditions, oxygen or oxygen-containing gas mixtures, for example ambient air, are fed into the culture. The temperature of the culture is normally in the range from 20° C. to 45° C. The culture is continued until a maximum of the desired product has formed. This target is normally reached within 10 hours to 160 hours.

The fermentation broth is then processed further. Depending on requirements, the biomass can be removed from the fermentation broth completely or partially by separation techniques, for example centrifugation, filtration, decanting or a combination of these methods or can be left in it completely.

If the polypeptides are not secreted in the culture medium, the cells can also be lysed and the product can be obtained from the lysate by known methods for isolation of proteins. The cells can optionally be disrupted with high-frequency ultrasound, high pressure, for example in a French press, by osmolysis, by the action of detergents, lytic enzymes or organic solvents, by means of homogenizers or by a combination of several of the aforementioned methods.

The polypeptides can be purified by known chromatographic techniques, such as molecular sieve chromatography (gel filtration), such as Q-sepharose chromatography, ion exchange chromatography and hydrophobic chromatography, and with other usual techniques such as ultrafiltration, crystallization, salting-out, dialysis and native gel electrophoresis. Suitable methods are described for example in Cooper, T. G., Biochemische Arbeitsmethoden [Biochemical processes], Verlag Walter de Gruyter, Berlin, New York or in Scopes, R., Protein Purification, Springer Verlag, New York, Heidelberg, Berlin.

For isolating the recombinant protein, it can be advantageous to use vector systems or oligonucleotides, which lengthen the cDNA by defined nucleotide sequences and therefore code for altered polypeptides or fusion proteins, which for example serve for easier purification. Suitable modifications of this type are for example so-called “tags” functioning as anchors, for example the modification known as hexa-histidine anchor or epitopes that can be recognized as antigens of antibodies (described for example in Harlow, E. and Lane, D., 1988, Antibodies: A Laboratory Manual. Cold Spring Harbor (N.Y.) Press). These anchors can serve for attaching the proteins to a solid carrier, for example a polymer matrix, which can for example be used as packing in a chromatography column, or can be used on a microtiter plate or on some other carrier.

At the same time these anchors can also be used for recognition of the proteins. For recognition of the proteins, it is moreover also possible to use usual markers, such as fluorescent dyes, enzyme markers, which form a detectable reaction product after reaction with a substrate, or radioactive markers, alone or in combination with the anchors for derivatization of the proteins.

### 5. Polypeptide Immobilization

The enzymes or polypeptides according to the invention can be used free or immobilized in the method described herein. An immobilized enzyme is an enzyme that is fixed to an inert carrier. Suitable carrier materials and the enzymes immobilized thereon are known from EP-A-1149849, EP-A-1 069 183 and DE-OS 100193773 and from the references cited therein. Reference is made in this respect to the disclosure of these documents in their entirety. Suitable carrier materials include for example clays, clay minerals, such as kaolinite, diatomaceous earth, perlite, silica, aluminum oxide, sodium carbonate, calcium carbonate, cellulose powder, anion exchanger materials, synthetic polymers, such as polystyrene, acrylic resins, phenol formaldehyde resins, polyurethanes and polyolefins, such as polyethylene and polypropylene. For making the supported enzymes, the carrier materials are usually employed in a finely-divided, particulate form, porous forms being preferred. The particle size of the carrier material is usually not more than 5 mm, in particular not more than 2 mm (particle-size distribution curve). Similarly, when using dehydrogenase as whole-cell catalyst, a free or immobilized form can be selected. Carrier materials are e.g. Ca-alginate, and carrageenan. Enzymes as well as cells can also be crosslinked directly with glutaraldehyde (cross-linking to CLEAs). Corresponding and other immobilization techniques are described for example in J. Lalonde and A. Margolin “Immobilization of Enzymes” in K. Drauz and H. Waldmann, Enzyme Catalysis in Organic Synthesis 2002, Vol. III, 991-1032, Wiley-VCH, Weinheim. Further information on biotransformations and bioreactors for carrying out methods according to the invention are also given for example in Rehm et al. (Ed.) Biotechnology, 2nd Edn, Vol 3, Chapter 17, VCH, Weinheim.

### 6. Reaction Conditions for Biocatalytic Production Methods of the Invention

The reaction of the present invention may be performed under in vivo or in vitro conditions.

The at least one polypeptide/enzyme which is present during a method of the invention or an individual step of a multistep-method as defined herein above, can be present in living cells naturally or recombinantly producing the enzyme or enzymes, in harvested cells. i.e. under in vivo conditions, or, in dead cells, in permeabilized cells, in crude cell extracts, in purified extracts, or in essentially pure or completely pure form, i.e. under in vitro conditions. The at least one enzyme may be present in solution or as an enzyme immobilized on a carrier. One or several enzymes may simultaneously be present in soluble and/or immobilised form.

The methods according to the invention can be performed in common reactors, which are known to those skilled in the art, and in different ranges of scale, e.g. from a laboratory scale (few millilitres to dozens of litres of reaction volume) to an industrial scale (several litres to thousands of cubic meters of reaction volume). If the polypeptide is used in a form encapsulated by non-living, optionally permeabilized cells, in the form of a more or less purified cell extract or in purified form, a chemical reactor can be used. The chemical reactor usually allows controlling the amount of the at least one enzyme, the amount of the at least one substrate, the pH, the temperature and the circulation of the reaction medium. When the at least one polypeptide/enzyme is present in living cells, the process will be a fermentation. In this case the biocatalytic production will take place in a bioreactor (fermenter), where parameters necessary for suitable living conditions for the living cells (e.g. culture medium with nutrients, temperature, aeration, presence or absence of oxygen or other gases, antibiotics, and the like) can be controlled. Those skilled in the art are familiar with chemical reactors or bioreactors, e.g. with procedures for up-scaling chemical or biotechnological methods from laboratory scale to industrial scale, or for optimizing process parameters, which are also extensively described in the literature (for biotechnological methods see e.g. Crueger and Crueger, Biotechnologie—Lehrbuch der angewandten Mikrobiologie, 2. Ed., R. Oldenbourg Verlag, München, Wien, 1984).

Cells containing the at least one enzyme can be permeabilized by physical or mechanical means, such as ultrasound or radiofrequency pulses, French presses, or chemical means, such as hypotonic media, lytic enzymes and detergents present in the medium, or combination of such methods. Examples for detergents are digitonin, n-dodecylmaltoside, octylglycoside, Triton® X-100, Tween 20, deoxycholate, CHAPS (3-[(3-Cholamidopropyl)dimethylammonio]-1-propansulfonate), Nonidet P40 (Ethylphenolpoly(ethyleneglycolether), and the like.

Instead of living cells biomass of non-living cells containing the required biocatalyst(s) may be applied of the biotransformation reactions of the invention as well.

If the at least one enzyme is immobilised, it is attached to an inert carrier as described above.

The conversion reaction can be carried out batch wise, semi-batch wise or continuously. Reactants (and optionally nutrients) can be supplied at the start of reaction or can be supplied subsequently, either semi-continuously or continuously.

The reaction of the invention, depending on the particular reaction type, may be performed in an aqueous, aqueous-organic or non-aqueous, in particular aqueous or aqueous-organic reaction medium.

An aqueous or aqueous-organic medium may contain a suitable buffer in order to adjust the pH to a value in the range of 5 to 11, like 6 to 10.

In an aqueous-organic medium an organic solvent miscible, partly miscible or immiscible with water may be applied. Non-limiting examples of suitable organic solvents are listed below. Further examples are mono- or polyhydric, aromatic or aliphatic alcohols, in particular polyhydric aliphatic alcohols like glycerol.

The concentration of the reactants/substrates may be adapted to the optimum reaction conditions, which may depend on the specific enzyme applied. For example, the initial substrate concentration may be in the 0.1 to 0.5 M, as for example 10 to 100 mM.

The reaction temperature may be adapted to the optimum reaction conditions, which may depend on the specific enzyme applied. For example, the reaction may be performed at a temperature in a range of from 0 to 70° C., as for example 0 to 50 or 5 to 35° C. Examples for reaction temperatures are about 10° C., about 15° C., about 20° C., about 25° C., about 30° C., and about 35° C.

The process may proceed until equilibrium between the substrate and then product(s) is achieved, but may be stopped earlier. Usual process times are in the range from 1 minute to 25 hours, in particular 10 min to 6 hours, as for example in the range from 1 hour to 4 hours, in particular 1.5 hours to 3.5 hours. These parameters are non-limiting examples of suitable process conditions.

If the host is a transgenic plant, optimal growth conditions can be provided, such as optimal light, water and nutrient conditions, for example.

### 7. Chemical Oxidation

According to the present invention, a particular class of oxidation catalyst systems are suitable for the region-specific and stereo-conserving chemical oxidation of the pyrrolidine substrates of above formula I, in particular of (S)-2-(pyrrolidin-1-yl)butanamide (2).

The catalyst may be a homogenous or a heterogeneous catalyst, as described in more detail below.

The chemical oxidation of step 3) is performed with particular oxidation catalysts capable of oxidizing the heterocyclic alpha-amino group in a compound of formula (Ia) or (Ib) under substantial retention of the stereo configuration at the asymmetric carbon atom in alpha-position to the amide group to provide the final product in an essentially stereo-chemically pure form.

The oxidation catalyst is selected from combinations of an inorganic ruthenium (+III), (+IV), (+V), or (+VI), in particular (+III) or (+IV) salts and at least one oxidant capable of in situ oxidizing ruthenium (+III), (+IV), (+V), or (+VI), in particular (+III) or (+IV), in particular to ruthenium (+VIII), and optionally in the presence of a mono- or polyvalent metal ligand, as for example sodium oxalate.

Said inorganic ruthenium (+III) or (+IV) salt is selected from RuCl₃, RuO₂ and the respective hydrates, in particular monohydrates, thereof.

Said inorganic ruthenium (+V) or (+VI) salt is selected from RuF₅ or RuF₆.

The oxidant may be selected from perhalogenates, hypohalogenites (in particular hypochlorite, NaClO), halogenates (in particular bromate, NaBrO₃) Oxone (KHSO₅·½ KHSO₄·½ K₂SO₄), tert-butyl hydroperoxide (t-BuOOH), hydrogen peroxide (H₂O₂), molecular iodine (I₂), N-methylmorpholin-N-oxide, potassium persulfate (K₂S₂O₈), (Diacetoxyiodo)benzene, N-Bromosuccinimide, tert-butyl peroxybenzoate, iron(III) chloride or combinations thereof. A preferred group of oxidants is selected from perhalogenates, preferably alkali perhalogenates, more preferably sodium or potassium perhalogenates, in particular sodium or potassium periodate, and specifically sodium meta-periodate or combinations thereof.

Another group of oxidants represents hypohalogenites and hydrates thereof, preferably alkali hypohalogenites, more preferably sodium or potassium hypohalogenites, in particular sodium or potassium hypochlorite pentahydrate, or combinations thereof.

Another group of oxidants represents combinations of the above described groups of hypohalogenites and perhalogenates.

### (i) Homogenous Oxidation Process

The oxidation reaction may be performed by dissolving the substrate of formula I in a suitable aqueous or organic solvent, either a non-polar aprotic, essentially water immiscible solvent, as for example carboxylic esters, like ethyl acetate, ethers or hydrocarbons (aliphatic or aromatic) or halogenated hydrocarbons (aliphatic or aromatic) or an organic solvent miscible with water, e.g. acetonitrile, acetone, N-methyl-2-pyrrolidone, or N,N-dimethylformamid. The solvent of the solution of the substrate of formula I preferably is selected from water, more preferably from a mixture of water and at least one of said organic solvents miscible with water, and even more preferably of at least one of said organic solvents or mixtures of at least two of said organic solvents. In another preferred embodiment, the substrate may be added neat.

Afterwards an aqueous solution or aqueous/organic solution mixture of the ruthenium salt and at least one oxidant for in situ oxidation of the ruthenium cation are added, optionally stepwise. Alternatively the aqueous or organic solution or aqueous/organic solution mixture of the substrate may be added, optionally stepwise, to the preformed aqueous solution or aqueous/organic solution mixture of the ruthenium salt and the at least one oxidant. The final solvent mixture is preferably composed of pure water, more preferably of a water/organic solvent mixture, in particular a mixture of water/acetone, water/ethyl acetate, water/acetonitrile, water/N-methyl-2-pyrrolidone, or water/N,N-dimethylformamid, and specifically water/acetonitrile. The final ratio of the water/organic solvent mixture is preferably from neat water to neat organic solvent, more preferably from 4:1 to 1:4 v/v, in particular 4:2 to 2:4 v/v, and specifically 1:1 v/v.

For performing the reaction, the initial substrate concentration may be chosen in a range depending on the solubility of the substrate in the respective solvent, as for example in a range of 0.001 to 1 mol/l. If the substrate is added neat, the initial substrate concentration is chosen in a range depending on the solubility of the substrate in the respective catalyst mixture, preferably in a range of 0.001 to 1 mol/l, more preferably from 0.01 to 0.5 mol/l, in particular from 0.1 to 0.2 mol/l, and specifically 0.107 mol/l. The substrate may also be added in amounts larger than the solubility product.

For performing the reaction it is preferred to apply the oxidant in a molar excess over the substrate, preferably in a 1 to 10-fold, more preferably in a 1.1 to 5-fold, in particular in a 2 to 3-fold, and specifically in a 2.6-fold excess.

For performing the reaction, it is preferred to apply the ruthenium salt in catalytic amounts relative to the substrate, as for example in a range of 0.001 to 100 mol %, preferably 0.005 to 10 mol %, in particular 0.05 to 1 mol %, and specifically 0.5 mol %.

The reaction is performed under stirring of the reaction mixture, or optionally the reaction may be performed without stirring. The generation of the active ruthenium catalyst may be aided by sonification.

The reaction is performed in an open or preferably closed reaction vessel.

The oxidation is carried out at pH value preferably between 2 and 12, more preferable between 4 and 10, in particular between 6 and 8, and specifically at pH 7.

The reaction temperature is chosen from a temperature in the range depending on the melting point of the respective solvent mixture, preferably from −20 to 80° C., more preferably −10 to 60° C., in particular −5 to 30 grade, and specifically at 0° C.

After termination of the reaction, preferably after 10 to 240 minutes, in particular after 20 to 60 minutes, and specifically after 30 minutes the reaction product may be isolated from the organic or the aqueous phase.

### (ii) Heterogeneous Oxidation Process

In another preferred embodiment, the stereospecific chemical oxidation of substrates of formula I, in particular of (S)-2-(pyrrolidin-1-yl)butanamide (2) is performed in a continuous, heterogeneous method. While in the batch (or discontinuous; time-related) method the electrolyte containing the substrate is subjected to oxidation and after a certain time this is stopped and the product is isolated from the reaction vessel, in a continuous process design the substrate solution is passed continuously through a catalyst-containing material, preferably containing the catalyst in immobilized form.

For the immobilization, the said ruthenium salt is immobilized on an inert solid carrier material. The ruthenium salt, preferably, Ru(III)Cl or RuO₂, in particular the respective hydrates, and specifically ruthenium dioxide hydrate is mixed with the carrier material, as for example aluminum oxide, char coal, polyacrylonitrile, or alkylated silica, or combinations thereof. The mass of the ruthenium salt per 25 g carrier material ranges from preferably 1 mg to 5 g, more preferably from 50 mg to 2 g, in particular from 100 mg to 1 g, and specifically 200 mg. The said carrier material was loaded on a column. The size of the column may be chosen in a range depending on the substrate concentration and/or the scale of the oxidation process, as for example a diameter of 1.5 cm and a length of 15 cm. Various designs and geometries of columns are known in the art and can be applied to the present method.

The substrate of formula I and at least one oxidant are dissolved in pure water, in an organic solvent, or in solvent mixtures thereof. The same solvents and mixtures as described above for the homogeneous process may be applied.

The concentration of the substrate ranges preferably from 0.001 to 10 mol/l, more preferably from 0.01 to 5 mol/l, in particular 0.1 to 1 mol/l, and specifically 0.05 mol/l.

The solvent mixture ratio ranges from preferably neat water to 2:4 v/v water:organic solvent, more preferably from 4:1 to 1:4 v/v, in particular 4:2 to 2:4 v/v, and specifically 1:1 v/v.

The oxidant(s) is/are used in a molar excess over the substrate, preferably in a 1 to 10-fold, more preferably in a 1.1 to 5-fold, in particular in a 2 to 3-fold, and specifically in a 2.6-fold excess.

For performing the reaction, the solution of the substrate is piped through the column by using a suitable pump or by another suitable pressure-generating arrangement. The flow rate is chosen in the range depending on the substrate concentration and/or the scale of the oxidation process, as for example 2 I/hand can easily adapted by one skilled in the art. The solution of the substrate may pass the column (material) once or multiple times.

The reaction temperature is chosen from a temperature in the range depending on the melting point of the respective solvent mixture, preferably from −20 to 80° C., more preferably −10 to 60° C., in particular −5 to 30° C., and specifically at 0° C.

The oxidation is carried out at pH value preferably between 2 and 12, more preferable between 4 and 10, in particular between 6 and 8, and specifically at pH 7.

### 8. Product Isolation

The methodology of the present invention can further include a step of recovering an end or intermediate product, optionally in stereoisomerically or enantiomerically substantially pure form.

The term “recovering” includes extracting, harvesting, isolating or purifying the compound from culture or reaction media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like. Identity and purity of the isolated product may be determined by known techniques, like High Performance Liquid Chromatography (HPLC), gas chromatography (GC), Spektroskopy (like IR, UV, NMR), Colouring methods, TLC, NIRS, enzymatic or microbial assays. (see for example: Patek et al. (1994) Appl. Environ. Microbiol. 60:133-140; Malakhova et al. (1996) Biotekhnologiya 11 27-32; und Schmidt et al. (1998) Bioprocess Engineer. 19:67-70. Ullmann's Encyclopedia of Industrial Chemistry (1996) Bd. A27, VCH: Weinheim, S. 89-90, S. 521-540, S. 540-547, S. 559-566, 575-581 und S. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Bd. 17.)

In all embodiments of the claimed process described herein, the isolation or the workup of a product depend on the desired product and the reaction conditions inter alia and are principally known to those skilled in the art.

For instance, to obtain the oxidation product of formula III, specifically (S)-α-ethyl-2-oxopyrrolidine acetamide (XIIIa), the generated iodate and residues of periodates are removed by precipitation. The precipitation is forced by less polar water miscible solvents or by reducing the temperature; if necessary after concentration of the reaction medium. Concentration, if required, can be carried out by usual means, such as evaporation of a part of the solvent, if desired under reduced pressure, partial freeze-drying, partial reverse osmosis etc. The precipitated product can be isolated by usual means, such as filtration or decantation of the supernatant. To remove catalyst or metal residues or undesired impurities, the product-containing filtrate or solution may be treated with charcoal. The charcoal is removed by usual means, such as filtration or decantation of the supernatant. The solvent of the product-containing solution is then concentrated or removed by usual means, such as evaporation, etc., and, if desired, the product is crystallized and/or recrystallized.

Alternatively, the solvent can be removed from the reaction medium, for example by evaporation of the solvent, if desired under reduced pressure, freeze-drying, reverse osmosis, etc. The residue can be purified by usual means, e.g. recrystallization, chromatography, or extraction.

If appropriate the reaction product may further processed by further purifying a particular stereoisomer, in case the product is composed of a mixture of two or more stereoisomers, as for example (S)- and (R)-enantiomers by applying conventional preparative separation methods like chiral chromatography or by resolution.

The intermediates and final products produced in any of the method described herein can be converted to derivatives such as, but not limited to esters, glycosides, ethers, epoxides, aldehydes, ketones, or alcohols. The derivatives can be obtained by a chemical method such as, but not limited to oxidation, reduction, alkylation, acylation and/or rearrangement. Alternatively, the compound derivatives can be obtained using a biochemical method by contacting the compound with an enzyme such as, but not limited to an oxidoreductase, a monooxygenase, a dioxygenase, a transferase. The biochemical conversion can be performed in-vitro using isolated enzymes, enzymes from lysed cells or in-vivo using whole cells.

### 9. Electrochemical Recycling of Halogenate/Iodate and the De Novo Synthesis of Petr Halogenate/Periodate from Halogenate/Iodate

In another preferred embodiment, the produced halogenate, preferably iodate is recovered from the reaction mixtures of the oxidation process of a substrate of formula I, and the oxidation of halogenate/iodate to perhalogenate/periodate is performed electrochemically by anodic oxidation. A related process, i.e. the anodic oxidation of iodide to periodate at boron-doped diamond electrodes was described in a European patent application in the name of PharmaZell GmbH (EP 19214206.5, filing date Dec. 6, 2019).

However, it is well understood, that the recycling process of an alkali iodate according to the present invention is not limited to the particular process described herein with respect to the oxidation process of a substrate of above formula I. Alkali iodate, as formed form alkali periodate by any type of oxidation reaction, may be recycled to generate the alkali periodate oxidant. In such reaction media the initial concentration c₀ of the halogenate, more particularly of alkali iodate, especially of sodium or potassium iodate, may be in the range of 0.001 to 1 M, in particular from 0.01 to 0.5 M or 0.01 to 0.4 M, and specifically from 0.05 to 0.25 M. As non-limiting example cellulose processing industry, like paper industry may be mentioned as a technical field for applying the present process. In paper industry cellulose may be treated by oxidation. Cellulose is effectively oxidized to dialdehyde cellulose (DAC) by consumption of sodium periodate and formation of sodium iodate, which may then be recycled electrochemically according to the present invention.

The recovery of the iodate for the recycling meaning the isolation or the work-up of such from the reaction medium of periodate-based oxidations, preferably from the reaction mixture of the oxidation of substrates of formula I, depends on the desired product or the reaction conditions inter alia and are principally known to those skilled in the art.

For instance, to obtain the generated halogenate, preferably iodate, and in particular sodium iodate, the reaction medium is mixed with less polar water miscible solvents, preferably alcohols, carboxylic acids, carboxylic esters, ethers, amides, pyrrolidones, carbonates, tetramethylurea or nitriles, in particular ethanol, iso-propanol or methanol, acetic acid, ethyl acetate, tetrahydrofuran, N-methylpyrrolidone, N,N-dimethylformamid, N,N-dimethylacetamide, or acetonitrile to force precipitation. The precipitated halogenate can be isolated by usual means, such as filtration or decantation of the supernatant. If desired, the precipitate can then be subjected to further purification steps in order to remove undesired side products etc., if any, such as by washing with organic solvent (mixtures), or by recrystallization.

The electrolysis cell in which the anodic oxidation is carried out comprises one or more anodes in one or more anode compartments and one or more cathodes in one or more cathode compartments, where the anode compartments are preferably separated from the cathode compartments. If more than one anode is used, the two or more anodes can be arranged in the same anode compartment or in separate compartments. If the two or more anodes are present in the same compartment, they can be arranged next to each other or on top of each other. The same applies to the case that one or more cathodes are used. In case of two or more electrolysis cells, they can be arranged next to each other or on top of each other. The separation of the anode compartment(s) from the cathode compartment(s) can be accomplished by using different electrolysis cells for cathode(s) and anode(s) and connecting these cells by a salt bridge for charge equalization. The separators separate the anolyte that is the liquid medium in the anode compartment(s) from the catholyte that is the liquid medium in the cathode compartment(s), but allow charge equalization. Diaphragms are separators comprising porous structures of an oxidic material, such as silicates, e.g. in the form of porcelain or ceramics. Due to the sensitivity of diaphragm materials to harsher conditions, semipermeable membranes are however generally preferred, especially if the reaction is carried out at basic pH, as it is preferred. Membrane materials, which resist harsher conditions, especially basic pH, are based on fluorinated polymers. Examples for suitable materials for this type of membranes are sulfonated tetrafluoroethylene based fluoropolymer-copolymers, such as the Nafion® brand from DuPont de Nemours or the Gore-Select® brand from W.L. Gore & Associates, Inc. If the reaction is carried out in batch, the anode and cathode compartments are generally designed as batch cells. If the reaction is carried out semi-continuously or continuously, the anode and cathode compartments are generally designed as flow cells. Various designs and geometries of electrolysis cells are known to those skilled in the art and can be applied to the present method.

As anode (or electrode, more generally speaking) carbon-comprising materials may be used. Carbon-comprising anodes/electrodes are well known in the art and include for example graphite electrodes, vitreous carbon (glassy carbon) electrodes, reticulated vitreous carbon electrodes, carbon fiber electrodes, electrodes based on carbonized composites, electrodes based on carbon-silicon composites, graphene-based electrodes and boron diamond-based electrodes.

Electrodes are not necessarily composed entirely of the mentioned material, but may consist of a coated carrier material, for instance silicon, self-passivating metals, such as germanium, zirconium, niobium, titanium, tantalum, molybdenum and tungsten, metal carbides, graphite, glassy carbon, carbon fibers and combinations thereof.

Suitable self-passivating metals are for example germanium, zirconium, niobium, titanium, tantalum, molybdenum and tungsten.

Suitable combinations are for example metal carbide layers on the corresponding metal (such an interlayer may be formed in situ when a diamond layer is applied to the metal support), composites of two or more of the above-listed support materials and combinations of carbon and one or more of the other elements listed above. Examples for composites are siliconized carbon fiber carbon composites (CFC) and partially carbonized composites.

Preferably, the support material is selected from the group consisting of elemental silicon, germanium, zirconium, niobium, titanium, tantalum, molybdenum, tungsten, carbides of the eight aforementioned metals, graphite, glassy carbon, carbon fibers and combinations (in particular composites) thereof.

More preference is given to elemental silicon, germanium, zirconium, niobium, titanium, tantalum, molybdenum, tungsten and a combination of one of the seven afore-mentioned metals with the respective metal carbide.

Among the anode materials, preference is given to boron-doped diamond. The boron-doped diamond comprises boron in an amount of preferably 0.02 to 1% by weight (200 to 10,000 ppm), more preferably of 0.04 to 0.2% by weight, in particular of 0.06 to 0.09% by weight, relative to the total weight of the doped diamond.

As already indicated above, such electrodes are generally not composed of doped diamond alone. Rather, the doped diamond is attached to a substrate. Most frequently, the doped diamond is present as a layer on a conducting substrate, but diamond particle electrodes, in which doped diamond particles are embedded into a conducting or non-conducting substrate are suitable as well. Preference is however given to anodes in which the doped diamond is present as a layer on a conducting substrate.

Doped diamond electrodes and methods for preparing them are known in the art and described, for example, in the above-mentioned Janssen article in Electrochimica Acta 2003, 48, 3959, in NL1013348C2 and the references cited therein. Suitable preparation methods include, for example, chemical vapour deposition (CVD), such as hot filament CVD or microwave plasma CVD, for preparing electrodes with doped diamond films; and high temperature high pressure (HTHP) methods for preparing electrodes with doped diamond particles. Doped diamond electrodes are commercially available.

The cathode material is not very critical, and any commonly used material is suitable, such as stainless steel, chromium-nickel steel, platinum, nickel, bronze, tin, zirconium or carbon-comprising electrodes. In a specific embodiment, a stainless steel electrode is used as cathode.

Suitably, the electrochemical oxidation of the iodate is carried out in aqueous medium. Thus, the method of the invention comprises subjecting an aqueous solution comprising the iodate, in particular a metal iodate to anodic oxidation.

The electrolysis may be carried out under galvanostatic control (i.e. the applied current is controlled; voltage may be measured, but is not controlled) or potentiostatic control (i.e. the applied voltage is controlled; current may be measured, but is not controlled), the former being preferred.

In case of the preferred galvanostatic control, the observed voltage is generally in the range of from 1 to 30 V, more frequently from 1 to 20 V and in particular from 1 to 10 V.

In case of potentiostatic control, the applied voltage is generally in the same range, i.e. from 1 to 30 V, preferably from 1 to 20 V, in particular from 1 to 10 V.

The anodic oxidation is preferably carried out at a current density in the range of from 10 to 500 mA/cm², more preferably from 50 to 150 mA/cm², in particular from 80 to 120 mA/cm² and specifically of ca. 100 mA/cm².

To maximize the conversion of iodate to periodate, a charge of preferably at least 2 Farad, more preferably of at least 2.5 Farad, in particular of at least 2.75 Farad, and specifically of at least 3 Farad is applied. More particularly, a charge in the range of preferably 1 to 10 Farad, more preferably from 2 to 6 F, in particular from 2.5 to 4 F, and specifically 2.75 to 3.5 Farad is applied.

The electrolysis may be performed under acidic, neutral or basic conditions. Preferably the electrolysis is performed under basic conditions. Suitable bases to be used in the present method of the invention are all those which form hydroxide anions in the aqueous phase. Preferred are inorganic bases, such as metal hydroxides, metal oxides and metal carbonates, in particular alkali and earth alkali hydroxides. Preference is given to metal hydroxides where the metal of the base corresponds to the metal of the halogenate. The anodic oxidation is carried out at a pH of at least 8, preferably of at least 10, in particular of at least 12 and specifically of at least 14. Water is generally used as solvent.

The initial molarity of the iodate or halogenate solution is preferably from 0.0001 to 10 M, more preferably from 0.001 to 5 M, in particular from 0.01 to 2 M, and specifically from 0.1 to 1 M. The initial molarity of the base in the alkaline solution is 0.3 to 5 M, preferably 0.6 to 3 M, in particular 0.9 to 2 M and specifically 1 M. The ratio of base to halogenate is preferably from 10:1 to 1:1, more preferably from 8:1 to 2:1, in particular 6:1 to 3:1, specifically 5:1 to 4:1

The anodic oxidation is preferably carried out at a temperature of from 0 to 80° C., more preferably from 10 to 60° C., in particular from 20 to 30° C. and specifically from 20 to 25° C. The reaction pressure is not critical.

Under alkaline conditions, metal periodates are formed that are the metal salts of the various periodic acids. Periodate anions consist of an iodine in the oxidation state of +VII and include various structures, as for example ortho-periodate (IO₆⁵⁻), meta-periodate (IO₄⁻), para-periodate (H₂IO₆³⁻), mesoperiodates (IO₅³⁻), or dimesoperiodates (I₂O₉⁴⁻) inter alia, depending on the pH of the medium. Meta-periodate, may be obtained specifically by acid recrystallization as described by C. L. Mehltretter, C. S. Wise, U.S. Pat. No. 2,989,371A, 1961, or H. H. Willard, R. R. Ralston, Trans. Electrochem. Soc. 1932, 62, 239.

Periodate in form of the para-periodate is isolated from the anolyte by filtration. If necessary the precipitation is forced by concentration of the solvent, by addition of less polar water-miscible solvents, by increasing the pH value, or by decreasing the temperature inter alia. Concentration, if required, can be carried out by usual means, such as evaporation of a part of the solvent, if desired under reduced pressure, partial freeze-drying, partial reverse osmosis etc. For the addition of water-miscible solvent, if required, preferably alcohols, carboxylic acids, carboxylic esters, ethers, amides, pyrrolidones, carbonates, tetramethylurea or nitriles, in particular ethanol, iso-propanol or methanol, acetic acid, ethyl acetate, tetrahydrofuran, N-methylpyrrolidone, N,N-dimethylformamid, N,N-dimethylacetamide, or acetonitrile are used. For increasing the pH value of the anodic media, if required, a suitable base, preferably metal hydroxides having a metal corresponding to the metal in the metal peroxohalogenate. The precipitated product can be isolated by usual means, such as filtration or decantation of the supernatant. Residual solvent in the product may be removed by usual means, such as evaporation, storing it in a desiccator etc., and, if desired, the product is crystallized and/or recrystallized.

Alternatively, the solvent can be removed from the reaction medium, for example by evaporation of the solvent, if desired under reduced pressure, freeze-drying, reverse osmosis, etc. The residue can be purified by usual means, e.g. recrystallization, chromatography, or extraction.

The following examples are illustrative only and are not intended to limit the scope of the embodiments described herein.

The numerous possible variations that will become immediately evident to a person skilled in the art after heaving considered the disclosure provided herein also fall within the scope of the invention.

### Experimental Part

Unless stated otherwise, the cloning steps carried out in the context of the present invention, for example restriction cleavage, agarose gel electrophoresis, purification of DNA fragments, transfer of nucleic acids onto nitrocellulose and nylon membranes, linkage of DNA fragments, transformation of microorganisms, culturing of microorganisms, multiplication of phages and sequence analysis of recombinant DNA are, if not otherwise sated, carried out by applying well-known techniques, as for example described in Sambrook et al. (1989) op. cit.

### A. Biochemical Section

### 1. General

This section describes the work aiming at the identification of a robust, (S)-selective NHase for the synthesis of the target molecule (S)-2-(pyrrolidine-1-yl)butane amide. (2). Among the identified candidates capable of the target reaction, the best enzyme should be selected and engineered to improve its properties such as enantioselectivity.

The target molecule should be converted from the respective nitrile by the use of such enantioselective NHase by dynamic kinetic resolution of the starting materials (cf. Scheme 1 above).

The reaction conditions allowing racemization of the substrate were undetermined prior to the present invention but were predicted by the inventors to be at high temperature and/or pH values

### 2. Materials & Methods

### 2.1. Strains, Vectors, Enzymes and Primers

2.1.1 E. coli Strains

The cloning part of this work was done with Escherichia coli Top10F′ as the host. For protein expression, the strain E. coli BL21 Gold (DE3) was used. Both strains were obtained from Life Technologies (Carlsbad, Calif., USA).

2.1.2 Vectors

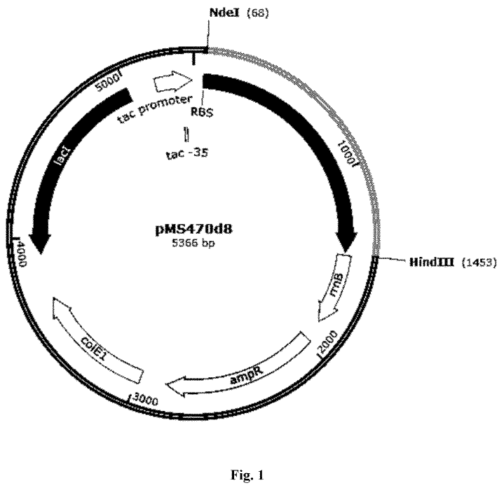

The expression vector used in this project was pMS470d8 [C. Reisinger, A. Kern, K. Fesko, H. Schwab, An efficient plasmid vector for expression cloning of large numbers of PCR fragments in Escherichia coli., Appl. Microbiol. Biotechnol. 77 (2007) 241-4. doi:10.1007/s00253-007-1151-1] (see FIG. 1). The plasmid encodes for the bacterial origin ColE1, an ampicillin resistance gene (ampR) and the gene regulator lacI. The system of tac promoter and rrnB terminator allows inducible expression. Using the restriction sites NdeI and HindIII removes the stuffer fragment d8 to give a suitable vector backbone.

2.1.3 Enzymes

TABLE 1
Gene and protein entries of tested nitrile hydratases.

| Organism | Abbr. | α subunit | β subunit | CDS |
|---|---|---|---|---|
| Afipia | Ab | EKS37369.1 | EKS37368.1 | AGWX01000004.1 |
| broomeae | | | | |
| Acinetobacter | Ac | ENV54396.1 | ENV54397.1 | |
| baylyi | | | | |
| Aurantimonas | Am | WP_ | WP_ | (2) |
| manganoxydans | | 009208459.1 | 009208458.1 | |
| Bradyrhizobium | Bj | BAC49763.1 | WP_ | (1) |
| japonicum | | | 028174056.1 | |
| Bacillus sp. | Br | AAO23015.1 | AAO23014.1 | AY184492.2 |
| Caballeronia | Cj | WP_ | WP_ | NZ_ |
| jiangsuensis | | 035501544.1 | 035501545.1 | JFHF01000013.1 |
| Comamonas | Ct | AAU87542.1 | AAU87543.1 | AY743666.1 (3) |
| testosteroni | | | | |
| Gordonia | Gh | WP_ | WP_ | NZ_ |
| hydrophobica | | 066163464.1 | 066163466.1 | BCWU01000002.1 |
| Klebsiella | Ko | OSY94202.1 | OSY94201.1 | MPJL01000034.1 |
| oxytoca | | | | |
| Microvirga | Ml | EIM25394.1 | EIM25395.1 | JH660647.1 |
| lotononidis | | | | |
| Nitriliruptor | Na | WP_ | WP_ | NZ_KQ033901.1 |
| alkaliphilus | | 052668589.1 | 052668588.1 | |
| Paenibacillus | Pc | WP_ | WP_ | NZ_ |
| chondroitinus | | 047675415.1 | 047675418.1 | JUGY01000006.1 |
| Pseudomonas | Pk | | | In-house sequence |
| kilonensis | | | | |
| Pseudomonas | Pm | WP_ | WP_ | |
| marginalis | | 074846646.1 | 074846644.1 | |
| Pseudonocardia | Pt | WP_ | WP_ | NZ_ |
| thermophila | | 073455624.1 | 073455623.1 | FRAP01000003.1 |
| Rhodococcus | Re | P13448.3 | P13449.1 | |
| erythropolis | | | | |
| Rhizobium | Rl | EJC80161.1 | EJC80160.1 | JH719395.1 |
| leguminosarum | | | | |
| Roseobacter sp. | Rm | OIQ35619.1 | OIQ35618.1 | MPCZ01000001.1 |
| Ralstonia | Rs | AMP38431.1 | AMP38430.1 | CP014702.1 |
| solanacearum | | | | |
| Tardiphaga | Tr | KZD20487.1 | KZD20486.1 | LVYV01000056.1 |
| robiniae | | | | |
| Variovorax sp. | Vv | WP_ | WP_ | AKIW01000013.1 |
| | | 042672800.1 | 007829432.1 | |

(1) J.L. Tucker, L. Xu, W. Yu, R.W. Scott, L. Zhao, N. Ran, Chemoenzymatic processes for preparation of levetiracetam, WO2009009117, 2009.
(2) X. Pei, Z. Yang, A. Wang, L. Yang, J. Wu, Identification and functional analysis of the activator gene involved in the biosynthesis of Co-type nitrile hydratase from Aurantimonas manganoxydans, J. Biotechnol. 251 (2017) 38-46. doi:10.1016/J.JBIOTEC.2017.03.016.
(3) K.L. Petrillo, S. Wu, E.C. Hann, F.B. Cooling, A. Ben-Bassat, J.E. Gavagan, R. DiCosimo, M.S. Payne, Over-expression in Escherichia coli of a thermally stable and regio-selective nitrile hydratase from Comamonas testosteroni 5-MGAM-4D, Appl. Microbiol. Biotechnol. 67 (2005) 664-670. doi:10.1007/s00253-004-1842-9

2.1.4 Primers

TABLE 2
List of primers used in this project

| SEQ | | |
|---|---|---|
| ID | | |
| NO | Name | sequence |
| | For generation of site-saturation libraries | |
| 28 | Ct-aQ93X_for | GGGTA [symbol] GGCGAGGACATG |
| 29 | Ct-aQ93X_rev | GCC [symbol] TACCCCGGAGAAG |
| 30 | Ct-aW120X_for | TACCCA [symbol] CCGACGCTGG |
| 31 | Ct-aW120X_rev | CAGCGTCGG [symbol] TGGGTAG |
| 32 | Ct-aP126X_for | TGGGCTTG [symbol] CCTGCCTG |
| 33 | Ct-aP126X_rev | GTACCAGGCAGG [symbol] CAAG |
| 34 | Ct-aK131X_for | GTAC [symbol] GCCCCGCCCTAC |
| 45 | Ct-aK131X_rev | GGGC [symbol] GTACCAGGCAG |
| 46 | Ct-aR169X_for | CGAATTG [symbol] TACATGGTGCTG |
| 37 | Ct-aR169X_rev | CAGCACCATGTA [symbol] CAATTCG |
| 38 | Ct-bM34X_for | CGGTC [symbol] TCCCTGTTCCC |
| 39 | Ct-bM34X_rev | CAGGGA [symbol] GACCGTTTTTTC |
| 40 | Ct-bF37X_for | CCCTG [symbol] CCGGCGCTGTTC |
| 41 | Ct-bF37X_rev | GCCGG [symbol] CAGGGACATGAC |
| 42 | Ct-bL48X_for | CAAC [symbol] GATGAGTTTCGACAC |
| 43 | Ct-bL48X_rev | GTCGAAACTCATC [symbol] GTTGAAG |
| 44 | Ct-bF51X_for | CGATGAG [symbol] CGACACGGC |
| 45 | Ct-bF51X_rev | GCCGTGTCG [symbol] CTCATCG |
| 46 | Ct-bY68X-long_for | AAGGGAACC [symbol] TACGAACACTGGATCCATTC |
| 47 | Ct-bY68X-long_rev | TGTTCGTA [symbol] GGTTCCCTTCAGGTAGTCG |
| | For site-directed mutagenesis of pMS470-CtNHase | |
| 48 | Ct-aW120F_for | GCTACCCA [symbol] CCGACGCTGG |
| 49 | Ct-aW120F_rev | GCGTCGG [symbol] TGGGTAGCAAGAG |
| 50 | Ct-bM34L_for | AAACGGTC [symbol] TCCCTGTTCCCGGCGCTGTTC |
| 51 | Ct-bM34L_rev | AACAGGGA [symbol] GACCGTTTTTTCCCAGTCGTAG |
| 52 | Ct-bM34Q_for | AAACGGTCC [symbol] TCCCTGTTCCCGGCGCTGTTC |
| 53 | Ct-bM34Q_rev | AACAGGGA [symbol] GACCG TTTTTTCCCAGTCGTAG |
| | For generation of randomly mutated CtNHase gene fragments | |
| 54 | Ct-alpha1_for | TGGCCAAGGCCTGGGTGGAC |
| 55 | Ct-alpha1_rev | GCAAGAGCACAAGGTGCAAAC |
| 56 | Ct-alpha2_for | CCTTGTGCTCTTGCTACCCA |
| 57 | Ct-alpha2_rev | TTCAGTTCCCGCGGGCCG |
| 58 | Ct-beta1_for | CGTCTTTCGCTACGACTGG |
| 59 | Ct-beta1_rev | AGGTTTCGATGGAATGGATCCA |
| 60 | Ct-beta2_for | GCTTCTGCCGCCCGGGAG |
| 61 | Ct-beta2_rev | TTTCCGTGTGCCGCGGTGTC |
| | For generation of pMS470-CtNHase backbone strains (restriction sites in grey) | |
| 62 | Ct-A1bb-lig_for | AATTT [symbol] TTTGCACCTTGTGCTCTTGCTAC |
| 63 | Ct-A1bb-lig_rev | AATTT [symbol] TCCACCCAGGCCTTGGCC |
| 64 | Ct-A2bb-lig_for | AATTT [symbol] CGCCCGCGGGAACTGAAG |
| 65 | Ct-A2bb-lig_rev | AATTT [symbol] GGTAGCAAGAGCACAAGG |
| 66 | Ct-B1bb-lig_for | TTTAAA [symbol] TGGATCCATTCCATCGAAACCTTG |
| 67 | Ct-B1bb-lig_rev | TTTAAAA [symbol] AGTCGTAGCGAAAGACG |
| 68 | Ct-B2bb-lig_for | TTTAAA [symbol] ACCGCGGCACACGGAAAGG |
| 69 | Ct-B2bb-lig_rev | TTTAAA [symbol] TCCCGGGCGGCAGAAGCC |
| | For generation of pMS470-CtNHase-β1-focused library | |
| 70 | Ct-beta1-focused_for | ACTTCAAC [symbol] GATGAG [symbol] CGACAC [symbol] ATCGAGCGCATGAAC |
| 71 | Ct-beta1-focused_rev | CATGCGCTCGAT [symbol] GTGTCG [symbol] CTCATC [symbol] GTTGAAGTTGCCGTTGG |
| | For generation of pMS470-CtNHase-αP121X-βF51L mutants | |
| 72 | Ct-P121A_for | ATGG [symbol] ACGCTGGGCTTGC |
| 73 | Ct-P121A_rev | AGCGT [symbol] CCATGGGTAGCAAGAG |
| 74 | Ct-P121R_for | ATGG [symbol] ACGCTGGGCTTGC |
| 75 | Ct-P121R_rev | AGCGT [symbol] CCATGGGTAGCAAGAG |
| 76 | Ct-P121N_for | ATGG [symbol] ACGCTGGGCTTGC |
| 77 | Ct-P121N_rev | AGCGT [symbol] CCATGGGTAGCAAGAG |
| 78 | Ct-P121D_for | ATGG [symbol] ACGCTGGGCTTGC |
| 79 | Ct-P121D_rev | AGCGT [symbol] CCATGGGTAGCAAGAG |
| 80 | Ct-P121C_for | ATGG [symbol] CGCTGGGCTTGC |
| 81 | Ct-P121C_rev | AGCGT [symbol] CCATGGGTAGCAAGAG |
| 82 | Ct-P121Q_for | ATGG [symbol] ACGCTGGGCTTGC |
| 83 | Ct-P121Q_rev | AGCGT [symbol] CCATGGGTAGCAAGAG |
| 84 | Ct-P121E_for | ATGG [symbol] ACGCTGGGCTTGC |
| 85 | Ct-P121E_rev | AGCGT [symbol] CCATGGGTAGCAAGAG |
| 86 | Ct-P121G_for | ATGG [symbol] ACGCTGGGCTTGC |
| 87 | Ct-P121G_rev | AGCGT [symbol] CCATGGGTAGCAAGAG |
| 88 | Ct-P121H_for | ATGG [symbol] ACGCTGGGCTTGC |
| 89 | Ct-P121H_rev | AGCGT [symbol] CCATGGGTAGCAAGAG |
| 90 | Ct-P121I_for | ATGG [symbol] ACGCTGGGCTTGC |
| 91 | Ct-P121I_rev | AGCGT [symbol] CCATGGGTAGCAAGAG |
| 92 | Ct-P121L_for | ATGG [symbol] ACGCTGGGCTTGC |
| 93 | Ct-P121L_rev | AGCGT [symbol] CCATGGGTAGCAAGAG |
| 94 | Ct-P121K_for | ATGG [symbol] CGCTGGGCTTGC |
| 95 | Ct-P121K_rev | AGCGT [symbol] CCATGGGTAGCAAGAG |
| 96 | Ct-P121M_for | ATGG [symbol] ACGCTGGGCTTGC |
| 97 | Ct-P121M_rev | AGCGT [symbol] CCATGGGTAGCAAGAG |
| 98 | Ct-P121F_for | ATGG [symbol] ACGCTGGGCTTGC |
| 99 | Ct-P121F_rev | AGCGT [symbol] CCATGGGTAGCAAGAG |
| 100 | Ct-P121W_for | ATGG [symbol] ACGCTGGGCTTGC |
| 101 | Ct-P121W_rev | AGCGT [symbol] CCATGGGTAGCAAGAG |
| 102 | Ct-P121Y_for | ATGG [symbol] ACGCTGGGCTTGC |
| 103 | Ct-P121Y_rev | AGCGT [symbol] CCATGGGTAGCAAGAG |
| | For generation of pMS470-CtNHase combination mutants | |
| 104 | Ct-P121T_for | ATGG [symbol] ACGCTGGGCTTGC |
| 105 | Ct-P121T_rev | AGCGT [symbol] CCATGGGTAGCAAGAG |
| 106 | Ct-V110I_for | AACGTCA [symbol] GTTTGCACCTTGTGCTCTTG |
| 107 | Ct-V110I_rev | AAAC [symbol] GACGTTGTGGACGGC |
| 108 | Ct-L48R-G54C_for | ACTTCAAC [symbol] GATGAG [symbol] CGACAC [symbol] ATCGAGCGCATGAAC |
| 109 | Ct-L48R-G54C_rev | CATGCGCTCGAT [symbol] GTGTCG [symbol] CTCATC [symbol] GTTGAAGTTGCCGTTGG |
| 110 | Ct-L48R-G54R_for | ACTTCAAC [symbol] GATGAG [symbol] CGACAC [symbol] ATCGAGCGCATGAAC |
| 111 | Ct-L48R-G54R_rev | CATGCGCTCGAT [symbol] GTGTCG [symbol] CTCATC [symbol] GTTGAAGTTGCCGTTGG |
| 112 | Ct-L48R-G54V_for | ACTTCAAC [symbol] GATGAG [symbol] CGACAC [symbol] ATCGAGCGCATGAAC |
| 113 | Ct-L48R-G54V_rev | CATGCGCTCGAT [symbol] GTGTCG [symbol] CTCATC [symbol] GTTGAAGTTGCCGTTGG |
| 114 | Ct-L48P-G54C_for | ACTTCAAC [symbol] GATGAG [symbol] CGACAC [symbol] ATCGAGCGCATGAAC |
| 115 | Ct-L48P-G54C_rev | CATGCGCTCGAT [symbol] GTGTCG [symbol] CTCATC [symbol] GTTGAAGTTGCCGTTGG |
| 116 | Ct-L48P-G54R_for | ACTTCAAC [symbol] GATGAG [symbol] CGACAC [symbol] ATCGAGCGCATGAAC |
| 117 | Ct-L48P-G54R_rev | CATGCGCTCGAT [symbol] GTGTCG [symbol] CTCATC [symbol] GTTGAAGTTGCCGTTGG |
| 118 | Ct-L48P-G54V_for | ACTTCAAC [symbol] GATGAG [symbol] CGACAC [symbol] ATCGAGCGCATGAAC |
| 119 | Ct-L48P-G54V_rev | CATGCGCTCGAT [symbol] GTGTCG [symbol] CTCATCAGGGTTG [symbol] TTGCCGTTGG |
| 120 | Ct-L48F-G54C_for | ACTTCAAC [symbol] GATGAG [symbol] CGACAC [symbol] ATCGAGCGCATGAAC |
| 121 | Ct-L48F-G54C_rev | CATGCGCTCGAT [symbol] GTGTCG [symbol] CTCATC [symbol] GTTGAAGTTGCCGTTGG |
| 122 | Ct-L48F-G54R_for | ACTTCAAC [symbol] GATGAG [symbol] CGACAC [symbol] ATCGAGCGCATGAAC |
| 123 | Ct-L48F-G54R_rev | CATGCGCTCGAT [symbol] GTGTCG [symbol] CTCATC [symbol] GTTGAAGTTGCCGTTGG |
| 124 | Ct-L48F-G54V_for | ACTTCAAC [symbol] GATGAG [symbol] CGACAC [symbol] ATCGAGCGCATGAAC |
| 125 | Ct-L48F-G54V_rev | CATGCGCTCGAT [symbol] GTGTCG [symbol] CTCATC [symbol] GTTGAAGTTGCCGTTGG |
| 126 | Ct-L48P-F51V-G54V_for | ACTTCAAC [symbol] GATGAG [symbol] CGACAC [symbol] ATCGAGCGCATGAAC |
| 127 | Ct-L48P-F51V-G54V_rev | CATGCGCTCGAT [symbol] GTGTCG [symbol] CTCATC [symbol] GTTGAAGTTGCCGTTGG |
| 128 | Ct-L48F_for | CAAC [symbol] GATGAGTTTCGACAC |
| 129 | Ct-L48F_rev | GTCGAAACTCATC [symbol] GTTGAAG |
| 130 | Ct-F51L_for | CGATGAG [symbol] CGACACGGC |
| 131 | Ct-F51L_rev | GCCGTGTCG [symbol] CTCATCG |

Altered triplet codons are underlined. N can be A, G, T, or C; M can be A or C; K can be G or T; Y can be T or C; S can be C or G; R can be G or A; V can be A, G, or C; D can be A, G, or T; H can be A, C, or T; B can be G, C, or T.

### 2.2. Cloning

This section summarizes cloning protocols.

2.2.1 Ordered Genes

The genes coding for α and β subunit as well as the accessory proteins (see SEQ ID NOs in Table 34 below) for selected NHases were ordered as double-stranded DNA fragments. Genes for BjNHase, CtNHase, KoNHase, M/NHase, NaNHase, PcNHase, R/NHase, RmNHase and RsNHase were purchased as gBlocks from IDT (Leuven/Belgium), GenParts were obtained for AbNHese, AmNHase, BrNHase, TrNHase and VvNHase from GenScript (New Jersey/USA), and CjNHase, GhNHase and PtNHase were purchased as GeneArt Strings from ThermoFisher Scientific (Waltham/USA). Relevant sequences are listed in a separate section below.

2.2.2 Preparation of Vector Backbone

Desired plasmids were amplified in E. coli Top10F′ and isolated with the GeneJET® Miniprep Plasmid Kit (ThermoFisher Scientific). For generation of the empty vector backbone, 20 μg of pMS470d8 were digested with 6 μL of NdeI and 6 μL of HindIII (NEB) (Ipswitch/USA), in 1× CutSmart Buffer at 37° C. overnight. The 3981 bp fragment was purified using a preparative agarose gel and the Wizard® SV Gel and PCR Clean-Up System (Promega). (Madison/USA),

For preparation of pMS470-CtNHase/pMS470-CtNHase-βF51L backbones lacking of α1, α2, β1, β2 or β1-β2 regions, 5 μg of vector were cut with 6 μL of NheI-FD or XhoI-FD in 1×FD Green Buffer (ThermoFisher Scientific) in a total volume of 50 μL at 37° C. for 2 h. Linearized vectors were purified using a preparative agarose gel and the Wizard® SV Gel and PCR Clean-Up System and dephosphorylated by the recombinant shrimp alkaline phosphatase (NEB) and desalted before further use.

2.2.3 Gibson Cloning

Gibson cloning was performed with the Gibson Assembly HiFi 1-Step Kit (Synthetic Genomics) (La Jolla/USA), according to the manufacturer's protocol.

For cloning of the NHase panel into pMS470d8, 20-40 ng of vector were used and 10-15 ng of insert in a ratio 1:1. For cloning of smaller fragments into the vector pMS470-CtNHase, e.g. for generation of random libraries, 1 equivalent of vector backbone was applied with 3 equivalents of the insert. Also, after Overlap Extension PCR, 3 eq. of the insert (synthesized by PCR) were used per 1 eq. of the vector.

2.2.4 QuikChange PCR

Single positions were mutated in QuikChange PCR either to introduce a specific triplet codon or the degenerated codon NNK.

The PCR reactions contained 10 ng or 116 ng of template DNA (pMS470-CtNHase or a mutant thereof), 0.2 μM of forward and reverse primer (e.g. Ct-aQ93X_for and Ct-aQ93X_rev, Table 2) 0.2 mM of dATP, dCTP, dGTP and dTTP, 1×Q5 Reaction buffer, 1×Q5 High GC Enhancer and 1 U Q5 High-Fidelity DNA polymerase. The PCR program was as follows: 30 s at 98° C., 30 cycles at 98° C. for 10 s, 56/58° C. for 30 s and 72° C. for 3 min and a final extension step at 72° C. for 6 min.

The PCR products were either cleaned-up (Wizard® SV PCR and Clean-Up System) right after the PCR and purified products were digested by 20 U DpnI in 1× Tango buffer (ThermoFisher Scientific) for 2 h at 27° C. and desalted or 10 U DpnI were added directly to the reaction right after PCR, incubated for 2 h at 37° C. and afterwards purified. Table 3 summarizes the PCR conditions for all pMS470-CtNHase constructs.

TABLE 3
QuikChange PCR conditions for different constructs.

| Group | 1 | 2 | 3 |
|---|---|---|---|
| description | NNK libraries | double mutants | combination |
| | based | of rational | mutants |
| | on wild type | hits, NNK | |
| | | library based on | |
| | | rational hit | |
| examples | CtNHase- | CtNHase- | CtNHase- |
| for mutants | αQ93X, | βM34L/βF51L | αV110I/βL48R, |
| | CtNHase- | CtNHase- | CtNHase- |
| | βF51X | βM34X/βF51L | αP121T/βL48R |
| template | 116 ng | 10 ng | 10 ng |
| amount | | | |
| annealing | 56 | 56 | 58 |
| temperature | | | |
| Dpnl digest | after clean-up | after PCR | after PCR |

Template amount, annealing temperature as well as the time point of Dpnl digest were varied for different pMS470-CtNHase constructs.

2.2.5 Random Mutagenesis

Random mutagenesis libraries of four regions of the CtNHase gene were constructed, two in the alpha subunit and two in the beta subunit, through amplification with Mutazyme II (Agilent Technologies) (SantaClara/USA), and addition of MnCl₂. The 50 μL PCR reactions contained 5 ng of template DNA (pMS470-CtNHase), 0.4 μM of the forward and the reverse primer (e.g. Ct-alpha1_for and Ct-alpha1_rev, Table 2), 0.2 mM of dATP, dCTP, dGTP and dTTP, 0.5-1 mM MnCl₂, 1× Mutazyme II reaction buffer and 2.5 U Mutazyme II DNA polymerase. The PCR program was as follows: 2 min at 95° C., 30 cycles at 95° C. for 30 s, 56° C. for 30 s and 72° C. for 1 min and a final extension step at 72° C. for 10 min. The size of the PCR products was analyzed by gel electrophoresis and the products were purified (Wizard® SV PCR and Clean-Up System, Promega).

50-70 ng of PCR product were cloned into pJET1.2/blunt (CloneJET PCR Cloning Kit, ThermoFisher Scientific) using the sticky end protocol. Electro-competent E. coli Top10F′ cells were transformed with the resulting plasmids. After amplification in E. coli and isolation (GeneJET® Miniprep Plasmid Kit, ThermoFisher Scientific), some of those plasmids were sent for sequencing (Microysynth AG) to determine the mutation rate.

2.2.6 Backbone Strains

Vector backbones were generated in 50 μL PCR reactions that consisted of 1 ng of template DNA (pMS470-CtNHase or pMS470-CtNHase-βF51L), 0.2 μM of both primers (e.g. Ct-A1bb-lig_for and Ct-A1bb-lig_rev, Table 2), 0.2 mM of dATP, dCTP, dGTP and dTTP, 1×Q5 Reaction buffer, 1×Q5 High GC Enhancer and 1 U Q5 High-Fidelity DNA polymerase. The PCR program was as follows: 30 s at 98° C., 30 cycles at 98° C. for 10 s, 60° C. for 30 s and 72° C. for 2 min and a final extension step at 72° C. for 2 min. The PCR products were digested with 10 U DpnI for 2 h and purified (Wizard® SV PCR and Clean-Up System). The ends of 1 μg PCR product were digested with XhoI or NheI for 15 min at 37° C. and heat inactivated for 20 min at either 65° C. or 80° C. The cut PCR products were purified and ligated (T4 DNA Ligase, ThermoFisher Scientific) for 10 min at 22° C.

2.2.7 Overlap Extension PCR

For the introduction of multiple mutations in the β1 region, overlap extension PCR had to be performed. In the first round, the forward and the reverse fragment were amplified, which were joined in a second PCR reaction using the outer primers. This strategy was used for targeting the positions βL48, βF51 and βG54 at the same time.

The first PCR reactions with a total volume of 50 μL consisted of 1 ng of template DNA (pMS470-CtNHase or a mutant thereof), 0.2 μM of both primers (e.g. Ct-b1-focused_for and Ct-b1_rev (Tab.2), 0.2 mM of dATP, dCTP, dGTP and dTTP, 1×Q5 Reaction buffer, 1×Q5 High GC Enhancer and 1 U Q5 High-Fidelity DNA polymerase. The PCR program was as follows: 30 s at 98° C., 30 cycles at 98° C. for 10 s, 60° C. for 30 s and 72° C. for 30 s and a final extension step at 72° C. for 2 min. The PCR reactions were stopped by addition of 10 μL of 6× Loading Dye and loaded onto a preparative agarose gel. PCR products with the correct size were cut out and purified using the Wizard® SV PCR and Clean-Up System.

A second PCR was conducted to join the forward and the reverse fragment. The PCR reaction, totally 50 μL, contained of 2.5 μL of purified forward fragment, 2.5 μL of purified reverse fragment, 0.2 μM of both outer primers, 0.2 mM of dATP, dCTP, dGTP and dTTP, 1×Q5 Reaction buffer, 1×Q5 High GC Enhancer and 1 U Q5 High-Fidelity DNA polymerase. The PCR program was as the same as for the first PCR. 10 μL of 6× Loading Dye were added to the reactions and loaded onto a preparative agarose gel. Desired PCR products were cut out and cleaned-up using the Wizard® SV PCR and Clean-Up System.

2.2.8 Confirmation of New Constructs

Electro-competent E. coli Top10F′ cells were transformed with newly generated plasmids after QuikChange PCR or Gibson cloning.

After colony PCR (2.2.9), clones with the correct insert size were streaked out for plasmid isolation or random clones if colony PCR was not performed. Plasmids were isolated with the GeneJET® Plasmid Miniprep Kit (ThermoFisher Scientific) and analyzed by Sanger sequencing (Microsynth). Eventually, electro-competent E. coli BL21 Gold (DE3) cells were transformed with the confirmed plasmids for enzyme expression.

2.2.9 Colony PCR

2. Colony PCR was Performed after Gibson Cloning (2.2.3) or QuikChange PCR (2.2.4) to Confirm the Right Insert Size of the Newly Constructed Plasmids.

The PCR reactions contained 0.2 μM of forward and reverse primer (KST_foropt and KST_rev1, Table 2) 0.2 mM of dATP, dCTP, dGTP and dTTP, 1× DreamTaq Reaction buffer and 0.025 U DreamTaq DNA Polymerase. A small amount of cell material of E. coli containing the newly generated plasmids was added by touching the target colony with a toothpick and then swirled in the reaction mixture. The PCR program was as follows: 10 min at 95° C., 30 cycles at 95° C. for 30 s, 53° C. for 30 s and 72° C. for 1 min and a final extension step at 72° C. for 7 min. The size of products was analyzed by gel electrophoresis.

### 2.3 Protein Expression

2.3.1 Shake Flask Expression

10 mL LB medium containing 100 μg/mL ampicillin (LB-Amp) were inoculated with either a single colony or a small amount of cell material from a cryo-conserved sample. Overnight cultures (ONCs) were grown at 37° C. under shaking (appr. 110-150 rpm) overnight. The next day, 400 mL of LB-Amp media were inoculated with 4 mL of ONC and incubated at 37° C. and 120 rpm until an OD₆₀₀ of 0.8-1 was reached. Protein expression was induced with 0.1 mM IPTG and 1 mM CoCl₂ or 0.1 mM FeSO₄, according to the NHases metal dependence. Induced cells were cultivated at 20° C. at 120 rpm for 18-22 h and harvested by centrifugation in a JA-10 rotor for 15 min at 4° C. and 5,000 g. The pellets were stored at −20° C. until further use.

2.3.2 Deep Well Plate Cultivation

96-well deep well plates were filled with 750 μL LB medium containing 100 μg/mL ampicillin per well and inoculated either with single colonies or from cryo-preserved cultures. Overnight cultures were grown at 37° C. and 320 rpm. On the next day, 750 μL of LB-Amp media were inoculated with 25 μL of ONC and incubated at 37° C. and 320 rpm for 6 h. Cells were induced with 50 μL LB-Amp medium containing IPTG and CoCl₂ to give final concentrations of 0.1 mM and 1 mM, respectively. Temperature was reduced to 20° C. After 22 h, cells were harvested by centrifugation in a 5810R Eppendorf centrifuge for 15 min at 2970 g. Supernatant was decanted and the cell pellets stored at −20° C.

The expression of NHases in deep well plates was optimized. In the improved protocol, only 10 μL of ONC were used to inoculate the main culture and induction was done after 2¾ h instead of six. For cryo-conservation, 300 μL of 50% glycerol was added to the ONCs.

2.3.3 Cell Lysis

Cell pellets, usually from 1-3 g per flask, were resuspended in 25 mL 50/40 mM Tris-butyrate buffer, pH 7.2, and lysed on ice by sonication for 6 min at 70-80% duty cycle and 7-8 output control. Cell-free extracts were obtained after centrifugation at 48,250 g and 4° C. for 1 h and filtered through 0.45 μM syringe filters. Protein concentration was determined using the Pierce™ BCA Protein Assay Kit (ThermoFisher Scientific).

2.3.4 SDS-PAGE

The BugBuster® Protein Extraction Reagent (Novagen) was applied for the determination of NHase content in cell-free extracts. Samples were analyzed by SDS-PAGE analysis and evaluated with the GeneTool software.

13 μL of protein sample were mixed with 5 μL of NuPAGE® LDS sample buffer (4×) and 2 μL NuPAGE® sample reducing agent (10×), denatured at 95° C. for 10 min and shortly centrifuged. Ten μL were loaded onto a NuPAGE® 4-12% Bis-Tris gel whereas only 4 μL of PageRuler™ prestained protein ladder were used. The gel was run for 35 min with 1× NuPAGE® MES SDS running buffer. Staining was performed either with SimplyBlue™ SafeStain (ThermoFisher Scientific) or InstantBlue™ (Expedeon Protein Solutions).

When whole cells were analyzed by SDS-PAGE, the 20 μL samples included cells approx. 13.6 mg/mL, 2× NuPAGE® LDS sample buffer and 1× NuPAGE® sample reducing agent. These samples were denatured at 95° C. for 20 min before they were loaded onto the gel.

2.3.5 Heat Purification

Cell-free extracts were incubated at 40° C., 50° C. and 60° C., respectively, for 10 min at 900 rpm in a thermomixer device. After centrifugation at 4° C. and 21.13 g for 10 min, supernatants were filtered through 0.45 μm syringe filters. Heat purified cell-free extract (CFEs) were analyzed by SDS-PAGE (2.3.4) and assayed for methacrylonitrile hydrolysis (2.4.1).

### 2.4 Activity Assays and Analytics

2.4.1 Photometric Assay for Methacrylonitrile Hydration Activity

Physicochemical characteristics of NHases were determined by monitoring the hydration of methacrylonitrile (MAN). Therefore, 10 μL NHase CFE (diluted in Tris-butyrate buffer 50/40 mM pH 7.2) were mixed with 100 μL of 125 mM MAN in Tris-butyrate buffer 50/40 mM (pH 7.2) in 96-well UV star plates. The formation of methacrylamide (MAD) was monitored at 224 nm on a Synergy Mx Platereader (BioTek) at 25° C. for 5 min. The activity of the sample in Units/mL was calculated with the following formula:

$$\frac{U}{ml} = \frac{Velocity ( {min}^{- 1} ) \times 0.11 mL \times dilution}{2.551 mM {cm}^{- 1} \times 0.1 mL \times pathlength ( cm )}$$

Appropriate blank reactions were carried out in parallel and each reaction was carried out at least in triplicate.

Temperature and pH Studies

For the determination of the pH optimum, the standard assay as described above was used with the following buffers: 100 mM citrate-phosphate buffer pH 5-6, 100 mM sodium phosphate buffer pH 7-8, 100 mM Tris-HCl buffer pH 8.5, 100 mM carbonate buffer pH 9-10.

For stability tests, NHase-CFEs were incubated at different temperatures and/or at different pH for up to six hours under shaking (300 rpm) before they were assayed for methacrylonitrile hydration.

Inhibition Studies

To assay potential inhibition, the standard assay as described above was used with minor adaptions. MAN was dissolved in 0-50 mM KCN solutions or in 0-50 mM propanal solutions. Also protein samples were diluted with the respective KCN or propanal solutions.

2.4.2 Biocatalytic Conversions with Target Substrate

The standard set-up of biocatalytic hydration of rac-1 by NHases was 500 μL reaction volume comprising either NHase-CFE (total protein in the range of about 5 to 15 mg/ml, NHase content of about 5 to 40% in CFE determined by SDS PAGE using Gene Tool Software, corresponding to about 0.0125 to 6 mg/ml NHase) or cells, the substrate and buffer. Incubation was done at 25° C. in a thermomixer device. Numerous different experiments have been performed in which many parameters were altered (one at a time) or additives were used.

Reactions were stopped by the addition of 2 volumes of ethanol and mixed thoroughly for 1 min. Reactions were left at room temperature overnight for protein precipitation before they were centrifuged for at least 20-40 min at max speed in a table-top centrifuge. 500 μL of the supernatant were transferred into HPLC vials.

### Screening NHase Panel

For screening of the NHase panel for conversion of rac-1, 500 μL reactions were set up with 10 mM of rac-1 and 50 μL of NHase-CFE in 50 mM sodium phosphate buffer, pH 7.2. Reactions were incubated at 25° C. and 300 rpm overnight.

### Temperature Studies

Different reaction temperatures were investigated for NHases capable of hydration of 1, but the reaction set-up was slightly altered compared to the screening set-up described above. 80 μL of NHase-CFE was used for the conversion of 10 mM of rac-1 and incubation was done at 37° C. or 50° C.

### Conversions of Higher Concentrations of Rac-1

Promising candidates were tested for higher substrate concentrations. Therefore, 50 μL of CFE were applied in 500 μL reactions in 50 mM sodium phosphate buffer, pH 7.2, with either 50 or 100 mM of rac-1.

### Time Studies

A time study was performed with CFEs of CtNHase, KoNHase and GhNHase. Thus, 900 μL reactions with 90 μL CFE and 20 mM of rac-1 were incubated at 25° C. and 300 rpm in duplicates. 100 μL samples were taken after 30 min, 1 h, 2 h, 4 h, 6 h and overnight.

### Effect of Co-Solvents

Moreover, also organic co-solvents were tested. Reactions were carried out with 50 mM rac-1, 10% (v/v) NHase-CFE and 5% of co-solvent in 50 mM sodium phosphate buffer, pH 7.2. Samples were incubated at 25° C. and 1000 rpm overnight.

### Effect of Catalyst Amount

The hydration of rac-1 was performed using different catalyst amounts of CtNHase-CFE and GhNHase-CFE. 50 mM substrate were applied and 0.5-20% (v/v) of CFE in 200 mM Tris-HCl buffer, pH 7, at 500 rpm for 2 h. The reaction temperature was 5° C. for CtNHase and 25° C. for GhNHase.

### Effect of Low Conversion Temperature

A lower reaction temperature was investigated in 500 μL scale reactions. 50 mM rac-1 were applied in 50 mM sodium phosphate buffer, pH 7.2 using 100 μL of CFE. Incubation was done at 5° C. and 25° C. (for controls) and 300 rpm overnight. For the time study, 1 mL reactions were set up with 20 mM of rac-1 and 10% (v/v) of CFE in 100 mM sodium phosphate buffer, pH 8. Samples were taken after 1, 2, 5, 10, 20 and 30 min, respectively. In addition, 60 min samples were analyzed for the reactions at 5° C.

### Effect of Enzyme Feeding

Enzyme feeding experiments were performed in 500 μL reactions containing 50 mM rac-1 in 50/40 mM Tris-butyrate, pH 7.2. At the beginning of the reaction, 50 μL of CFE were added and incubation started at 25° C. and 300 rpm. After 1 h, additional 50 μL of CFE were applied and incubation was done for another hour before the reactions were stopped. In a similar experiment, 50 mM rac-1 were converted in 50 mM sodium phosphate (NaPi) or 50/40 mM Tris-butyrate buffer, pH 7.2, at 5° C. and 300 rpm overnight. Also here, reactions were started with 50 μL of CFE and for the feeding reactions, another 50 μL were added after 1 h.

### Conversion of Rac-1 by CtNHase and GhNHase at Different pH

Reactions with CtNHase-CFE were carried on 500 μL scale in triplicates with 50 μL CFE and 50 mM rac-1 in 200 mM sodium phosphate or Tris-HCl buffer, pH 7-8.5. Reactions with GhNHase-CFE were slightly altered. 100 μL of CFE were used and buffers ranging from pH 6.5 to 8.5.

### Effect of Low Catalyst Amount

A time study was conducted with GhNHase. The 1 mL reaction contained 50 mM of rac-1 and 20 μL of CFE in 200 mM Tris-HCl buffer, pH 7. Incubation was done at 25° C. and 500 rpm. 100 μL samples were taken after 5, 10, 15, 30, 45, 60 and 120 min.

Substrate Feeding Reactions with GhNHase Cells

Conversion of rac-1 was investigated for lyophilized GhNHase cells in substrate feeding reaction. E. coli BL21 Gold (DE3) [pMS470-GhNHase] cultivated in shaking flasks (protocol 2.3.1) were resuspended in 50/40 mM Tris-butyrate buffer, pH 7.2, to an OD₆₀₀ of 45.7 per mL which correlates to 77.7 mg/mL wet cell weight. Aliquots of 100 μL were frozen at −80° C. and lyophilized overnight. For the biocatalytic reaction, one aliquot of lyophilized cells was resuspended in 200 mM Tris-HCl, pH 7, and rac-1 was added to start the reaction. Duplicates were done in following set-ups: Control reactions with 25 mM substrate as batch reactions, and feeding reactions started with 25 mM rac-1 and addition of 25 mM after 2 h. The same was conducted for 50 mM reactions, where the feeding reaction contained 100 mM of substrate. All reactions were incubated at 25° C. and 500 rpm for 4 h.

### Conversion of Rac-1 by Whole Cells

The hydration of rac-1 by whole cell catalysts was tested at various pH values and for different catalyst amount. Cells were resuspended in 50/40 mM Tris-butyrate buffer, pH 7.2, and concentrations of 8.5, 4.25, 1.7, 0.85, 0.34 and 0.17 were tested. The 500 μL reactions, containing 50 mM of rac-1, were incubated at 25° C. and 500 rpm for 2 h in 200 mM Tris-HCl buffer, pH 7, 7.5 or 8, respectively.

Time Study with Whole Cells

E. coli BL21 Gold (DE3) cells carrying [pMS470-CtNHase] or [pMS470-GhNHase] were resuspended in 50/40 mM Tris-butyrate buffer, pH 7.2, to 85 mg/mL and also an 1:5 dilution was prepared. One mL scale reactions with 50 mM of rac-1 and 8.5 or 1.7 mg/mL cells in 200 mM Tris-HCl buffer, pH 7, were incubated at 25° C. and 500 rpm. Samples were taken after 30 min, 1 h, 2 h, 3 h, 4 h, 6 h, 8 h and 25 h.

### Substrate Feeding

E. coli BL21 Gold (DE3) [pMS470-CtNHase] and [pMS470-GhNHase] expressing cells were resuspended in 50/40 mM Tris-butyrate buffer, pH 7.2, to 85 mg/mL for substrate feeding reactions. The first set-up was done on 10 mL scale containing 1.7 mg/mL cells in 200 mM Tris-HCl buffer, pH 7. To start the reaction, 50 mM of rac-1 were added and shaken at 25° C. and 750 rpm. After one hour, 100 μL sample were taken, again 50 mM of rac-1 were added and also 100 μL of 1 M HCl to maintain the pH. This procedure war repeated after 2 and 3 h. After 4 and 5 h of incubation, 100 μL of sample were taken. The second reaction was set-up on 2 mL scale with 8.5 mg/mL cells in 200 mM Tris-HCl buffer, pH 7.10 mM of rac-1 were added to start the reaction and incubation was done at 25° C. and 1400 rpm. After 1, 2, 3 and 4 h, respectively 100 μL of sample were taken for analysis and 10 mM of rac-1 were added. The last 100 μL sample was taken after 5 h.

### Conversion of High Rac-1 Concentrations

CtNHase and GhNHase were tested for rac-1 hydration up to 200 mM of nitrile. The 500 μL scale reactions contained 8.5 mg/mL cells and 50, 75, 100, 150 or 200 mM rac-1, respectively, in 200 mM Tris-HCl buffer, pH 7, 7.5 or 8.1 M HCl was added to the reaction to maintain the pH, depending on the substrate concentration. Incubation was done at 25° C. and 700 rpm for 1 h.

### Addition of Pyrrolidine and Propanal

Hydration of rac-1 by CtNHase was investigated in the presence of additional pyrrolidine or propanal. The 500 μL reactions contained 8.5 mg/mL cells and 150 mM rac-1 in 500 mM Tris-HCl buffer, pH 7.5.

### Re-Screening of Site-Saturation CtNHase Clones

A screening plate containing CtNHase-βF51X clones was assayed for rac-1 hydration. Therefore, cell pellets from deep well plate cultivation were resuspended in 200 μL of 200 mM Tris-HCl buffer, pH 7, to approximately an OD₆₀₀ of 20, which corresponds to approximately 34 mg/mL wet cell weight. 125 μL of cell suspension were transferred to a fresh microcentrifuge tube. To start the reaction, 375 μL of 66.67 mM rac-1 in 200 mM Tris-HCl buffer, pH 7, were added ending in 8.5 mg/mL of cells and 50 mM of substrate. Incubation was done at 25° C. and 500 rpm for 2 h.

### Re-Screening of Potential CtNHase Hits

The re-screening of promising CtNHase clones was done on 500 μL scale. Reactions were composed of 8.5 mg/mL cells and 100 mM of rac-1 in 500 mM Tris-HCl buffer, pH 7, and incubated at 25° C. and 700 rpm for 2 h. Reactions were supplemented with 75 or 150 mM pyrrolidine or propanal. One reaction was done with additional 75 mM pyrrolidine and propanal. All reactions were done in triplicates (except for the one with additional 150 mM propanal) and incubated at 25° C. and 700 rpm for 1 h.

2.4.3 HPLC Analytics

(R)-2 and (S)-2 were separated by a Chiralpak AD-RH (150×4.6 mM, 5 μm) using 20 mM Na-borate buffer, pH 8.5, and acetonitrile in a ratio 70:30 as the mobile phase, at a flow rate of 0.5 mL/min for 15 min. The compounds were detected at 210 nm (DAD). A calibration curve of rac-2 was used for quantification by linear interpolation and peak areas were used to calculate the enantiomeric excess (ee). Retention times of (R)- and (S)-2 were 5.8 min and 6.4 min, respectively. A systematic impurity was introduced by the buffer that was not baseline separated from (R)-2 (retention time: 5.7 min).

2.4.4 Substrate Stability Experiments

The decomposition of rac-1 at different pH was investigated using Feigl-Anger filter paper [F. Feigl, V. Anger, Replacement of benzidine by copper ethylacetoacetate and tetra base as spot-test reagent for hydrogen cyanide and cyanogen, Analyst. 91 (1966) 282. doi:10.1039/an9669100282] Therefore, 10 mM solutions of α-ethyl-1-pyrrolidineacetonitrile rac-1 were prepared in different buffers: 100 mM carbonate buffer, pH 9 and 10, 100 mM sodium phosphate buffer, pH 7 and 8 and 100 mM citrate phosphate buffer, pH 5 and 6. In addition, the substrate was dissolved in ethanol as control. 100 μL of the rac-1 solutions were pipetted into a 96-well micro titer plate and covered with Feigl-Anger filter paper. Color development was documented by taking pictures (see FIG. 3).

To investigate the velocity of rac-1 decomposition at different pH, rac-1 was dissolved in different buffer, ranging from pH 5 to pH 10, and extracted immediately, after 2 and after 60 min, respectively, with 1 volume ethyl acetate. Extracts were dried over Na₂SO₄ and the supernatant evaporated using a vacuum centrifuge. The remaining substances (including rac-1) were dissolved in 100 μL of ethanol.

(R)- and (S)-1 were separated by a Chiralpak AD-H using n-heptane and ethanol in a ratio 90:10 as the mobile phase, at a flow rate of 1 mL/min for 8 min. The compounds were detected at 210 nm (DAD). Retention times of (R)- and (S)-1 were 4.2 min and 4.7 min, respectively.

2.4.5 Homology Modelling

YASARA's homology modeling experiment (version 18.2.7.W.64) with following parameters:

Modelling speed: Fast

PS-BLAST iterations: 4

E-value: 0.01

Max Number of Templates: 8 (same sequence: 1)

Max OligoState: 2 (dimeric)

Max alignment per template: 5

Conformations per loop: 50

Max Number of residues added to termini: 10

### 2.5 Screening Assays

A high-throughput assay for determination of nitrile hydratase activity was applied for the screening of CtNHase libraries. This coupled assay is suitable for colony screening or liquid reactions in well plates. In the first phase, NHases convert the nitrile to the respective amide in an aqueous system. The amide is further converted to the hydroxamic acid in the amidase phase, where partially purified amidase cell free extract of Rhodococcus erythropolis and hydroxyl ammonium chloride are added. During the detection phase, hydroxamic acids form colored complexes in presence of iron and hydrogen ions. In order to get visible signals using rac-1 as the substrate, many assay parameters had to be optimized, in particular the ratio between substrate and hydroxyl ammonium chloride, the amount of ReAmidase, incubation times and temperatures, growing conditions, filter material and detection mode. The final protocols are described in the following paragraphs.

2.5.1 Colony Based Screening Assay

E. coli BL21 Gold (DE3) cells containing libraries of pMS470-CtNHase were grown on LB agar plates containing 100 μg/mL ampicillin (LB-Amp plates) for 72 h at room temperature or for 24 at 37° C. and 20 h at room temperature before they were attached to sterilized Amersham Protran nitrocellulose membranes. The membranes were placed on LB-Amp plates containing 0.5 mM IPTG and 1 mM CoCl₂ with the colonies facing upwards. After 24-48 h of induction, colonies were used for the screening assay.

Filter paper (Whatman cellulose) was soaked with 100 mM rac-1 (α-ethyl-1-pyrrolidineacetonitrile) in 200 mM Tris-HCl, pH 7 and the membrane with colonies was placed on top. This NHase phase was conducted for 15 min at room temperature. After that, the membrane was transferred to a new filter soaked with amidase reaction solution containing 4 parts 27 mg/mL partially purified ReAmidase-CFE in 100 mM sodium phosphate buffer, pH 7.5 and 1 part 1 M hydroxyl ammonium chloride in 200 mM Tris-HCl, pH 7. Incubation was done at 30° C. for 30 min. For detection, the membranes were transferred to fresh filter papers soaked with 0.6 M FeCl₃ in 1 M HCl. Active clones turned red on the yellow background and were detected by eye, although pictures were also taken.

Promising clones were picked into sterile 96-well polystyrene plates filled with 100 μL LB medium containing 100 μg/mL ampicillin and 50% glycerol in a ratio 2:1, sealed with aluminum foil, shaken for 15 min at room temperature and frozen at −20° C.

2.5.2 Liquid Screening Assay

E. coli BL21 Gold (DE3) [pMS470-CtNHase] cells or mutants thereof were cultivated in deep well plates (protocol see 2.3.2). Frozen pellets were resuspended in 200 μL 200 mM Tris-HCl, pH 7, to give OD₆₀₀ values of around 20. 12.5 μL of cells were mixed with 37.5 μL of 133.33 mM rac-1 (100 mM final concentration) and incubated at ambient temperature for 30 min at 700 rpm on a Titramax device. Afterwards, 50 μL of 200 mM hydroxyl ammonium chloride were added as well as 50 μL of 27 mg/mL ReAmidase-CFE, partially purified by ammonium sulfate precipitation. After one hour of incubation at 30° C., 50 μL of 0.6 M FeCl₃ in 1 M HCl were added causing a yellow coloration of blank reactions whereas high nitrile hydratase activity resulted in a red color.

The evaluation was done on the computer. In principle, the red color was quantified by measuring the grey values of the wells. Photos were converted to greyscale using IrfanView (Version 4.38). In ImageJ, the wells were marked as regions of interest (ROI) and mean grey values were calculated for these ROIs, giving numbers from 0 to 255,000. These values were divided by 1,000 and subtracted from 255. In doing so, the dark wells (red color) obtained higher numbers than control wells, usually having a yellow color. In addition, grey values were normalized by the cell density (OD₆₀₀ values). The wells with the highest mean grey value as well as the ones with the highest normalized grey value were selected for re-screening.

2.5.3 Preparation of ReAmidase-CFE for Coupled Assays

Two flasks filled with 50 mL LB-Amp were inoculated with a small amount of cell material from a cryo-conserved sample. ONCs were grown at 37° C. under shaking (appr. 110-150 rpm) overnight. The next day, 400 mL of LB-Amp media were inoculated with 4 mL of ONC and incubated at 37° C. and 120 rpm until an OD₆₀₀ of 0.8-1 was reached. Protein expression was induced with 0.3 mM IPTG. Induced cells were cultivated at 25° C. at 120 rpm for 18-22 h and harvested by centrifugation in a JA-10 rotor for 15 min at 4° C. and 5,000 g. Supernatant was decanted and pellets of two flasks were frozen at −20° C.

A cell pellet of 800 mL main culture (appr. 3.5-5 g) was resuspended in 30 mL of 100 mM sodium phosphate buffer, pH 7.5 and lysed on ice by sonication for 8 min at 70-80% duty cycle and 7-8 output control. Cell-free extracts were obtained after centrifugation at 48,250 g and 4° C. for 1 h and filtered through 0.45 μM syringe filters.

The ReAmidase was enriched in the cell-free extract by ammonium sulfate precipitation. Thus, ammonium sulfate was added slowly at 4° C. to reach a saturation of 35%. The required amount of ammonium sulfate at given room temperature was calculated by an online tool (http://www.encorbio.com/protocols/AM-SO4.htm). Once the ammonium sulfate was completely dissolved, the CFE was stirred for one hour at 4° C. Centrifugation was done in a JA-10 rotor for 15 min at 10,000 g and 4° C. to remove precipitated background proteins. The supernatant, which contained the ReAmidase, was very carefully decanted. Again, ammonium sulfate was added slowly to reach a saturation of 60% and stirred for another hour at 4° C. The centrifugation step was repeated and the pellet stored at 4° C. overnight.

The next day, the ReAmidase containing protein pellet was dissolved in 100 mM sodium phosphate buffer, pH 7.5. Around 25% of the volume of the applied CFE was added to obtain a four-fold enrichment. Protein concentration was determined using the Pierce BCA Protein Assay Kit (ThermoFisher Scientific). This partially purified ReAmidase-CFE was diluted to 27 mg/mL and frozen at −20° C.

### 3. Experiments

### Example 3.1. Provision of a NHase Panel

3.1.1 Selection of Suitable Enzymes

The NHase panel consisted of 21 enzymes (Table 1). From already known enzymes, thermostable, well expressible and (S)-selective NHases were chosen. Predicted NHase sequences were analyzed regarding their probability for PEST degradation or membrane regions. Critical sequences were not accepted. Also, NHases of thermostable organism were favored and psychrophilic ones were rejected. Of closely related NHases only one was chosen.

Expression plasmids were available for four Fe-type NHases, the remaining 17 NHase genes had to be cloned into the pMS470d8 vector. Genes coding for both subunits (see 2.2.1) as well as the accessory protein were ordered as synthetic DNA fragments and cloned via Gibson cloning (2.2.3).

3.1.2 Expression

Functional recombinant expression of nitrile hydratases requires three peptide chains (α and β subunit and accessory protein), correct assembly of α and β subunit and efficient metal uptake. Therefore, inducible protein expression was applied with a low induction temperature to avoid formation of inclusion bodies (see 2.3.1).

SDS-PAGE analysis was done of soluble and insoluble fractions (see 2.3.4). High expression levels were achieved for M/NHase, PcNHase, ReNHase, CjNHase, GhNHase, BrNHase. Expression of BjNHase, AmNHase and PtNHase was less successful. The KoNHase showed imbalanced overexpression with significant amounts of the β subunit but almost no α subunit. NaNHase was predominantly found in the insoluble fraction in our hands. A putative NHase from Rhizobium leguminosarum bv. trifolii (R/NHase) was found neither in the soluble nor the insoluble fraction. Overexpression was also achieved for AbNHase, CtNHase, RmNHase and VvNHase. The putative NHases from Ralstonia solanacearum (RsNHase) and from Tardiphaga robiniae (TrNHase) showed higher expression of the α subunit as compared to the β subunit. The iron-type PkNHase was well expressed whereas AcNHase was found neither in the soluble nor in the insoluble fraction. PmNHase showed imbalanced expression with higher amount of the β subunit. (Data not shown)

3.1.3 Activity in Methacrylonitrile Hydrolysis

The activity towards methacrylonitrile (MAN) was tested photometrically (see 2.4.1) for all NHases. In the first attempt, all CFEs were used in 1:10 and 1:100 dilutions in single measurement to find applicable dilutions for each NHase. For the second assay, one or two dilutions of each NHase-CFE were chosen and applied in triplicate for the activity calculation.

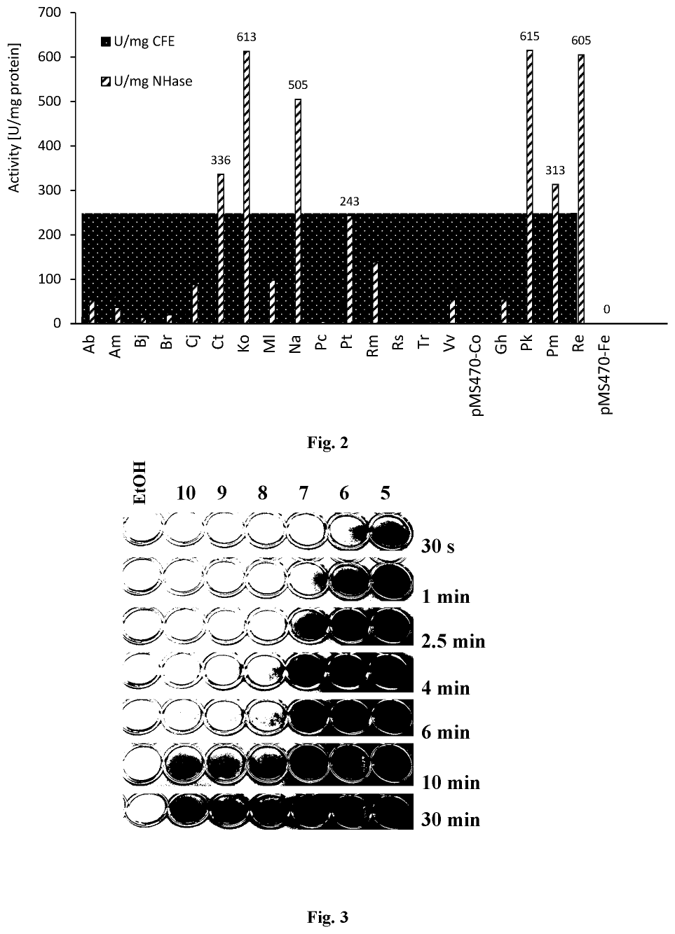

Out of 21 NHases, fourteen enzymes showed adequate hydrolysis activity for methacrylonitrile (FIG. 2). CtNHase, KoNHase, NaNHase, PtNHase, PkNHase, PmNHase and ReNHase showed high activity in this assay. The novel nitrile hydratases AbNHase, AmNHase, CjNHase, M/NHase, RmNHase, VvNHase and GhNHase were found to be active towards methacrylonitrile.

AcNHase and R/NHase being inactive was not surprising as there were no NHase bands visible in SDS-PAGE for these enzymes. Also, two literature-known NHases were not active in our hands: BjNHase and BrNHase. Moreover, PcNHase, RsNHase and TrNHase, all three putative enzymes, could be expressed but were not active towards MAN hydrolysis.

3.1.4 Hydration of rac-1

All NHase-CFE preparations were screened for hydrolysis of α-ethyl-1-pyrrolidineacetonitrile rac-1 (2.4.2) independent of their activity towards methacrylonitrile. Reactions with 10 mM substrate were run at 25° C. overnight and analyzed by HPLC (see 2.4.3). Seven nitrile hydratases were able to convert the target substrate (Table 4), with sometimes high enantioselectivities for the (S)-product. All expressed iron-type NHases showed the ability to convert the target substrate but only some cobalt-type ones. According to literature, iron-type NHases are more likely to convert aliphatic nitriles whereas cobalt-type NHases prefer aromatic nitriles [S. Prasad, T. C. Bhalla, Nitrile hydratases (NHases): At the interface of academia and industry, Biotechnol. Adv. 28 (2010) 725-741. doi:10.1016/J.BIOTECHADV.2010.05.020].

TABLE 4
Screening NHase-CFEs for hydrolysis of α-ethyl-1-pyrrolidineacetonitrile

| | | | Enantioselectivity ee_{P} |
|---|---|---|---|
| Sample | NHase [μg/mL] | Conversion [%] | [%] |
| Ab | 160 | 0 | — |
| Am | 76 | 0 | — |
| Bj | 147 | 0 | — |
| Br | 62 | 0 | — |
| Cj | 289 | 0 | — |
| Ct | 340 | 10.9 | 89 (S) |
| Ko | 289 | 5.3 | 79 (S) |
| Ml | 388 | 0 | — |
| Na | 102 | 1.1 | 22 (S) |
| Pc | 174 | 0 | — |
| Pt | 116 | 0 | — |
| Rl | n.d. | 0 | — |
| Rm | 419 | 0 | — |
| Rs | 200 | 0 | — |
| Tr | 166 | 0 | — |
| Vv | 146 | 0 | — |
| Ac | n.d. | 0 | — |
| Gh | 340 | 14.3 | 78 (S) |
| Pk | 291 | 7.8 | 71 (S) |
| Pm | 190 | 2.7 | 61 (S) |
| Re | 287 | 18.0 | 16 (S) |

n.d.: No protein bands of this NHase were detected on the SDS-PA gel.
10 mM of rac-1 were converted in 500 μL reactions at pH 7.2 and 25° C. and 300 rpm overnight.

### Example 3.2 Characterization of Potential NHase Candidates

As mentioned, among the 21 tested NHases, merely seven enzymes were capable to form amide 2: CtNHase, KoNHase, NaNHase, GhNHase, PkNHase, PmNHase and ReNHase.

These seven nitrile hydratases were investigated in more detail. Temperature and pH scope were identified and stability studies performed. Moreover, effects of metals were investigated and inhibition studies conducted. After all these experiments, the limitations of the target reaction were classified and the most suitable enzyme for the target reaction was selected.

3.2.1 Temperature Studies

The conversion of rac-1 was performed at 37° C. and 50° C. in overnight reactions (see section 2.4.2) and in parallel, NHase-CFEs were incubated at these temperatures for SDS-PAGE analysis (method 2.3.4). After overnight incubation, samples were centrifuged for removal of denatured protein and analyzed.

The production of 2 seemed to be quite independent of the reaction temperature (see Table 5), although not all enzymes were thermostable. The results indicate that most of the substrate is converted immediately when the thermolabile NHases were still functional and the reaction stops at some point for some reason.

TABLE 5
Conversion of 1 by promising NHases at higher temperatures.

| | [μg/ | 37° C. | | 50° C. | |
|---|---|---|---|---|---|
| NHase | mL] | conversion [%] | ee_{P} [%] | conversion [%] | ee_{P} [%] |
| Ct | 543 | 9.0 | 87 (S) | 6.6 | 86 (S) |
| Ko | 462 | 4.4 | 83 (S) | 3.8 | 78 (S) |
| Na | 163 | 0.6 | —^{a} | 0.5 | —^{a} |
| Gh | 544 | 13.4 | 78 (S) | 16.0 | 77 (S) |
| Pk | 466 | 9.6 | 66 (S) | 8.4 | 62 (S) |
| Pm | 305 | 3.3 | 75 (S) | 3.1 | 72 (S) |
| Re | 459 | 17.5 | 14 (S) | 17.0 | 14 (S) |

^{a}only (S)-amide was detected
10 mM of rac-1 were converted in 500 μL reactions at pH 7.2 and 37 or 50° C. and 300 rpm overnight.

3.2.2 pH Studies

A pH study was conducted with the seven promising NHases. Therefore, the photometric assay (see 2.4.1), in which the production of methacrylamide is followed at 224 nm, was performed at different pH values. NHase-CFE was diluted in the same buffer as then the substrate was dissolved. Following buffers were chosen: 100 mM citrate phosphate, pH 5 and 6, 100 mM sodium phosphate, pH 7 and 8, 100 mM Tris-HCl, pH 8.5 and 100 mM carbonate, pH 9, 9.5 and 10.

The pH optimum is clearly around pH 7. At pH 6 and 8, all NHases show still acceptable activities. At pH 5, there is a significant activity loss for all tested NHases. At higher pH, especially the Fe-type NHases lose activity while the Co-type NHases show still moderate activity levels. CtNHase and KoNHase are the most stable ones. They were still active at pH 10 but with a significant lower activity than at pH 7 (data not shown).

CtNHase and KoNHase, both still active at high pH, were also assayed (2.4.1) after longer incubation at high pH in order to test their stability. The samples were diluted 1:10 with the reaction buffer (either pH 9 or 9.5) and incubated at 25° C. After certain time points, the samples were further diluted for the assay which was performed at the same pH as the incubation. CtNHase is stable up to pH 9.5 at 25° C., whereas KoNHase loses activity with time at pH 9.5 (data not shown).

3.2.3 Elevated Temperature and pH

Thermal stability of promising NHases was investigated at higher pH in the photometrical assay of methacrylonitrile hydrolysis (2.4.1). NHase-CFEs were incubated at 37° C. or 50° C. at pH 8 for 1 and 6 h. After the incubation, the samples were assayed at pH 8 and 25° C. for methacrylonitrile hydration.

CtNHase and KoNHase seem stable at 37° C. Also, NaNHase shows residual activity after one hour of incubation, but none after six hours. The Fe-type NHases, Gh, Pk, Pm and Re lost their activities completely after one hour. According to literature, Co-type NHases are the more stable ones, as confirmed in this experiment [S. Prasad, T. C. Bhalla, Nitrile hydratases (NHases): At the interface of academia and industry, Biotechnol. Adv. 28 (2010) 725-741. doi:10.1016/J.BIOTECHADV.2010.05.020.]. The incubation at 50° C. led to an activity loss of all seven NHases. Only CtNHase showed at least some residual activity after one hour incubation (data not shown).

We concluded that CtNHase is the most stable NHase among the seven tested ones, followed by KoNHase. The most promising Fe-type NHase is GhNHase due its high enantioselectivity.

3.2.4 Conversion of Higher Concentrations of Rac-1

50 mM and 100 mM of rac-1 were treated with the seven promising NHases to assess their ability to handle higher substrate concentrations (see 2.4.2). For higher substrate concentrations, the conversion rate was decreased (Table 6). In total amounts, more amide was produced in the 50 mM reactions than in the 10 mM reactions. The conversion of the 100 mM reactions was around half as much as of the 50 mM reactions, meaning that in total nearly the same amide concentration was reached.

TABLE 6
Conversion of different concentrations of 1 by NHase-CFEs.

| | 10 mM substrate | | 50 mM substrate | | 100 mM substrate | |
|---|---|---|---|---|---|---|
| NHase | Conv. [%] | ee_{P} [%] | Conv. [%] | ee_{P} [%] | Conv. [%] | ee_{P} [%] |
| Ct | 10.9 | 89 (S) | 8 | 85 (S) | 4 | 83 (S) |
| Ko | 5.3 | 79 (S) | 6.2 | 79 (S) | 2.6 | 75 (S) |
| Na | 1.1 | 22 (S) | 0.7 | 64 (S) | 0.4 | 54 (S) |
| Gh | 14.3 | 78 (S) | 10 | 78 (S) | 6.1 | 77 (S) |
| Pk | 7.8 | 71 (S) | 5.3 | 70 (S) | 2.9 | 69 (S) |
| Pm | 2.7 | 67 (S) | 1.8 | 76 (S) | 1 | 69 (S) |
| Re | 18 | 16 (S) | 15.2 | 11 (S) | 7.2 | 10 (S) |

Overnight reactions were performed at 25° C. and pH 7.2 for 50 and 100 mM of substrate. The product was analyzed by HPLC.

3.2.5 Time Studies

A time study was performed (see 2.4.2) to investigate when the target reaction slows down or if the reaction is extremely slow. Therefore, an overnight reaction was analyzed at different time points: After 30 min, 1, 2, 4, 6 and 21 h of incubation. Ct, Ko and GhNHase were used to convert 20 mM substrate in 900 μL scale at 25° C. and pH 7.2.

Most of the product was synthesized during the first half hour. After that, the product concentration increased only slightly (for GhNHase) or not at all. This time, 20% conversion were achieved by GhNHase. The enantiomeric excess of the product did not change during time of incubation and was as high as usual for these NHases: CtNHase with 88%, KoNHase with 81% and GhNHase with 79% for (S)-2. (data not shown)

3.2.6 Effect of Metals

The activity towards methacrylonitrile or rac-1 hydration was investigated with metal pre- or co-incubation for different reasons.

Cobalt Pre-Incubation

Pre-incubation with their metal cofactor can enhance the enzymatic activity, particularly if the enzyme was not fully loaded or the metal is only loosely bound in the active site. Thus, NHase-CFEs were incubated with CoCl₂ before they were assayed for methacrylonitrile conversion (2.4.1). CtNHase-CFE and KoNHase-CFE were incubated with 1 or 2 mM CoCl₂ for 2 h at 25° C. before they were tested in the photometrical assay. The Cobalt-pre-incubated samples showed lower activity than the control reaction that was incubated without additional Cobalt (data not shown).

Cobalt-pre-incubation did not increase the activity of tested NHases. This fact leads to the conclusion that the tested NHases already had a high and stable metal content which could not be increased further by pre-incubation.

Effect of Fe and Mn

As the target nitrile dissociates in aqueous solution, cyanide is released and inhibits then the nitrile hydratase. One idea to circumvent this effect was to add metals to the reaction solution which should form complexes with the released cyanide. For this reason, the NHase activity in methacrylonitrile hydration (2.4.1) was monitored in presence of Fe(III), Mn(II) and Fe(II) (data not shown).

FeCl₃ inhibited CtNHase dramatically even in small amounts, whereas GhNHase showed higher activity in presence of 1 mM FeCl₃. This can be explained by its metal dependence: Fe(III). At higher concentrations of FeCl₃ (2 mM), GhNHase's activity was also decreased (data not shown).

Up to 2 mM, FeCl₂ did not influence GhNHase activity but 5 mM FeCl₂ inhibited this enzyme completely. CtNHase lost continuously activity in presence of FeCl₂ (data not shown).

CtNHase was only a little inhibited by MnCl₂. It did not lose activity up to 2 mM, and in presence of 10 mM MnCl₂ CtNHase showed still 72% activity for methacrylonitrile conversion. GhNHase was rapidly inhibited by MnCl₂, even if only 1 mM is present (data not shown).

3.2.7 Effect of Co-Solvents

Co-solvents can alter the enantioselectivity of biotransformations [Y. Mine, K. Fukunaga, K. Itoh, M. Yoshimoto, K. Nakao, Y. Sugimura, Enhanced enzyme activity and enantioselectivity of lipases in organic solvents by crown ethers and cyclodextrins, J. Biosci. Bioeng. 95 (2003) 441-447. doi:10.1016/S1389-1723(03)80042-7; K. Watanabe, S. Ueji, Dimethyl sulfoxide as a co-solvent dramatically enhances the enantioselectivity in lipase-catalysed resolutions of 2-phenoxypropionic acyl derivatives, J. Chem. Soc. Perkin Trans. 1. (2001) 1386-1390. doi:10.1039/b100182p]. Methanol, DMSO and ethyl acetate were tested for Ct, Ko and GhNHase. 50 mM of rac-1 were treated with NHases overnight at 25° C. and pH 7.2 in the presence of 5% co-solvent (see 2.4.2).

Compared to the reaction without co-solvent, less amide was produced. However, the enantiomeric excess did not change. The tested co-solvents did not enhance the enantioselectivity in the tested concentrations (data not shown).

3.2.8 Effect of Catalyst Amount

The target reaction (2.4.2) was performed with the double amount of enzyme hopefully reaching higher conversion rates this way. More product was generated if more enzyme was applied. The enantioselectivity for (S)-2 was independent of the catalyst amount: 86% for CtNHase, 78% for KoNHase and 76% for GhNHase (data not shown).

3.2.9 Large Scale Production of Amide 2

For the synthesis of (S) enantiomerically enriched 2, the biocatalytic reaction was scaled up to 20 mL. 20 mM of rac-1 was converted in 3 h by GhNHase, as this NHase had shown the highest conversion level in previous experiments. The reaction was performed at 25° C. and pH 7.2. Next to a batch reaction, a substrate feeding reaction was also performed to hopefully achieve higher product amount. This reaction was started with 4 mM substrate with the increase of substrate by 4 mM every 30 min, until 20 mM substrate was consumed in the end. After conversion, the batches were frozen at −20° C. and lyophilized. Synthesis was also done using CtNHase. Therefore, two 20 mL reactions were conducted with 50 mM rac-1 and 20% (v/v) of CFE at 5° C. and pH 7.2 overnight.

HPLC analysis revealed that in the batch reaction 2.7 mM amide was obtained (13.4% conversion, 78% ee), while only 1.6 mM was obtained in the substrate feeding reaction (7.9 conversion, 76% ee). Summarizing, around 13 mg of 2 were synthesized, predominantly the (S) enantiomer.

In the next reactions with more substrate that were performed in duplicate (2.4.2), CtNHase reached conversion levels of 31.3 and 34 respectively. The ees for (S)-2 were at 82%.

3.2.10 Substrate Stability

The results of previous amide synthesis experiments prompted that the substrate might be unstable under reaction conditions. In literature, α-aminonitriles are described as unstable in aqueous solutions [Z.-J. Lin, R.-C. Zheng, Y.-J. Wang, Y.-G. Zheng, Y.-C. Shen, Enzymatic production of 2-amino-2,3-dimethylbutyramide by cyanide-resistant nitrile hydratase, J. Ind. Microbiol. Biotechnol. 39 (2012) 133-141. doi:10.1007/s10295-011-1008-6]. If so, the α-aminonitrile would dissociate into pyrrolidine, propanal and cyanide. The dissociation of α-aminonitrile can be tested with Feigl-Anger paper [F. Feigl, V. Anger, Replacement of benzidine by copper ethylacetoacetate and tetra base as spot-test reagent for hydrogen cyanide and cyanogen, Analyst. 91 (1966) 282. doi:10.1039/an9669100282], a sensitive filter paper, which detects small amounts of cyanide that evaporates to the gas phase (see 2.4.4).

At low pH, the nitrile decomposes immediately and after 30 s cyanide can be detected (FIG. 3). Also at higher pH values, cyanide was detected within three minutes. However, the substrate is quite stable in ethanol.

This experiment confirms the assumption that the substrate is not stable under reaction conditions as it decomposes in aqueous solutions. However, it is much more stable in organic solvents. The decomposition itself is a requirement for the envisaged dynamic kinetic resolution to allow for reconstitution of rac-1 from unreacted (R)-1.

The stability of rac-1 was further investigated at different pH values. Samples were extracted immediately as well as after 2 and 60 min. Subsequently, they were analyzed by HPLC (protocol 2.4.4). The extraction efficiency is dependent on the pH as the nitrile can be protonated. Therefore, the reactions were only compared to their zero values. The chromatograms showed the substrate dissociates quickly in buffer and cannot be recovered. The lower the pH, the more rac-1 is dissociated. At low pH the decrease in substrate concentration is much higher than at higher pH (Table 7). Moreover, at pH 9 and 10 there is hardly any difference between 2 min and 1 h incubation, indicating fast formation of the equilibrium.

TABLE 7
HPLC analysis of rac-1 incubated for various time frames at different pH.

| | Zero value | | 2 min | | 1 hour | |
|---|---|---|---|---|---|---|
| pH | Peak area | Percent | Peak area | Percent | Peak area | Percent |
| 5 | 11477.7 | 100 | 6556.7 | 57 | 3698.9 | 32 |
| 6 | 16666.6 | 100 | 9048.1 | 54 | 3863.2 | 23 |
| 6.5 | 16459.5 | 100 | 7764.2 | 47 | 3682.6 | 22 |
| 7 | 16597.9 | 100 | 9379.6 | 57 | 7384.5 | 44 |
| 7.5 | 15434.9 | 100 | 10179.5 | 66 | 7778.8 | 50 |
| 9 | 16901.9 | 100 | 14093.2 | 83 | 14777.5 | 87 |
| 10 | 18207 | 100 | 14145.8 | 78 | 15573.8 | 86 |

3.2.11 Inhibition Studies

Inhibition studies were performed to identify the limitations of the target reaction. The industrial substrate, α-ethyl-1-pyrrolidineacetonitrile, is instable in aqueous solution and dissociates into pyrrolidine, propanal and cyanide. The dissociation and formation of the nitrile is an equilibrium reaction dependent on the pH (Table 7). The addition of one of the three components could shift the equilibrium towards the nitrile and thus, suppress cyanide inhibition. If propanal or pyrrolidine would not harm the enzyme, they could be added in excess to the reaction.

Effect of Cyanide

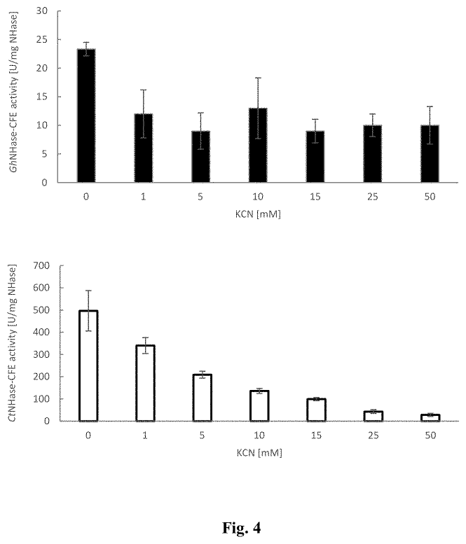

It is literature known that cyanide may inhibit NHases [Z.-J. Lin, R.-C. Zheng, Y.-J. Wang, Y.-G. Zheng, Y.-C. Shen, Enzymatic production of 2-amino-2,3-dimethylbutyramide by cyanide-resistant nitrile hydratase, J. Ind. Microbiol. Biotechnol. 39 (2012) 133-141. doi:10.1007/s10295-011-1008-6]. The effect of KCN on CtNHase and GhNHase activity was investigated using the photometrical assay (2.4.1) in which the formation of methacrylamide is followed at 224 nm. Cyanide decreased the activity of GhNHase by around 55% but the inhibitory effect seems to be independent of the cyanide concentration (FIG. 4). CtNHase showed decreasing activity with increasing cyanide concentration. At 50 mM cyanide, CtNHase showed only 6% of its initial activity. Nevertheless, even at 50 mM cyanide CtNHase with 28 U/mg NHase had a higher activity than GhNHase with 23 U/mg NHase.

Another possible reason for the low conversion levels would be product inhibition. Therefore, NHase-CFEs were incubated with rac-2 before they were assayed for methacrylonitrile hydrolysis in the photometric assay (2.4.1). Neither CtNHase nor GhNHase were inhibited by 2 up to a concentration of 5 mM (data not shown). This assay does not allow to use higher concentrations of rac-2. Although product inhibition is unlikely at concentrations up to 50 mM, as judged by determined product concentrations by HPLC in other experiments, inhibitory effects of higher product concentrations may only be determined by the HPLC based assay in the presence of increasing amounts of amide 2.

Effect of Propanal

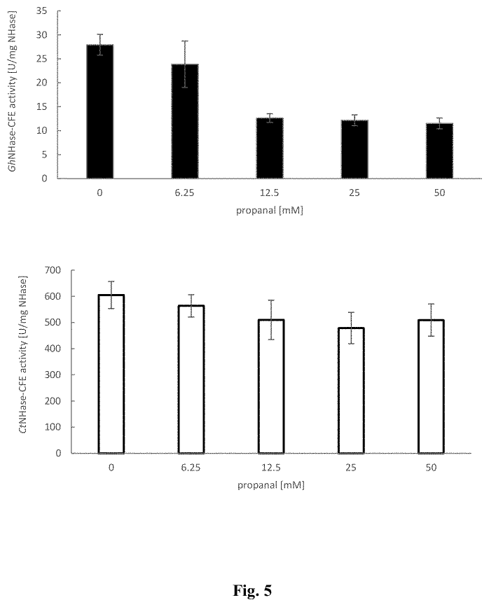

The activity towards methacrylonitrile hydration was determined in the presence of propanal (2.4.1). Propanal decreased GhNHase activity but not CtNHase activity (FIG. 5). Whereas GhNHase activity was reduced by around 50%, CtNHase showed still more than 80 activity in the presence of 50 mM propanal.

3.2.12 Effect of Low Conversion Temperature

Conversion of rac-1 at 5° C. (see 2.4.2) yielded more of 2 for CtNHase-CFE but not for GhNHase (FIG. 6). The enantiomeric excess was nearly the same for both temperatures for CtNHase (81 and 82% for (S), respectively) whereas GhNHase showed a higher enantioselectivity at 25° C. (65 vs. 61% ee for (S), respectively).

The time study revealed that the reactions as 25° C. were very fast (data not shown). Product was only synthesized in the first 5-10 minutes. At 5° C., even after 30 min amide was formed by CtNHase whereas GhNHase stopped after 20 min. GhNHase achieved a higher product amount at 25° C., but CtNHase produced more amide at 5° C. (Table 8).

TABLE 8
Conversion rates and enantiomeric excess for the (S)-amide
for CtNHase and GhNHase at 5° C. and 25° C.

| NHase | Ct | Ct | Ct | Gh | Gh | Gh |
|---|---|---|---|---|---|---|
| temperature [° C.] | 25 | 5 | 5 | 25 | 5 | 5 |
| time [min] | 30 | 30 | 60 | 30 | 30 | 60 |
| amide 2 [mM] | 2.1 | 4.8 | 6.6 | 4.7 | 3.5 | 4.1 |
| conversion [%] | 10.3 | 24.1 | 33 | 23.3 | 17.4 | 20.5 |
| enantiomeric excess [%] | 78 (S) | 82 (S) | 83 (S) | 63 (S) | 65 (S) | 67 (S) |

3.2.13 Effect of Low Enzyme Feeding

CtNHase and GhNHase were tested in an enzyme feeding reaction for synthesis of 2 (2.4.2). More product was achieved if additional CFE was supplemented (Table 9) and also the ee was higher for the feeding reactions. The results indicate that the enzymes lost their functionality during the reaction.

TABLE 9
Conversion rate and enantiomeric excess for the
(S)-amide in enzyme feeding experiment.

| | CtNHase | | GhNHase | |
|---|---|---|---|---|
| | control | feeding | control | feeding |
| Conversion [%] | 6.9 | 10 | 22 | 34.7 |
| Enantiomeric excess [%] | 73 (S) | 78 (S) | 69 (S) | 73 (S) |

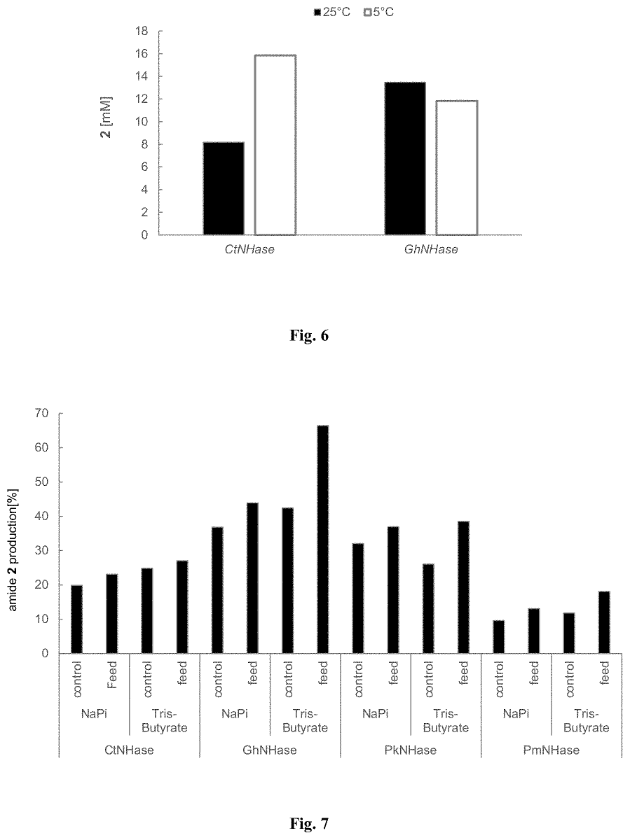

A similar experiment was done with four NHases and two buffers at 5° C. The best NHase in those reactions was GhNHase, which reached over 60% conversion in the enzyme feeding reaction (FIG. 7). The enzyme feed resulted in a higher product amount indicating that there is a problem with the enzyme's stability or enzyme inactivation. Nevertheless, 66% conversion were achieved with an enantiomeric excess of 68% (S).

3.2.14 Conversion of Rac-1 by CtNHase and GhNHase at Different pH

The pH turned out to be a critical factor in biocatalytic (S)-2 synthesis. First of all, both the nitrile 1 and the amide 2 are basic compounds and might increase the pH upon addition if the buffer capacity is insufficient. Second, nitrile hydratases show the highest activity from pH 7 to 8 and third, the substrate 1, being an α-aminonitrile, is in equilibrium with its three building blocks pyrrolidine, propanal and cyanide. The higher the pH, the more stable is the α-aminonitrile. Therefore, several hydration reactions of rac-1 were carried out at different pH values (method 2.4.2) to find the pH most suitable for the reaction.

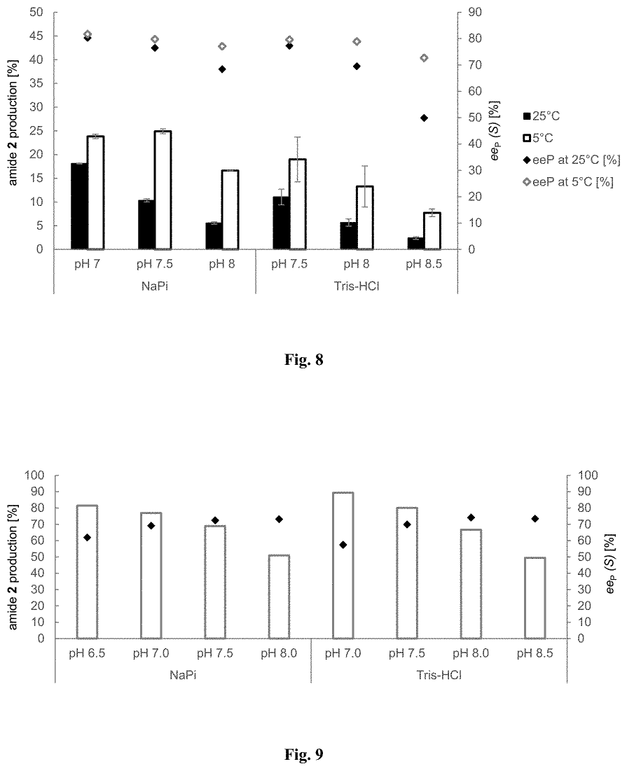

One has to keep in mind that the pH of Tris-HCl buffer strongly depends on the temperature. At 5° C. the pH was 0.5 units higher than at 25° C. The highest conversion was achieved at 5° C. in sodium phosphate buffer, pH 7.5 (FIG. 8). At 25° C., pH 7 was clearly better. Also the Tris-HCl buffer showed an obvious trend: The lower the pH, the higher the conversion rate. The enantiomeric excess was also the best at pH 7. Higher conversions at low temperature might be explained by the fact that substrate dissociation is faster at higher temperature, introducing more inhibiting cyanide to the mixture.

The same experiment as above was also performed with GhNHase. In the previous experiment the best results were reached at the lowest pH and therefore, in this experiment also pH 6.5 was tested. The reactions with GhNHase-CFE were carried out at 25° C. only.

Also, GhNHase-CFE shows the highest conversion at pH 7 (FIG. 9). In contrast to CtNHase, its enantioselectivity increased with increasing pH, with significant differences (57.5-74.2% (S)). A possible explanation for this is that GhNHase converts the substrate really fast and overrules the chemical dissociation (to some extent) so that at pH 7 more of the (R)nitrile is converted as at higher pH values, where its reaction rate is slower.

3.2.15 Effect of Low Catalyst Amount

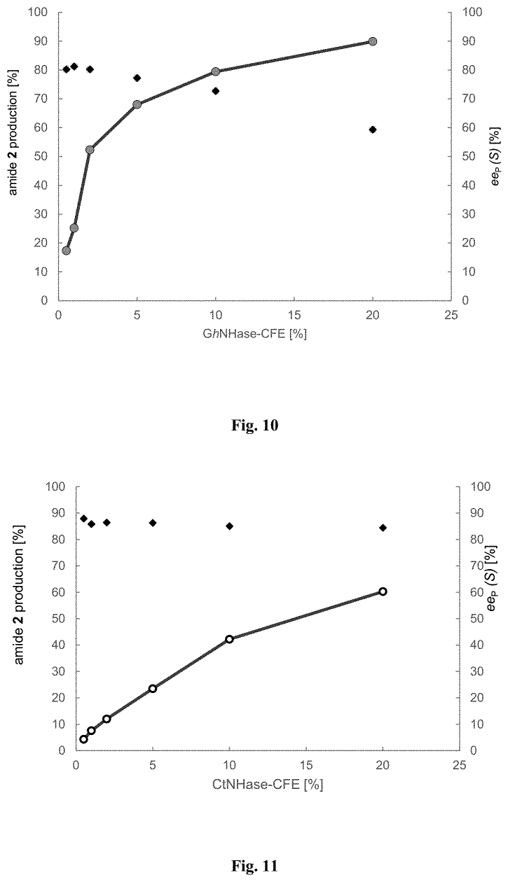

The more GhNHase-CFE was applied in hydration reactions of rac-1 (2.4.2), the more product was obtained (FIG. 10). However, the correlation was not linear as the substrate was already limited at higher CFE concentrations. Interestingly, the enantiomeric excess was dependent on the catalyst amount. The more catalyst we applied, the more (S)-2 was synthesized but the worse was the ee value. NHase catalyzed hydration was apparently faster than the chemical dissociation and reformation of rac-1.

The correlation between CtNHase-CFE amount and product concentration was almost linear (FIG. 11). This time, the enantiomeric excess was quite the same for all tested catalyst concentrations. CtNHase showed less activity than GhNHase on the target substrate and therefore chemical dissociation and reformation of rac-1 did not become limiting. Likely, more product would have been synthesized in a prolonged reaction.

In the time study with 2% (v/v) GhNHase-CFE, most of the substrate was converted within 45 min (FIG. 12). It looks like the conversion was complete within 2 h. The enantiomeric excess of 77% was quite high for GhNHase-CFE.

3.2.16 Substrate Feeding Reactions with GhNHase Cells

Within this experiment, the stability of GhNHase under reaction conditions was investigated (method 2.4.2). Batch reactions were performed as controls. In the feeding reactions, the concentration of rac-1 was doubled after 2 h. Incubation was done at 25° C. and 500 rpm for 4 h.

In general, GhNHase cells were still active after 2 h although less substrate was converted of the second substrate portion. The enantioselectivity was higher, when substrate was fed to the reaction (Table 10).

TABLE 10
Evaluation of substrate feeding experiment
with lyophilized GhNHase cells.

| | 25 mM | 25 mM | 50 mM | 50 mM |
|---|---|---|---|---|
| Reaction | control | feed | control | feed |
| product [mM] | 22.7 | 41.2 | 44.7 | 79.1 |
| theoretical | 25 | 50 | 50 | 100 |
| yield [mM] | | | | |
| conversion [%] | 90.8 | 82.4 | 89.4 | 79.1 |
| ee_{P} [%] | 51.3 (S) | 62.2 (S) | 56.2 (S) | 66.0 (S) |

3.2.17 First Conversion of Rac-1 by Whole Cells

Reactions with NHase-CFE were yet not fully satisfying as the conversions stopped at some point although the pH was still low enough to not denature the enzymes. Another possible reason for the reaction stop might be enzyme inactivation by cyanide. Cyanide inhibits nitrile hydratases by forming complexes with their metal ion [S. van Pelt, M. Zhang, L. G. Otten, J. Holt, D. Y. Sorokin, F. van Rantwijk, G. W. Black, J. J. Perry, R. A. Sheldon, Probing the enantioselectivity of a diverse group of purified cobalt-centred nitrile hydratases, Org. Biomol. Chem. 9 (2011) 3011. doi:10.1039/c0ob01067g; T. Gerasimova, A. Novikov, S. Osswald, A. Yanenko, Screening, Characterization and Application of Cyanide-resistant Nitrile Hydratases, Eng. Life Sci. 4 (2004) 543-546. doi:10.1002/elsc.200402160]. Using whole cells might solve this problem by protecting the enzymes with the cell membranes against released cyanide. Reactions with rac-1 were performed at different pH values and catalyst amount (method 2.4.2).

The following trends were observed for GhNHase (Table 11): The more catalyst was used, the more product was synthesized although this correlation was not linear. The higher the pH, the less product. This correlation was true especially for reactions with little catalyst. However, the higher the pH, the higher was the enantiomeric excess towards (S)-2. Also, the less catalyst was used, the higher the ee values were.

TABLE 11
Whole cell conversions with GhNHase.

| Reaction conditions | conversion [%] | ee_{P} (S) [%] |
|---|---|---|
| 8.5 mg/mL cells, pH 7 | 79.3 | 65.6 |
| 8.5 mg/mL cells, pH 7.5 | 98.0 | 68.5 |
| 8.5 mg/mL cells, pH 8 | 81.3 | 71.5 |
| 4.25 mg/mL cells, pH 7 | 81.1 | 71.7 |
| 4.25 mg/mL cells, pH 7.5 | 75.2 | 74.4 |
| 4.25 mg/mL cells, pH 8 | 68.2 | 76.4 |
| 1.7 mg/mL cells, pH 7 | 64.3 | 77.8 |
| 1.7 mg/mL cells, pH 7.5 | 59.1 | 78.5 |
| 1.7 mg/mL cells, pH 8 | 32.0 | 79.0 |
| 0.85 mg/mL cells, pH 7 | 54.0 | 79.4 |
| 0.85 mg/mL cells, pH 7.5 | 30.5 | 79.6 |
| 0.85 mg/mL cells, pH 8 | 10.6 | 81.4 |
| 0.34 mg/mL cells, pH 7 | 20.4 | 80.0 |
| 0.34 mg/mL cells, pH 7.5 | 7.1 | 82.2 |
| 0.34 mg/mL cells, pH 8 | 2.6 | 81.1 |
| 0.17 mg/mL cells, pH 7 | 5.4 | 81.8 |
| 0.17 mg/mL cells, pH 7.5 | 4.8 | 84.6 |
| 0.17 mg/mL cells, pH 8 | 3.2 | 82.9 |

Conversion of 50 mM rac-1 were performed at different pH and with different catalyst amount at 25° C. and 500 rpm for 2 h.

With little catalyst, only low conversion levels were achieved. Maybe the reaction time was too short for the low catalyst amount. Also, substrate dissociation might be the reason for the low conversions. Released HCN can inactivate the enzyme, aldehyde might evaporate and become unavailable or also react with proteins.

The same experiment was done with CtNHase. In general, CtNHase is less active towards rac-1 than GhNHase, but shows a higher enantioselectivity (Table 12). The highest conversions were reached at pH 7. The enantiomeric excess was higher when less catalyst was used although these values should be handled with care as for small product concentrations hardly any (R)-2 was detected.

The correlation between catalyst and product amount was linear. This finding presumes that the substrate concentration was not limiting the reaction rate.

TABLE 12
Whole cell conversions with CtNHase.

| Reaction conditions | conversion [%] | eeₚ (S) [%] |
|---|---|---|
| 8.5 mg/mL cells, pH 7 | 46.9 | 85.7 |
| 8.5 mg/mL cells, pH 7.5 | 40.7 | 85.6 |
| 8.5 mg/mL cells, pH 8 | 25.7 | 86.1 |
| 4.25 mg/mL cells, pH 7 | 28.3 | 86.9 |
| 4.25 mg/mL cells, pH 7.5 | 22.2 | 86.9 |
| 4.25 mg/mL cells, pH 8 | 15.8 | 86.3 |
| 1.7 mg/mL cells, pH 7 | 13.1 | 88.0 |
| 1.7 mg/mL cells, pH 7.5 | 10.2 | 87.9 |
| 1.7 mg/mL cells, pH 8 | 5.6 | 90.6 |
| 0.85 mg/mL cells, pH 7 | 7.5 | 87.8 |
| 0.85 mg/mL cells, pH 7.5 | 5.5 | 89.8 |
| 0.85 mg/mL cells, pH 8 | 3.5 | 90.4 |
| 0.34 mg/mL cells, pH 7 | 2.6 | 92.8 |
| 0.34 mg/mL cells, pH 7.5 | 2.4 | 90.6 |
| 0.34 mg/mL cells, pH 8 | 1.5 | 90.6 |
| 0.17 mg/mL cells, pH 7 | 1.3 | 96.1 |
| 0.17 mg/mL cells, pH 7.5 | 2.3 | 94.1 |
| 0.17 mg/mL cells, pH 8 | 1.3 | 88.3 |

Conversion of 50 mM rac-1 were performed at different pH and with different catalyst amount at 25° C. and 500 rpm for 2 h.

3.2.18 Time Study with Whole Cells

A time study of rac-1 hydration was also done for whole cell catalysis (see 2.4.2). Independent on the catalyst amount, nearly the whole product was synthesized within one hour. The reaction conditions somehow terminated amide synthesis. Either the enzyme was inactivated/inhibited by HCN or substrate became unavailable due to aldehyde evaporation or side reactions (data not shown).

3.2.19 Continuous Substrate Feeding

Substrate feeding studies were conducted in order to find out, how long NHase cells are active (see 2.4.2). In the first set-up, only little amounts of catalyst was used (1.7 mg/mL wet cell weight) for a high substrate concentration (200 mM total). In the second set-up, a high catalyst amount (8.5 mg/mL cells) was applied for 50 mM substrate. Both set-ups were tested with whole cells as well as with CFE.

Using biocatalyst in low amount, most of the product was synthesized before the first feeding step within the first hour. GhNHase produced 33 mM product in the first hour (of theoretical 50 mM) while CtNHase only synthesized 7 mM product. After one hour hardly any product was produced. The enzymes might be inactivated by the substrate or more specifically, the substrate dissociation and released HCN. In the case of GhNHase, still 17 mM substrate were not converted after one hour and in the case of CtNHase, even 43 mM. In total, GhNHase produced 42 mM product (21% conversion) and CtNHase only 10 mM of 2 (5% conversion). There was hardly any difference between GhNHase whole cells and GhNHase-CFE in this experiment whereas CtNHase cell performed better than CtNHase-CFE (Table 13).

TABLE 13
Amide concentration and enantiomeric excess of (S)-2 for the
substrate feeding study with 1.7 mg/mL cells.

| | GhNHase cells | | | GhNHase-CFE | | | CtNHase cells | | | CtNHase-CFE | | |
|---|---|---|---|---|---|---|---|---|---|---|---|---|
| Time | amide | | | amide | | | amide | | | amide | | |
| [h] | [mM] | ee_{P} [%] | | [mM] | ee_{P} [%] | | [mM] | ee_{P} [%] | | [mM] | ee_{P} [%] | |
| 1 | 33.1 | 1. | 79.6 | 32.2 | 2. | 79.6 | 6.9 | 3. | 87.2 | 4.0 | 4. | 90.1 |
| 2 | 40.6 | 5. | 79.8 | 40.1 | 6. | 79.7 | 8.3 | 7. | 87.3 | 4.9 | 8. | 90.7 |
| 3 | 41.2 | 9. | 79.8 | 40.2 | 10. | 79.9 | 8.7 | 11. | 87.9 | 5.2 | 12. | 91.6 |
| 4 | 40.9 | 13. | 75.3 | 40.9 | 14. | 75.3 | 10.2 | 15. | 68.3 | 6.4 | 16. | 60.5 |
| 5 | 42.1 | 17. | 75.3 | 41.5 | 18. | 75.3 | 10.4 | 19. | 68.2 | 6.5 | 20. | 60.8 |

The experiments with more catalyst and less substrate gained a higher conversion. In total, GhNHase reached 34% conversion and CtNHase 43%. Although GhNHase produced more amide in the first three hours, finally CtNHase showed a higher conversion. Nevertheless, reaction conditions for full conversion still had to be found.

In this experiment, whole cells performed significantly better than CFE. The difference was visible especially in the later phase of the reaction (Table 14).

TABLE 14
Amide concentration and enantiomeric excess of (S)-2 for the
substrate feeding study by 8.5 mg/mL cells.

| | GhNHase cells | | | GhNHase-CFE | | | CtNHase cells | | | CtNHase-CFE | | |
|---|---|---|---|---|---|---|---|---|---|---|---|---|
| Time | amide | | | amide | | | amide | | | amide | | |
| [h] | [mM] | ee_{P} [%] | | [mM] | ee_{P} [%] | | [mM] | ee_{P} [%] | | [mM] | ee_{P} [%] | |
| 1 | 6.7 | 21. | 58.6 | 6.4 | 22. | 58.2 | 6.5 | 23. | 88.5 | 5.0 | 24. | 89.6 |
| 2 | 13.5 | 25. | 67.5 | 11.8 | 26. | 68.5 | 10.5 | 27. | 88.8 | 8.9 | 28. | 89.6 |
| 3 | 16.3 | 29. | 68.9 | 12.6 | 30. | 68.5 | 15.7 | 31. | 88.2 | 11.8 | 32. | 89.3 |
| 4 | 17.0 | 33. | 69.5 | 12.8 | 34. | 68.6 | 19.3 | 35. | 88.0 | 13.6 | 36. | 89.1 |
| 5 | 17.2 | 37. | 69.7 | 12.6 | 38. | 68.6 | 21.8 | 39. | 88.1 | 15.4 | 40. | 88.9 |

3.2.20 Heat Purification of CtNHase and GhNHase

CtNHase was described as thermostable enzyme [K. L. Petrillo, S. Wu, E. C. Hann, F. B. Cooling, A. Ben-Bassat, J. E. Gavagan, R. DiCosimo, M. S. Payne, Over-expression in Escherichia coli of a thermally stable and regio-selective nitrile hydratase from Comamonas testosterone 5-MGAM-4D, Appl. Microbiol. Biotechnol. 67 (2005) 664-670. doi:10.1007/s00253-004-1842-9] and showed no loss of activity after 6 h at 37° C. in a previous experiment (see 3.2.3). Hence, heat purification (method 2.3.5) of CtNHase and also GhNHase was investigated.

CtNHase is quite thermostable and could be nicely purified at 60° C. The specific activity of heat purified CtNHase was approximately the same as for CtNHase in CFE (data not shown). Heat purification did not work for GhNHase which denatures almost completely at temperatures higher than 50° C., which is reflected by the disappearance of the 2 protein bands corresponding to the two subunits in SDS PA gel electrophoresis (data not shown).

3.2.21 Conversion of High Rac-1 Concentrations

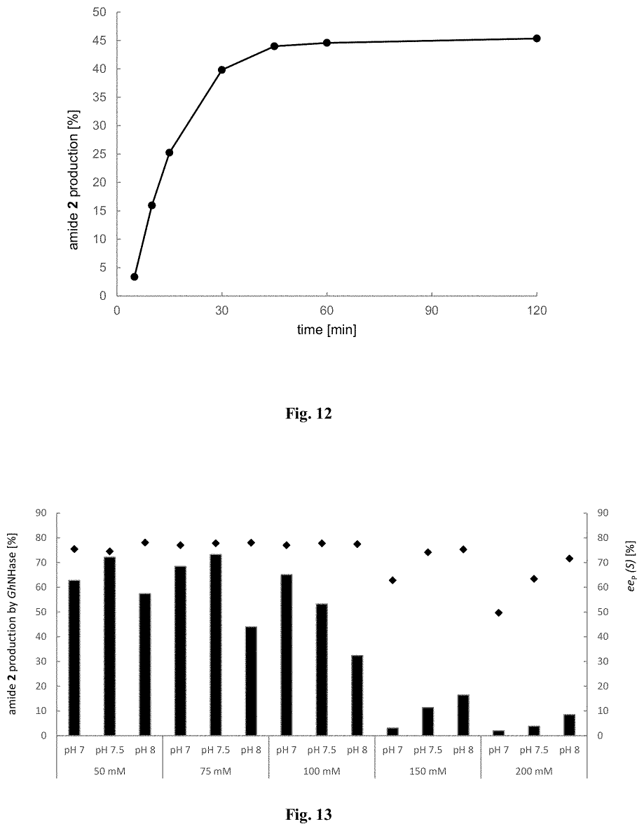

CtNHase and GhNHase were applied in rac-1 hydration reactions with up to 200 mM substrate (protocol in 2.4.2). GhNHase can handle up to 100 mM substrate quite well but of 150 mM substrate hardly any was converted (FIG. 13). Interestingly, at higher substrate concentrations more product was generated at higher pH. More substrate is dissociated at lower pH which might explain this effect. Also, the enantiomeric excess for the reactions with 150 and 200 mM substrate at pH 7 were the lowest which indicates that the enzyme did not work properly anymore but could also be an effect of peak integration (peaks close to detection limit in some cases).

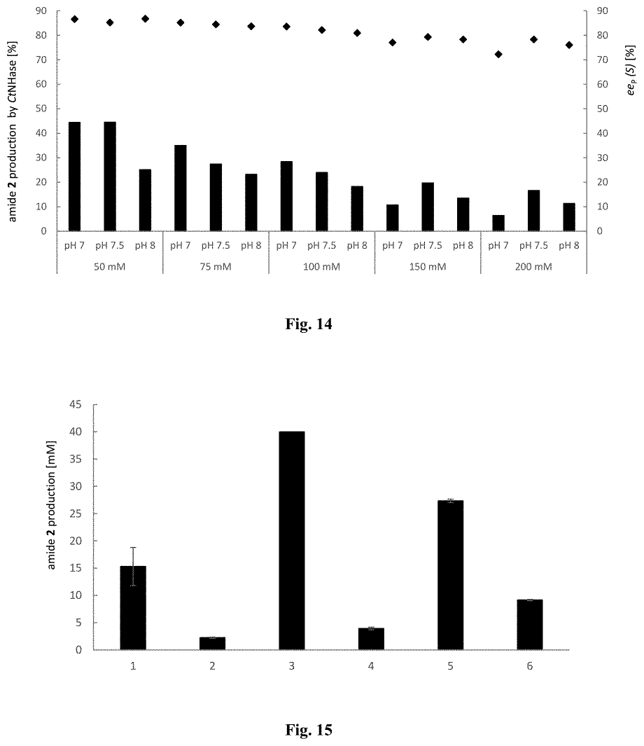

This wide difference between lower and higher substrate concentrations like for GhNHase was not observed for CtNHase (FIG. 14). The conversion level decreased but the difference in product amount itself was not that big. Again, for higher substrate concentrations pH 7 is not the best choice. Reactions with 50 mM substrate showed the highest enantiomeric excess which means that higher substrate concentrations do harm the enzyme, by influencing the pH and concomitantly the cyanide concentration. Anyway, at high substrate concentrations CtNHase produced more of the desired amide than GhNHase, which supports observations from the MAN assay, where Co-dependent NHases outperformed Fe-NHases at elevated pH values. Comparing GhNHase and CtNHase, average ees were again higher for CtNHase whereas conversions were higher using GhNHase.

3.2.22 Addition of Pyrrolidine and Propanal

Hydration of rac-1 by CtNHase was tested with additional pyrrolidine and/or propanal (see 2.4.2). The highest product amount was achieved in the presence of 150 mM propanal (FIG. 15). The addition of propanal might have several effects. First, the equilibrium between substrate dissociation and formation is shifted towards the nitrile side. Second, propanal might evaporate during the reaction so that the nitrile substrate becomes unavailable. Additional propanal can circumvent this problem. Thirdly, propanal might also react with protein and enzymes of the cells and therefore the NHase might become inactivated, too. This effect was already investigated for the conversion of methacrylonitrile and did not show a high influence on CtNHase activity. And lastly, propanal influences the pH of the reaction (Table 15). However, the addition of propanal increased the conversion level of CtNHase. Also the reaction with 75 mM propanal led to more product than without propanal.

Pyrrolidine did not improve the conversion rate of CtNHase so far but its effect on the pH has not been compensated, however, it is not negligible (Table 15). On the one hand, the low conversion levels can be explained by the elevated pH but on the other hand, the comparison of reactions 4 and 6 show that higher product amounts are possible at the same pH. Moreover, the enantiomeric excess of the reactions with pyrrolidine is quite low which indicates non-optimal conditions for the enzyme. Again, the pH is not the only reason shown by reaction 4 and 6. The addition of propanal seems to increase the product amount significantly.

TABLE 15
Evaluation of synthesis of 2 by CtNHase in
presence of pyrrolidine and propanal.

| reaction | 1 | 2 | 3 | 4 | 5 | 6 |
|---|---|---|---|---|---|---|
| substrate [mM] | 150 | 150 | 150 | 150 | 150 | 150 |
| pyrrolidine [mM] | — | 150 | — | 75 | — | 75 |
| propanal [mM] | — | — | 150 | — | 75 | 75 |
| product [mM] | 23.0 | 3.4 | 60.0 | 5.9 | 41.0 | 13.7 |
| conversion [%] | 15.3 | 2.3 | 40.0 | 3.9 | 27.3 | 9.2 |
| ± | 3.49 | 0.11 | — | 0.24 | 0.32 | 0.11 |
| ee_{P} (S) [%] | 79.7 | 57.0 | 78.7 | 67.2 | 79.8 | 75.4 |
| pH after 1 h | 8.2 | 8.6 | 7.9 | 8.4 | 8.2 | 8.4 |

Reactions with 8.5 mg/mL cells were performed in triplicates and incubated at 25° C. and 700 rpm for 1 h.

3.2.23 Comparison of CtNHase and GhNHase

By comparing and evaluating the different results on enzymatic characteristics and behavior of CtNHase and GhNHase as described above, CtNHase was chosen as the target protein to be engineered. However, considering the fact that GhNHase achieved for example significantly higher product concentrations, GhNHase may be chosen as a further promising target for protein engineering within the context of the present invention, which may be performed in analogy to the now in more detail described CtNHase-based engineering experiments.

### Example 3.3 Site-Saturation Mutagenesis of CtNHase

Semi-rational engineering methods were applied to find CtNHase mutants producing high amounts of enantiopure (S)-2.

Promising candidates were applied in bioconversions and reaction products were analyzed by HPLC, showing improved mutants with significantly higher enantioselectivities for (S)-2. The best mutant from this approach, CtNHase-βF51L, reached 73% conversion of 50 mM 1 with an enantiomeric excess of 93% for (S)-2.

3.3.1 Identification of Important Amino Acid Residues

Using structural biology methods, amino acid residues with potentially high influence on activity and enantioselectivity were identified. Amino acids in close proximity to the substrate binding site are meant to be crucial for substrate positioning and therefore also enantioselectivity. In order to perform docking studies, a structure model of CtNHase was created.

As nitrile hydratases are hetero-dimers, the modelling was tailored to hetero-dimer templates. Several close homologs for CtNHase were available which could be used as template. Homology modeling was done using YASARA's homology modeling experiment (see 2.4.5).

In the modeling process eight homology models were built using different templates and alignments where two gave a good overall model quality based on the templates with pdb codes 3QYH and 3QZ5, both crystal structures of Pseudomonas putida nitrile hydratase.

Work was continued with the homology model based on pdb 3QYH. In this alignment, 411 of 428 target residues (96.0%) were aligned to template residues. Among these aligned residues, the sequence identity is 95.4% and the sequence similarity is 96.1%.

With the model, docking studies were then performed with (S)- and (R)-2. Potential key amino acid residues were identified during said study. They are summarized in Table 16. In the docking mode of the (R) product, it seems that the nitrogen of the Trp120 (i.e. W120) of the alpha chain has some interaction with the (R) product when rotated. This may be a candidate for mutation (e.g. to Phenylalanine) to disturb binding of the (R) docking mode. All other residues in the vicinity of the docked products are expected to influence both docking modes (R and S). Anyhow, the residues in the vicinity of the docking modes, except those required for the Cobalt binding, may be altered as well in a more semi-rational approach

TABLE 16
Residues of CfNHase within 4 Å of the docking modes.

| Residue | subunit | additional information |
|---|---|---|
| Q93 | α | |
| C112 | α | metal binding site |
| C115 | α | metal binding site |
| S116 | α | metal binding site |
| C117 | α | metal binding site |
| Y118 | α | metal binding site |
| W120 | α | |
| P126 | α | |
| K131 | α | |
| R169 | α | |
| M34 | β | |
| F37 | β | |
| L48 | β | |
| F51 | β | |
| R52 | β | involved in proton transfer |
| | | (KH Hopmann, full reaction mechanism of Nitrile hy- |
| | | dratase: a cyclic intermediate and an unexpected di- |
| | | sulfide switch, Inorg. Chern. 53, 2014, 2760-62) |
| Y68 | β | |

As the cobalt ion is part of the binding pocket, the residues of the metal binding site are also in close vicinity to the substrate binding site. These residues are evidently important for cobalt-binding and in further consequence for the enzymatic activity and must not be altered. Moreover, R52 of the beta subunit is proposed to be involved in proton transfer and is not target of protein engineering.

3.3.2 Screening of 10 NNK Libraries

Amino acid residues within 4 Å of the docked product were identified and all positions not being part of the metal binding site were selected to be investigated by site-saturation (2.2.4), with the aim to increase activity and enantioselectivity: αQ93, αW120, αP126, αK131, αR169, βM34, βF37, βL48, βF51 and βY68.

Approximately 200 clones of each library were screened for increased production of (S)-2 using a liquid screening system (see section 2.5.2). A particular amidase, namely the amidase of Rhodococcus erythropolis (ReAmidase) (UniProtKB/Swiss-Prot: P22984.2) SEQ ID NO:135 was applied in the screening assay which is strictly (S)-selective for 2. By this particular enzyme selection, surprisingly only mutants producing high amounts of (S)-2 and not (R)-2 and give strong signals in the assay could be identified.

Clones which resulted in a higher content of red color than the wild type enzyme were mostly identified by eye. Alteration of brightness, contrast and saturation of the photos helped to improve the color differences and identify clones that produced more of the desired product.

Altogether, 1936 CtNHase clones were screened of 10 different site-saturation libraries. 120 putatively improved clones were selected and re-screened in triplicates using the same assay.

With HPLC analysis, improved CtNHase mutants were identified. Therefore, also the 120 picked clones of the screening were applied in bioconversions (2.4.2) and analyzed by HPLC (2.4.3).

Some mutants performed better than the wild-type regarding conversion level and enantioselectivity. E.g. eight of the βF51X library were chosen and sent for sequencing. All of the selected clones had an amino acid exchange (Table 17). The leucine mutant occurred seven times and the valine mutant twice. One isoleucine mutant was also found. These three amino acids are all aliphatic, hydrophobic amino acid residues, a fact that confirms the hypothesis. Enhanced mutants were found also for position βM34 (Table 18).

TABLE 17
Improved mutants of βF51X library and their sequencing result.

| Biocatalytic reactions | | | Sequencing result |
|---|---|---|---|
| # | conv. [%] | eeₚ (S) [%] | βF51X |
| CtNHase WT (1) | 2.2 | 92.2 | — |
| 3 | 6.8 | 95.8 | L |
| 16 | 8.8 | 95.2 | L |
| 20 | 10.9 | 94.5 | L |
| 25 | 6.7 | 94.7 | V |
| 32 | 14.6 | 93.7 | L |
| 42 | 19.9 | 92.3 | V |
| CtNHase WT (43) | 4.5 | 90.1 | — |
| 57 | 9.6 | 95.4 | L |
| 60 | 9.5 | 94.2 | L |
| 63 | 9.9 | 93.1 | I |
| 75 | 10.9 | 93.9 | L |

Wild type reactions are shown for a better comparison. 50 mM of rac-1 were converted by 8.5 mg/mL cells (expressed in deep well plate) for 2 h at 25° C. and 500 rpm at pH 7.

TABLE 18
Performance in conversion of rac-1 by improved
CtNHase mutants and their sequencing results.

| HβLC reactions | | | Sequencing |
|---|---|---|---|
| # | conv. [%] | eeₚ (S) [%] | result |
| CtNHase wild-type 1 | 10.5 | 80.8 | — |
| CtNHase wild-type 2 | 15.5 | 81.6 | — |
| BM34X-1 C2 | 8.0 | 84.3 | M34Q |
| BM34X-1 F9 | 10.4 | 85.2 | M34L |
| BF51X-1 B7 | 29.6 | 90.7 | F51I |
| BF51X-2 C1 | 33.2 | 91.2 | F51V |
| BF51X-2 C8 | 30.9 | 92.1 | F51L |
| BF51X-2 D6 | 32.3 | 91.0 | F51V |
| BF51X-2 F3 | 31.3 | 90.6 | F51I |

50 mM ofrac-1 were converted by 8.5 mg/mL cells (expressed in deep well plate) for 2 h at 25° C. and 700 rpm at pH 7.

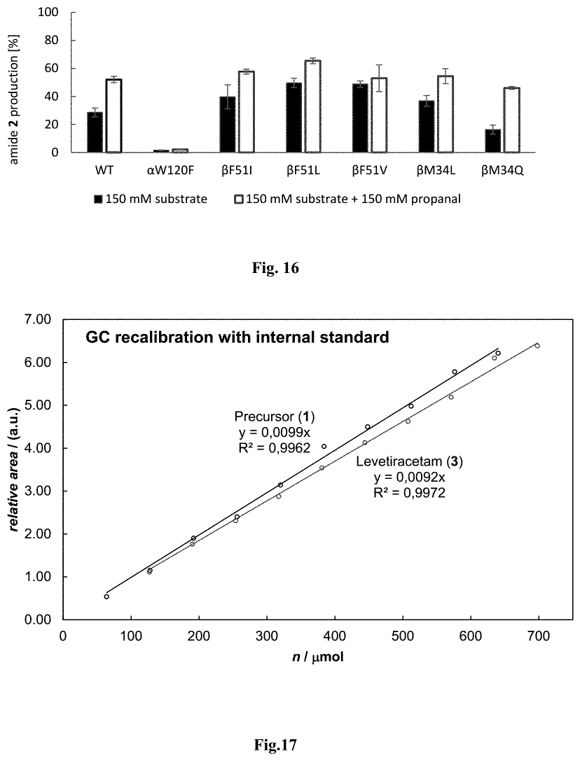

The five improved mutants of site-saturation libraries were expressed in shaking flasks (2.3.1) and used for bioconversions of the target nitrile (2.4.2). All five mutants showed a higher enantioselectivity than the wild type and except for βM34Q, all of them gained a higher conversion as well (see FIG. 16 and Table 19). In addition, FIG. 16 shows a rational design mutant, W120F, which was, unfortunately inactive. Since propanal, one of the products of 1 dissociation, is highly volatile, we reasoned that its supplementation may push the equilibrium towards reformation of rac-1. As demonstrated by the right-hand bars of each pair of bars in FIG. 16 indeed, conversions increased. In case of mutant ßF51V, 65% conversion at >90% ee clearly shows that a dynamic kinetic resolution occurs.

TABLE 19
Conversions and enantioselectivities for single CtNHase mutants in the target
reaction.

| | WT | | BF51 I | | BF51L | | PF51V | | BM34L | | BM34Q | |
|---|---|---|---|---|---|---|---|---|---|---|---|---|
| | w/o | | w/o | | w/o | | w/o | | w/o | | w/o | |
| | P | wP | P | wP | P | wP | P | wP | P | wP | P | wP |
| conversion | 28.6 | 52.0 | 39.7 | 57.7 | 49.6 | 65.4 | 48.8 | 53.0 | 36.8 | 54.4 | 16.3 | 46.0 |
| [%] | | | | | | | | | | | | |
| ± | 3.1 | 2.2 | 8.6 | 1.8 | 3.3 | 2.0 | 2.3 | 9.5 | 3.8 | 5.3 | 3.3 | 1.0 |
| ee_{P} (S) [%] | 82.1 | 80.4 | 88.9 | 89.0 | 92.2 | 90.8 | 89.6 | 88.4 | 86.3 | 85.5 | 84.3 | 85.5 |

150 mM of nitrile rac-1 were hydrated with and without additional 150 mM propanal.

3.3.3 Combination of Beneficial Mutations

The next step was the combination of beneficial amino acid exchanges.

The three possible substitutes for βF51 were combined with both mutations for βM34, resulting in six double Mutants of CtNHase. They were expressed in shaking flasks and tested in biotransformation reactions for amide 2 formation. All of the double mutants showed a conversion below that of the best single Mutant F51L Double mutant βM34L/βF51L had a slightly higher enantioselectivity (Table 20).

TABLE 20
Conversion rates and enantioselectivities for CtNHase
double Mutants in the target reaction.

| | Reaction without propanal | | Reaction with pro panal | |
|---|---|---|---|---|
| Mutant | conversion [%] | ee_{P} (S) [%] | conversion [%] | ee_{P} (S) [%] |
| Wild type | 32.5 | 82.4 | 44.8 | 80.9 |
| βF51L | 49.6 | 92.2 | 65.4 | 90.8 |
| βM34Q/βF51I | 31.2 | 90.7 | 50.2 | 91.2 |
| βM34Q/βF51L | 29.3 | 92.2 | 56.3 | 92.2 |
| βM34Q/βF51V | 29.3 | 91.0 | 52.8 | 91.2 |
| βM34L/βF51I | 43.4 | 92.0 | 45.9 | 91.8 |
| βM34L/βF51L | 38.5 | 93.1 | 57.7 | 92.2 |
| βM34L/βF51V | 35.8 | 91.6 | 46.1 | 91.3 |

150 mM of nitrile rac-1 were hydrated with and without additional 150 mM propanal.

3.3.4 NNK Library Based on Mutant

To this point, only two superior substitutes for βM34 have been found. Other residues at this position might also be beneficial, particularly if they are combined with the amino acid exchange βF51L. Therefore, another site-saturation library for βM34 was constructed based on CtNHase-βF51L, the best single mutant at that time.

176 CtNHase-βM34X/βF51L clones were picked and screened for NHase activity with the liquid screening assay. All 110 active clones were applied in bioconversions of the target nitrile and analyzed by HPLC-UV. The best clones were sequenced (Table 21). Next to βM34L and βM34Q, three further amino acid exchanges were found: βM34I/βF51L, βM34T/βF51L and βM34V/βF51L.

TABLE 21
Promising CtNHase-M34X/F51L clones and
their performance in the target reaction

| | | | Sequencing |
|---|---|---|---|
| HβLC reactions | | | result |
| # | conv. [%] | ee_{P} (S [%] | βM34X |
| CtNHase wildtype 01 | 30.7 | 84.6 | — |
| CZNHase-βF51 L 02 | 58.0 | 92.9 | — |
| 3 | 40.8 | 94.0 | L |
| 10 | 60.7 | 93.7 | L |
| 11 | 19.7 | 93.8 | T |
| 12 | 61.6 | 92.7 | M |
| 19 | 58.3 | 93.7 | L |
| 27 | 66.0 | 93.5 | V |
| 31 | 61.0 | 93.6 | V |
| 38 | 59.0 | 93.7 | L |
| 39 | 54.2 | 93.8 | L |
| 40 | 59.1 | 93.6 | V |
| 55 | 62.9 | 92.6 | M |
| C/NHase wildtype 04 | 20.7 | 84.4 | — |
| C/NHase-βF51L 05 | 49.4 | 91.8 | — |
| 67 | 49.2 | 93.4 | L |
| 69 | 53.0 | 93.5 | V |
| 73 | 50.0 | 93.5 | L |
| 77 | 51.4 | 92.8 | V |
| 80 | 50.3 | 93.0 | V |
| 92 | 53.2 | 93.5 | I |
| 94 | 50.4 | 93.1 | Q |
| 105 | 53.5 | 93.0 | L |
| 110 | 54.3 | 93.4 | L |

50 mM of rac-1 were converted by 8.5 mg/mL cells (expressed in deep well plate) for 2 h at 25° C. and 700 rpm at pH 7.

Bioconversions of rac-1 were performed with the five βM34X/βF51L mutants, expressed in shaking flasks. Double mutant βM34V/βF51L showed the highest conversion. This mutant had also the second best enantiomeric excess with 92.9% for the (S)-enantiomer (Table 22). Only M34I/F51L reached a higher ee with 93.1%, but its conversion with 29.7% was lower than that of the parent. As evident by the standard deviations from three parallel biotransformation reactions, enantiomeric excesses are highly reproducible, while conversions show much more variation.

TABLE 22
Conversions and enantiomeric excess for CtNHase-M34X/F51L double mutants.

| | | | PM34I/ | PM34U | PM34Q/ | PM34T/ | PM34V/ |
|---|---|---|---|---|---|---|---|
| CtNHase | WT | PF51L | PF51L | PF51L | PF51L | PF51L | PF51L |
| conversion | 26.4 | 34.2 | 29.7 | 26.6 | 20.1 | 19.2 | 37.6 |
| [%] | | | | | | | |
| ee_{P} (S) [%] | 82.2 | 91.5 | 93.1 | 92.4 | 91.9 | 91.2 | 92.9 |

150 mM rac-1 was hydrated by 8.5 mg/mL cells at 25° C. and 700 rpm at pH 7 for 2 h.

3.3.5 ReScreening of βL48X Library

Library βL48 was screened again with the optimized assay (2.5.2).

Potential hits were picked and applied in bioconversion reactions. After HPLC and sequencing analysis, also CtNHase-βL48P turned out to be highly selective for (S)-1 (Table 23).

TABLE 23
Conversions and enantiomeric excess for CtNHase-L48 mutants

| | | | Amino acid |
|---|---|---|---|
| CtNHase Clone | conversion [%] | ee_{P} (S) [%] | at βL48 |
| Wild type | 12.5 | 84.1 | L |
| Control BL48R | 19.9 | 98.1 | R |
| BL48X-1-C4 | 16.5 | 98.7 | P |
| BL48X-1-A12 | 13.4 | 98.3 | P |
| BL48X-1-C12 | 12.8 | 99.0 | P |
| BL48X-2-E2 | 25.4 | 98.3 | R |
| BL48X-2-F9 | 21.6 | 97.9 | P |
| βL48X-2-C1 | 21.7 | 98.0 | R |
| βL48X-2-C11 | 19.5 | 98.2 | P |
| βL48X-2-D11 | 21.4 | 98.4 | P |
| βL48X-2-D2 | 19.7 | 98.5 | P |

100 mM rac-1 were converted by 8.5 mg/mL cells at 25° C., 700 rpm and pH 7 for 2 h.

### Example 3.4 Random Mutagenesis of CtNHase

Conversion levels and enantioselectivity for synthesis of (S)-2 by CtNHase were also enhanced in a random approach. Catalytic activity of NHases is dependent on a metal ion and the metal binding site must not be targeted in the screening. As we were especially interested in more enantioselective enzymes, we focused on the region of the binding pocket.

3.4.1 Screening of α1, α2, β1 and β2 Region

Four stretches in CtNHase lining the active site were defined and random libraries were constructed for each stretch. Specifically, the regions were amino acid 70-110 (α1) and 120-175 (α2) in the α-, and 30-71 (β1) and 124-170 (β2) in the β-subunit, respectively. The β1 library was based on wild type CtNHase, whereas the other three libraries were generated on β1 mutant βF51L (which lies in the β1 stretch). At least 11,000 clones per library were screened on colony level.

Around 50,000 clones of five different libraries were screened on colony level (protocol 2.5.1). Approximately the best 10% were identified by eye and re-screened using the liquid assay for nitrile hydratase activity (protocol 2.5.2). Again, approximately 10% of these clones were picked and applied in bioconversion reactions (protocol 2.4.2) which were analyzed by HPLC (2.4.3).

The most improved clones with higher conversion and also increased enantioselectivity were found in the library CtNHase-β1. Clones of the other three libraries which were all based on CtNHase-βF51L showed minor increases.

The position βL48 had the strongest impact on the enantioselectivity. Mutant βL48R reached 96.1% ee for the (S)-product. Interestingly, this position was already investigated in a site-saturation library where no improved hits were found and only one single mutant was found by screening the random libraries. This can be explained by the fact that ‘only’ enantioselectivity was improved but activity retained wild-type level. Hence, the improvement of the signal is not as pronounced and may be overlooked. Especially in this case, only the use of (S)-selective amidase allowed us to find this hit.

The library CtNHase-β1 revealed many enhanced mutants. Most prominent were amino acid exchanges at position at βF51 (Table 24), which had already been targeted in site-saturation mutagenesis. The same substitutes were found as in the NNK libraries: Ile, Leu and Val. The highest enantioselectivity was achieved by βF51L. Also mutation of βG54 occurred multiple times, either to Cys, Asp or Val. The comparison of these three substitutes was difficult as no single mutants were found. However, position βG54 also has a strong influence on activity and enantioselectivity.

Stretch β2 revealed many amino acid exchanges and some of these occurred multiple times (Table 25). However, the mutants showed only minor improvements. Only mutant CtNHase-βF51L/βH146L/βF167Y with a conversion of 49.6% and an ee of 94.5% was declared as hit in this region. Notably, although amino acid exchanges in βF51 and βG54 often have been found, they never occurred in combination. Only one amino acid exchange turned up multiple times in region al. CtNHase-αV110I-βF51L had the same enantioselectivity as its parent CtNHase-βF51L but reached a higher conversion (Table 26). One position was prominent in α2 region: αP121. The residues Ser, Thr and Val at this position caused a significantly higher conversion of 1 however, the ee was decreased (Table 27). CtNHase-αP121T-βF51L was found to be the best as it showed the least loss of enantioselectivity.

TABLE 24
Sequenced CtNHase-β1 clones.

| | | | Sequence (β subunit) | | | | | | | | | | | | | | | | | | | |
|---|---|---|---|---|---|---|---|---|---|---|---|---|---|---|---|---|---|---|---|---|---|---|
| | | e.e. | 30 | 31 | 32 | 33 | 34 | 35 | 36 | 37 | 38 | 39 | 40 | 41 | 42 | 43 | 44 | 45 | 46 | 47 | 48 | 49 |
| # | conversion | for (S) | E | K | T | V | M | S | L | F | P | A | L | F | A | N | G | N | F | N | L | D |
| 21 | 44.5% | 90.4% | | | | X | | | | | | | | | | | | | | | | |
| 23 | 20.6% | 92.2% | | | X | | | | | | | | | | X | | | | | | | |
| 34 | 24.3% | 92.6% | | | X | | | | | | | | | | | | | X | | | | |
| 42 | 54.3% | 90.1% | | | X | | | | | | | | | | | | | | | | | |
| 46 | 62.0% | 90.2% | | | | | | | | | | | | | | | | | | | | |
| 55 | 20.3% | 93.1% | | | | | | | | | | | | | | | | | | | | |
| 57 | 54.3% | 87.6% | | | | | | | | | | | | | | | | | | I | | |
| 58 | 58.5% | 90.9% | | | | | | | | | | | | | | | | | | | | |
| 65 | 29.2% | 91.4% | | | | | | | M | | | | | | | | | | | | | |
| 70 | 47.3% | 90.2% | | | X | | | | | | | | | | | | | | | | | |
| 71 | 52.7% | 90.0% | | | | | | | X | | | | | | | | | | | | | |
| 73 | 54.8% | 89.6% | | | | | | | M | | | | | | | | | | | | | |
| 81 | 29.5% | 91.4% | | | | | | | | | | | | | | | | | | | | |
| 82 | 16.8% | 90.2% | | | | | | | | | | | | | | | | | | | | |
| 83 | 49.5% | 90.6% | | | | | | | | | | | | | | | | | | | | |
| 84 | 37.4% | 91.0% | | | S | | | X | | | | | | | | | | | | | | |
| 89 | 31.7% | 91.1% | | | | | | | | | | | | | | | | | | | | |
| 96 | 53.2% | 90.3% | | | | | | | | | | | | | | | | | | | | |
| 108 | 23.5% | 92.4% | | | | | | | | | | | | | | | | | | | | |
| 110 | 38.6% | 90.7% | | | | | L | | | | | | | | | | | | | | | |
| 117 | 43.6% | 90.1% | | | | | | | | | | | | | T | | | Y | | | | |
| 122 | 54.4% | 88.9% | | | | | | | | | | | | | | | | K | | | | |
| 127 | 44.9% | 90.2% | | | | | | | | | | | | | | I | | | | | | |
| 129 | 30.9% | 92.1% | | | | | | | | | | | | | | | | | L | | | |
| 134 | 41.6% | 92.3% | | | | | | | | | | | X | | | | | | | | | |
| 137 | 25.8% | 96.1% | | | | | | | | | | | | | | | | | | | R | |
| | Sequence (β subunit) | | | | | | | | | | | | | | | | | | | | | |
| | 50 | 51 | 52 | 53 | 54 | 55 | 56 | 57 | 58 | 59 | 60 | 61 | 62 | 63 | 64 | 65 | 66 | 67 | 68 | 69 | 70 | 71 |
| # | E | F | R | H | G | I | E | R | M | N | P | I | D | Y | L | K | G | T | Y | Y | E | H |
| 21 | | I | | | | | | | | | | | | | | | | | | | | |
| 23 | | L | | | | | | | | | | | | | | | | | | | | |
| 34 | | | | | V | | | | | X | | X | | | | | | | | | | |
| 42 | | I | | | | | | | | | | | | | | | | | | | | |
| 46 | | I | X | | | | | | | | | | | | | | | | | | | |
| 55 | | L | | | | | | L | | | | | | | | | X | | | | | |
| 57 | | | | | C | | | | | | | | | | | | X | | | | | |
| 58 | | I | | | | | | | | | | | | | | | | X | | | | |
| 65 | | L | | | | | | | | | | | | | | | | | | | | |
| 70 | | I | | | | | | | | | | | | | | | | | | | | |
| 71 | | I | | | | | | | | | | | | | | | | | | | | |
| 73 | | I | | | | | X | | | | | | | | | | | | | | | |
| 81 | | L | | | | | | | | | | | | | | | | | | | | |
| 82 | | I | | | | | | | | | | | | | | | | | | | | |
| 83 | | V | | | | | | | | | | | | | X | | | | | | L | |
| 84 | | | | | V | | | | | | | | | | | | | | | | | |
| 89 | | I | | | | | | S | | | | | | | | R | | | | | | |
| 96 | | I | | | | | | | | | | | | | | | | | | | | |
| 108 | | | | L | V | | | | | | | | | | | | | | | | | |
| 110 | | | | | C | | | | | X | | | | | | R | | | | | | |
| 117 | | | | X | V | | | | | | | | | | | | | | | | | |
| 122 | | | | | C | | | | | | | | | | X | | | | | | | |
| 127 | | | | | C | | | | | | | | | | | | | | | | | |
| 129 | X | | | | D | | | S | | | | | | | | | | | | | | |
| 134 | | L | | | | | | | | | | | G | | X | | | | | | | |
| 137 | | | | | | | | | | | | | | | | | | | | | | |

X for silent mutations

The results of bioconversions of 100 mM rac-1 are shown and the respective amino acid exchanges.

TABLE 25
Sequenced CtNHase-βf51L-β2 clones.

| | | | Sequence (β subunit) | | | | | | | | | | | | | | | | | | | | | | |
|---|---|---|---|---|---|---|---|---|---|---|---|---|---|---|---|---|---|---|---|---|---|---|---|---|---|
| | | e.e. | [symbol] | | | | | | | | | | | | | | | | | | | | | | |
| # | conversion | for (S) | E | G | A | R | A | R | F | A | V | G | D | K | V | R | V | L | N | K | N | P | V | G | H |
| F51L | 40.8% | 92.2% | | | | | | | | | | | | | | | | | | | | | | | |
| 65 | 41.6% | 92.1% | | | G | | | | | | | | | | | | | | | | | | | | |
| 66 | 39.0% | 92.4% | | | | | | | | | | | | | X | | | | | | | | | | |
| 70 | 38.4% | 92.3% | | | | | X | | | | | | | | | | | | | M | | | | | |
| 71 | 35.2% | 92.5% | | | | X | | | | | | | | | | | | | | | | | | | |
| 73 | 39.1% | 92.5% | | | | | | | | | | | | | | | | | | | | | | | |
| 87 | 31.7% | 92.8% | | | G | | X | | | | | | | | | | | | | | | | | | |
| 90 | 33.6% | 92.1% | | | C | X | | X | | | E | | | | | | | | | | | | | S | |
| 91 | 33.4% | 92.7% | | | | | S | | | | | | | | | | | | | | | | | | |
| 93 | 40.0% | 92.0% | | | | | M | | | | | | | | | | | | | | | | | | |
| 94 | 37.1% | 92.5% | | | V | | | | | | | | | | | | | | | | | | | | |
| 98 | 49.0% | 92.5% | | | | | X | | | | | | | | | | | | | | | | | | |
| 103 | 47.5% | 92.7% | | | | | | Q | | | | | | | | | | | | | | X | X | | |
| 105 | 46.3% | 92.2% | | | | | | W | | | | | | | | | | | | | | | | | |
| 112 | 41.0% | 92.6% | | | | X | | L | | | | | | | | | | | | | | | | | |
| 113 | 40.3% | 92.1% | | | | | | | | | | | | | | | | | | | | | | | |
| 126 | 45.4% | 92.4% | | | | | E | X | | T | | W | | | | | | | | | | | | | |
| 128 | 46.4% | 92.7% | | | | | | | | | | | | | | | | | | | | | | D | |
| 130 | 39.3% | 92.1% | | | | | | | | | | | | | | | | | | | | | | | |
| 131 | 35.0% | 99.1% | | | | | | | | | | | | | | | | | | | | | L | | |
| 1 | 29.3% | 92.8% | | | | | | | | | | | | | | | | | | | | | | | |
| 3 | 49.6% | 94.5% | | | | | | | | | | | | | | | | | | | | | X | | L |
| 4 | 51.8% | 92.1% | | | | | V | | | | E | | | | | | | | | | | | | | |
| 24 | 44.4% | 98.2% | | | | | | X | | | | | | | | | | | | | | | | | |
| 26 | 33.7% | 92.8% | | X | | | | | | | | | | | | | | | | | | | | | |
| 29 | 40.9% | 93.1% | | | | | | | | | | | | | | | H | | | | | | L | | |
| 36 | 57.7% | 92.8% | | | | | | | | | | | | | | | | | | | | | E | | |
| 38 | 52.9% | 92.5% | | | | | | | | | | | | | | | | | | | | | | | |
| 39 | 50.4% | 92.5% | | | | | | | | | | | | | | | | | | | | | | | |
| 42 | 54.8% | 92.1% | | A | | | | | | | | | | | | | | | | | | | | | |
| 52 | 55.1% | 92.4% | | | V | | | | | | | | | | | | | | | | | | | | |
| 56 | 50.8% | 92.6% | | | | | | | | | | | | | | | | | | | | | | | |

| | Sequence (β subunit) | | | | | | | | | | | | | | | | | | | | | | | |
|---|---|---|---|---|---|---|---|---|---|---|---|---|---|---|---|---|---|---|---|---|---|---|---|---|
| | [symbol] | | | | | | | | | | | | | | | | | | | | | | | |
| # | T | R | M | P | R | Y | T | R | G | K | V | G | T | V | V | I | D | H | G | V | F | V | T | P |
| 65 | | | | | | | | | | | | | | | | | | | | | | | | |
| 66 | | | | | | | | | | | | | | | | | | | | G | | | | |
| 70 | | | | | | | | | | | | | | | | | | P | | | | | | |
| 71 | | | | | | | | | | | | | | | | | | | | | | | | S |
| 73 | | | | | | | | | D | | | | | | | | | | C | | | | | |
| 87 | | | | | | F | | | | | | | | | | | | | | | | | | |
| 90 | | | | | | | | | | | | | | | | | | | | | | | | |
| 91 | | | | | | | | | | N | | | | | | | | | | | | | | |
| 93 | | | | | | | | | | | | | | | | | | | | | | | | |
| 94 | | | | | | | A | | | | | | | | | | | Q | | | | | | |
| 98 | | | | | | | | | | | X | | | | | | | L | | | | | | |
| 103 | | | | | | | | | | | | | | | | | | | | | Y | | K | |
| 105 | | | | | | | | | | | | | | | | | | | | | | | | |
| 112 | | | | | | | | | | | X | | X | | | | | | X | | | | | |
| 113 | | | | | | | | | | | | | | | | | | | | | | | | |
| 126 | | | | | | | | | | | | | | | | | | | | | | | | |
| 128 | | | | | | | | | | | | | | | | | | | | | | | | |
| 130 | | | | | | | | | | | | | | | | | | | | | | | | |
| 131 | | | | | | | | | | | | | X | | | | | | | | | | M | A |
| 1 | | | | | | | | | | | | | | | | | | Q | | | | | | S |
| 3 | | | | | | | | | | | | | | | | | | | | | Y | | | |
| 4 | | | | | | | | | | | | | | | | | | | | | | | | |
| 24 | | | | | | | | | | | | | | | | | | | | | | | | S |
| 26 | | | | | | | | | S | | | | | | | L | | | S | | | X | | |
| 29 | | | | | | F | | | S | | | | | | | | | | | | | | | |
| 36 | | | | | | | | | | | | | | | | X | | | C | | | | | |
| 38 | | | | | | | | | | | | | | | | | | | | | | | S | |
| 39 | | | | | | | | | | | | | | | | | | | | | | | | |
| 42 | | | | | | | X | | | | | | | | | | | | | | | | | |
| 52 | | | | | | | | | | | E | | | | | | | | | | | | | |
| 56 | | | | X | | | | | | | | | | | | V | | L | | | | | | |

X for silent mutations [symbol]
indicates data missing or illegible when filed

The results of bioconversions of 100 mM rac-1 are shown and the respective amino acid exchanges.

TABLE 26
Sequenced CtNHase-βF51L-α1 clones.

| | | | Sequence (α subunit) | | | | | | | | | | | | | | | | | | | |
|---|---|---|---|---|---|---|---|---|---|---|---|---|---|---|---|---|---|---|---|---|---|---|
| | | e.e. | 70 | 71 | 72 | 73 | 74 | 75 | 76 | 77 | 78 | 79 | 80 | 81 | 82 | 83 | 84 | 85 | 86 | 87 | 88 | 89 |
| # | conversion | for (S) | P | A | Y | K | A | R | L | L | A | D | G | T | A | G | I | A | E | L | G | F |
| F51L | 40.8% | 92.2% | | | | | | | | | | | | | | | | | | | | |
| 57 | 56.9% | 92.6% | | | | | | | | | | | | | | | | | | | | |
| 59 | 58.6% | 92.8% | | | | | | | | | | | | | | | | | | | | |
| 63 | 49.8% | 92.6% | | | | | | | | | | | | | | | | | | | | |
| 70 | 57.2% | 92.8% | | | | | | | | | | | | | | | | | | | | |
| 71 | 42.2% | 92.6% | | | | | | | | | | | | | | | | | | | | |
| 72 | 48.2% | 92.6% | | | | | | | | | | | | | | | | | | | | |
| 73 | 52.5% | 92.5% | | | | | | | | | | | | | | | | | | | | |
| 74 | 47.2% | 92.5% | | | | | | | | | | | | | | | | | | X | | |
| 80 | 48.9% | 92.5% | | | | | | | | | | | | | | | | | | | | |
| 83 | 48.9% | 92.5% | | | | | | | | | | | | | | | | | | | | |
| 86 | 53.8% | 93.1% | | | | | | | | | | | | I | | | | | | | | |
| 99 | 50.4% | 92.5% | | | | | | | | | | | | | | | | | | | | |
| 106 | 49.6% | 92.5% | | X | | | | | | | | | | | | | | | | | | |
| 124 | 44.6% | 92.8% | | | | | | | | | | X | | | | | | | | | | |
| 128 | 36.8% | 92.8% | | | | X | | | | | | X | | S | | | | | | | | |

| | Sequence (α subunit) | | | | | | | | | | | | | | | | | | | | |
|---|---|---|---|---|---|---|---|---|---|---|---|---|---|---|---|---|---|---|---|---|---|
| | 90 | 91 | 92 | 93 | 94 | 95 | 96 | 97 | 98 | 99 | 100 | 101 | 102 | 103 | 104 | 105 | 106 | 107 | 108 | 109 | 110 |
| # | S | G | V | Q | G | E | D | M | V | I | L | E | N | T | P | A | V | H | N | V | V |
| F51L | | | | | | | | | | | | | | | | | | | | | |
| 57 | | | | | | | | | | | | | | | | | | | | | I |
| 59 | | | | | | | | | | | | | | | | | | | | | I |
| 63 | | | | | | | | | | | | | | | | | | | | | I |
| 70 | | | | | | | | | | | | | X | X | | | | | | | I |
| 71 | | | | | | | | | | | | | | | | | | | | | |
| 72 | | | | | | | | | | | | | | | | | | | | | |
| 73 | | | | | | | | | | | | | | | | | | | | | |
| 74 | | | | | | | | | | | | | | | | | | | | | |
| 80 | | | | | | | | | | | | | | | | T | | | | | I |
| 83 | | | | | X | | | | | | | X | X | | | | | | | | I |
| 86 | | | | | | | | | | | | | | | | | | | | | I |
| 99 | | | | | | | | | | | | | | | | | | | | | |
| 106 | | | | | | | | | | | | | | | | | | | | | |
| 124 | | | | | | | | | | | | | | | | | X | | | | |
| 128 | | | | | | | | | S | | | | | | | | | | | | |

X for silent mutations

The results of bioconversions of 100 mM rac-1 are shown and the respective amino acid exchanges.

TABLE 27
Sequenced CtNHase-βF51L-α2 clones.

| | | | Sequence (α subunit) | | | | | | | | | | | | |
|---|---|---|---|---|---|---|---|---|---|---|---|---|---|---|---|
| | | e.e. | 120 | 121 | 122 | 123 | 124 | 125 | 126 | 127 | 128 | 129 | 130 | 131 | 132 |
| # | conversion | for (S) | W | P | T | L | G | L | P | P | A | W | Y | K | A |
| 145 | 50.7% | 92.2% | | | | | X | | | | | | | | |
| 146 | 44.3% | 92.3% | | | | | | | | | | | | | |
| 149 | 44.4% | 92.3% | | | | | | | | | | | | | |
| 151 | 57.6% | 89.5% | | | T | | | | | | | | | | |
| 27 | 52.8% | 80.3% | | | S | | | | | | | | | | |
| 33 | 60.6% | 84.8% | | | V | | | | | | | | | | |
| 34 | 42.6% | 92.0% | | | | | | | | | | | | | |
| 35 | 69.6% | 80.2% | | | S | | | | | | | | | | |
| 40 | 64.5% | 89.5% | | | T | | | | | | | | | | |
| 41 | 52.2% | 92.2% | | | | | | | | | | | | | |
| 45 | 36.8% | 92.4% | | | | | | | | | | | | | |
| 46 | 42.6% | 92.0% | | | | | | | | | | | | | |
| 48 | 40.1% | 92.5% | | | | | | | | | | | | | |
| 49 | 41.0% | 92.3% | | | | | | | | | | | | | |
| 51 | 42.3% | 92.3% | | | | | | | | | | | | | |
| | Sequence (α subunit) | | | | | | | | | | | | | | |
| | 133 | 134 | 135 | 136 | 137 | 138 | 139 | 140 | 141 | 142 | 143 | 144 | 145 | 146 | 147 |
| # | P | P | Y | R | S | R | M | V | S | D | P | R | G | V | L |
| 145 | | | T | | | | | | | | | | | | |
| 146 | | | | | | | | | | | | | | | |
| 149 | | | | | | | | | | | | | | | |
| 151 | | | | | | | | | | | | | | | |
| 27 | | | | | | | | | | | | | | | |
| 33 | | | | | | | | | | | | | | | |
| 34 | | | | | | | | L | | | | | | | |
| 35 | | | | | | | | | | | | | | | |
| 40 | | | | | | | | | | | | | | | |
| 41 | | | | | | | | M | | | | | | | |
| 45 | | | | | | | | | | | | | | | X |
| 46 | | | | | | | | | | | | | | | |
| 48 | | | | | | | | | | | | | | | |
| 49 | | | | | | | | | | | | | | | |
| 51 | | | | | | | | | | | | | | | |
| | Sequence (α subunit) | | | | | | | | | | | | | | |
| | 148 | 149 | 150 | 151 | 152 | 153 | 154 | 155 | 156 | 157 | 158 | 159 | 160 | | |
| # | A | E | F | G | L | V | I | P | A | K | E | I | R | | |
| 145 | | | | | | E | | | | | | | | | |
| 146 | | | | | | | | | | | | | | | |
| 149 | | | | | | | | | | | | | | | |
| 151 | | | | | | | | | | | | | | | |
| 27 | | | | | | | | | | | | | | | |
| 33 | | | | | | | | | | | | | | | |
| 34 | | | | | | | | | | | | | | | |
| 35 | | | | | | | | | | | | | | | |
| 40 | | | | | | | | | | | | | | | |
| 41 | | | | | | | | | | | | | | | |
| 45 | | | | | | | | | | | | | | | |
| 46 | | | | | | | | | | | | | | | |
| 48 | | | | | | | | | | | | | | | |
| 49 | | | | | | | | | | | | | | | |
| 51 | | | | | | | | | | | | | | | |
| | Sequence (α subunit) | | | | | | | | | | | | | | |
| | 161 | 162 | 163 | 164 | 165 | 166 | 167 | 168 | 169 | 170 | 171 | 172 | 173 | 174 | 175 |
| # | V | W | D | T | T | A | E | L | R | Y | M | V | L | P | E |
| 145 | | | | | | | | | | | | | | | |
| 146 | | | | | X | | | | | | | | | | |
| 149 | | | | | | | | | | | | | | | |
| 151 | | | | | | | | | | | | | | X | |
| 27 | | | | | | | | | | | | | | | |
| 33 | | | | | | | | | | | | | | | |
| 34 | | | | | | | | | | | | | | | |
| 35 | | | | | | | | | | | | | X | | |
| 40 | | | | | | | | | | | | | | | |
| 41 | | | | | | | | | | | | | | | |
| 45 | | | | | | | | | | | | | | | |
| 46 | | | | | | | | | | | | | | | |
| 48 | | | | | | | | | | | | | | | |
| 49 | | | | | | | | | | | | | | | |
| 51 | | | | | | | | | | | | | | | |

X for silent mutations

The results of bioconversions of 100 mM rac-1 are shown and the respective amino acid exchanges.

3.4.2 β1 Region Focused Library

Beneficial amino acid exchanges of β1 region were combined. As they are in close vicinity to each other, they all could be represented within one oligonucleotide. At the time of designing this library, for position βL48 only the Arg mutant was known. Therefore, bulky amino acids were allowed at this position as tryptophan. The required codon was YKS to achieve Leu (the wild type amino acid), Arg or Trp (Table 28). The YKS codon enables also cysteine or phenylalanine.

TABLE 28
Codons for focused CtNHase-31 library and their possible amino acids.

| position | βL48 | βF51 | βG54 |
|---|---|---|---|
| desired amino acids | L, R, W | I, L, V | variety |
| codon | YKS | VTT | NDT |
| possible amino acids | L, R, W, C, F | I, L, V | F, L, I, V, Y, |
| | | | H, N, D, C, R, S G |

The improved mutants βF51L, βF51I and βF51V were also included into the focused library. The wild type codon was not allowed, only Ile, Leu and Val. Position βG54 also had a strong impact on the enzymatic activity. So far, Cys, Asp and Val were found at this position. However, they were mostly in combination with other amino acid exchanges and no detailed studies have been performed for this residue. Therefore, a variety of amino acids should be tested for this position. Triplet codon NNK would have given all canonical amino acids but would also have increased the variability by 32. Therefore, codon NDT was used instead, resulting in representatives of all chemical groups.

The library was constructed by overlap extension PCR with degenerated oligonucleotides (chapter 2.2.7) and screened in the colony-based assay (2.5.1). Potential hits were rescreened using the liquid assay (2.5.2), the promising mutants of which were applied in biocatalytic reactions (2.4.2).

The highest enantioselectivities for (S)-2 were achieved by mutants with amino acid exchanges in βL48 (Table 29), for example #76, 56, 90 and so on. Unfortunately, these mutants showed low conversion levels, with 17% at most (#57).

The most effective mutants were identified as βF51I/βG54R, βF51V/βG54I, βF51V/βG54R and βF51V/βG54V. They all occurred multiple times and achieved a higher conversion than CtNHase-βF51L. Moreover, the mutants βF51I/βG54R, βF51V/βG54I and βF51V/βG54R showed higher enantiomeric excess than the single mutant βF51L.

TABLE 29
Conversion levels and enantioselectivities of potential
hits of library CtNHase-β1-focused

| | | | Amino acid exchanges at | | |
|---|---|---|---|---|---|
| clone | conversion | ee_{P} (S)^{a} | βL48 | βF51 | βG54 |
| CINH-βF51L | 26.7% | 93.6% | | L | |
| CINH-βL48R | 19.9% | 98.1% | R | | |
| 76 | 11.9% | 99.0% | F | I | C |
| 88 | 41.0% | 93.4% | | I | F |
| 43 | 28.3% | 95.2% | | I | I |
| 56 | 8.6% | 98.3% | R | I | I |
| 55 | 23.3% | 95.9% | X (TTG) | I | L |
| 72 | 47.7% | 93.0% | X (CTG) | I | N |
| 73 | 53.6% | 92.5% | | I | N |
| 44 | 36.9% | 94.4% | | I | R |
| 50 | 51.0% | 93.9% | | I | R |
| 52 | 45.4% | 94.0% | | I | R |
| 53 | 47.9% | 93.9% | X (TTG) | I | R |
| 59 | 45.0% | 94.1% | X (TTG) | I | R |
| 74 | 37.0% | 94.5% | X (CTG) | I | R |
| 87 | 42.9% | 94.1% | X (CTG) | I | R |
| 90 | 10.5% | 97.9% | R | L | F |
| 91 | 39.1% | 95.0% | X (TTG) | L | F |
| 46 | 23.4% | 96.8% | X (CTG) | L | I |
| 39 | 32.6% | 93.9% | | V | C |
| 62 | 46.1% | 93.6% | X (CTG) | V | C |
| 67 | 60.8% | 93.6% | | V | C |
| 68 | 38.4% | 94.9% | X (TTG) | V | D |
| 63 | 42.7% | 93.9% | | V | F |
| 66 | 66.6% | 91.2% | X (CTG) | V | H |
| 71 | 45.9% | 95.8% | X (TTG) | V | I |
| 82 | 48.2% | 95.6% | X (TTG) | V | I |
| 83 | 51.2% | 95.6% | X (CTG) | V | I |
| 65 | 47.6% | 94.2% | X (TTG) | V | N |
| 40 | 37.9% | 95.2% | X (TTG) | V | R |
| 60 | 42.4% | 95.3% | X (TTG) | V | R |
| 69 | 30.0% | 95.6% | | V | R |
| 84 | 69.3% | 94.8% | X (TTG) | V | R |
| 57 | 17.0% | 97.2% | C | V | S |
| 38 | 56.5% | 93.2% | | V | V |
| 54 | 50.7% | 93.3% | | V | V |
| 61 | 49.8% | 93.6% | | V | V |
| 64 | 65.6% | 93.6% | X (TTG) | V | V |
| 70 | 62.5% | 93.6% | X (CTG) | V | V |
| 75 | 5.6% | 99.0% | F | V | V |
| 79 | 54.1% | 93.4% | X (CTG) | V | V |

X for silent mutations, codons in brackets
^{a}Enantiomeric excess was corrected by subtraction of peak of unknown compound which overlaps with the peak of the (R)-amide
Clones are grouped depending on their sequencing results. 100 mM of 1 have been converted by 8.5 mg/mL cells at pH 7 for 2 h at 25° C. and 700 rpm.

3.4.3 Site-Saturation of αP121

Amino acid exchanges at αP121 were found several times in the screening of CtNHase-βF51L-α2. Substitutions to Thr, Ser or Val increased the conversion levels significantly whereas the enantiomeric excess was slightly reduced (Table 27). This position had a strong effect on activity and also influenced the enantioselectivity. In error-prone PCR, it is very unlikely that all 20 amino acid residues are generated for one position and included in the library. The question was if other amino acid exchanges would give a more active and more selective mutant.

Next to the parent and the Thr, Ser and Val mutants, all other CtNHase-αP121X-βF51L were generated by QuikChange PCR (Method 2.2.4). All remaining mutants were obtained except for Gly because the PCR reaction did not work in this case.

The CtNHase-αP121X-βF51L mutants were expressed in deep well plates (improved protocol 2.3.2) and applied in bioconversion of 100 mM of rac-1 (2.4.2). The best mutant was still the Thr mutant (Table 30). Most of the newly constructed mutants showed a significant loss of enantioselectivity. The best among the new mutants was Ile with 89.2% ee and 50.6 conversion, however, the Thr mutant was still better.

TABLE 30
Conversion levels and enantioselectivities of
CtNHase-αP121X-βF51L mutants

| Amino acid at αP121 | conversion [%] | ee_{P} (S) [%]^{a} |
|---|---|---|
| P (parent) | 33.2 | 93.5 |
| S | 65.7 | 81.5 |
| T | 57.0 | 90.6 |
| V | 54.5 | 86.2 |
| A | 52.8 | 77.9 |
| R | 0.0 | — |
| N | 30.6 | 79.2 |
| D | 0.7 | 79.0 |
| C | 30.6 | 77.8 |
| Q | 7.5 | 76.8 |
| E | 0.2 | — |
| H | 5.8 | 72.9 |
| I | 50.6 | 89.2 |
| L | 48.7 | 66.6 |
| K | 0.2 | 62.3 |
| M | 27.4 | 66.1 |
| F | 0.0 | — |
| W | 0.0 | — |
| Y | 0.0 | — |
| empty vector | 0.0 | — |

^{a}Enantiomeric excess was corrected by subtraction of peak area of unknown compound which overlaps with the peak of the (R)-amide
100 mM of 1 were converted by 8.5 mg/mL cells at pH 7, for 2 h at 25° C. and 700 rpm.

3.4.4 Key Residues of CtNHase—Summary

Around 50,000 randomly mutagenized CtNHase clones, based on the wild type or the single mutant βF51L have been screened, where four stretches have been targeted. Seven amino acid positions were identified as key residues for activity and/or enantioselectivity Key residues αV110 and αP121 on the α subunit enhance the conversion. αP121 exchanges cause, however, a slight decrease in enantioselectivity. For stretch β2, a combination was found which leads to increased activity and enantioselectivity: βH146L/βF167Y. The most influential region was β2. Amino acid exchanges in βL48, βF51 and β54 resulted in much higher ees and also much higher conversion levels for βF51 and βG54.

### Example 3.5. Combination of Beneficial Amino Acid Exchanges

After screening of more than 50,000 CtNHase clones and identification of key residues for conversion of rac-1, beneficial amino acid exchanges were combined. Doing so, 31 new CtNHase mutants were designed (Table 31) of which 28 could be constructed within the project timeframe (cloning protocols see chapter 2.2).

TABLE 31
CtNHase combination mutants and their amino acid exchanges.

| | | | β1 | |
|---|---|---|---|---|
| # | α1^{a} | α2^{b} | L48^{c}\|F51^{d}\|G54^{e} | β2^{f} |
| 1 | V110I | P121T | F51L | H146L/F167Y |
| 2 | V110I | | L48R | |
| 3 | V110I | | L48P | |
| 4 | V110I | | L48F | |
| 5 | | P121T | L48R | |
| 6 | | P121T | L48P | |
| 7 | | P121T | L48F | |
| 8 | | | L48R | H146L/F167Y |
| 9 | | | L48P | H146L/F167Y |
| 10 | | | L48F | H146L/F167Y |
| 11 | | | -\|I\|R | H146L/F167Y |
| 12 | | | -\|V\|I | H146L/F167Y |
| 13 | | | -\|V\|R | H146L/F167Y |
| 14 | | | -\|V\|V | H146L/F167Y |
| 15 | V110I | | -\|V\|V | |
| 16 | | P121T | -\|V\|V | |
| 17^{g} | V110I | P121T | -\|V\|V | |
| 18^{g} | V110I | | -\|V\|V | H146L/F167Y |
| 19^{g} | | P121T | -\|V\|V | H146L/F167Y |
| 20 | V110I | P121T | -\|V\|V | H146L/F167Y |
| 21 | | | P\|V\|V | |
| 22 | | | R\|-\|C | |
| 23 | | | R\|-\|R | |
| 24 | | | R\|-\|V | |
| 25 | | | P\|-\|C | |
| 26 | | | P\|-\|R | |
| 27 | | | P\|-\|V | |
| 28 | | | F\|-\|C | |
| 29 | | | F\|-\|R | |
| 30 | | | F\|-\|V | |
| 31 | V110I | P121T | F51I | H146L/F167Y |

^{a}Codon for amino acid exchanges αV1101 is ATC
^{b}Codon for amino acid exchange αP121T is ACG
^{c}Codon for R at position 348 is CGT, for P it is CCT and for F it is TTC
^{d}Codon for L at position 351 is CTT, for I it is ATT and for V it is GTT
^{e}Codon for I at position 354 is ATT, for R it is CGT and for V it is GTT. The combination F51V/G54I also in eluded a silent mutation TTG in position βL48.Codons for amino acid exchanges βH146L/βF167Y were CTT and TAC, respectively
^{g}Mutant could not be constructed by QuikChange PCR
Separate columns are shown for each stretch targeted in random mutagenesis for a better readability.

The 28 final combination mutants were expressed in shaking flasks (2.3.1) and screened for hydration of rac-1 (see section 2.4.2). Next to the new mutants, some control mutants were analyzed. Highest ee values were achieved for mutants including an amino acid exchange in position βL48 (Table 32). The controls βL48R and βL48P showed an ee for the (S) enantiomer of 98.3% or 98.6%, respectively, whereas more than 99.0% were reached if an amino acid exchange in βG54 was combined (mutants V24-27). In general, βL48/8G54 double mutants showed high ees at acceptable conversion levels (V22-30). The highest conversion among the combination mutants was achieved by V5 with 42% and an ee of 98.1%. All other mutants, for which a βL48 substitution was combined either with αV110I or αP121T, did not show such a strong effect on the conversion (V2-7).

The combination of the amino acid exchanges βH146L/βF167Y with others than βF51L led to significant loss of activity (V8-14 and V20). Moreover, the attempt to increase the activity of CtNHase-βF51V/βG54I by combining it either with αV110I or αP121T failed (V15 and V16). Clones with amino acid exchanges in all targeted stretches (V1, V20 and V31) were disappointing too. Notably, reduced activity was not a result of reduced expression levels, as analyzed by NUPAGE. In fact, all mutations we had analyzed did not decrease the level of soluble expression (data not shown).

Biocatalytic reactions of rac-1 were repeated with the best combination mutants and respective controls. This time, propanal was added and triplicates were evaluated (method 2.4.2). Higher conversion levels were determined (Table 33) than in reactions without supplementary propanal (Table 32). 66.9% conversion were reached by CtNHase-αP121T/βL48R (V5) at a high enantiomeric excess of 97.59%. The mutants CtNHase-βL48P combined with βG54C, βG54R or βG54V (V25-27) achieved exceptionally high ee values ≥99.8%.

TABLE 32
Conversion rates and ee values for hydration of
rac-1 by CtNHase combination mutants.

| Sample info | | HβLC result | |
|---|---|---|---|
| # | mutant | conversion [%] | eeₚ (S) [%]^{a} |
| C1 | wild type | 24.0 | 84.6 |
| C2 | βF51L | 43.7 | 93.6 |
| C3 | βF51I | 28.5 | 91.7 |
| C4 | βF51V | 32.2 | 91.0 |
| C5 | βL48R | 20.7 | 98.3 |
| C6 | βL48P | 19.1 | 98.6 |
| C7 | αV110I/βF51L | 34.1 | 93.9 |
| C8 | αP121T/βF51L | 58.2 | 90.9 |
| C9 | βF51L/βH146L/βF167Y | 46.5 | 95.6 |
| C10 | βF51V/βG54V | 15.8 | 94.1 |
| C11 | βF51V/βG54I | 27.8 | 95.6 |
| C12 | βF51V/βG54R | 20.1 | 95.3 |
| C13 | βF51I/pG54R | 22.2 | 94.8 |
| V1 | αV110I/αP121T/βF51L/ | 34.2 | 93.9 |
| | βH146L/pF167Y | | |
| V2 | αV110I/pL48R | 26.4 | 97.6 |
| V3 | αV110I/pL48P | 26.1 | 98.7 |
| V4 | αV110I/pL48F | 19.6 | 98.2 |
| V5 | αP121T/pL48R | 42.4 | 98.1 |
| V6 | αP121T/pL48P | 24.2 | 98.7 |
| V7 | αP121T/pL48F | 26.3 | 97.4 |
| V8 | βL48R/βH146L/βF167Y | 3.2 | 89.2 |
| V9 | βL48P/βH146L/βF167Y | 10.0 | 94.1 |
| V10 | βL48F/βH146L/βF167Y | 5.9 | 94.6 |
| V11 | βF51I/pG54R/βH146L/βF167Y | 0.3 | 80.2 |
| V12 | βF51V/βG54I/βH146L/βF167Y | 1.0 | 95.0 |
| V13 | βF51V/βG54R/βH146L/pF167Y | 2.9 | 96.5 |
| V14 | βF51V/βG54V/βH146L/βF167Y | 8.7 | 97.4 |
| V15 | αV110l/βF51V/pG54l | 20.2 | 96.3 |
| V16 | αP121T/βF51V/βG54l | 27.8 | 92.3 |
| V20 | αV110l/αP121T/βF51V/ | 0.0 | 0.0 |
| | pG54l/βH146L/βF167Y | | |
| V21 | βL48P/βF51V/βG54V | 13.8 | 98.3 |
| V22 | βL48R/βG54C | 30.5 | 98.6 |
| V23 | βL48R/βG54R | 25.5 | 98.8 |
| V24 | βL48R/βG54V | 31.3 | 99.0 |
| V25 | βL48P/βG54C | 21.9 | 99.2 |
| V26 | βL48P/βG54R | 22.0 | 99.2 |
| V27 | βL48P/βG54V | 24.5 | 99.2 |
| V28 | βL48F/βG54C | 18.0 | 98.8 |
| V29 | βL48F/βG54R | 21.8 | 98.7 |
| V30 | βL48F/βG54V | 23.0 | 98.4 |
| V31 | αV110l/αP121T/βF51I/ | 8.0 | 91.7 |
| | βH146L/βF167Y | | |

^{a}Enantiomeric excess was corrected by subtraction of peak area of unknown compound which over-laps with the peak of the (R)-amide
100 mM rac-1 were converted by 8.5 mg/mL cells for 2 h at 25° C. and pH 7.

TABLE 33
Conversions and ee values for CtNHase combination mutants.

| | | HPLC result | | | |
|---|---|---|---|---|---|
| Sample info | | conversion | | eeₚ (S)a | |
| # | mutant | [%] | std dev± | [%] | std dev± |
| C1 | wild type | 33.3 | 3.2 | 84.08 | 0.07 |
| C5 | βL48R | 47.1 | 1.1 | 98.05 | 0.05 |
| C6 | βL48P | 37.3 | 1.0 | 99.04 | 0.07 |
| V3 | αV110l/pL48P | 42.3 | 4.1 | 98.92 | 0.11 |
| V5 | αP121T/pL48R | 66.9 | 2.6 | 97.59 | 0.08 |
| V6 | αP121T/pL48P | 53.8 | 1.7 | 98.99 | 0.10 |
| V22 | βL48R/βG54C | 53.8 | 1.2 | 98.51 | 0.12 |
| V23 | βL48R/βG54R | 46.6 | 3.4 | 99.05 | 0.05 |
| V24 | βL48R/βG54V | 55.9 | 2.5 | 99.01 | 0.06 |
| V25 | βL48P/βG54C | 38.0 | 1.8 | 99.80 | 0.17 |
| V26 | βL48P/βG54R | 35.7 | 4.2 | 99.80 | 0.16 |
| V27 | βL48P/βG54V | 37.3 | 5.2 | 99.94 | 0.03 |
| V28 | βL48F/βG54C | 30.2 | 7.6 | 98.79 | 0.28 |
| V29 | βL48F/βG54R | 38.1 | 1.7 | 98.96 | 0.03 |
| V30 | βL48F/βG54V | 35.4 | 5.4 | 98.93 | 0.31 |

^{a}Enantiomeric excess was corrected by subtraction of peak area of unknown compound which overlaps with the peak of the (R)-amide
100 mM rac-1 were converted by 8.5 mg/mL cells at 25° C. and pH 7 in 2 h. Additional 100 mM propanal were applied.

### Example 3.6. Enzymatic Hydration of Rac-1 to (S)-2 on Preparative Scale

On the preparative scale, rac-1 was hydrated to (S)-2 in using the P121T/L48R double mutant of CtNHase in form of a whole cell biocatalyst.

Frozen cell pellet was thawed and approximately 500 mg suspended in 50 mL of phosphate buffer (100 mM, pH 7.1). The reaction was carried out in a Mettler Toledo T50 pH stat at 22° C. using 1 M phosphoric acid titration. The reaction was started by addition of 200 μL of rac-1, 100 μL of propanal and 100 μL of pyrrolidine (each added as pure substance). Reaction progress translates in pH increase and can be monitored on basis of acid consumption. Every 10-15 minutes, acid addition ceased and pulses of 100 μL of propanal and 200 μL of rac-1 were added.

Propanal and pyrrolidine are added in order to shift the equilibrium of rac-1 decomposition towards rac-1, so that free cyanide is bound, and thus NHase inhibition is minimized.

To analyze reaction progress, 250 μL samples were withdrawn repeatedly and treated as described in 2.4.2 (Materials & Methods) before analysis as described in 2.4.3 (Materials & Methods). FIG. 19 shows the result of a typical experiment.

In total, 1.3 g of rac-1 were hydrated to (S)-2 in 50 mL reaction volume within 80 min in 73.3% conversion and 95.2% ee.

### B. Chemical Section

### 1. Apparatuses and Devices

Electrochemical reactions were carried out at boron-doped diamond (BDD) anodes. The BDD electrodes were obtained in DIACHEM© quality from CONDIAS GmbH, Itzehoe, Germany. The BDD had a 15 μm diamond layer on silicon support. Stainless steel of the type EN1.4401; AISI/ASTM was used as cathodes. Nafion™ from DuPont was used as membrane. A galvanostate HMP4040 from Rhode&Schwarz was employed.

NMR spectra were recorded on a Bruker Avance III HD 300 (300 MHz) equipped with 5 mm BBFO head with z gradient and ATM at 25° C. Chemical shifts (δ) are reported in parts per million (ppm) relative to traces of CHCl₃ in CDCl₃ as deuterated solvent.

Liquid chromatography photodiode array analysis was performed by using a DUGA-20A₃ device from Shimadzu, which was equipped with a C18 column from Knauer (Eurospher II, 100-5 C18, 150×4 mm). The column was conditioned to 25° C. and the flow rate was set to 1 mL/min. The aqueous eluent was buffered with formic acid (0.8 mL/2.5 L) and stabilized with Acetone (5 vol %).

Gaschromatography was performed using a GC 2010 device from Shimadzu, which was equipped with a Varian capillary column ZB-5MSi (Serial No. 334634), operating with H₂ as carrier gas. Infrared spectra were recorded on an ATR IR device of the type ALPHA from Bruker.

Thin Layer Chromatography was performed using commercially available aluminum plates coated with silica.

Cyclic voltammetry was conducted on an AUTO LAB PGstat 204 from Metrohm AG, Herisau, Switzerland. Design of Experiments plans were planned and analyzed with the software Minitab19 from Minitab Inc.

### 2. Chemicals

(S)-2-Aminobutanamide hydrochloride [7682-20-4] abcr GmbH, 98%

### 1,4-Dibromobutane [110-52-1] Merck-Schuchardt

### Acetonitrile [75-05-8] Fisher Scientific, ≥99.9%

### Dichloromethane [75-09-2] Fisher Scientific, ≥99.8%

### Levetiracetam [102767-28-2] Acros Organics

Magnesium sulphate [7487-88-9] Acros Organics, 97%

### Methanol [67-56-1] VWR Chemicals, 100.0%

Sodium bisulfate monohydrate [10034-88-5] Merck, >99%; Acros Organics, 99+%
Sodium thiosulfate pentahydrate [10102-17-7] Acros Organics, 99.5%
Sodium carbonate [497-19-8] Acros Organics, 99.5%
Sodium hydroxide [1310-73-2] Honeywell, Pellets, ≥98%;
VWR Chemicals, Pellets, 99.2%
   Sodium iodate [7681-55-2] Alfa Aesar, 99%
   Sodium periodate [7790-28-5] Fisher Scientific, 99%
   Sodium sulphate [7757-82-6] Merck
   Phosphorus(V)-oxide [1314-56-3] Alfa Aesar, 98%
   Ruthenium(IV)-oxide hydrate [12036-10-1] Aldrich
   Nitric acid (≥65%) [7697-37-3] Sigma-Aldrich
   Hydrochloric acid (˜37%) [7647-01-0] Fisher Scientific
   Sulphuric acid (>95%) [7664-93-9] Fisher Scientific
   Caffeine [58-08-2] BASF (donation)

All chemicals as used herein, except for those who were synthesized internally (as described herein), were of analytical grade and are obtained from commercial suppliers.

A stock solution of RuCl₃·H₂O (Alfa Aesar 47182, 7.9 mg) in H₂O (10 mL) was prepared daily to be used freshly (1 mL used for each reaction contained 0.79 mg RuCl₃·H₂O).

### 3. Methods

### 3.1 Gas Chromatography:

Conditions: 150° C.—5 min—25° C./min—300° C.—5 min; T^{a} det: 300° C.; T^{a} inj: 220° C.; split 50:1; flux: 1.5 mL/min; carrier: He

Column: HP-5; 5% Phenyl Methyl Siloxane; 30 m, 0.2 mm ID, 0.33 μm

5 mg/mL in MeOH Method: Injection volume=1.5 μL, inlet temperature=200° C., initial column temperature=50° C. (holding time=1 min), ramping rate=15° C./min (gradient time=11.5 min), final temperature=220° C. (hold-up time=12 min). The system was calibrated for the precursor and for Levetiracetam using caffeine as internal standard (FIG. 17).

### 3.2 Liquid Chromatography Photodiode Array (LC-PDA)

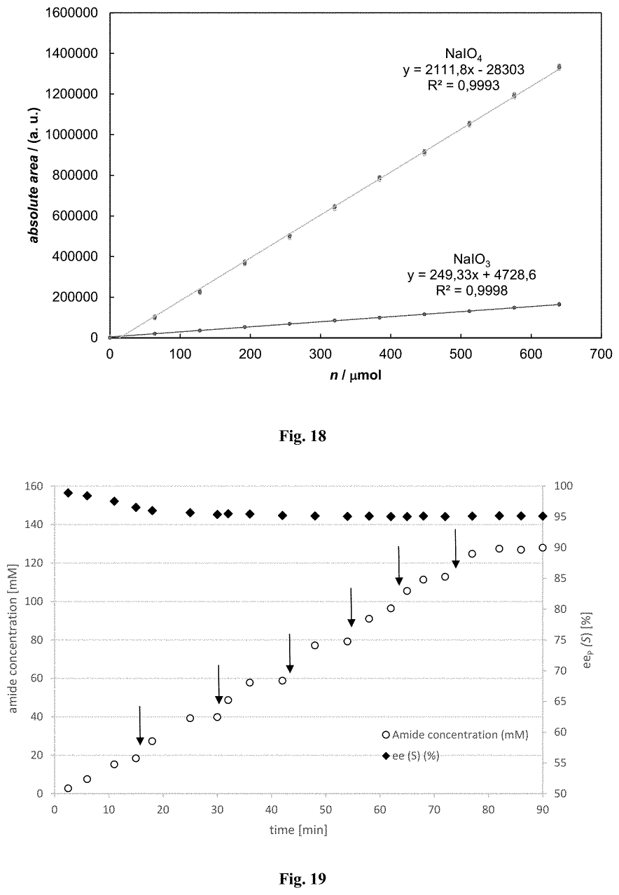

A buffered and diluted sample solution (C=5.341 mM) was subjected to LC-PDA analysis using an injection volume of 3 μL. The separation was isocratically carried out. I⁻, IO₃⁻ and IO₄⁻ were detected by the PDA detector at 1.58 min, 1.47 min and 1.92 min at a wavelength of λ=254 nm. Yields were determined by external calibration (FIG. 18).

### 3.3 Chiral HPLC

Conditions: Heptane:EtOH (90:10), 25° C., λ=215 nm, flux: 1.0 mL/min

Column: Chiralpak ADH 250×4.6 mm, 5 u

1 mg/mL in heptane:EtOH

Chiral HPLC was performed with a Waters 2695 separation module with UV detector (Waters 996 photodiode array detector) with a CHIRALPAK IB-3 column (250×4.6 mm, particle size 3 μm, flow rate: 1.0 mL/min) and a guard column (10×4.0 mm) from Daicel Chiral Technologies. The system was operated with an isocratic program. The injection volume was V=10 μL, and the eluent was composed of 10% isopropanol and 90% hexane/ethanol. The detection followed by a photodiode array detector at λ=210.1 nm.

### 3.4 TLC

Thin Layer Chromatography (TLC) was performed using commercially available aluminum plates from Merck coated with silica (60 F254) or 60-RP-18 F254 reversed-phase plates on aluminum from Merck KGaA. All samples were applied after dilution in a suitable solvent with ring caps 1-5 μL obtained from company Hirschmann and the chromatography was carried out in an eluent mixture. The TLC plate was viewed under UV light (λ=254 nm and 365 nm) and then developed in an iodine chamber or with coloring reagents and a hot air dryer:
Ninhydrin reagent: 0.3 g ninhydrin in 2.0 mL glacial acetic acid and 100 mL methanol
Dragendorff-Munier reagent: 20.0 g potassium iodide, 3.0 g bismuth (III) nitrate, 40.0 g (+)-tartaric acid and 240 mL water
KMnO₄ reagent: 3.0 g potassium permanganate and 20.0 g sodium carbonate in 300 mL water and 5.0 mL 5% sodium hydroxide solution
Seebach reagent: 10.0 g cerium (IV) sulfate, 25.0 g phosphoromolybdic acid, 940 mL water and 60 mL conc. sulfuric acid
Vanillin reagent: 1.0 g vanillin, 100 mL methanol, 12.0 mL glacial acetic acid and 4.0 mL conc. sulfuric acid
Dinitrophenylhydrazine reagent: 1.0 g 2,4-dinitrophenylhydrazine, 25 mL abs. ethanol, 8.0 mL water and 5.0 mL conc. sulfuric acid.
p-anisaldehyde reagent: 4.1 mL p-anisaldehyde, 5.6 mL conc. sulfuric acid, 1.7 mL glacial acetic acid and 150 mL ethanol
Bromocresol green reagent: 50 mg bromocresol green, 250 mL isopropanol and 0.15 mL 2 M sodium hydroxide solution.

### 4. Examples

### Reference Example 1: Synthesis of 2-(Pyrrolidin-1-yl)butanenitrile (rac-1)

The preparation has been done according to the modified procedure of Orejarena Pacheco et al (J. C. Orejarena Pacheco, T. Opatz, The Journal of Organic Chemistry 2014, 79, 5182-5192).

Propanal (17.97 g, 22.5 mL, 309.3 mmol, 1.1 eq.) was dissolved in a water methanol mixture (2000 mL, 4:1, ˜7 mL/mmol) and NaHSO₃ (32.19 g, 309.3 mmol, 1.1 eq.) was added in one portion. The solution was stirred for 2 h and pyrrolidin (20.0 g, 23.53 mL, 281.2 mmol, 1.0 eq.) was carefully added (big batches >0.1 mol needed cooling with an ice bath). KCN (36.62 g, 562.4 mmol, 2.0 eq.) was added carefully and the mixture stirred for an additional 16 h. The reaction mixture was extracted with ethyl acetate in a Kutscher-Steudel. (F. Kutscher, H. Steudel, in Hoppe-Seyler's Zeitschrift far physiologische Chemie, Vol. 39, 1903, p. 473.) The organic extract was dried over sodium sulfate, filtered and concentrated in vacuo to yield the crude product. The alpha-aminonitrile was purified by distillation (95° C., 23 mbar) to yield a colorless oil (51%-86%). The reaction was scaled from 10 mmol (711 mg Pyrrolidine) up to 2.0 mol (142 g Pyrrolidine).

Bp: 95° C. (23 mbar).

IR (ATR): ν=2970 (s), 2939 (m), 2879 (m). 2810 (m), 2222 (w), 1461 (m), 1355 (w), 1151 (m), 1085 (m), 872 (m) cm⁻¹.

¹H-NMR, COSY (Correlated Spectroscopy) (300 MHz, CDCl₃): δ=3.63 (t, ³J_{H-2, H-1}=7.8 Hz, 1H, H-1), 2.75-2.52 (m, 4H, H-2′, H-5′), 1.88-1.71 (m, 6H, H-2, H-3′, H-4′), 1.05 (t, ³J_{H-2, H-3j}=7.4 Hz, 3H, H-3).

¹³C-NMR, HMBC (Heteronuclear Multiple Bond Correlation), HSQC (Heteronuclear Single Quantum Coherence) (75 MHz, CDCl₃): δ=117.7 (CN), 57.2 (C-1), 50.1 (C-2′, C-5′), 26.2 (C2), 23.5 (C-3′, C-4′), 10.9 (C-3).

ESI-MS: m/z (%)=139.1 (100) [C₈H₁₅N₂]⁺, 112.3 (10) [C₇H₁₄N]⁺.

In the subsequent section experiments are described wherein it was investigated whether different oxidation catalyst systems are suitable for the regioselective chemical oxidation of the pyrrolidine substrate (S)-2-(pyrrolidin-1-yl)butane amide (2) under retention of the stereo configuration (Scheme 2). The formation of the oxidized lactam product (S)- and (R)-2-(2-oxopyrrolidin-1-yl)butane amide (4) and optionally of the intermediary heminaminal (6) oxidation product was analyzed.

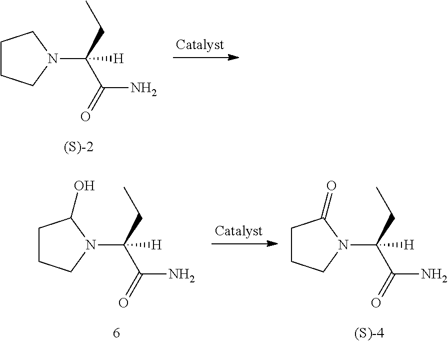

### Scheme 2: Regioselective Chemical Oxidation of (S)-2-(pyrrolidin-1-yl)butane Amide (2) to (S)-2-(2-oxopyrrolidin-1-yl)butane Amide (4)

### Synthesis Example 1: Synthesis of (S)-2-(2-oxopyrrolidin-1-yl)butane Amide (4) with RuCl₃·H₂O and NaIO₄

The oxidizing ruthenium species was obtained in situ from RuCl₃ H₂O and NaIO₄.

To a preformed solution of RuCl₃·H₂O in H₂O (1 mL, 0.79 mg, 3.52 μmol) was added a solution of NaIO₄ 5 wt % (278 mg, 1.3 mmol, 2.6 eq, in 5 mL H₂O). To the yellowish mixture formed, (S)-2-(pyrrolidin-1-yl)butane amide (2) (78.1 mg, 0.5 mmol) dissolved in EtOAc (2.5 mL) and H₂O (1 mL) was added. The reaction vial was vigorously stirred at room temperature for 30 minutes.

After this time, 2-propanol (2 mL) was added and the mixture was stirred for additional 30 minutes. The solid precipitated in the interphase was filtered and discarded. The aqueous layer was extracted with EtOAc, dried (MgSO₄) and concentrated to obtain desired product (33 mg, crude). Low recovery possibly due to presence of product in aqueous layer (as confirmed by HPLC/MS and GC).

HPLC/MS: 33% final product (4)
GC: 58% final product (4), no starting material (2)
Chiral HPLC (crude): ee 79% (S)-enantiomer of (4)

### Synthesis Example 2: Synthesis of (S)-2-(2-oxopyrrolidin-1-yl)butane Amide (4) with RuCl₃·H₂O and NaIO₄

The oxidizing ruthenium species was obtained in situ from RuCl₃ H₂O and NaIO₄.

To a preformed solution of RuCl₃·H₂O in H₂O (1 mL, 0.79 mg, 3.52 μmol) was added a solution of NaIO4 5 wt % (356 mg, 1.66 mmol, 2.6 eq, in 5 mL H₂O). To the yellowish mixture formed (S)-2-(pyrrolidin-1-yl)butane amide (2) (100 mg, 0.64 mmol) dissolved in EtOAc (2.5 mL) and H₂O (1 mL) was added. The reaction vial was vigorously stirred at room temperature for 10 minutes.

After this time, 2-propanol (2 mL) was added and the mixture was stirred for additional 30 minutes. The solid precipitated in the interphase was filtered and discarded. The aqueous layer was extracted with EtOAc, dried (MgSO₄) and concentrated to obtain desired product (36 mg, crude). Low recovery possibly due to presence of product in aqueous layer (as confirmed by HPLC/MS and GC).

HPLC/MS: 46% final product (4).
GC: 75% final product (4), no starting material (2).
Chiral HPLC (crude): ee 92% (S)-enantiomer of (4).

Aqueous layer was extracted with isobutanol (×3), dried and concentrated to obtain additional 22 mg product.

### Synthesis Example 3: Synthesis of (S)-2-(2-oxopyrrolidin-1-yl)butane Amide (4) with RuCl₃·H₂O, NaIO₄ and Sodium Oxalate

The oxidizing ruthenium species was obtained in situ from RuCl₃ H₂O and NaIO₄.

To a preformed solution of RuCl₃·H₂O in H₂O (1 mL, 0.79 mg, 3.52 μmol) was added sodium oxalate (8.6 mg, 0.1 eq) and a solution of NaIO₄ 5 wt % (356 mg, 1.66 mmol, 2.6 eq. in 5 mL H₂O). To the yellowish mixture formed, was added (S)-2-(pyrrolidin-1-yl)butane amide (2) (100 mg, 0.64 mmol) dissolved in EtOAc (2.5 mL) and H₂O (1 mL). The reaction vial was vigorously stirred at room temperature for 10 minutes.

After this time, 2-propanol (2 mL) was added and the mixture was stirred for additional 30 minutes. The solid precipitated in the interphase was filtered and discarded. Then, the mixture was concentrated to dryness.

GC: 6% starting material (2), 7% intermediary product (6), 68% final product (4)
Chiral HPLC (crude): ee 95% (S)-enantiomer of (4)

TABLE 34
Results of Synthesis Examples 1 to 3: Synthesis of (S)-2-(2-oxopyrrolidin-1-yl)butane amide (4)
with RuCl₃•H₂O and NalO₄ and optionally NaCLO and optionally sodium oxalate

| | | | | HPLC chiral | | | |
|---|---|---|---|---|---|---|---|
| Synthesis | | | | (crude) (S)-FP | GC results (a/a)³⁾ | | |
| Example | [Ru salt]¹⁾ | [Ox] | t | ee (%)²⁾ | SM | INT | FP |
| 1 | RuCl₃•H₂O 0,55% eq | NalO₄ (2.6 eq) | 30′ | 79% | — | — | 58 |
| | EtOAc/H₂O (2.5:6) | | | | | | |
| 2 | RuCl₃•H₂O 0.55% eq | NalO₄ (2.6 eq) | 10′ | 92% | — | 4 | 75 |
| | EtOAc/H₂O (2.5:6) | | | | | | |
| 3 | RuCl₃•H₂O 0.55% eq | NalO₄ (2.6 eq) + | 10′ | 95% | 6 | 7 | 68 |
| | EtOAc/H₂O (2.5:6) | NaC₂O₄ 10% (0.1 eq) | | | | | |

¹⁾preform Ru (IV) catalyst by mixing Ru salt precursor [Ru salt] and oxidant [Ox]
²⁾estimated values from crude reaction
³⁾SM = Starting material = (2); FP = Final product = (4); INT = Intermediary Product = (6)

### Synthesis Example 4: Synthesis of 2-(2-oxopyrrolidin-1-yl)butane Amide (4) with RuO₄

The oxidizing ruthenium species was obtained in situ from RuO₂ and NaIO₄ in a process modified.

2-(Pyrrolidin-1-yl)butane amide (2, 78.1 mg, 0.5 mmol, 1.0 eq.) was dissolved in ethyl acetate (2.5 mL) under sonification (5 min), RuO₂ (366 μg, 2.75 μmol, 0.55 mol %) and NaIO₄ solution (5 wt %, 5 mL, =2.6 eq.) was added. The reaction vial was closed immediately and the slurry stirred at room temperature for 30 min. The layers were separated and the aqueous layer was extracted with ethyl acetate (5×3 mL). The combined organic layers were treated with 2-propanol (2 mL) for 30 min and carefully concentrated in vacuo to yield the crude product. The product was by purified by column chromatography (silica gel 35-70 μm, Arcos Organics) (cyclohexane/acetic acid ethyl ester=3:1, 0.4 bar nitrogen overpressure).

2-(2-Oxopyrrolidin-1-yl)butane amide (4) was isolated with 76% yield (53.4 mg, 0.382 mmol) as colorless oil, which formed crystals.

IR (ATR): ν=3274 (m_{B}), 2969 (m), 2938 (m), 2878 (m), 1682 (vs), 1462 (m), 1422 (m), 1288 (m) cm⁻¹.

¹H-NMR, COSY (300 MHz, CDCl₃): δ=6.43 (s_{B}, 1H, NH₂), 5.75 (s_{B}, 1H, NH₂), 4.45 (dd, ³J_{H-2a, H-1}=8.9 Hz, ³J_{H-3b, H-2}=6.8 Hz 1H, H-2), 3.50-3.33 (m, 2H, H-5′), 2.47-2.36 (m, 2H, H-3′), 2.09-1.99 (m, 2H, H-4′), 2.00-1.87 (m, 1H, H-3ₐ), 1.68 (ddq, ⁴J_{H-3a, H-3b}=14.5 Hz, ³J_{H-2, H-3b}=8.9 Hz, ³J_{H-4, H-3b}=7.4 Hz, 1H, H-3_{b}), 0.89 (t, ³J_{H-3, H-4}=7.4 Hz, 3H, H-4).

¹³C-NMR, HMBC, HSQC (75 MHz, CDCl₃): δ=176.2 (C-2′), 172.5 (C-1), 56.2 (C-2), 44.0 (C5), 31.2 (C-3′), 21.2 (C-3), 18.3 (C-4′), 10.6 (C-4).

ESI-MS: m/z (%)=193.1 (100) [C₈H₁₄N₂O₂Na]⁺, 126.1 (27) [C₇H₁₂NO]⁺.

### Synthesis Example 5: Synthesis of (S)-2-(2-oxopyrrolidin-1-yl)butane Amide (4) with RuO₄

The experiment of Synthesis Example 4 was repeated with substrate 2-(pyrrolidinyl)butane amide (2) predominantly consisting of the (S)-enantiomer ((S) enantiomer 89.34%; (R) enantiomer 10.66%). The chiral HPLC (A=210 nm; CHIRALPAK IB-3 column (250×4.6 mm, particle size 3 μm, 4.6×250 mm; hexane:ethanol (0.1% EDA)=90:10) of the crude product revealed a full preservation of chirality without racemization

### Synthesis Example 6: Synthesis of (S)-2-(2-oxopyrrolidin-1-yl)butane Amide (4) with RuO₄

0.5-1 mol % RuO₂H₂O (0.5-1.0 mg, 3.20-6.40 μmol) and 2.60 eq. of NaIO₄ (356 mg, 1.66 mmol) were suspended in acetonitrile/water (2:1) until the solution showed a pale-yellow color. (S)-2 (100 mg, 640 μmol) was added and the reaction was stirred at room temperature for 0.5 h. Levetiracetam (4) was obtained in 66% GC-yield. The product was isolated by flash column chromatography on silica gel (12×2 cm, CH₂Cl₂/MeOH=10:1). Levetiracetam was obtained in 49% isolated yield and in 99.6% ee.

TLC (SiO₂, Ninhydrin stain, strong heating), R_{f}(CH₂Cl₂/MeOH=10:1)=0.56, R_{f}(CH₂Cl₂/MeOH=20:1)=0.13; GC: R_{f}=8.98 min at ˜180° C.; LC-MS (HR): calculated for C₈H₁₄N₂O₂ 170.1055 Da, found: [M+H]⁺ 171.1128; ¹H NMR (400 MHz, Chloroform-d) δ 6.58 (s, 1H), 6.11-5.88 (m, 1H), 4.46 (dd, J=9.1, 6.6 Hz, 1H), 3.41 (dddd, J=34.1, 9.8, 8.0, 6.1 Hz, 2H), 2.47-2.29 (m, 2H), 2.10-1.85 (m, 3H), 1.65 (ddq, J=14.6, 9.1, 7.4 Hz, 1H), 0.86 (t, J=7.4 Hz, 3H); ¹³C NMR (101 MHz, Chloroform-d) δ 176.10 (C_{q}), 172.64 (C_{q}), 56.07 (CH), 43.89 (CH₂), 31.15 (CH₂), 21.21 (CH₂), 18.20 (CH₂), 10.59 (CH₃).

### Synthesis Example 7: Synthesis of (S)-2-(2-oxopyrrolidin-1-yl)butane Amide (4) with Immobilized RuO₄

For the catalyst immobilization, RuO₂H₂O (200 mg) was mixed with aluminum oxide, C18 reversed phase material, polyacrylonitrile, charcoal, or mixtures thereof (m=25 g). The prepared material was loaded on a glass column (12×1.5 cm) and the column was connected to a Fink pump (Ritmo R033) or was alternatively pressurized using a flash adapter. For the oxidation, (S)-2 (100 mg, 640 μmol) and 2.60 eq. of NaIO₄ (356 mg, 1.66 μmol) were dissolved in water/acetonitrile (2:1 v/v, 25 mL), and the solution was pumped through the column. The system was rinsed with another 10 mL of water. The yield of Levetiracetam (4) was determined by GC versus caffeine as internal standard. Levetiracetam was obtained in a maximum yield of 22%.

### Synthesis Example 8: Electrochemical Recycling of Sodium Iodate

Sodium iodate was recovered from the ruthenium-catalysis by the addition of methanol to the reaction mixture. The precipitated fine crystalline needles were filtered off and were dried under reduced pressure. Iodate was isolated in >95% yield.

In a divided beaker cell equipped with a Nafion membrane, both chambers were filled with 6 mL of aqueous NaOH solution (1.0 M). NaIO₃ (127 mg, 640 μmol) was added to the anodic chamber and the electrolysis was started using BDD (boron-doped diamond) as anode, stainless steel as cathode, a charge amount of Q=3 F, and a current density of j=10 mA/cm². After the electrolysis was completed, the content of the anode chamber was acidified with a 1.0 M NaHSO₄ aqueous solution and analyzed by LC-PDA. Sodium periodate was obtained in 86% yield. For the isolation of the para-periodate, the precipitate was filtered off by vacuum filtration and was dried over phosphorus pentoxide under vacuum. The purity was controlled by LC-PDA and IR analysis.

For the isolation of the para-periodate, sodium hydroxide was added and the precipitate was filtered off by vacuum filtration. The solid residue was washed with water and subsequently dried in a desiccator over phosphorus pentoxide under vacuum. The conversion/purity was controlled by LC-PDA and IR analysis. The isolation of meta-periodate was carried out as according to the procedures of Mehltretter et al. and Willard et al. (H. H. Willard, R. R. Ralston, Trans. Electrochem. Soc. 1932, 62, 239; C. L. Mehltretter, C. S. Wise, U.S. Pat. No. 2,989,371A, 1961) The para-periodate was neutralized either by sulfuric or nitric acid and crystallized or recrystallized as mentioned.

### Synthesis Example 9: Synthesis of (S)-2-(2-oxopyrrolidin-1-yl)butane Amide (4) with RuO₄ Using Electrochemically Produced NaIO₄

According to the procedure in synthesis example 6, RuO₂·H₂O (1 mg) and electrochemically generated NaIO₄ (550 mg, ˜4 eq.) were suspended. (S)-2 (100 mg, 640 μmol) was added and the reaction was stirred at room temperature for 0.5 h. Levetiracetam was obtained in 57% GC yield using caffeine as internal standard.

### Synthesis Example 10: Recrystallization of Paraperiodate to Metaperiodate

Sodium paraperiodate (4.00 g, 13.6 mmol), HNO₃ (2.2 mL, 65%) and water (8 mL) were refluxed at 130° C. for several minutes. Water was distilled off until crystallisation started. The mixture was cooled to 4° C. and was kept at this temperature overnight. The crystals were filtered off and were dried under vacuum. Sodium metaperiodate was obtained as colourless crystals (2.057 g, 9.62 mmol, 71%). IR data were in accordance with the Bio-Rad database (Infrared spectral data were obtained from the Bio-Rad/Sadtler IR Data Collection, Bio-Rad Laboratories, Philadelphia, Pa. (US) and can be found under https://spectrabase.com. Spectrum ID (meta-periodate): 3ZPsHGmepSu).

### Synthesis Example 11: Electrolysis with Recovered Sodium Iodate

Recovered sodium iodate (2.08 g, 10.5 mmol) from the ruthenium-catalysed step in levetiracetam synthesis and sodium hydroxide (2.00 g, 50.0 mmol) were dissolved in water (50 mL) and were electrolysed according to RP 3. A current density of j=50 mA cm⁻² and a charge amount of Q=4 F (4055 C) were applied. Sodium paraperiodate was obtained in reproducible 83% yield as determined by LC-PDA.

### Synthesis Example 12: One-Pot Enzymatic Dynamic Kinetic Resolution by Strecker Reaction from Three Precursors

KCN (3.65 mg/mL) was dissolved in Tris-HCl buffer (300 mM, pH 7.5) and mixed with pyrrolidine (3.22 mg/mL) and propanal (4.2 μL/mL or 12.4 μL/mL, resp.). KH₂PO₄ (200 mM) was used to adjust the pH to 7.3. Final buffer concentrations were 150 mM TrisHCl and 100 mM potassium phosphate. Per reaction, 50 μL of cell free extract of recombinantly produced GhNHase or CtNHase wild-type enzymes was mixed with 450 μL of this reaction mixture. Each reaction was carried out in triplicate. The reactions proceeded at 25° C. and 500 rpm in an Eppendorf thermomixer. Reactions were terminated and analyzed as described herein. The results are summarized in Table 35.

TABLE 35
One-pot enzymatic dynamic kinetic resolution by Strecker reaction from three pre-cursors.
Conditions: pH 7.3, 25° C., 10% CFE, 30 min reaction time

| | | | pyrrolidine | propanal | (S)-2 | ee (S)-2 |
|---|---|---|---|---|---|---|
| entry | NHase | KCN [mM] | [mM] | [mM] | [mM] | [%] |
| 1 | GhHNase | 26.9 | 29.4 | approx. 30¹ | 2.3 ± 0.0 | 75.9 ± 0.2 |
| 2 | CtNHase | 26.9 | 29.4 | approx. 30¹ | 0.1 ± 0.0 | n.d.² |
| 3 | GhHNase | 26.9 | 29.4 | approx. 90¹ | 8.4 ± 0.4 | 73.9 ± 0.1 |
| 4 | CtNHase | 26.9 | 29.4 | approx. 90¹ | 4.9 ± 0.1 | 82.1 ± 0.1 |

¹propanal volatility impairs correct pipetting of small volumes.
²peak area of (R)-2 below detection limit

As can be seen, the excess of propanal had a pronounced effect on product formation

TABLE 36
Assignment of SEQ ID NOs:

| | | | |
|---|---|---|---|
| SEQ | | | |
| ID NO | Name | Source | Type |

β-Subunits

| | | | |
|---|---|---|---|
| 1 | CtNHase β-subunit_AAU87543.1 | Comamonas testosteroni | NA |
| 2 | CtHNase β-subunit_AAU87543.1 | Comamonas testosteroni | AA |
| 3 | KoNHase β-subunit_OSY94201.1 | Klebsiella oxytoca | NA |
| 4 | KoNHase β-subunit_OSY94201.1 | Klebsiella oxytoca | AA |
| 5 | NaNHase β-subunit_WP 052668588.1 | Nitriliruptor alkaliphilus | NA |
| 6 | NaNHase β-subunit_WP 052668588.1 | Nitriliruptor alkaliphilus | AA |
| 7 | GhNHase β-subunit_WP 066163466.1 | Gordonia hydrophobica | NA |
| 8 | GhNHase β-subunit_WP 066163466.1 | Gordonia hydrophobica | AA |
| 9 | PmNHase β-subunit_WP 074846644.1 | Pseudomonas marginalis | AA |
| 10 | PmNHase β-subunit_WP 074846644.1 | Pseudomonas marginalis | NA |
| 11 | ReNHase β-subunit_P13449.1 | Rhodococcus erythropolis | NA |
| 12 | ReNHase β-subunit_P13449.1 | Rhodococcus erythropolis | AA |
| 13 | PkNHase β-subunit_partial sequence | Pseudomonas kilonensis | AA |

α-Subunits

| | | | |
|---|---|---|---|
| 14 | CtNHase α-subunit_AAU87542.1 | Comamonas testosteroni | NA |
| 15 | CtNHase α-subunit_AAU87542.1 | Comamonas testosteroni | AA |
| 16 | KoNHase α-subunit_OSY94202.1 | Klebsiella oxytoca | NA |
| 17 | KoNHase α-subunit_OSY94202.1 | Klebsiella oxytoca | AA |
| 18 | NaNHase α-subunit_WP 052668589.1 | Nitriliruptor alkaliphilus | NA |
| 19 | NaNHase α-subunit_WP 052668589.1 | Nitriliruptor alkaliphilus | AA |
| 20 | GhNHase α-subunit_WP 066163464.1 | Gordonia hydrophobica | NA |
| 21 | GhNHase α-subunit_WP 066163464.1 | Gordonia hydrophobica | AA |
| 22 | PmNHase α-subunit_WP 074846646.1 | Pseudomonas marginalis | NA |
| 23 | PmNHase α-subunit_WP 074846646.1 | Pseudomonas marginalis | AA |
| 24 | ReNHase α-subunit_P13448.3 | Rhodococcus erythropolis | NA |
| 25 | ReNHase α-subunit_P13448.3 | Rhodococcus erythropolis | AA |
| 26 | PkNHase α-subunit | Pseudomonas kilonensis | NA |
| 27 | PkNHase α-subunit | Pseudomonas kilonensis | AA |

Primer
For generation of site-saturation libraries

| | | | |
|---|---|---|---|
| 28 | Ct-aQ93X_for | Artificial sequence | NA |
| 29 | Ct-aQ93X_rev | Artificial sequence | NA |
| 30 | Ct-aW120X_for | Artificial sequence | NA |
| 31 | Ct-aW120X_rev | Artificial sequence | NA |
| 32 | Ct-αP126X_for | Artificial sequence | NA |
| 33 | Ct-aP126X_rev | Artificial sequence | NA |
| 34 | Ct-aK131X_for | Artificial sequence | NA |
| 35 | Ct-aK131X_rev | Artificial sequence | NA |
| 36 | Ct-aR169X_for | Artificial sequence | NA |
| 37 | Ct-aR169X_rev | Artificial sequence | NA |
| 38 | Ct-bM34X_for | Artificial sequence | NA |
| 39 | Ct-bM34X_rev | Artificial sequence | NA |
| 40 | Ct-bF37X_for | Artificial sequence | NA |
| 41 | Ct-bF37X_rev | Artificial sequence | NA |
| 42 | Ct-bL48X_for | Artificial sequence | NA |
| 43 | Ct-bL48X_rev | Artificial sequence | NA |
| 44 | Ct-bF51X_for | Artificial sequence | NA |
| 45 | Ct-bF51X_rev | Artificial sequence | NA |
| 46 | Ct-bY68X-long_for | Artificial sequence | NA |
| 47 | Ct-bY68X-long_rev | Artificial sequence | NA |

For site-directed mutagenesis of pMS470-CtNHase

| | | | |
|---|---|---|---|
| 48 | Ct-aW120F_for | Artificial sequence | NA |
| 49 | Ct-aW120F_rev | Artificial sequence | NA |
| 50 | Ct-bM34L_for | Artificial sequence | NA |
| 51 | Ct-bM34L_rev | Artificial sequence | NA |
| 52 | Ct-bM34Q_for | Artificial sequence | NA |
| 53 | Ct-bM34Q_rev | Artificial sequence | NA |

For generation of randomly mutated CtNHase gene fragments

| | | | |
|---|---|---|---|
| 54 | Ct-alpha1_for | Artificial sequence | NA |
| 55 | Ct-alpha1_rev | Artificial sequence | NA |
| 56 | Ct-alpha2_for | Artificial sequence | NA |
| 57 | Ct-alpha2_rev | Artificial sequence | NA |
| 58 | Ct-beta1_for | Artificial sequence | NA |
| 59 | Ct-beta1_rev | Artificial sequence | NA |
| 60 | Ct-beta2_for | Artificial sequence | NA |
| 61 | Ct-beta2 rev | Artificial sequence | NA |

For generation of pMS470-CtNHase backbone strains

| | | | |
|---|---|---|---|
| 62 | Ct-A1bb-lig_for | Artificial sequence | NA |
| 63 | Ct-A1bb-lig_rev | Artificial sequence | NA |
| 64 | Ct-A2bb-lig_for | Artificial sequence | NA |
| 65 | Ct-A2bb-lig_rev | Artificial sequence | NA |
| 66 | Ct-B1bb-lig_for | Artificial sequence | NA |
| 67 | Ct-B1bb-lig_rev | Artificial sequence | NA |
| 68 | Ct-B2bb-lig_for | Artificial sequence | NA |
| 69 | Ct-B2bb-lig_rev | Artificial sequence | NA |

For generation of pMS470-CtNHase-β1-focused library

| | | | |
|---|---|---|---|
| 70 | Ct-beta1-focused_for | Artificial sequence | NA |
| 71 | Ct-beta1-focused_rev | Artificial sequence | NA |

For generation of pMS470-CtNHase-αP121X-βF5IL mutants

| | | | |
|---|---|---|---|
| 72 | Ct-P121A_for | Artificial sequence | NA |
| 73 | Ct-P121A_rev | Artificial sequence | NA |
| 74 | Ct-P121R_for | Artificial sequence | NA |
| 75 | Ct-P121R_rev | Artificial sequence | NA |
| 76 | Ct-P121N_for | Artificial sequence | NA |
| 77 | Ct-P121N_rev | Artificial sequence | NA |
| 78 | Ct-P121D_for | Artificial sequence | NA |
| 79 | Ct-P121D_rev | Artificial sequence | NA |
| 80 | Ct-P121C_for | Artificial sequence | NA |
| 81 | Ct-P121C_rev | Artificial sequence | NA |
| 82 | Ct-P121Q_for | Artificial sequence | NA |
| 83 | Ct-P121Q_rev | Artificial sequence | NA |
| 84 | Ct-P121E_for | Artificial sequence | NA |
| 85 | Ct-P121E_rev | Artificial sequence | NA |
| 86 | Ct-P121G_for | Artificial sequence | NA |
| 87 | Ct-P121G_rev | Artificial sequence | NA |
| 88 | Ct-P121H_for | Artificial sequence | NA |
| 89 | Ct-P121H_rev | Artificial sequence | NA |
| 90 | Ct-P121I_for | Artificial sequence | NA |
| 91 | Ct-P121I_rev | Artificial sequence | NA |
| 92 | Ct-P121L_for | Artificial sequence | NA |
| 93 | Ct-P121L_rev | Artificial sequence | NA |
| 94 | Ct-P121K_for | Artificial sequence | NA |
| 95 | Ct-P121K_rev | Artificial sequence | NA |
| 96 | Ct-P121M_for | Artificial sequence | NA |
| 97 | Ct-P121M_rev | Artificial sequence | NA |
| 98 | Ct-P121F_for | Artificial sequence | NA |
| 99 | Ct-P121F_rev | Artificial sequence | NA |
| 100 | Ct-P121W_for | Artificial sequence | NA |
| 101 | Ct-P121W_rev | Artificial sequence | NA |
| 102 | Ct-P121Y_for | Artificial sequence | NA |
| 103 | Ct-P121Y_rev | Artificial sequence | NA |

For generation of pMS470-CtNHase combination variants

| | | | |
|---|---|---|---|
| 104 | Ct-P121Tfor | Artificial sequence | NA |
| 105 | Ct-P121T rev | Artificial sequence | NA |
| 106 | Ct-V110I_for | Artificial sequence | NA |
| 107 | Ct-V110I_rev | Artificial sequence | NA |
| 108 | Ct-L48R-G54C_for | Artificial sequence | NA |
| 109 | Ct-L48R-G54C_rev | Artificial sequence | NA |
| 110 | Ct-L48R-G54R_for | Artificial sequence | NA |
| 111 | Ct-L48R-G54R_rev | Artificial sequence | NA |
| 112 | Ct-L48R-G54V_for | Artificial sequence | NA |
| 113 | Ct-L48R-G54V_rev | Artificial sequence | NA |
| 114 | Ct-L48P-G54C_for | Artificial sequence | NA |
| 115 | Ct-L48P-G54C_rev | Artificial sequence | NA |
| 116 | Ct-L48P-G54R_for | Artificial sequence | NA |
| 117 | Ct-L48P-G54R_rev | Artificial sequence | NA |
| 118 | Ct-L48P-G54V_for | Artificial sequence | NA |
| 119 | Ct-L48P-G54V_rev | Artificial sequence | NA |
| 120 | Ct-L48F-G54C_for | Artificial sequence | NA |
| 121 | Ct-L48F-G54C_rev | Artificial sequence | NA |
| 122 | Ct-L48F-G54R_for | Artificial sequence | NA |
| 123 | Ct-L48F-G54R_rev | Artificial sequence | NA |
| 124 | Ct-L48F-G54V_for | Artificial sequence | NA |
| 125 | Ct-L48F-G54V_rev | Artificial sequence | NA |
| 126 | Ct-L48P-F51V-G54V_for | Artificial sequence | NA |
| 127 | Ct-L48P-F51V-G54V_rev | Artificial sequence | NA |
| 128 | Ct-L48F_for | Artificial sequence | NA |
| 129 | Ct-L48F_rev | Artificial sequence | NA |
| 130 | Ct-F51L_for | Artificial sequence | NA |
| 131 | Ct-F51L_rev | Artificial sequence | NA |

Inserts for vector cloning

| | | | |
|---|---|---|---|
| 132 | CNHase | Artificial sequence | NA |
| 133 | KoNHase | Artificial sequence | NA |
| 134 | NaNHase | Artificial sequence | NA |

Amidase

| | | | |
|---|---|---|---|
| 135 | ReAmindase | Rhodococcus erythropolis | AA |

Accessory proteins

| | | | |
|---|---|---|---|
| 136 | CfNHase accessory protein | Comamonas testosteroni | NA |
| 137 | CfNHase accessory protein | Comamonas testosteroni | AA |
| 138 | KoNHase accessory protein | Klebsiella oxytoca | NA |
| 139 | KoNHase accessory protein | Klebsiella oxytoca | AA |
| 140 | NaNHase accessory protein | Nitriliruptor alkaliphilus | NA |
| 141 | NaNHase accessory protein | Nitriliruptor alkaliphilus | AA |
| 142 | GhNHase accessory protein | Gordonia hydrophobica | NA |
| 143 | GhNHase accessory protein | Gordonia hydrophobica | AA |
| 144 | PmNHase accessory protein | Pseudomonas marginalis | AA |
| 145 | ReNHase accessory protein | Rhodococcus erythropolis | AA |

AA = amino acid sequence
NA = nucleic acid sequence

Reference is made expressly to the disclosure of the documents mentioned herein.

1. A biocatalytic process for preparing an alpha-amino amide of the general formula I 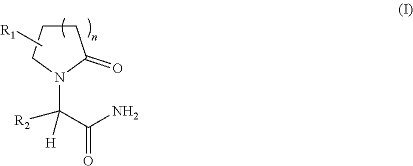
wherein
n is 0 or an integer of 1 to 4; and
R₁ and R₂ independently of each other represent H or a straight-chain or branched, saturated or non-saturated hydrocarbon group having 1 to 6 carbon atoms; in particular H or C₁-C₆ or C₁-C₃ alkyl;
optionally in essentially stereoisomerically pure form or as a mixture of stereoisomers; in particular in essentially stereoisomerically pure form,
which method comprises
1) contacting an alpha-amino nitrile of the general formula II 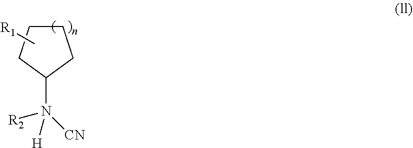
wherein n, R₁ and R₂ are as defined above,
with a polypeptide having nitrile hydratase (NHase) activity, whereby said nitrile compound of the general formula II is converted to said compound of general formula I; and
2) optionally isolating a compound of formula I, wherein
a nitrile of formula II is applied, wherein n and R₁ are as defined above and R₂ represents a straight-chain or branched, saturated or non-saturated hydrocarbon group having 1 to 6 carbon atoms, in particular C₁-C₆ or C₁-C₃ alkyl.

2. The process of claim 1, wherein a nitrile of the general formula IIa 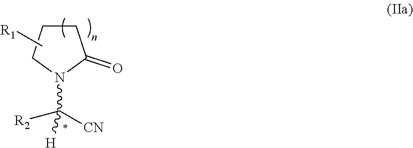
is applied, which comprises an asymmetric carbon atom in alpha-position to the cyano group
and
wherein
n and R₁ are as defined above and
R₂ represents a straight-chain or branched, saturated or non-saturated hydrocarbon group having 1 to 6 carbon atoms, in particular C₁-C₆ or C₁-C₃ alkyl,
wherein said nitrile is applied in the form of a mixture of stereoisomers, in particular as mixture of isomers comprising an (S)- or (R)-configuration at the carbon atom in alpha-position to the cyano group, and wherein said stereoisomeric mixture is converted via dynamic kinetic resolution to a reaction product containing a stereoisomeric excess either of a compound of formula Ia or of a compound of formula Ib. 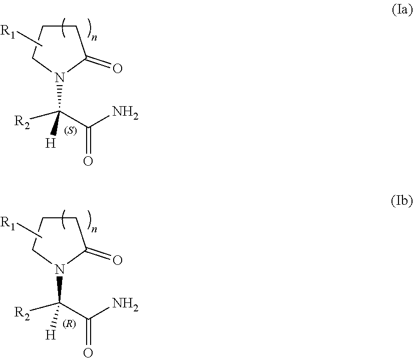
wherein
n, R₁ and R₂ are as defined above.

3. The process of claim 2, wherein a reaction product is obtained containing a stereoisomeric excess either of a compound of formula XIa or of a compound of formula XIb

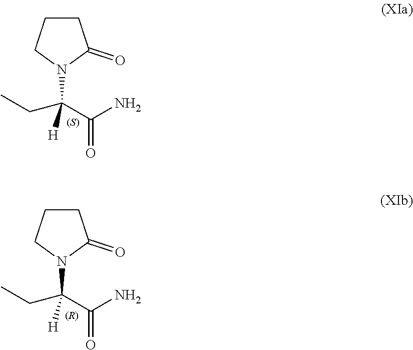

4. The process of claim 1, wherein step 1) is performed in the presence of an isolated, enriched or crude NHase enzyme, or in the presence of a recombinant microorganism functionally expressing said enzymes, or disrupted cells or a cell homogenate obtained therefrom.

5. The process of claim 4, wherein the NHase is a (S)—NHase and is selected from the enzymes:
a) CtNHase, comprising an α-polypeptide subunit according to SEQ ID NO: 15 or a sequence having at least 50% sequence identity to SEQ ID NO: 15 and a β-polypeptide subunit according to SEQ ID NO: 2 or a sequence having at least 50% sequence identity to SEQ ID NO: 2, while retaining (S)—NHase activity;
b) KoNHase, comprising an α-polypeptide subunit according to SEQ ID NO: 17 or a sequence having at least 50% sequence identity to SEQ ID NO: 17 and a β-polypeptide subunit according to SEQ ID NO: 4 or a sequence having at least 50% sequence identity to SEQ ID NO: 4, while retaining (S)—NHase activity;
c) NaNHase, comprising an α-polypeptide subunit according to SEQ ID NO: 19 or a sequence having at least 50% sequence identity to SEQ ID NO: 19 and a β-polypeptide subunit according to SEQ ID NO: 6 or a sequence having at least 50% sequence identity to SEQ ID NO: 6, while retaining (S)—NHase activity;
d) GhNHase, comprising an α-polypeptide subunit according to SEQ ID NO: 21 or a sequence having at least 50% sequence identity to SEQ ID NO: 21 and a β-polypeptide subunit according to SEQ ID NO: 8 or a sequence having at least 50% sequence identity to SEQ ID NO: 8, while retaining (S)—NHase activity;
e) PkNHase, comprising an α-polypeptide subunit according to SEQ ID NO: 27 or a sequence having at least 50% sequence identity to SEQ ID NO: 27 and a β-polypeptide subunit comprising a partial polypeptide sequence according to SEQ ID NO: 13 or a sequence having at least 50% sequence identity to said partial sequence of SEQ ID NO: 13, while retaining (S)—NHase activity;
f) PmNHase comprising an α-polypeptide subunit according to SEQ ID NO: 23 or a sequence having at least 50% sequence identity to SEQ ID NO: 23 and a β-polypeptide subunit according to SEQ ID NO: 10 or a sequence having at least 50% sequence identity to SEQ ID NO: 10, while retaining (S)—NHase activity; and
g) ReNHase. comprising an α-polypeptide subunit according to SEQ ID NO: 25 or a sequence having at least 50% sequence identity to SEQ ID NO: 25 and a β-polypeptide subunit according to SEQ ID NO: 12 or a sequence having at least 50% sequence identity to SEQ ID NO: 12, while retaining (S)—NHase activity.

6. The process of claim 5, wherein the (S)—NHase is selected from CtNHase mutants, containing at least one amino acid mutation in its α-polypeptide subunit according to SEQ ID NO: 15 and/or at least one amino acid mutation in its β-polypeptide subunit according to SEQ ID NO: 2, while retaining (S)—NHase activity.

7. The process of claim 6, wherein the CtNHase mutant is selected from mutants having at least one mutation (in particular amino acid substitution) in its α-polypeptide subunit according to SEQ ID NO: 15 in a sequence position selected from
the α sequence positions
αA71X, αK73X, αD79X, αT81X, αL87X, αG94X, αV98X,
αE101X, αN102X, αT103X, αA105X, αV106X, αV110X;
αP121X, αG124X, αY135X, αV140X, αL147X, αV153X, αA156X, αL173X,
αP174X;
in particular αV110X, and αP121X.
wherein X is selected from natural amino acids;
and/or
at least one mutation (amino acid substitution) in its β-polypeptide subunit according to SEQ ID NO: 2 in a sequence position selected from
the β sequence positions
βT32X, βV33X, βM34X, βS35X, βL36X, βL40X, βA42X, βN43X, βN45X,
βF46X, βN47X, βL48X, βE50X, βF51X, βR52X, I3H53X, βG54X, βE56X,
βR57X, βN59X, βI61X, βD62X, βL64X, βK65X, βG66X, βT67X, βE70X;
βG125X, βA126X, βR127X, βA128X, βR129X, βA131X, βV132X, βG133X,
βV136X, βR137X, βK141X, βP143X, βV144X, βG145X, βH146X, βP150X,
βY152X, βT153X, βG155X, βK156X, βV157X, βT159X, βI162X, βH164X,
βG165X, βV166X, βF167X, βV168X, βT169X, βP170X;
in particular βL48X, βF51X, βG54X, βH146X, and βF167X,
wherein X is selected from natural amino acids.

8. The process of claim 7, wherein the CtNHase mutant is selected from:
a) the single mutants: βF51L, βF51I, βF51V, βL48R and βL48P
b) the double mutants:
αV110I/βF51L,
αP121T/βF51L,
βF51V/βG54V,
βF51V/βG54I,
βF51V/βG54R,
βF51I/βG54R,
βN43I/βG54C
βF51I/βE70L,
βH53L/βG54V,
αV110I/βL48R,
αV110I/βL48P,
αV110I/βL48F,
αP121T/βL48R,
αP121T/βL48P,
αP121T/βL48F, βH146L/βF167Y,
βL48R/βG54C,
βL48R/βG54R,
βL48R/βG54V,
βL48P/βG54C,
βL48P/βG54R,
βL48P/βG54V,
βL48F/βG54C,
βL48F/βG54R, and
βL48F/βG54V;
in particular
αV110I/βF51L,
αP121T/βF51L,
βF51V/βG54V,
βN43I/βG54C
βF51I/βE70L,
βH53L/βG54V,
αP121T/βL48R,
βH146L/βF167Y, and
βL48R/βG54V.
c) the triple mutants:
βF51L/βH146L/βF167Y,
βL48R/βH146L/βF167Y,
βL48P/βH146L/βF167Y,
βL48F/βH146L/βF167Y,
αV110I/βF51V/βG54I,
αP121T/βF51V/βG54I,
βL48P/βF51V/βG54V and
βL48R/βF51I/βG54I
and
d) the multiple mutants:
βF51I/βG54R/βH146L/βF167Y,
βF51V/βG54I/βH146L/βF167Y,
βF51V/βG54R/βH146L/βF167Y,
βF51V/βG54V/βH146L/βF167Y,
αV110I/αP121T/βF51I/βH146L/βF167Y,
αV110I/αP121T/βF51L/βH146L/βF167Y

9. An isolated (S)—NHase enzyme is selected from
KoNHase, comprising an α-polypeptide subunit according to SEQ ID NO: 17 and/or a β-polypeptide subunit according to SEQ ID NO: 4 having (S)—NHase activity.

10. An isolated (S)—NHase enzyme is selected from
a mutant of CtNHase retaining (S)—NHase activity, and comprising a mutated α-polypeptide subunit, differing from SEQ ID NO: 15 in at least one amino acid residue and having at least 97% sequence identity to SEQ ID NO: 15 and/or a mutated β-polypeptide subunit, differing from SEQ ID NO: 2 in at least one amino acid residue and having at least 97% sequence identity to SEQ ID NO: 2.

11. A CtNHase mutant having (S)—NHase activity, comprising an α-polypeptide subunit according to SEQ ID NO: 15 or a sequence having at least 50% sequence identity to SEQ ID NO: 15 and a β-polypeptide subunit according to SEQ ID NO: 2 or a sequence having at least 50% sequence identity to SEQ ID NO: 2, while retaining (S)—NHase activity;
and further comprising at least one mutation selected from
a) the single mutants: βF51L, βF51I, βF51V, βL48R and βL48P
b) the double mutants:
αV110I/βF51L,
αP121T/βF51L,
βF51V/βG54V,
βF51V/βG54I,
βF51V/βG54R,
βF51I/βG54R,
βN43I/βG54C
βF51I/βE70L,
βH53L/βG54V,
αV110I/βL48R,
αV110I/βL48P,
αV110I/βL48F,
αP121T/βL48R,
αP121T/βL48P,
αP121T/βL48F, βH146L/βF167Y,
βL48R/βG54C,
βL48R/βG54R,
βL48R/βG54V,
βL48P/βG54C,
βL48P/βG54R,
βL48P/βG54V,
βL48F/βG54C,
βL48F/βG54R, and
βL48F/βG54V;
in particular
αV110I/βF51L,
αP121T/βF51L,
βF51V/βG54V,
βN43I/βG54C
βF51I/βE70L,
βH53L/βG54V,
αP121T/βL48R,
βH146L/βF167Y, and
βL48R/βG54V.
c) the triple mutants:
βF51L/βH146L/βF167Y,
βL48R/βH146L/βF167Y,
βL48P/βH146L/βF167Y,
βL48F/βH146L/βF167Y,
αV110I/βF51V/βG54I,
αP121T/βF51V/βG54I,
βL48P/βF51V/βG54V and
βL48R/βF51I/βG54I
and
d) the multiple mutants:
βF51I/βG54R/βH146L/βF167Y,
βF51V/βG54I/βH146L/βF167Y,
βF51V/βG54R/βH146L/βF167Y,
βF51V/βG54V/βH146L/βF167Y,
αV110I/αP121T/βF51I/βH146L/βF167Y,
αV110I/αP121T/βF51L/βH146L/βF167Y

12. A nucleic acid molecule comprising a nucleotide sequence encoding the polypeptide-subunits of a functional (S)—NHase enzyme as defined in claim 9.

13. A chemo-biocatalytic process for the preparation of a lactam compound of the formula IIIa or IIIb 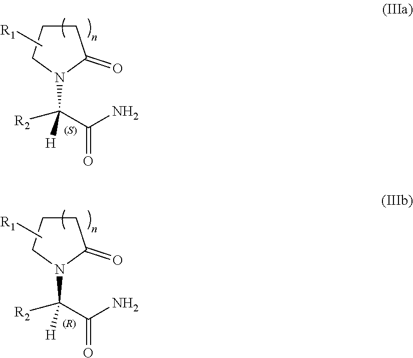
wherein
n is 0 or an integer of 1 to 4; and
R₁ and R₂ independently of each other represent H or a straight-chain or branched, saturated or non-saturated hydrocarbon group having 1 to 6 carbon atoms, in particular C₁-C₆ or C₁-C₃ alkyl;
which process comprises the following steps:
1) optionally the chemical synthesis of a stereoisomeric mixture of an alpha amino nitrile of the formula IIc 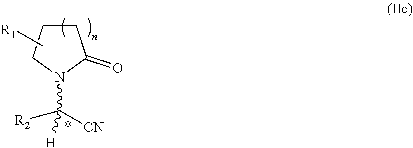
wherein n, R₁ and R₂ are as defined above,
by a Strecker synthesis, in particular by reacting a cyanide compound, in particular HCN or an alkali or alkaline earth metal cyanide, like more particularly NaCN or KCN, an aldehyde of the formula R₂—CHO, wherein R₂ is as defined above, and a cyclic amine of the formula (IV) 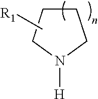
wherein n and R₁ are as defined above;
2) the enantioselective biocatalytic conversion of the compound of formula IIc, optionally as obtained according to step 1), by a process as defined in claim 1 via dynamic kinetic resolution in order to obtain a reaction product containing a stereoisomeric excess either of a compound of formula Ia or of a compound of formula Ib: 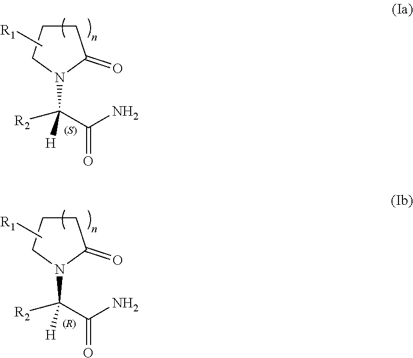
wherein n, R₁ and R₂ are as defined above;
and
3) the chemical oxidation of said alpha-amino amide of the formula Ia or Ib to the corresponding lactam derivative of the general formula IIIa or IIIb. 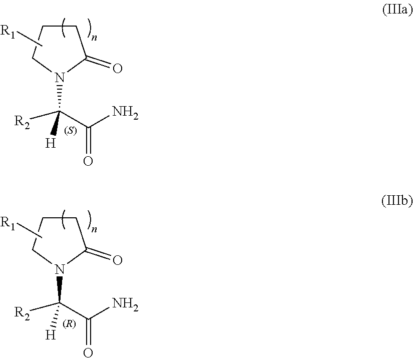
wherein n, R₁ and R₂ are as defined above.

14. The process of claim 13, wherein the chemical oxidation of step 3) is performed with an oxidation catalyst oxidizing the heterocyclic alpha-amino group in a compound of formula (Ia) or (Ib) under substantial retention of the stereochemistry at the asymmetric carbon atom in alpha-position to the amide group.

15. The process of claim 14, wherein the oxidation catalyst is selected from combinations of an inorganic ruthenium (+III) or (+IV) salt and at least one oxidant capable of in situ oxidizing ruthenium (+III) or (+IV), in particular to ruthenium (+VIII), and optionally in the presence of a mono- or polyvalent metal ligand, as for example sodium oxalate, in particular wherein the inorganic ruthenium (+III) or (+IV) salt is selected from RuCl₃, RuO₂ and the respective hydrates, in particular monohydrates, thereof; and wherein the oxidant is selected from alkali perhalogenates, alkali hypohalogenites their hydrates; or combinations thereof; in particular alkali perhalogenates.

16. The process of claim 15, wherein the oxidant is selected from
a) alkali periodates, particularly alkali meta-periodates, in particular NaIO₄
b) alkali hypochlorite, in particular NaOCl, the hydrates thereof, in particular NaOCl*5 H₂O; and
c) mixtures of a) and b).

17. The process of claim 15, wherein the oxidation catalyst is selected from
a) RuO₂/NaIO₄
b) RuO₂*H₂O/NaIO₄
c) RuCl₃*H₂O/NaIO₄
d) RuCl₃*H₂O/NaOCl*5 H₂O
e) RuCl₃*H₂O/NaIO₄/NaOCl*5 H₂O and
f) each of a) to d) in combination with a mono- or polyvalent metal ligand, as for example sodium oxalate.

18. The process of claim 13, wherein the obtained lactam derivative is selected from Levetiracetam of the formula XIIIa and Brivaracetam of the formula XXIa and Piracetam of the formula XX.

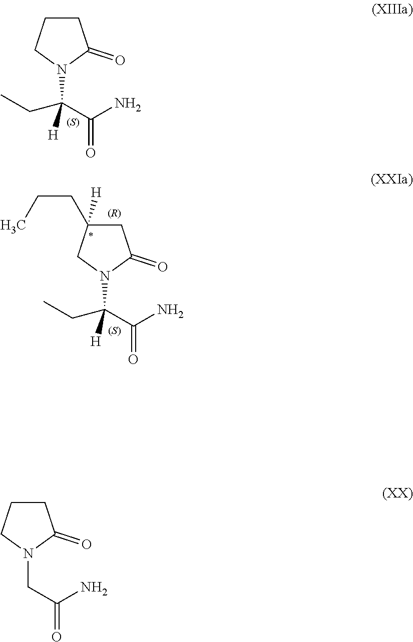

19. The process of claim 13, further comprising the recovering, in particular by precipitation, and electrochemical recycling of the spent oxidant, in particular of the electrochemical oxidation of an alkali halogenate back to an alkali perhalogenate oxidant.

20. A process for the preparation of at least one sodium periodate, which process comprises the electrochemical anodic oxidation of at least one sodium iodate to at least one sodium periodate, wherein a boron-doped diamond anode is applied.

21. The process of claim 20, wherein the anodic oxidation is performed under at least one of the following conditions:
a) aqueous solution of at least one sodium iodate at an initial concentration of 0.001 to 10 M,
b) pH of the aqueous solution of 7 or more,
c) temperature in the range of 0 to 80° C.,
d) voltage in the range of 1 to 30V,
e) current density in the range of 10 to 500 mA/cm²; and
f) applied charge in the range of 1 to 10 Farad,
in particular a combination comprising at least features a), b), e) and f).

22. The process of claim 20, wherein the anodic oxidation is performed under at least one of the following conditions or a combination of all of these conditions:
current density j in the range of 50 to 100 mA/cm² in batch electrolysis; or current density j in the range of 400 to 500 mA/cm² in flow electrolysis (as for example observed at a flow rate of 7.5 L/h and 48 cm² anode surface area)
applied charge Q in the range of 3 to 4 F
initial concentration cₒ (NaIO₃) of about 0.21 M
initial concentration cₒ (NaOH) of about 1.0 M
ratio of cₒ (NaIO₃):c₀ (NaOH) of about 1:5

23. A process for the preparation of a lactam compound of the formula IIIa or IIIb 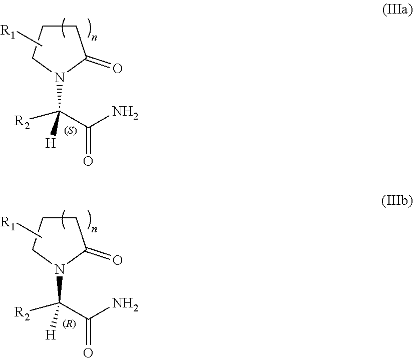
wherein
n is 0 or an integer of 1 to 4; and
R=₁ and R₂ independently of each other represent H or a straight-chain or branched, saturated or non-saturated hydrocarbon group having 1 to 6 carbon atoms, in particular C₁-C₆ or C₁-C₃ alkyl;
which process comprises
the regioselective chemical oxidation of an alpha-amino amide of the formula Ia or Ib 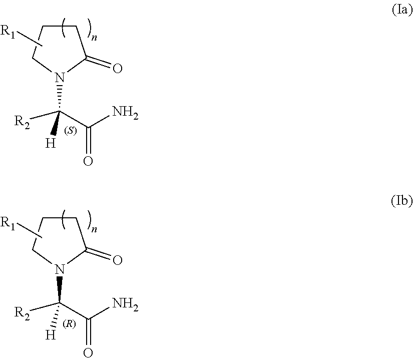
wherein n, R₁ and R₂ are as defined above;
to the corresponding lactam derivative of the general formula IIIa or IIIb.
wherein the reaction is performed as defined above in claim 14.

24. The process of claim 23, wherein the reaction is performed in the presence of an oxidation catalyst as defined in claim 15.


SEQUENCE LISTING (145 sequences)

SEQ ID NO: 1 - DNA, 657 bp, Comamonas testosteroni
  misc_feature: CtNHase beta-subunit_AAU87543.1
  CDS (1)..(657)
atgaatggcattcacgatactgggggagcacatggttatgggccggtttacagagaaccgaacgaacccgtctttcgctacgactgggaaaaaacggtcatgtccctgttcccggcgctgttcgccaacggcaacttcaacctcgatgagtttcgacacggcatcgagcgcatgaaccccatcgactacctgaagggaacctactacgaacactggatccattccatcgaaaccttgctggtcgaaaagggtgtgctcacggcaacggaactcgcgaccggcaaggcatctggcaagacagcgacaccggtgctgacgccggccatcgtggacggactgctcagcaccggggcttctgccgcccgggaggagggtgcgcgggcgcggttcgctgtgggggacaaggttcgcgtcctcaacaagaacccggtgggccatacccgcatgccgcgctacacgcggggcaaagtggggacagtggtcatcgaccatggtgtgttcgtgacgccggacaccgcggcacacggaaagggcgagcacccccagcacgtttacaccgtgagtttcacgtcggtcgaactgtgggggcaagacgcctcctcgccgaaggacacgattcgcgtcgacttgtgggatgactacctggagccagcgtga

SEQ ID NO: 2 - protein, 218 aa, Comamonas testosteroni
MNGIHDTGGAHGYGPVYREPNEPVFRYDWEKTVMSLFPALFANGNFNLDEFRHGIERMNPIDYLKGTYYEHWIHSIETLLVEKGVLTATELATGKASGKTATPVLTPAIVDGLLSTGASAAREEGARARFAVGDKVRVLNKNPVGHTRMPRYTRGKVGTVVIDHGVFVTPDTAAHGKGEHPQHVYTVSFTSVELWGQDASSPKDTIRVDLWDDYLEPA

SEQ ID NO: 3 - DNA, 654 bp, Klebsiella oxytoca
  misc_feature: KoNHase beta-subunit_OSY94201.1WP
  CDS (1)..(654)
atgaacgggatacatgatctgggggggatgcacggccttggcccgatccctaccgaggaaaacgagccctatttccatcatgagtgggaacgccgggtatttcctctgttcgcctcgttgttcgtcggcggacattttaacgtcgatgaatttcgccacgccatcgaacgtatggcgccgaccgaatatttgcagtcgagctactacgagcactggctgcatgcattcgaaacgctgctgctggcaaagggggcgatcaccgttgaagaactgtggggtggcgcgaagcctgccccttgcaagcctggcacacctgtgctgacgcaggagatggtgtcgatggtggtcagcaccggcgggtctgctcgggtcagtcacgatgttgcgccccgcttccgggtgggcgattgggtacgaacgaaaaatttcaacccgaccacccatacccgcctgccacgctacgcacgcgataaagtcggtcgcatagagatcgctcacggtgtgtttatcacgccagatactgcggcgcacgggctgggcgaacatccccagcatgtttacagcgtcagtttcaccgcgcaggcgctgtggggagagccgcgccctgacaaagtgttcatcgatctgtgggacgactatctggaggaagcataa

SEQ ID NO: 4 - protein, 217 aa, Klebsiella oxytoca
MNGIHDLGGMHGLGPIPTEENEPYFHHEWERRVFPLFASLFVGGHFNVDEFRHAIERMAPTEYLQSSYYEHWLHAFETLLLAKGAITVEELWGGAKPAPCKPGTPVLTQEMVSMVVSTGGSARVSHDVAPRFRVGDWVRTKNFNPTTHTRLPRYARDKVGRIEIAHGVFITPDTAAHGLGEHPQHVYSVSFTAQALWGEPRPDKVFIDLWDDYLEEA

SEQ ID NO: 5 - DNA, 684 bp, Nitriliruptor alkaliphilus
  misc_feature: NaNHase beta-subunit_WP_052668588.1
  CDS (1)..(684)
atgaacggcgtccatgatcttggcggcaccgacggcctcggcccggtgatcaccgaggagaacgagccggtctggcacagcgagtgggagaaggccgtcttcacgatgttccccaccaacttcgccaagggacatttcaacgtcgactcgttccgcttcgggatcgagaagatccatccggcggactacctgtcgtcccggtactacgagcactggctgcactccatcgagcatgcggtggtggatcagggcgtcgtcgacgccgaggagctggaggcgcgcactcgtcactacctcgagaaccccgacgcgccgttgccggaccgccaggaccctgatctggtgccactggtggagacgatctcgcggggtggtggctccgcacgtcgcgagagcgacaaggcgccgaggttcgaggtcggtgaccgagtccgcgtcaagcgcgacgaggtgccccacggccacacgcgccgcgcccgctacgtgcaggggcgcgaaggcgtgatcgtccaggcccacggggcgttcatctacccggacagcgcaggcaacggcgggccggaggaccccgagcacgtctacacgatcctgttcgaggcctcgcacctgtggggcgaacggacgggtgactccaacggaaccgtgaccttcgacgcgtgggagccctacctcgagctggtctga

SEQ ID NO: 6 - protein, 227 aa, Nitriliruptor alkaliphilus
MNGVHDLGGTDGLGPVITEENEPVWHSEWEKAVFTMFPTNFAKGHFNVDSFRFGIEKIHPADYLSSRYYEHWLHSIEHAVVDQGVVDAEELEARTRHYLENPDAPLPDRQDPDLVPLVETISRGGGSARRESDKAPRFEVGDRVRVKRDEVPHGHTRRARYVQGREGVIVQAHGAFIYPDSAGNGGPEDPEHVYTILFEASHLWGERTGDSNGTVTFDAWEPYLELV

SEQ ID NO: 7 - DNA, 654 bp, Gordonia hydrophobica
  misc_feature: GhNHase beta-subunit_WP_066163466.1
  CDS (1)..(654)
atgcacggaattcacgatctcggcggcgtccagaacttcgggccggttccgcatcgggtcaatgagtaccccgacggtccgttccagacggccgagtaccacgaggactgggagcctctggcctacggtctgctgttcgcctgcgccgacgccgatcagttcagcgtcgatcagctacggcactcgatcgagcgtatggagccgcgcgactacatgacgagcagctacttcgaacgcattctggtcggcaccgccactctgatggtggagaacggcatgctcacccaggaagagctcgaagacttggccggcggaccgtttccgctctcgagaccagttagctcggccgggcggccggcgaatccgaatctgcagcagttcgaggtcggcgaccgggtacgggtggcgaccgaacaggtacccggccacattcgggtgcccggatactgcttcggcaaggagggcgtgatcgaccaccgcaccgcacacgagtggcggttccccgacgcgatcggacacggccgcgacgacggtggttccgaacccacctaccacgtccgattcgatgccaccgacgtcttcggcaccgacaccgaggccgagtccatcgtcgtcgacctcttcggcggctacctggagccggtgccggcctga

SEQ ID NO: 8 - protein, 217 aa, Gordonia hydrophobica
MHGIHDLGGVQNFGPVPHRVNEYPDGPFQTAEYHEDWEPLAYGLLFACADADQFSVDQLRHSIERMEPRDYMTSSYFERILVGTATLMVENGMLTQEELEDLAGGPFPLSRPVSSAGRPANPNLQQFEVGDRVRVATEQVPGHIRVPGYCFGKEGVIDHRTAHEWRFPDAIGHGRDDGGSEPTYHVRFDATDVFGTDTEAESIVVDLFGGYLEPVPA

SEQ ID NO: 9 - DNA, 663 bp, Pseudomonas marginalis
  misc_feature: PmNHase beta-subunit_WP_074846644.1
  CDS (1)..(663)
atggatggctttcacgatctcggcggtttccaaggctttggaaaagtccctcacaccatcaacagcctgagctacaaacaggtgttcaagcaggactgggagcatctggcctacagcttgatgttcatcggtgccgaccacttgaaaaagttcagcgtggacgaagtgcgtcacgccgtcgaacgcctggatgtgcgccagcatgtcggcacccagtactacgaacgctacgtcatcgcgaccgccaccctgctggtcgaaaccggcgtgatcacccaggcggagcttgatcaggccttgggctcccacttcaagctggcgaatcccgcccatgccgagggccgcccggcgatcacggggcggccgcccttcgaggtgggggatcgggtggtggtgcgagacgaatatgtggctgggcacatccgcatgcccgcctacgtgcgcggcaaggaaggcgtggtcctgcaccgcacgtcagagaaatggccgttccccgacgcgattgggcatggcgatgtaagcgccgcccatcaacccacctaccacgtcgagttttccgtgaaagacctgtggggggatgcggccgatgagggttttgtggtggtcgacctgttcgaaagctacctggacaaggccgccggcgcgcacgcggtgaacccatga

SEQ ID NO: 10 - protein, 220 aa, Pseudomonas marginalis
MDGFHDLGGFQGFGKVPHTINSLSYKQVFKQDWEHLAYSLMFIGADHLKKFSVDEVRHAVERLDVRQHVGTQYYERYVIATATLLVETGVITQAELDQALGSHFKLANPAHAEGRPAITGRPPFEVGDRVVVRDEYVAGHIRMPAYVRGKEGVVLHRTSEKWPFPDAIGHGDVSAAHQPTYHVEFSVKDLWGDAADEGFVVVDLFESYLDKAAGAHAVNP

SEQ ID NO: 11 - DNA, 639 bp, Rhodococcus erythropolis
  misc_feature: ReHNase beta-subunit_P13449.1
  misc_feature: ReNHase beta-subunit_P13449.1
  CDS (1)..(639)
atggatggagtacacgatcttgccggagtacaaggcttcggcaaagtcccgcataccgtcaacgccgacatcggccccacctttcacgccgaatgggaacacctgccctacagcctgatgttcgccggtgtcgccgaactcggggccttcagcgtcgacgaagtgcgatacgtcgtcgagcggatggagccgcgccactacatgatgaccccgtactacgagaggtacgtcatcggtgtcgcgacattgatggtcgaaaagggaatcctgacgcaggacgaactcgaaagccttgcggggggaccgttcccactgtcacggcccagcgaatccgaagggcggccggcacccgtcgagacgaccaccttcgaagtcgggcagcgagtacgcgtacgcgacgagtacgttccggggcatattcgaatgcctgcatactgccgtggacgagtgggaaccatctctcatcgaactaccgagaagtggccgtttcccgacgcaatcggccacgggcgcaacgacgccggcgaagaaccgacgtaccacgtgaagttcgccgccgaggaattgttcggtagcgacaccgacggtggaagcgtcgttgtcgacctcttcgagggttacctcgagcctgcggcctga

SEQ ID NO: 12 - protein, 212 aa, Rhodococcus erythropolis
MDGVHDLAGVQGFGKVPHTVNADIGPTFHAEWEHLPYSLMFAGVAELGAFSVDEVRYVVERMEPRHYMMTPYYERYVIGVATLMVEKGILTQDELESLAGGPFPLSRPSESEGRPAPVETTTFEVGQRVRVRDEYVPGHIRMPAYCRGRVGTISHRTTEKWPFPDAIGHGRNDAGEEPTYHVKFAAEELFGSDTDGGSVVVDLFEGYLEPAA

SEQ ID NO: 13 - protein, 119 aa, Pseudomonas kilonensis
  misc_feature: PkNHase beta-subunit_partial sequence
  misc_feature (92)..(92): Xaa can be any naturally occurring amino acid
  misc_feature (107)..(107): Xaa can be any naturally occurring amino acid
  misc_feature (117)..(117): Xaa can be any naturally occurring amino acid
MDGFHDLGGFQGFGKVPHTINSLSYKQVFKQDWEHLAYSLMFVGVDQLKKFSVDEVRHAVERLDVRQHVGTQYYERYVIATATLLVETGVIXQAELDQALGSHFKLXKPCSCQRSPXDH

SEQ ID NO: 14 - DNA, 633 bp, Comamonas testosteroni
  misc_feature: CtNHase alpha-subunit_AAU87542.1
  CDS (1)..(633)
atggggcaatcacacacgcatgaccaccatcacgacgggtaccaggcaccgcccgaagacatcgcgctgcgggtcaaggccttggagtctctgctgatcgagaaaggtcttgtcgacccagcggccatggacttggtcgtccaaacgtatgaacacaaggtaggcccccgaaacggcgccaaagtcgtggccaaggcctgggtggaccctgcctacaaggcccgtctgctggcagacggcactgccggcattgccgagctgggcttctccggggtacagggcgaggacatggtcattctggaaaacacccccgccgtccacaacgtcgtcgtttgcaccttgtgctcttgctacccatggccgacgctgggcttgccccctgcctggtacaaggccccgccctaccggtcccgcatggtgagcgacccgcgtggggttctcgcggagttcggcctggtgatccccgcgaaggaaatccgcgtctgggacaccacggccgaattgcgctacatggtgctgccggaacggcccgcgggaactgaagcctacagcgaagaacaactggccgaactcgttacccgcgattcgatgatcggcaccggcctgcccatccaacccaccccatctcattaa

SEQ ID NO: 15 - protein, 210 aa, Comamonas testosteroni
MGQSHTHDHHHDGYQAPPEDIALRVKALESLLIEKGLVDPAAMDLVVQTYEHKVGPRNGAKVVAKAWVDPAYKARLLADGTAGIAELGFSGVQGEDMVILENTPAVHNVVVCTLCSCYPWPTLGLPPAWYKAPPYRSRMVSDPRGVLAEFGLVIPAKEIRVWDTTAELRYMVLPERPAGTEAYSEEQLAELVTRDSMIGTGLPIQPTPSH

SEQ ID NO: 16 - DNA, 609 bp, Klebsiella oxytoca
  misc_feature: KoNHase alpha-subunit_OSY94202.1
  CDS (1)..(609)
atgagccataaacacgaccacgaccatacccacccccccgttgatatcgagctacgcgtccgcgcactggaatccctgctgcaggaaaaaggcctgatcgacccggctgcgctggatgagctgattgacacctacgagcacaaagtcggcccccgaaacggcgcacaggttgtcgccagagcgtggagcgacccggaatacaaacgtcgactgatggaaaacgccactgccgctattgctgaactgggtttctccggaatacagggcgaagacatgctggtcgtggagaacacgccggacgtgcataacgtcaccgtttgtacgctgtgttcctgctacccctggccggtgctgggtctgccgccggtgtggtacaaatcagcgccctatcgttcgcgtatcgtcatcgacccgcgcggcgttctcgccgagttcgggttacacataccagaaaacaaagagattcgcgtctgggacagcagcgccgagctgcgctatctggtcctgcctgaacgtccggcaggcacggaaggctggagcgaagcgcagttgagcgaactcatcacgcgcgattcgatgattggcaccggtgtggttagcgcaccataa

SEQ ID NO: 17 - protein, 202 aa, Klebsiella oxytoca
MSHKHDHDHTHPPVDIELRVRALESLLQEKGLIDPAALDELIDTYEHKVGPRNGAQVVARAWSDPEYKRRLMENATAAIAELGFSGIQGEDMLVVENTPDVHNVTVCTLCSCYPWPVLGLPPVWYKSAPYRSRIVIDPRGVLAEFGLHIPENKEIRVWDSSAELRYLVLPERPAGTEGWSEAQLSELITRDSMIGTGVVSAP

SEQ ID NO: 18 - DNA, 636 bp, Nitriliruptor alkaliphilus
  misc_feature: NaNHase alpha-subunit_WP_052668589.1
  CDS (1)..(636)
atgagccagacccagagcccaccccccgtcaacttcagcctgccccgcacggaggagcaggtcgcggcgcgcgtgaaggcgctcgagtcgatcatgatcgagaagggcatcatgacgacggacgccgtcgaccgcctcgccgagatctacgagaacgaggtcggaccacagctcggcgcaagggtcgtggcccgcgcctggtctgaccccgacttccacgatcggctgctcgccaacgccaccgaggcctgcgaggagctggacatcggcggcctgcagggcgaggacatggtcgtcatcgagaacaccgacgacgtgcacaacctcatcgtgtgcacgctctgctcctgctacccctggccaacgctcggcctgccgcccaactggtacaagtaccccgcctaccgggccaaggccgtgcgcgaaccgcgggcgatgctgcgcgacgacttcggcctcgacctgcccgactcggttgagatccgcgtctgggataccagcgccgagcttcggtactgggtcctgcccaagcgcccggaaggtaccgagagcatgacggaggacgaattggccgagctggtcacccgcgactccatgatcggtgtcggcgtcgcccgtacccccgaggcggtctga

SEQ ID NO: 19 - protein, 211 aa, Nitriliruptor alkaliphilus
MSQTQSPPPVNFSLPRTEEQVAARVKALESIMIEKGIMTTDAVDRLAEIYENEVGPQLGARVVARAWSDPDFHDRLLANATEACEELDIGGLQGEDMVVIENTDDVHNLIVCTLCSCYPWPTLGLPPNWYKYPAYRAKAVREPRAMLRDDFGLDLPDSVEIRVWDTSAELRYWVLPKRPEGTESMTEDELAELVTRDSMIGVGVARTPEAV

SEQ ID NO: 20 - DNA, 624 bp, Gordonia hydrophobica
  misc_feature: GhNHase alpha-subunit_WP_066163464.1
  CDS (1)..(624)
atgtctgtcaccatcgatcacgcagtcgacaacgatctcggcgagaaggctccgcgctcggcgcgtggcgaagcgctccgtcgtgctctggaggccaagggcctgctgcccgagggctatctcgacgagtggaacaacaccgcagagaacgtcttcagtccgcggcgcggcgccgaactcgtcgcacacgcatggaccgacccggaattccgcgagctcttgctcactgacgggacggctgcggtgggccagtacggttacctcggcccgcagggcgagtacatcgtcgcgttggaggacacgccgacgctcaagcacgtcatcgtgtgctcgctctgctcgtgcacggcctggccgattctgggactctcgccgtcgtggtacaagagcttcgagtaccggtcccgggtggtgcgtgaaccgcgcacggtgctcgccgagatgggcacccagattccggacggcgtcgagatccgggtcgtcgatgtcaccgcggacacccgcttcatggttctcccgcagcggccggcgggcacagaggggtggagccgtgaacagctggccgagatcgtgaccaaggactgcttgatcggcgtggcggtcccacaggtcgaatccgtctga

SEQ ID NO: 21 - protein, 207 aa, Gordonia hydrophobica
MSVTIDHAVDNDLGEKAPRSARGEALRRALEAKGLLPEGYLDEWNNTAENVFSPRRGAELVAHAWTDPEFRELLLTDGTAAVGQYGYLGPQGEYIVALEDTPTLKHVIVCSLCSCTAWPILGLSPSWYKSFEYRSRVVREPRTVLAEMGTQIPDGVEIRVVDVTADTRFMVLPQRPAGTEGWSREQLAEIVTKDCLIGVAVPQVESV

SEQ ID NO: 22 - DNA, 585 bp, Pseudomonas marginalis
  misc_feature: PmNHase alpha-subunit_WP_074846646.1
  CDS (1)..(585)
atgaatacagcgacttcaacgcccggcgaaagagcctgggcattgtttcaagtcctcaagagcaaggaactcatcccggagggctatgtcgagcagctcacgcaattgatggagcacggctggagccccgagaacggcgcccgtgtggtggccaaggcatgggtcgatccgcagttccgggcactgttgctcaaggacggcaccgcggcctgcgcccagttcggctacaccggcccccagggcgaatacatcgtcgccctggaggatacgccgacgctgaagaacgtgatcgtctgcagcctgtgctcctgcaccaactggccggtcctcggcctgccgccggagtggtacaagggtttcgagttccgcgcacgcctggtccgggaggggcgcacggtactgcgcgagctggggacggagttgccccgggacatggtggtcaaggtctgggataccagcgccgaaagccgctacctggtgctgccggtcaggccggaaggctcagaacacatgagcgaagagcagcttcaagcgctggtgaccaaagacgtgctgatcggcgtcgccctgccccgcgtgggctga

SEQ ID NO: 23 - protein, 194 aa, Pseudomonas marginalis
MNTATSTPGERAWALFQVLKSKELIPEGYVEQLTQLMEHGWSPENGARVVAKAWVDPQFRALLLKDGTAACAQFGYTGPQGEYIVALEDTPTLKNVIVCSLCSCTNWPVLGLPPEWYKGFEFRARLVREGRTVLRELGTELPRDMVVKVWDTSAESRYLVLPVRPEGSEHMSEEQLQALVTKDVLIGVALPRVG

SEQ ID NO: 24 - DNA, 624 bp, Rhodococcus erythropolis
  misc_feature: ReNHase alpha-subunit_P13448.3
  CDS (1)..(624)
atgtcagtaacgatcgaccacacaacggagaacgccgcaccggcccaggcgccggtctccgaccgggcgtgggcactgttccgcgcactcgacggtaagggattggtacccgacggttacgtcgagggatggaagaagaccttcgaggaggacttcagtccaaggcgcggagcggaattggtagcgcgcgcatggaccgaccccgagttccggcagctgcttctcaccgacggtaccgccgcagttgcccagtacggatacctgggcccccagggcgaatacatcgtggcagtcgaagacaccccgacactcaagaacgtgatcgtgtgctcgctgtgttcatgcaccgcgtggcccatcctcggtctgccacccacctggtacaagagcttcgaataccgtgcgcgcgtggtccgcgaaccacggaaggttctctccgagatgggaaccgagatcgcgtcggacatcgagattcgcgtctacgacaccaccgccgaaactcgctacatggtcctcccgcagcgtcccgccggcaccgaaggctggagccaggaacaactgcaggaaatcgtcaccaaggactgcctgatcggggttgcaatcccgcaggttcccaccgtctga

SEQ ID NO: 25 - protein, 207 aa, Rhodococcus erythropolis
MSVTIDHTTENAAPAQAPVSDRAWALFRALDGKGLVPDGYVEGWKKTFEEDFSPRRGAELVARAWTDPEFRQLLLTDGTAAVAQYGYLGPQGEYIVAVEDTPTLKNVIVCSLCSCTAWPILGLPPTWYKSFEYRARVVREPRKVLSEMGTEIASDIEIRVYDTTAETRYMVLPQRPAGTEGWSQEQLQEIVTKDCLIGVAIPQVPTV

SEQ ID NO: 26 - DNA, 603 bp, Pseudomonas kilonensis
  misc_feature: PkNHase alpha-subunit
  CDS (1)..(603)
atgagtacatctatttccacgactgcgacgacttcaacgcccggagagcgggcacgggcactgtttcaggtgctcaagagcaaagacctcatcccgcagggctatgtcgagcaactgactgagttgatggaacacggctggagcccggagaacggcgcccgagtggtcgccaaagcatgggtcgatccgcagttccgggcactgttgctcaaggacggcaccgccgcgtgcgcccagttcggctacaccggtccgcagggcgagtacatcgtcgctctggaggatacgcccgaggtgaaaaacgtcattgtctgcagcctttgctcctgcaccaactggccggtcctcggcctgccacccgagtggtacaagggctttgagtttcgtgctcgcctggtccgggaaggacgcaccgtactgcgtgaactggggacagagttaccgaacgacagggttgtcaaggtctgggacaccagcgccgaaacccgctacctggtgttgccggtaagaccagaaggctgcgagcacatgaccgaagagcagcttcggacactggtgaccaaggacgtgctgattggcgtcgccctgccccaggttggctga

SEQ ID NO: 27 - protein, 200 aa, Pseudomonas kilonensis
MSTSISTTATTSTPGERARALFQVLKSKDLIPQGYVEQLTELMEHGWSPENGARVVAKAWVDPQFRALLLKDGTAACAQFGYTGPQGEYIVALEDTPEVKNVIVCSLCSCTNWPVLGLPPEWYKGFEFRARLVREGRTVLRELGTELPNDRVVKVWDTSAETRYLVLPVRPEGCEHMTEEQLRTLVTKDVLIGVALPQVG

SEQ ID NO: 28 - DNA, 20 bp, Artificial Sequence
  Ct-aQ93X_for
  misc_feature (6)..(8): N can be any nucleic acid (A, T, G, or C); K can be G or T
gggtannkggcgaggacatg

SEQ ID NO: 29 - DNA, 19 bp, Artificial Sequence
  Ct-aQ93X_rev
  misc_feature (4)..(6): N can be any nucleic acid (A, T, G, or C); M can be A or C
gccmnntaccccggagaag

SEQ ID NO: 30 - DNA, 19 bp, Artificial Sequence
  Ct-aW120X_for
  misc_feature (7)..(9): N can be any nucleic acid (A, T, G, or C); K can be G or T
tacccannkccgacgctgg

SEQ ID NO: 31 - DNA, 19 bp, Artificial Sequence
  Ct-aW120X_rev
  misc_feature (10)..(12): N can be any nucleic acid (A, T, G, or C); M can be A or C
cagcgtcggmnntgggtag

SEQ ID NO: 32 - DNA, 19 bp, Artificial Sequence
  Ct-aP126X_for
  misc_feature (9)..(11): N can be any nucleic acid (A, T, G, or C); K can be G or T
tgggcttgnnkcctgcctg

SEQ ID NO: 33 - DNA, 19 bp, Artificial Sequence
  Ct-aP126X_rev
  misc_feature (14)..(16): N can be any nucleic acid (A, T, G, or C); M can be A or C
gtaccaggcaggmnncaag

SEQ ID NO: 34 - DNA, 19 bp, Artificial Sequence
  Ct-aK131X_for
  misc_feature (5)..(7): N can be any nucleic acid (A, T, G, or C); K can be G or T
gtacnnkgccccgccctac

SEQ ID NO: 35 - DNA, 18 bp, Artificial Sequence
  Ct-aK131X_rev
  misc_feature (5)..(7): N can be any nucleic acid (A, T, G, or C); M can be A or C
gggcmnngtaccaggcag

SEQ ID NO: 36 - DNA, 22 bp, Artificial Sequence
  Ct-aR169X_for
  misc_feature (8)..(10): N can be any nucleic acid (A, T, G, or C); K can be G or T
cgaattgnnktacatggtgctg

SEQ ID NO: 37 - DNA, 22 bp, Artificial Sequence
  Ct-aR169X_rev
  misc_feature (14)..(16): N can be any nucleic acid (A, T, G, or C); M can be A or C
cagcaccatgtamnncaattcg

SEQ ID NO: 38 - DNA, 19 bp, Artificial Sequence
  Ct-bM34X_for
  misc_feature (6)..(8): N can be any nucleic acid (A, T, G, or C); K can be G or T
cggtcnnktccctgttccc

SEQ ID NO: 39 - DNA, 21 bp, Artificial Sequence
  Ct-bM34X_rev
  misc_feature (7)..(9): N can be any nucleic acid (A, T, G, or C); M can be A or C
cagggamnngaccgttttttc

SEQ ID NO: 40 - DNA, 20 bp, Artificial Sequence
  Ct-bF37X_for
  misc_feature (6)..(8): N can be any nucleic acid (A, T, G, or C); K can be G or T
ccctgnnkccggcgctgttc

SEQ ID NO: 41 - DNA, 20 bp, Artificial Sequence
  Ct-bF37X_rev
  misc_feature (6)..(8): N can be any nucleic acid (A, T, G, or C); M can be A or C
gccggmnncagggacatgac

SEQ ID NO: 42 - DNA, 22 bp, Artificial Sequence
  Ct-bL48X_for
  misc_feature (5)..(7): N can be any nucleic acid (A, T, G, or C); K can be G or T
caacnnkgatgagtttcgacac

SEQ ID NO: 43 - DNA, 23 bp, Artificial Sequence
  Ct-bL48X_rev
  misc_feature (15)..(17): N can be any nucleic acid (A, T, G, or C); M can be A or C
gtcgaaactcatcmnngttgaag

SEQ ID NO: 44 - DNA, 19 bp, Artificial Sequence
  Ct-bF51X_for
  misc_feature (8)..(10): N can be any nucleic acid (A, T, G, or C); K can be G or T
cgatgagnnkcgacacggc

SEQ ID NO: 45 - DNA, 19 bp, Artificial Sequence
  Ct-bF51X_rev
  misc_feature (10)..(12): N can be any nucleic acid (A, T, G, or C); M can be A or C
gccgtgtcgmnnctcatcg

SEQ ID NO: 46 - DNA, 32 bp, Artificial Sequence
  Ct-bY68X-long_for
  misc_feature (10)..(12): N can be any nucleic acid (A, T, G, or C); K can be G or T
aagggaaccnnktacgaacactggatccattc

SEQ ID NO: 47 - DNA, 30 bp, Artificial Sequence
  Ct-bY68X-long_rev
  misc_feature (9)..(11): N can be any nucleic acid (A, T, G, or C); M can be A or C
tgttcgtamnnggttcccttcaggtagtcg

SEQ ID NO: 48 - DNA, 21 bp, Artificial Sequence
  Primer Ct-aW120F_for
gctacccatttccgacgctgg

SEQ ID NO: 49 - DNA, 23 bp, Artificial Sequence
  Primer Ct-aW120F_rev
gcgtcggaaatgggtagcaagag

SEQ ID NO: 50 - DNA, 32 bp, Artificial Sequence
  Primer Ct-bM34L_for
aaacggtcttgtccctgttcccggcgctgttc

SEQ ID NO: 51 - DNA, 33 bp, Artificial Sequence
  Primer Ct-bM34L_rev
aacagggacaagaccgttttttcccagtcgtag

SEQ ID NO: 52 - DNA, 32 bp, Artificial Sequence
  Primer Ct-bM34Q_for
aaacggtccagtccctgttcccggcgctgttc

SEQ ID NO: 53 - DNA, 33 bp, Artificial Sequence
  Primer Ct-bM34Q_rev
aacagggactggaccgttttttcccagtcgtag

SEQ ID NO: 54 - DNA, 20 bp, Artificial Sequence
  Ct-alpha1_for
tggccaaggcctgggtggac

SEQ ID NO: 55 - DNA, 21 bp, Artificial Sequence
  Ct-alpha1_rev
gcaagagcacaaggtgcaaac

SEQ ID NO: 56 - DNA, 20 bp, Artificial Sequence
  Ct-alpha2_for
ccttgtgctcttgctaccca

SEQ ID NO: 57 - DNA, 18 bp, Artificial Sequence
  Ct-alpha2_rev
ttcagttcccgcgggccg

SEQ ID NO: 58 - DNA, 19 bp, Artificial Sequence
  Ct-beta1_for
cgtctttcgctacgactgg

SEQ ID NO: 59 - DNA, 22 bp, Artificial Sequence
  Ct-beta1_rev
aggtttcgatggaatggatcca

SEQ ID NO: 60 - DNA, 18 bp, Artificial Sequence
  Ct-beta2_for
gcttctgccgcccgggag

SEQ ID NO: 61 - DNA, 20 bp, Artificial Sequence
  Ct-beta2_rev
tttccgtgtgccgcggtgtc

SEQ ID NO: 62 - DNA, 34 bp, Artificial Sequence
  Ct-A1bb-lig_for
  misc_feature (6)..(11): restriction site
aatttctcgagtttgcaccttgtgctcttgctac

SEQ ID NO: 63 - DNA, 29 bp, Artificial Sequence
  Ct-A1bb-lig_rev
  misc_feature (6)..(11): restriction site
aatttctcgagtccacccaggccttggcc

SEQ ID NO: 64 - DNA, 28 bp, Artificial Sequence
  Ct-A2bb-lig_for
  misc_feature (6)..(11): restriction site
aatttctcgaggcccgcgggaactgaag

SEQ ID NO: 65 - DNA, 29 bp, Artificial Sequence
  Ct-A2bb-lig_rev
  misc_feature (6)..(11): restriction site
aatttctcgagggtagcaagagcacaagg

SEQ ID NO: 66 - DNA, 36 bp, Artificial Sequence
  Ct-B1bb-lig_for
  misc_feature (7)..(12): restriction site
tttaaagctagctggatccattccatcgaaaccttg

SEQ ID NO: 67 - DNA, 30 bp, Artificial Sequence
  Ct-B1bb-lig_rev
  misc_feature (7)..(12): restriction site
tttaaagctagccagtcgtagcgaaagacg

SEQ ID NO: 68 - DNA, 31 bp, Artificial Sequence
  Ct-B2bb-lig_for
  misc_feature (7)..(12): restriction site
tttaaagctagcaccgcggcacacggaaagg

SEQ ID NO: 69 - DNA, 30 bp, Artificial Sequence
  Ct-B2bb-lig_rev
  misc_feature (7)..(12): restriction site
tttaaagctagctcccgggcggcagaagcc

SEQ ID NO: 70 - DNA, 44 bp, Artificial Sequence
  Ct-beta1-focused_for
  misc_feature (9)..(9): Y can be T or C
  misc_feature (10)..(10): K can be G or T
  misc_feature (11)..(11): S can be C or G
  misc_feature (19)..(19): V can be A, G or C
  misc_feature (27)..(27): n is a, c, g, or t
  misc_feature (29)..(29): N can be A, G, T or C
  misc_feature (30)..(30): D can be A, G or T
acttcaacyksgatgagvttcgacacndtatcgagcgcatgaac

SEQ ID NO: 71 - DNA, 50 bp, Artificial Sequence
  Ct-beta1-focused_rev
  misc_feature (15)..(15): H can be A, C or T
  misc_feature (16)..(16): N can be A, G, T or C
  misc_feature (26)..(26): B can be G, C or T
  misc_feature (34)..(34): S can be C or G
  misc_feature (35)..(35): M can be A or C
  misc_feature (36)..(36): R can be G or A
catgcgctcgatahngtgtcgaabctcatcsmrgttgaagttgccgttgg

SEQ ID NO: 72 - DNA, 20 bp, Artificial Sequence
  Primer Ct-P121A_for
atgggcgacgctgggcttgc

SEQ ID NO: 73 - DNA, 24 bp, Artificial Sequence
  Primer Ct-P121A_rev
agcgtcgcccatgggtagcaagag

SEQ ID NO: 74 - DNA, 20 bp, Artificial Sequence
  Primer Ct-P121R_for
atggcgtacgctgggcttgc

SEQ ID NO: 75 - DNA, 24 bp, Artificial Sequence
  Primer Ct-P121R_rev
agcgtacgccatgggtagcaagag

SEQ ID NO: 76 - DNA, 20 bp, Artificial Sequence
  Primer Ct-P121N_for
atggaacacgctgggcttgc

SEQ ID NO: 77 - DNA, 24 bp, Artificial Sequence
  Primer Ct-P121N_rev
agcgtgttccatgggtagcaagag

SEQ ID NO: 78 - DNA, 20 bp, Artificial Sequence
  Primer Ct-P121D_for
atgggatacgctgggcttgc

SEQ ID NO: 79 - DNA, 24 bp, Artificial Sequence
  Primer Ct-P121D_rev
agcgtatcccatgggtagcaagag

SEQ ID NO: 80 - DNA, 20 bp, Artificial Sequence
  Primer Ct-P121C_for
atggtgcacgctgggcttgc

SEQ ID NO: 81 - DNA, 24 bp, Artificial Sequence
  Primer Ct-P121C_rev
agcgtgcaccatgggtagcaagag

SEQ ID NO: 82 - DNA, 20 bp, Artificial Sequence
  Primer Ct-P121Q_for
atggcagacgctgggcttgc

SEQ ID NO: 83 - DNA, 24 bp, Artificial Sequence
  Primer Ct-P121Q_rev
agcgtctgccatgggtagcaagag

SEQ ID NO: 84 - DNA, 20 bp, Artificial Sequence
  Primer Ct-P121E_for
atgggaaacgctgggcttgc

SEQ ID NO: 85 - DNA, 24 bp, Artificial Sequence
  Primer Ct-P121E_rev
agcgtttcccatgggtagcaagag

SEQ ID NO: 86 - DNA, 20 bp, Artificial Sequence
  Primer Ct-P121G_for
atggggcacgctgggcttgc

SEQ ID NO: 87 - DNA, 24 bp, Artificial Sequence
  Primer Ct-P121G_rev
agcgtgccccatgggtagcaagag

SEQ ID NO: 88 - DNA, 20 bp, Artificial Sequence
  Primer Ct-P121H_for
atggcatacgctgggcttgc

SEQ ID NO: 89 - DNA, 24 bp, Artificial Sequence
  Primer Ct-P121H_rev
agcgtatgccatgggtagcaagag

SEQ ID NO: 90 - DNA, 20 bp, Artificial Sequence
  Primer Ct-P121I_for
atggattacgctgggcttgc

SEQ ID NO: 91 - DNA, 24 bp, Artificial Sequence
  Primer Ct-P121I_rev
agcgtaatccatgggtagcaagag

SEQ ID NO: 92 - DNA, 20 bp, Artificial Sequence
  Primer Ct-P121L_for
atggctgacgctgggcttgc

SEQ ID NO: 93 - DNA, 24 bp, Artificial Sequence
  Primer Ct-P121L_rev
agcgtcagccatgggtagcaagag

SEQ ID NO: 94 - DNA, 20 bp, Artificial Sequence
  Primer Ct-P121K_for
atggaaaacgctgggcttgc

SEQ ID NO: 95 - DNA, 24 bp, Artificial Sequence
  Primer Ct-P121K_rev
agcgttttccatgggtagcaagag

SEQ ID NO: 96 - DNA, 20 bp, Artificial Sequence
  Primer Ct-P121M_for
atggatgacgctgggcttgc

SEQ ID NO: 97 - DNA, 24 bp, Artificial Sequence
  Primer Ct-P121M_rev
agcgtcatccatgggtagcaagag

SEQ ID NO: 98 - DNA, 20 bp, Artificial Sequence
  Primer Ct-P121F_for
atggtttacgctgggcttgc

SEQ ID NO: 99 - DNA, 24 bp, Artificial Sequence
  Primer Ct-P121F_rev
agcgtaaaccatgggtagcaagag

SEQ ID NO: 100 - DNA, 20 bp, Artificial Sequence
  Primer Ct-P121W_for
atggtggacgctgggcttgc

SEQ ID NO: 101 - DNA, 24 bp, Artificial Sequence
  Primer Ct-P121W_rev
agcgtccaccatgggtagcaagag

SEQ ID NO: 102 - DNA, 20 bp, Artificial Sequence
  Primer Ct-P121Y_for
atggtatacgctgggcttgc

SEQ ID NO: 103 - DNA, 24 bp, Artificial Sequence
  Primer Ct-P121Y_rev
agcgtataccatgggtagcaagag

SEQ ID NO: 104 - DNA, 20 bp, Artificial Sequence
  Ct-P121T_for
atggacgacgctgggcttgc

SEQ ID NO: 105 - DNA, 24 bp, Artificial Sequence
  Ct-P121T_rev
agcgtcgtccatgggtagcaagag

SEQ ID NO: 106 - DNA, 29 bp, Artificial Sequence
  Ct-V110I_for
aacgtcatcgtttgcaccttgtgctcttg

SEQ ID NO: 107 - DNA, 22 bp, Artificial Sequence
  Ct-V110I_rev
aaacgatgacgttgtggacggc

SEQ ID NO: 108 - DNA, 44 bp, Artificial Sequence
  Ct-L48R-G54C_for
acttcaaccgtgatgagtttcgacactgtatcgagcgcatgaac

SEQ ID NO: 109 - DNA, 50 bp, Artificial Sequence
  Ct-L48R-G54C_rev
catgcgctcgatacagtgtcgaaactcatcacggttgaagttgccgttgg

SEQ ID NO: 110 - DNA, 44 bp, Artificial Sequence
  Ct-L48R-G54R_for
acttcaaccgtgatgagtttcgacaccgtatcgagcgcatgaac

SEQ ID NO: 111 - DNA, 50 bp, Artificial Sequence
  Ct-L48R-G54R_rev
catgcgctcgatacggtgtcgaaactcatcacggttgaagttgccgttgg

SEQ ID NO: 112 - DNA, 44 bp, Artificial Sequence
  Ct-L48R-G54V_for
acttcaaccgtgatgagtttcgacacgttatcgagcgcatgaac

SEQ ID NO: 113 - DNA, 50 bp, Artificial Sequence
  Ct-L48R-G54V_rev
catgcgctcgataacgtgtcgaaactcatcacggttgaagttgccgttgg

SEQ ID NO: 114 - DNA, 44 bp, Artificial Sequence
  Ct-L48P-G54C_for
acttcaaccctgatgagtttcgacactgtatcgagcgcatgaac

SEQ ID NO: 115 - DNA, 50 bp, Artificial Sequence
  Ct-L48P-G54C_rev
catgcgctcgatacagtgtcgaaactcatcagggttgaagttgccgttgg

SEQ ID NO: 116 - DNA, 44 bp, Artificial Sequence
  Ct-L48P-G54R_for
acttcaaccctgatgagtttcgacaccgtatcgagcgcatgaac

SEQ ID NO: 117 - DNA, 50 bp, Artificial Sequence
  Ct-L48P-G54R_rev
catgcgctcgatacggtgtcgaaactcatcagggttgaagttgccgttgg

SEQ ID NO: 118 - DNA, 44 bp, Artificial Sequence
  Ct-L48P-G54V_for
acttcaaccctgatgagtttcgacacgttatcgagcgcatgaac

SEQ ID NO: 119 - DNA, 50 bp, Artificial Sequence
  Ct-L48P-G54V_rev
catgcgctcgataacgtgtcgaaactcatcagggttgaagttgccgttgg

SEQ ID NO: 120 - DNA, 44 bp, Artificial Sequence
  Ct-L48F-G54C_for
acttcaacttcgatgagtttcgacactgtatcgagcgcatgaac

SEQ ID NO: 121 - DNA, 50 bp, Artificial Sequence
  Ct-L48F-G54C_rev
catgcgctcgatacagtgtcgaaactcatcgaagttgaagttgccgttgg

SEQ ID NO: 122 - DNA, 44 bp, Artificial Sequence
  Ct-L48F-G54R_for
acttcaacttcgatgagtttcgacaccgtatcgagcgcatgaac

SEQ ID NO: 123 - DNA, 50 bp, Artificial Sequence
  Ct-L48F-G54R_rev
catgcgctcgatacggtgtcgaaactcatcgaagttgaagttgccgttgg

SEQ ID NO: 124 - DNA, 44 bp, Artificial Sequence
  Ct-L48F-G54V_for
acttcaacttcgatgagtttcgacacgttatcgagcgcatgaac

SEQ ID NO: 125 - DNA, 50 bp, Artificial Sequence
  Ct-L48F-G54V_rev
catgcgctcgataacgtgtcgaaactcatcgaagttgaagttgccgttgg

SEQ ID NO: 126 - DNA, 44 bp, Artificial Sequence
  Ct-L48P-F51V-G54V_for
acttcaaccctgatgaggttcgacacgttatcgagcgcatgaac

SEQ ID NO: 127 - DNA, 50 bp, Artificial Sequence
  Ct-L48P-F51V-G54V_rev
catgcgctcgataacgtgtcgaacctcatcagggttgaagttgccgttgg

SEQ ID NO: 128 - DNA, 22 bp, Artificial Sequence
  Ct-L48F_for
caactttgatgagtttcgacac

SEQ ID NO: 129 - DNA, 23 bp, Artificial Sequence
  Ct-L48F_rev
gtcgaaactcatcaaagttgaag

SEQ ID NO: 130 - DNA, 19 bp, Artificial Sequence
  Ct-F51L_for
cgatgagcttcgacacggc

SEQ ID NO: 131 - DNA, 19 bp, Artificial Sequence
  Ct-F51L_rev
gccgtgtcgaagctcatcg

SEQ ID NO: 132 - DNA, 1638 bp, Artificial Sequence
  CtNHase_Insert for vector cloning
  misc_feature (1)..(36): Overhang for Gibson cloning
  misc_feature (37)..(669): alpha-subunit
  misc_feature (680)..(1336): beta-subunit
  misc_feature (1389)..(1603): accessory protein
  misc_feature (1604)..(1638): Overhang for Gibson cloning
aataattttgtttaactttaagaaggagatatacatatggggcaatcacacacgcatgaccaccatcacgacgggtaccaggcaccgcccgaagacatcgcgctgcgggtcaaggccttggagtctctgctgatcgagaaaggtcttgtcgacccagcggccatggacttggtcgtccaaacgtatgaacacaaggtaggcccccgaaacggcgccaaagtcgtggccaaggcctgggtggaccctgcctacaaggcccgtctgctggcagacggcactgccggcattgccgagctgggcttctccggggtacagggcgaggacatggtcattctggaaaacacccccgccgtccacaacgtcgtcgtttgcaccttgtgctcttgctacccatggccgacgctgggcttgccccctgcctggtacaaggccccgccctaccggtcccgcatggtgagcgacccgcgtggggttctcgcggagttcggcctggtgatccccgcgaaggaaatccgcgtctgggacaccacggccgaattgcgctacatggtgctgccggaacggcccgcgggaactgaagcctacagcgaagaacaactggccgaactcgttacccgcgattcgatgatcggcaccggcctgcccatccaacccaccccatctcattaaggagttcgtcatgaatggcattcacgatactgggggagcacatggttatgggccggtttacagagaaccgaacgaacccgtctttcgctacgactgggaaaaaacggtcatgtccctgttcccggcgctgttcgccaacggcaacttcaacctcgatgagtttcgacacggcatcgagcgcatgaaccccatcgactacctgaagggaacctactacgaacactggatccattccatcgaaaccttgctggtcgaaaagggtgtgctcacggcaacggaactcgcgaccggcaaggcatctggcaagacagcgacaccggtgctgacgccggccatcgtggacggactgctcagcaccggggcttctgccgcccgggaggagggtgcgcgggcgcggttcgctgtgggggacaaggttcgcgtcctcaacaagaacccggtgggccatacccgcatgccgcgctacacgcggggcaaagtggggacagtggtcatcgaccatggtgtgttcgtgacgccggacaccgcggcacacggaaagggcgagcacccccagcacgtttacaccgtgagtttcacgtcggtcgaactgtgggggcaagacgcctcctcgccgaaggacacgattcgcgtcgacttgtgggatgactacctggagccagcgtgatcatgaaagacgaacggtttccattgccagagggttcgctgaaggacctcgatggccctgtgtttgacgagccttggcagtcccaggcgtttgccttggtggtcagcatgcacaaggccggtctctttcagtggaaagactgggccgagaccttcaccgccgaaatcgacgcttccccggctctgcccggcgaaagcgtcaacgacacctactaccggcaatgggtgtcggcgctggaaaagttggtggcgtcgctggggcttgtaagcttggctgttttggcggatgagagaagattttca

SEQ ID NO: 133 - DNA, 1834 bp, Artificial Sequence
  KoNHase_Insert for vector cloning
  misc_feature (1)..(36): Overhang for Gibson cloning
  misc_feature (37)..(645): alpha-subunit
  misc_feature (661)..(1314): beta-subunit
  misc_feature (1329)..(1799): Accessory protein
  misc_feature (1800)..(1834): Overhang for Gibson cloning
aataattttgtttaactttaagaaggagatatacatatgagccataaacacgaccacgaccatacccacccccccgttgatatcgagctacgcgtccgcgcactggaatccctgctgcaggaaaaaggcctgatcgacccggctgcgctggatgagctgattgacacctacgagcacaaagtcggcccccgaaacggcgcacaggttgtcgccagagcgtggagcgacccggaatacaaacgtcgactgatggaaaacgccactgccgctattgctgaactgggtttctccggaatacagggcgaagacatgctggtcgtggagaacacgccggacgtgcataacgtcaccgtttgtacgctgtgttcctgctacccctggccggtgctgggtctgccgccggtgtggtacaaatcagcgccctatcgttcgcgtatcgtcatcgacccgcgcggcgttctcgccgagttcgggttacacataccagaaaacaaagagattcgcgtctgggacagcagcgccgagctgcgctatctggtcctgcctgaacgtccggcaggcacggaaggctggagcgaagcgcagttgagcgaactcatcacgcgcgattcgatgattggcaccggtgtggttagcgcaccataacgaaaggagaaaaccatgaacgggatacatgatctgggggggatgcacggccttggcccgatccctaccgaggaaaacgagccctatttccatcatgagtgggaacgccgggtatttcctctgttcgcctcgttgttcgtcggcggacattttaacgtcgatgaatttcgccacgccatcgaacgtatggcgccgaccgaatatttgcagtcgagctactacgagcactggctgcatgcattcgaaacgctgctgctggcaaagggggcgatcaccgttgaagaactgtggggtggcgcgaagcctgccccttgcaagcctggcacacctgtgctgacgcaggagatggtgtcgatggtggtcagcaccggcgggtctgctcgggtcagtcacgatgttgcgccccgcttccgggtgggcgattgggtacgaacgaaaaatttcaacccgaccacccatacccgcctgccacgctacgcacgcgataaagtcggtcgcatagagatcgctcacggtgtgtttatcacgccagatactgcggcgcacgggctgggcgaacatccccagcatgtttacagcgtcagtttcaccgcgcaggcgctgtggggagagccgcgccctgacaaagtgttcatcgatctgtgggacgactatctggaggaagcataaaaggagatatacatatgaataatacggtagcacaacacgattacgccgccctcgggttaccgcgcgatgaggaagggccggtgttcgataagccctggcaggcaaaagcgttctccctgatagtccatctccaccgggccgggctgttcccgtgggcagaatgggtacagacattcagtaaagagatcaacgcggcgccggcgcaaccgggtgaaagcgcgaatgatgcctactatcgtcagtggacggcggcgatggaaaacatgatgacggcactcaacctgacggtgccggatgaaatcaaccgacggacgcaggagtggcgcaaggcgtatctcaacacgccccacggccagccgattgtactggcgaacgccagttgcccgccggcacatagccatcatcacctttcgccgggcgtgccggtcacggtgagtccggcactctctatcaacagcaaaatcgataatggagttacaccataagcttggctgttttggcggatgagagaagattttca

SEQ ID NO: 134 - DNA, 1855 bp, Artificial Sequence
  NaNHase_Insert for vector cloning
  misc_feature (1)..(36): Overhang for Gibson cloning
  misc_feature (37)..(720): alpha-subunit
  misc_feature (791)..(1426): beta-subunit
  misc_feature (1428)..(1820): accessory protein
  misc_feature (1821)..(1855): Overhang for Gibson cloning
aataattttgtttaactttaagaaggagatatacatatgaacggcgtccatgatcttggcggcaccgacggcctcggcccggtgatcaccgaggagaacgagccggtctggcacagcgagtgggagaaggccgtcttcacgatgttccccaccaacttcgccaagggacatttcaacgtcgactcgttccgcttcgggatcgagaagatccatccggcggactacctgtcgtcccggtactacgagcactggctgcactccatcgagcatgcggtggtggatcagggcgtcgtcgacgccgaggagctggaggcgcgcactcgtcactacctcgagaaccccgacgcgccgttgccggaccgccaggaccctgatctggtgccactggtggagacgatctcgcggggtggtggctccgcacgtcgcgagagcgacaaggcgccgaggttcgaggtcggtgaccgagtccgcgtcaagcgcgacgaggtgccccacggccacacgcgccgcgcccgctacgtgcaggggcgcgaaggcgtgatcgtccaggcccacggggcgttcatctacccggacagcgcaggcaacggcgggccggaggaccccgagcacgtctacacgatcctgttcgaggcctcgcacctgtggggcgaacggacgggtgactccaacggaaccgtgaccttcgacgcgtgggagccctacctcgagctggtctgacggccagcgtcgtgccgtgtcgcgcactcagccacaacggccagcgatcacacatcagggagaagaaccgatgagccagacccagagcccaccccccgtcaacttcagcctgccccgcacggaggagcaggtcgcggcgcgcgtgaaggcgctcgagtcgatcatgatcgagaagggcatcatgacgacggacgccgtcgaccgcctcgccgagatctacgagaacgaggtcggaccacagctcggcgcaagggtcgtggcccgcgcctggtctgaccccgacttccacgatcggctgctcgccaacgccaccgaggcctgcgaggagctggacatcggcggcctgcagggcgaggacatggtcgtcatcgagaacaccgacgacgtgcacaacctcatcgtgtgcacgctctgctcctgctacccctggccaacgctcggcctgccgcccaactggtacaagtaccccgcctaccgggccaaggccgtgcgcgaaccgcgggcgatgctgcgcgacgacttcggcctcgacctgcccgactcggttgagatccgcgtctgggataccagcgccgagcttcggtactgggtcctgcccaagcgcccggaaggtaccgagagcatgacggaggacgaattggccgagctggtcacccgcgactccatgatcggtgtcggcgtcgcccgtacccccgaggcggtctgacatgccgacagccacgacccccgtcggggatgccctgcggcacgtgcaggacctcctcgagcagctgcccgagcgcgacaagtccttcgacaagccatgggagcttcgagccttcgccctggctgtcgcggcacacgacaacggccagtacgactggagcgagttccagcgctcgctcatcagctccatcaaggcgtgggagcagggtggatcgccagtgccgtggcagtactacgaccactggctgcaggccctcgagaacgtgctggccgagaccggcgccctgaccgccgatgagctcgacgcccgcatgcacaccgtgctctgtacgcccgtcaacagagaccaccacgaggcgcgccacgacccagtggccatcgaccccgcgcgctaagcttggctgttttggcggatgagagaagattttca

SEQ ID NO: 135 - protein, 521 aa, Rhodococcus erythropolis
MATIRPDDKAIDAAARHYGITLDKTARLEWPALIDGALGSYDVVDQLYADEATPPTTSREHAVPSASENPLSAWYVTTSIPPTSDGVLTGRRVAIKDNVTVAGVPMMNGSRTVEGFTPSRDATVVTRLLAAGATVAGKAVCEDLCFSGSSFTPASGPVRNPWDRQREAGGSSGGSAALVANGDVDFAIGGDQGGSIRIPAAFCGVVGHKPTFGLVPYTGAFPIERTIDHLGPITRTVHDAALMLSVIAGRDGNDPRQADSVEAGDYLSTLDSDVDGLRIGIVREGFGHAVSQPEVDDAVRAAAHSLTEIGCTVEEVNIPWHLHAFHIWNVIATDGGAYQMLDGNGYGMNAEGLYDPELMAHFASRRIQHADALSETVKLVALTGHHGITTLGGASYGKARNLVPLARAAYDTALRQFDVLVMPTLPYVASELPAKDVDRATFITKALGMIANTAPFDVTGHPSLSVPAGLVNGLPVGMMITGRHFDDATVLRVGRAFEKLRGAFPTPAERASNSAPQLSPA

SEQ ID NO: 136 - DNA, 216 bp, Comamonas testosteroni
  CDS (1)..(216)
atggccctgtgtttgacgagccttggcagtcccaggcgtttgccttggtggtcagcatgcacaaggccggtctctttcagtggaaagactgggccgagaccttcaccgccgaaatcgacgcttccccggctctgcccggcgaaagcgtcaacgacacctactaccggcaatgggtgtcggcgctggaaaagttggtggcgtcgctggggcttgtaa

SEQ ID NO: 137 - protein, 71 aa, Comamonas testosteroni
MALCLTSLGSPRRLPWWSACTRPVSFSGKTGPRPSPPKSTLPRLCPAKASTTPTTGNGCRRWKSWWRRWGL

SEQ ID NO: 138 - DNA, 471 bp, Klebsiella oxytoca
  CDS (1)..(471)
atgaataatacggtagcacaacacgattacgccgccctcgggttaccgcgcgatgaggaagggccggtgttcgataagccctggcaggcaaaagcgttctccctgatagtccatctccaccgggccgggctgttcccgtgggcagaatgggtacagacattcagtaaagagatcaacgcggcgccggcgcaaccgggtgaaagcgcgaatgatgcctactatcgtcagtggacggcggcgatggaaaacatgatgacggcactcaacctgacggtgccggatgaaatcaaccgacggacgcaggagtggcgcaaggcgtatctcaacacgccccacggccagccgattgtactggcgaacgccagttgcccgccggcacatagccatcatcacctttcgccgggcgtgccggtcacggtgagtccggcactctctatcaacagcaaaatcgataatggagttacaccataa

SEQ ID NO: 139 - protein, 156 aa, Klebsiella oxytoca
MNNTVAQHDYAALGLPRDEEGPVFDKPWQAKAFSLIVHLHRAGLFPWAEWVQTFSKEINAAPAQPGESANDAYYRQWTAAMENMMTALNLTVPDEINRRTQEWRKAYLNTPHGQPIVLANASCPPAHSHHHLSPGVPVTVSPALSINSKIDNGVTP

SEQ ID NO: 140 - DNA, 393 bp, Nitriliruptor alkaliphilus
  CDS (1)..(393)
atgccgacagccacgacccccgtcggggatgccctgcggcacgtgcaggacctcctcgagcagctgcccgagcgcgacaagtccttcgacaagccatgggagcttcgagccttcgccctggctgtcgcggcacacgacaacggccagtacgactggagcgagttccagcgctcgctcatcagctccatcaaggcgtgggagcagggtggatcgccagtgccgtggcagtactacgaccactggctgcaggccctcgagaacgtgctggccgagaccggcgccctgaccgccgatgagctcgacgcccgcatgcacaccgtgctctgtacgcccgtcaacagagaccaccacgaggcgcgccacgacccagtggccatcgaccccgcgcgctaa

SEQ ID NO: 141 - protein, 130 aa, Nitriliruptor alkaliphilus
MPTATTPVGDALRHVQDLLEQLPERDKSFDKPWELRAFALAVAAHDNGQYDWSEFQRSLISSIKAWEQGGSPVPWQYYDHWLQALENVLAETGALTADELDARMHTVLCTPVNRDHHEARHDPVAIDPAR

SEQ ID NO: 142 - DNA, 1218 bp, Gordona hydrophobica
  CDS (1)..(1218)
atgaccgacaaccggcttcccgtcaccgtgctctccggcttcctcggcgccggtaagacgaccctgctcaaccgagtgcttcacaaccgcgacggccgccggatcgccgtcgtcgtcaacgacatgagcgaggtgaacatcgacagcgccgagatcgagcgcgaggtgacgttgagccggtcccaggagaagatcgtcgagatgtccaacgggtgcatctgttgcaccctgcgagaagacctgctcgtggagatcaccgaactcgcagcgaaggggtccttcgactacctcctcatcgagtcgtccggcatctccgaaccactgccggtggccgagacgttcacgttcgtcgacaccgacggcaacgcattgtcggatgtggcacggctggacaccatggtcaccgttgtggacggctacagcttcctccgcgatttccgatctggcggcgacatcgtggccgaggcgccggaggatcagcgcgacttgtcggatctgctcgtcgatcaggtcgagttcgccgacgtcatcctggtcagcaaggccgacttgatcaacgcagccgagctggccgagctgacggcggtgctgcgttcgctcaacgccacggcccggatcgagccgatgatcgacggcgacgtccccctcgacaccatcatggacaccgggaggttctccctggtgaaggccgcacaggcgcccggctggctgcaggagttgcggggcacccatatacccgagacgttggagtacggagtcagctcggcggtgtatcgggagcgcgcgccgttccacccggtcaggttccacgacttcctcaccgcggagtggacgaacggtgtgctgctgcgcgccaagggctatttctggaatgcggctcggatgactgagatcggcagtgtctcgcaggccgggcatctcatccgccacggctacatcgggcgatggtggcagttcctgccgccgagctactggcccgacgacgaggagcggcgcaccgcgattctcgagaagtgggaagatcccgtcggtgactgccgccaggagatcgtgttcatcggacagggaatcgactgggacgtcctgttcgccgctctagacgcctgtctgctgacccaggaggagatcgaactcggcccggacgcgtgggaacagtggccggacccactcggcgacggcgacgccgccggtgtcacatcgaagatcgcacaggcataa

SEQ ID NO: 143 - protein, 405 aa, Gordona hydrophobica
MTDNRLPVTVLSGFLGAGKTTLLNRVLHNRDGRRIAVVVNDMSEVNIDSAEIEREVTLSRSQEKIVEMSNGCICCTLREDLLVEITELAAKGSFDYLLIESSGISEPLPVAETFTFVDTDGNALSDVARLDTMVTVVDGYSFLRDFRSGGDIVAEAPEDQRDLSDLLVDQVEFADVILVSKADLINAAELAELTAVLRSLNATARIEPMIDGDVPLDTIMDTGRFSLVKAAQAPGWLQELRGTHIPETLEYGVSSAVYRERAPFHPVRFHDFLTAEWTNGVLLRAKGYFWNAARMTEIGSVSQAGHLIRHGYIGRWWQFLPPSYWPDDEERRTAILEKWEDPVGDCRQEIVFIGQGIDWDVLFAALDACLLTQEEIELGPDAWEQWPDPLGDGDAAGVTSKIAQA

SEQ ID NO: 144 - protein, 146 aa, Pseudomonas marginalis
MNTSMRHDYKAVGLPLDDEGPVFDKPWQAQAFSLLVHLHQAGVFPWKDWVQVFSEEIKAAPAQPGEGVNDAYYRQWITAMERMVTTLGLTGMEDIVQRGEEWRQAYLNTPHGQPVVLLNASCPPAHGHTGEHLPHREPVAISRASN

SEQ ID NO: 145 - protein, 399 aa, Rhodococcus erythropolis
MVDTRLPVTVLSGFLGAGKTTLLNEILRNREGRRVAVIVNDMSEINIDSAEVEREISLSRSEEKLVEMTNGCICCTLREDLLSEISALAADGRFDYLLIESSGISEPLPVAETFTFIDTDGHALADVARLDTMVTVVDGHSFLRDYTAGGRVEADAPEDERDIADLLVDQIEFADVILVSKADLVSHQHLVELTAVLRSLNASAAIVPMTLGRIPLDTILDTGLFSLEKAAQAPGWLQELQGEHIPETEEYGIGSVVYRERAPFHPQRLHDFLSSEWTNGKLLRAKGYYWNAGRFTEIGSISQAGHLIRHGYVGRWWKFLPRDEWPADDYRRDGILDKWEEPVGDCRQELVFIGQAIDPSRLHRELDACLLTTAEIELGPDVWTTWSDPLGVGYTDQTV